SEQUENCE LISTING (744 sequences)

SEQ ID NO: 1 - DNA, 23346 bp, Homo sapiens
  source 1..23346: mol_type = genomic DNA
accctccacggagcacgctgggcagagggcgcggcaccagacactgaagaaatgtccaccgccttctcgccagcaggagcccaggtcttcccttcttagagtgtccgccccccaccgcggaagggtgatctgagggccttgtgagaaatgatggggatgggacccacagcggtgagactggccccaccccaggcgtctgagtttctttaatgacacctcggaaacatcactcatataccacacaattcacccacttacactgtacagttcagtgattttattttattattatttctttgagacggagtctggctctgtcacccagactggagtgcagtggtgcgatctcggctcactgcaaactctgcctccctggttcaagcgattctcctgcctcagcctcctgagtagctgggattacaggcacctgccaccacacccagctaatttttgtatttttagtagagacagagttttaccatgttggccaggctggtctcaaactcctgacctcgtgatctacctgcttctgcctcccaaagtgctgggattacaggcatgagccactgcacccagccagttcagtgattttagtacctcgtagagttgtgcaaccaccacgactgtctagttccagaagattccatcaccgcaagaagaagccccatcagctgtcactcatcccctctgccagcccccggcacccacacatccccttcctgcctctgtggatgggcctgttgtggacatttcatataaaggggatcacacactgtgtgtccttctgtgtctggcgtctttcactgagcatgacatcctcaaggtgcatccgcgctgtggctggggcagagccttgctccttttcacgcctgtaatcccagctactcaggaggctgaggcaggagaatctatacaacccaggaggcggaggttgcaatgagctgagatcatgccactgccctccagcctgggcgacagagcgagactctgtctcaaacaaaaacaacaacaacaaacaccgatcagggctgggctggcgcacgctttgtattgagtggtgtcttcctctttgtagcctctgtggttggcccaaaggtaagtccccaccctgatccctagaatgtgacatttgccttatttggaaaaagagtctcaatatattgacaccatgaagatctgaacgatatggagaagattgtcctggggcatcctaaacgcggtcacaatacggagacaggaggagatttcacacaggcagacgaggaggctgcagtgcgaccacagagaccgagacgggagtgatgtggccgcaggccaagcacacctggagccttcaaaagctgggagaggcaggaaggagccccagggcctctggagggagcgaggcctccacacaccttgacttcagccccgcagtcctgattttggacttttcatttccagaaatgtaagaaaatgaacgttgctttaaggcaccgagtttgtggtaatttgtcacaggggctccagggacctgggccatccccttccatctagcgattctcgactctgtggggtttgcttggttgggttttgttttgttttgtttttagacagagtctcgctctgtcgcccaggccggagggcagtggtgtgatctcggctccctgcaacctccgcctcccgggttcaagtgattcttctgcctcagcctcctgaatagctgggattacaggcacccaccatgcccggctaatttttgttatttttattggagatgagggtttcaccatgttagccaggctggtctccagctcccggcctcaggggatccgcccgcctcggcctcccgtagtgctgggatgacaggcgggagccaccacgcccggccagatatatggtttcccacttttgatggtgatattaagtctcttcgtaaaacttaagcaagaaaaataattaatgttgtatttattaatttatttttgagacggagtctcgctctgtcgttcaggctggagtgcagcggcgagatctcagctcactgcaacctcagtctcttggattcaagcatttctcttacctcagcctcccaagtagctgggattacaggcaccacccactaccacgtccggctaatttttgaatttttagtggagacagggtttcaccacgttggccaggccagtctcgaactcctgacctcaggggatctgcctgcctcagcctcccaaagtgctggaattacaggcatgagccaccatgaccagccaatattgcatttacttaatacaggagtggtggagacagatgtagttaaaataaatggtggggctggggggtggtggcggctacagagttgcgaggctggaatttgggcaatgagctgtttcagaaactgctgtcttattccaaaacccagattatgttcttttttttttgagacagggtctcgctctgtcgcccaggctggagtgcagtggctcaatcacagctcactgcagcctctgcctcctggctcaagcaattctacatcttcccacctcagcctcccaagtagatggggccacaggcaggcaccatcacagccaacaaaatttttttgtattatttgtagtgatggggtttggctacattgcccaaggctggagtgcagtggtgcgatcttggctcactgtaacctccacctctcaggttcaagtgattttcctcccttggcctcctgagtagctgggattacaggcacaggccaccatgcccggctaattttcgtatttttagtagagacggggtttcaccatgttggccaggctggtctcaaactcctgacctcacgtgatccgcccgcctcggcctcccaaagtgctgggatctcatcaaataaaagaaaagactaacgcataaggaatggtgaaatagttcagaagtcggtttccacccaagtttctggctctgccagcctgcaggggtgcagggggtgtctgtaggtcttggcacaccaggcctggctgattcagggccaggcaggtgaccccagtagtgggtgaggtctgaggtcacagcccgctggggcaccctgaggtggactgaggcaggtgagggagggtgctgccgtctgtcgaaaacattcccagccggtggcatcagtgcgctcacgcaggcgtcggcagcagcgtctggaatgtgctgtaaggaggctgccaggcccttccagagtgaggtcgagtcctcgttctgggctgatggggaaactgaagtgccagggtttggggatggaggatagcttgagctgaggcccccgagcccccgtgtggtcaggccccctaactgagccccagctacatctttgctccctctccccagccccactctgctcaaattgctcctctgtccacgcttgttcctgcctcagggcctttgcacttgttttgttttgagacagggtcctgttctgtcaacccaggctggagtacagtggcgccatctcagctcactgcagtctaaacctcctgggctcaagcagtcctcctgcctcagcctcccgagtatctgggaccacaggtgtgcaccaccatgcctggctaatttctttttttcctttggtggagactgggtctcagtatctcggttgcccaaggcttgtctggaactcctgggctcaagcgatcctcccgccgcagcctcccaaagtgctgggattacaggcctgaggcactgaactgacctccctttgcacttttttttttttgagacaggttctcgctctgtcacccaggctggagtgcagtggcgagatcttggctcactgcaacctccgcctcccgggttcaagcgattctcctgcctcagcctcccgagtagctgggattacaggtgcccgccaccacccccggataatttttgtatttttactagagacggggttttgccatgttgccaggcatgcttgcgtatttgccaagctggtctcgaactctgaccttaggtgatccgcccgcctcagcctcccaaagtgctgggatgacaggcctgagccaccaaaccagctcccctttgcaactgggatattagcctggaacacagttacccccagatattcctattctcacccttcccaagcctactgccccacagcaggcgctcccgcctctggcctgctgccccggctgaacgacacaggtgggcacaccttccaggttccattctaaagctcccgccctgctcctcctaggcaggatcacgcggagaccagggcaggctctaggctccgaggtttatttcattcccttgacctcaccccacttagcaggtgaatccaagcctgccgctccattcggctcctgcccctgagggcttggggattggagggtaaactgaagctggaggctgggagtctcgcggactggggctttctctccttgcagcctccctgaggtcgcacgtgctgtgtcttccagcaacttggtggccctctctgagcctcagcttcctccatgatagacggcatcctaatgactcttacccaaaagttgtcgttggagcaatgaaggttgcggacaggaatctctaaattgcagatgctgtcctcgttattagatgagtatgccttgaaaaaaaaaaaaaaaaagccgggcacggtggcggcgtctgcaatcccagcgctttgggaagccgaggggcgaggatctctttaccccagcagttcgaagctgcactgagctatgatcacgccactgcactccagcctgggccacaacgcgtccaaaacaaaaccaaaaaagaaagaaaaacagaaaaaaagtccggatttgaggtcctcgggaaaacagcctgagagtgcctcccagttgccacggcaacagctctgtcccgccccctccctttaaaaacagcaggaggaaccaatcagagcgtccaccggcgcatgccttcgcgcctccgctgcatcctgggagtctacttccggctggggtttcccttcgcagcctccgtcgaccatgaaaccacaactcccggcaggcggcgcgggcgcatgcgcaggcgcgggccggtggggtggcctggagcggcgtgcgtaatgtcggcactttgctggggccgcggagcggcggggctcaaacgagccctacggccttgcggccgcccgggactccccggcaaagaaggtaacacaaagggagacggccaggcagcccgggggccacggcgtgggctcctccccgcgcggccggggcctttggcgctttgacctttgcctctttgcacctgggcgtgcgcagcccaccgacccggccccgctttccactcgcccttgcccgcttttcgactgaggctcctccgtgcagtctgacctttgactcgtggtttcttatgcagcctcctgaccccaggtgtggtgtttagggctcttaacctttgacctgctgcacctcgagtgcgaggaaccctctctcggcggttccgcgcccgtgttcgccgcggcggggcccttccaccttcgctgtcttagtttctttccaacttaagcccgtgctccgagagtttccgctgcgtttagggctcctttgcccttcgacccttcggcttctgcctttatggaggcccccgcccacacgcccggccccgctcagccgctctcgtctttcccgcagggaccgccggtggcgtctgcggccccaggaggagctcgtccgccagcccccaggagcaagaccaagaccgcaggaaggactggggccacgtggagctgctggagggtgagcgcgacccccacggcggcccgggagaggagagtccgctccctgctgccgggagaaaaggcaaaggctcagattctgagacaggctgagggcggtctccacctctcgggtcccaggacacgcccagacggcgttgctgaaacatctcttgcaccgctcatgggccctgtccttgctgagcaggctccttcctggagctctcgtaattccccctccccagaaccccaccaggtgctcccactttccagggacataggtgaggcccaagattatgtctggagagaccccccccccggccccgccgccaaggccacatgtgcagagccaggatgtgaaccctgaactgactcagctgtcctggcttggctctggggggtaggcgcctaatggagactggatgactgcgaaacttgggagggacagcccaggctccagcggtggcaggccctcgtcctggcctggctggtggtgtgggagggaggtgggagcttccctgtgcttgcttcccgccggtgcttctgtcccacatcctgtctgcgtccattaggcagccgggaagggagaggcgggagaacctttataaagggaaaaaacctggccaggtgcggtggctcacgcctgtaatcccagcacttttgggtggccgaggtgggcggatcaagaggtcgggggttcgaggcctggtgcggtggctcacgcctgtactcccagcacttttgggtggccaaggtgggcggatcaagaggtcaagggtccgaggcctggtgcggtggctcacgcctgtaatcccagcactttgggaggccgaggcgggcggatcaagaggtcatgagttcaagaccagcctggccaacatggtgaaacactgtctctactaaaaatacaaaaattagctgggcatggtggtgaacgcctgcagtgccagctactcaggaggctgaggcaggaaaatcgcttgaacccggggagactgagacaggagaatcgcttgaacccggaaggtggaggttgcggtgagccgagatcatgccattgcactctagcctgggcaacgagggaaattccatctcaaaaacaaaacaaaacaaaacaaagctctcaactgttaatcctaaaagtatctcctgagttttggagcactctgaaggcggacatcacgcttgtctttttatttcagcaattttttttttcttgagacggattctcaatctgttgctaggctggagtgcaatggtgcgatcttggctcactgcagcctccgcctcccgagttcaagcaattctcctgcctcaacctcccgagtagctggaactacaggcgcgcaccaccacgcccggttaatttttcgtatttttagtagggacggggtttcaccgtgttagccaggatggtcttgatctcctgacctcgtgatccgcctgcctcggcctcctaaagtgctgggattacaggcgtgagccaccacatctggacacctgggtccccccccccctttttttttttgagacagggtcctgctctgtctctcaggctgcagcacggtggcctgatcacggctcacagcagccaggaactcctaggcttaggtgattctttcacctcagccaccggagtagctgggaccacaggtgcgcggccctgtacccggcttatttcaacagtcttttttttttagatggattattgttctgttgcccaggctggagttcaatggcgctatcttgagtcactgcaacctctgcctcccaggctcaagcaattctcctgcctcagcctcccaagtagctggtactatagacacatgccaccacgcccagctaattttttggatttctaatagagacggggtttcaccatgttggccaggctggtctcgaactcctgaccttaggtgatcttcctgcctcggcctcccaaagtgctgggatgaccgacgtgagccgccatgcccggccagcaatagatttccaacaaggcttctttaaagagatacagaacacttaggcattgattggttggcaaaaatgcaacctgaggcggcacgagcaggagccgcgttttccctgcgagctagtggctcagcgtccaccggtgtagacactgggagcctaggagtgtgtgtgtggcagctccaccagctgggggggtcttgctgaggcggggtgcgaacttgcaggccatggttgagccagggagtgggcggttcagggtcctgggagtctgtgcagaggccctgagctagacagtgagaggggcaggggaggcgggggccacctggatagattctggaggcaagcccagaggaggtactgagaggtggacggagggagttgtcacagaccccgggttctgggcctgtgcacctggagggtctgagcggccagtgaccggggagacgtggtgctgcaggtttcggtggcagcttgttagggctgctgagttctgtgaggctcgggtggggaggtgcggaggtggtggggtgcgtgtgtctggaaagcagcgggcagaccttacaacgttgttggcaagtatgaggaattcacgccttgggtcccagcgagctcagccacctgcacctcctggcctggctcagggtccagagagacgggtgctcgaatggggagggggtccctctcatgtcctcgccttctgcccctcacagtgctccaggcgcgggtgcggcagctgcaggctgagagcgtgtcggaggtggtggtgaacagggtggatgtggcgcggctcccagaatgtggcagtggagatggtagcctccagccacccaggaaggtccagatgggggccaaggatgccaccccggtgccctgtggccgctgggcaaagatactggagaaggataagcggacccagcagatgcgtatgcagcggttgaaggcgaagctgcagatgccattccagagcggggagttcaaggcgctgaccaggcgcctgcaggtggagccccggctcctgagcaagcagatggccgggtgcctggaggactgcacgcgccaggcccccgagagcccctgggaggagcagctggcccggctgctgcaggaggcccctgggaagctgagcctcgatgtggagcaggccccgtcggggcagcactcgcaggcccagctctcaggtcagcagcagaggctcctggccttcttcaagtgctgcctgctcactgaccagctgcccctcgcccaccacctgctggtcgtccaccacggccagcggcagaagcggaagctgctcacgctggacatgtacaacgccgtgatgcttggctgggcgcggcaggtgagtgcagccgggagccgggccacctgccctggtcgttggagacaggaacttgccctcatttagactggacttttctctcttagcccccaggttcaagtcctttgttttttattttaataattcttttaggcttttcaaatacaaatcacgtgtgactctcaaattttgaaaatgaccaaaaaagtagggagaaattctgctaatgccttgagcatttccttccgttctttctcggggctcgtgaatctgtctgggcaggggtgcttttggcctcgtcggggggcagcctgcagagccctcgtcaggatgaggctttggtgttggggttgactttgctcctttttctggtttcctgaggtggaaggtcgggctgctgtgctgagatccttctgtgtgaaatgtgggtgttcactggcgccgcattccctctgagcacagctttagatgtatcccacccatcttcgttgtggttcatctggagtattttccaatttcccttgccgttttctttttgtttgtttatttatttgttttctgggacatactctccctgttactaggttggagtgcggtggtgcgatctcggctcatggccacctccacctcccaggttcaagtgattatcctgcctcagcctcctaagtagctggagttacaggtgcccgccaccacccccgattaatttttgtattttgagtagagacgggggtttcaccgtgttggccaagctggtctcaaactcctgacctcgtgatctgcccccctgagcctcccgaggagctgagattacaggcgtgagccaccgtatccggccaaagtttctcctcggaactcttggttgttttggagggtgttgtttacacaccctttcaaagcagcaaacgttcccagctttctccccccccatgatccttcagccctaatttgagaaacaacagtaaaagcttctcttaatatttgggtaaacatgggatactattatattaaaatacagatatcgcaatattaagaccaattcgtggtagagaacttgatgtgcttctttattaacgcatcaaacagcgaaattcaggatcaggtggggccagtcttgcggtgaagtccgcagtgctcttaccgcgaggtggaggcggcgtgctgcctggttcctccttgctgcagggggcgccagactccgggaggactgaggatgagggtcgtgtattttcccctccaaggtcacaagccccctggattccctgggcagagagcccacactgtaacagggcgcttttgggatcggcccctacgaaagtgtcccaaacctggcggcttgcacagcagaaacgcactcttagcacgagcccagaagtctggaatcaagatgtctgcagacccgaagtctctccagaggctctagcaggggcccctcctgcctctcccagcgttccggggtggccagcagtcctggatgtcgctggactgcgagctcagcctcccgagcagctgggattacaggcgtgtgccaccacccccggataattgttttaatttttatttttagtagaggtggggtttcagcatgttagccaggctaaggcattgcttctatcttttttttttttttttttttgagatagactctctgtctgttgcccaggctagggtgcagtgccatgatctcggctcactacagcctccgcctcctgggttcaataattctccctgccgcagcctcctgagtggctgggattacagggactcaccaccacgcccggccaatttttgtatttttagtagagacgggcttttgccatgttggccgggctggtctcaaactcctgacctcaggtaatccgcccgcctcagcctccctaagtgctgggattacaggcgtgagcgaccgcgcccggccacgtattctaaccagcaccatccagtagaactttctatgatgttgggaattttccgcatctgccaacagctgtgtgtgctactgaacacttgaagctggtgaaagggaagaactgaatatttcaatttaaggactatgtgtggccagtgaggtctaccatacagaggttgtttagaaactgccccaaaaccggccgggcacagtggctcacgcctgtaatcccagcactttgggaggctgaggcgggcggatcacgaggtcaggagatgagaccatcctggctaacacagtgaaaccccgtctctactaaaaatatacaaaaaattagccgggtgtggtggcgggcgcctgcagtcccagctacttgggaggctgaggcaggaggatggagtgaacctgggaggcagagcttgcagtgagccgagatcgagccactgcactccagcctgggcgacagagcgagactccgtctcaaaaaaaaaaaaaaaaaaaaatgccccaaaactcatgtctctggtctctggtgtgtggccacctcatgtcacagcatgtcttcagctgcaggactatggggtgggaggatgtaagggaatttttttccctttggaatagtctgttacagttacaggccactcttaagcagccactgggtcaaaggagaaattgctcagaacagggatgtcaggaaaaatgaatacaagaaaaaaattatatatatatgttattttatatatatatatatatgtgtgtgtgtgtatattttaagacagagtctccaagtctgtcgacagcaaagtttctgtctcccaggctggagtgcagtggtgagatcccggcttaccgcaatttccgctttctgcgttcaagtgattctcctgccccaggctcccagtagctgagattacaggcatgtgccaccacgcccagctaatttttttttttttttgtatttttagtggagggagcacaggcatgagccaccacgcccagctaattttttttttttttttttgtatttttagtggagatggggtttcactatgttggccaggctggtcttaaactcctgaccttgtgatcctcctgtcttggcctcccaaagtgctggggttacaggcatgagccactgtgcctggccaaaaattaaatgaaaaaaaaaaaaattgcaggggatatttaagctatacatggctgggcgtggtggctcacacctgtaatcccagcactttgggaggccgaggcgggtggatcccctgaggtcaggagttggagaccagcctggccaacagggcgaaaccccatctctactaaaaatacaaaataattagctgggtgtggtggcatgtgcctgtagtcccagctactcaggaggctgaggcaggagaatcggttgaacccgggaggcggaggttgacagtgagctgagattgcgccactgcactccagcctgggcgacagagcgagactctatctaaaaaaataattaaataaaatttccaaagaagaagtaaactatgaagttcgaaaacacttggagataatgaaaatgaaaacaccacctgtcagaagtcctgggatgcagctgacacagtgctcgcagggtaatttatagtcacaaaatgcctatattcggaaaggtgatggccgggcgcggtggtgcacacctgtcatcccagcactttgggaggctgaggctggcggatcacgatgtcaggagttcgagaccagcctggccagcatggtgaaaccccgtatctgctaagaatacaaaaaattagctggtgtggtggcgtgtgcctgtaatcccagctactcaggaggctgaggcaggagaagtgctggaacctgggaggcagagaacccggcccagctgagattgtgccactgcactccagcctgggcaacagagcaagactccatcttggggggaaaaaaagtgaaacatctcagaccaaatacgtcacctttcaccttaggaaactagaagaagaagctgtcactaaacctaaagcaagtagatgagaggaagttacaaagattaaaacaaatcgatgaaatggaaggcagaaacagcagagaacagtcaagccaagtcagtcctttgaaaagatccacacaattgacaaatctggaggtttaacaagctgcctcccagaggaacgtgaacctcccacagtcccacctggcgggccatgcggctgcctctgcaggtggagcttgtgtgtcagaacctcagggggatgcatcggtctgcccagctggccactcctgcctggtgaccctgtcccctgctgagctggtggctacagagaaggggtctccagggagcagggaggatgtggaggttgggccatccccccagaccctcaccctggcttggtctcccagggtgccttcaaggagctggtatatgtgttattcatggtgaaggatgccggcttgactccggacctgctgtcctatgcggctgccctccagtgcatggggaggcaggaccaggacgccgggaccatcgaaaggtaggtggtgtcgggagggcccccaccccccaccatgtccctcccagggttttaagatctgggacggtgcctgggctgggggctggggacccccagagggcctgacagccacaccgagacctgcaggctagcaggaggtggtcacgaggttcccctggggtctgtggaagcccagcgtggggctggcccgtccctcgagcaggccgcctgcaccacaggtgtctggaacagatgagccaggaggggctgaagctgcaggcactcttcaccgccgttctgctgtctgaggaggatcgggccactgttctgaaggccgtgcacaaggtgaagcccaccttcagcctcccgccgcagctgccgcccccggtcaacacctccaagctgctcagggacgtgtatgccaaggtgagcccacgtgggccccggcagacttcagtccttatagctgccctttgggggttcctggagcctcagctcagaccaatgggcctccccggtgcccagggctcacctgggctgggccctgaattcctcccttcctcctctgagaagatgggaggacccagagctgggagggtcttggccctgagggtgtgggctcagggggacgaggtggtcgggggccgcagagagagggtggggtgacaggagagcagtgcgtttgagtggggctgccaggtgcacgcagtgggctcgtctgttactgaggtgtaattcacctgccataaaaccctcttaaaggtgtaattcagcgattttagtgtagtcacggttgtctaattctagaatgttccatcaccctaaaaggaagcctcgtccccatccccgtcactccccatcccctccccatctgtggatgagtctgctctggacattccatagaaatgggaccacacactgcgagaccttttgtgtttggcatctcccactgagcgtgacgtcctcatggtgcctccgggaggcgacctgggtcagggctgagtcatgctccggtgcgtggatggccgcgccgtgcccgtctgttcgtccgtttatgggacgttgggctgtttctacttatcgactgcgagtcatgcagctgtgaacgtttgtgtacaagcttttatgtggacgtgcgtctttgattctcaaagcgctaggctttagttgttatttttaattcatctatttctggatgggtcacattcatacagttctgagtttgaggagaaaacagccaacatctgggggtgcagtgaacggtctccccaggctctgccagctcccagcatcaccgcccctgccaccccctgcacggtgcccagtgtcccttctagaggggtcgaggccaggagggtcaaggcctttggtttgcactgctggcactgactttacccgcagcccgcctttccccgcgtcagcgatagccaccagcaacttgcaggagacgcagaaacgcgtgcacccatgctgtctcggggtcctgggagggcaggtcgccctctggcaagcccccaacctcactacccccatccctgcaggatgggcgtgtgtcctacccgaagctgcacctgcccttgaagaccctgcagtgcctctttgagaagcagctccacatggagctggccagcagggtgtgcgtggtgtccgtggagaagcccacgttgccaagcaaggaggtcaagcacgcggtaggggctgagccggggtcccgtgtccgccgcaggggctgagccggggtcgcgtgtccaccgcacgggctgagccggggtcccgtgtccgccgcaggggctgagccggggtcgcgtgtccgccgcaggggctgagctccgtgtatgcggttgaattgctcatgccctggtctggtcatgcagggcctaggaactcagcctggctgccgctcagggaaaacttgtggggtcccgaggttgggtcttggcctgtgcttctgcgcacactgcagctgggcttttgctcttggctgcgtcaggcacgctgaagccggcagagggcaggccggggtgaggagctacccggaacacacagcagggaccttgagggccatgaggtctctgcagggagctgcggtgacccccgtcctgtgagcctgggtcccaccagctgccacggggctcaccgggcccgttcatcccccgcagcggaagaccctgaagaccctgcgggaccaatgggagaaagcactgtgccgggcgctgcgggagaccaagaaccgcctagagcgcgaggtgtacgagggccggttctcactttaccccttcctgtgcctgctggacgagcgcgaggtggtgcggatgctcctgcaggtgcgtcttcctccgcgcggccgggtccccgggcggggcgggcaggcactcacggctgccttccgcaggtcctgcaggcgctgcccgcccaaggtgagtccttcaccaccctggcccgggagctgagtgcgcgcactttcagccggcacgtggtgcagaggcagcgggtcagtggccaggtgcaggcgctgcagaaccactacaggaagtacctctgcttgctggcctccgacgccgaggtgaggctcgcaccccctctcctcctggctggggccagtggtgggtgggcgctctccctccctggagcagccgggtttgcacccaagcttcagtctgttcatctgtatgcttgggctgacagatgccccaccgggcgtcaggcacgggcggtggggcatctagctcaggggccccggtgcccagcgccctgaccgccctgtcctacaggtgcccgagccctgcctgccgcggcagtactgggaggagctgggggcgcccgaggccctgcgggagcagccctggcccctgccagtgcagatggagctgggcaagctgctggcggagatgctggtgcaggctacgcagatgccatgcagcctggacaagccgcatcgttcctctcggcttgtccccgtgctctaccacgtgtattccttccgcaacgtccagcaggtgccaggcagtgccctcctgagctggggggcatcccgctgggaggaccagggaccccatggggttggcgcctttgggcggagccttgatctcagcgcagccgtcagtacctcccaggactcgggacacaccatgggtgtccatgcaggtgtccggccgtttctggaggtggcttctgttcgtgggaggccacactggtttcttgttggccggggagccggcccctctcacccaagtttaaggggtgggaacaggaaagccccagcaccggggccctgacccgtctgcctgtcgcaccccagatcggcatcctgaagccgcacccggcctacgtgcagctgctggagaaggccgcggagcccacgctgaccttcgaggcggtggatgtacccatgctttgccccccgctgccctggacatcgccgcactctggtgctttcctgctcagccccaccaagctgatgcgcacggtggaaggcgccacgcagcaccaggagctgctggaaacctgcccgcccaccgcgctgcatggcgcactggacgccctcacccaactgggcaactgcgcctggcgcgtcaacgggcgcgtgctggacctggtgctgcagctcttccaggccaagggctgcccccagctaggcgtgccggccccgccctccgaggcgccccagccgcccgaggcccacctgccgcacagcgccgcgcccgcccgcaaggccgagctgcgccgtgagctggcgcactgccagaaggtggcccgggagatgcacagcctgcgggcggaggcgctgtaccgcctctcgctggcgcagcacctgcgggaccgcgtcttctggctgccgcacaacatggacttccgcggccgcacctacccctgcccgccgcacttcaaccacctgggcagcgacgtggcgcgggccctgctggagttcgcccagggccgcccgctcggcccgcacggcctggattggctcaagatccacctggtcaatctcacggggttgaagaagcgggagccgctgcggaagcgcctggcctttgcggaggaggtgatggatgacatcctggactccgcggaccaacccttgacggtaggggcggggcccccgcattccccgccctcccggcacccccgcgccctcccccggcgcccccgcgccctcccccggcgcccccgcgccccctgcccgtcttcctcctcccctccccctcccctggcgccccctgcccgtcttcctcctcccctccccctcccctggcgcccccacgtcccccgcccgctctcctcccccgcgccccctgcccgtcctgcttctcccttctcccctgccccagggcccccgcgttccccacgttcccccctcccctgcatccgccgagctgcttcctcccccggcgcccccaggtcctctgtccaccctcctcccccggcagctccggacacgcttgtttcagcccggtccacccgcagctcccatcctccatccgtggctatggagaactcatccagccaccgccccgtctctcgcccacatgcgtcccgtgttcactatcacgggtgtgcgtctcctccccatttcccacctccccgcaacacagcctcccagcgggctcagggatcgggcccgggcctcactgccccccatccgctgtccccgcagggccgaaagtggtggatgggcgcggaggaaccctggcagacgctggcctgctgtatggaggtggcgaacgctgtgcgcgcctccgaccctgccgcctatgtctcccacctccccgtccatcaggtgagccagctgggtctggcccccgaggccgtgcagtgtgcactgggcctggggctcgacttgagggtgtgagatttcaccttcgctcgtggtattctctggaggtggtcctagagtgtgcgggagccatggctcctggtctctcccgggagcgtgcgggggctccggtgcacttggctggggggtgcacggccctaggaacggctcgacctggggggtggtttggggtcccacacgtctctgcctctctagcttccatctcccgtttgccgccccttcctcaggacggctcttgcaacggcctgcagcattatgctgctctgggccgcgacagcgtgggcgccgcctccgtcaacctggagccctcggatgtgccgcaggacgtgtacagcggcgtggccgcgcaggtagggtgtgccctgctgcccgggggcatctcggcgtgggggcaatgtgagccctgagtctcggccccaggtgccctcaccttggcctgtcctgaggatgtggtggggctggggcgggggcttcatgggtgcttggtggcccctgggctgctagcggggctgacccgcgcccccggcccacccgccgtgcaggtggaggtgttccgtaggcaggacgcccagcggggcatgcgggtggcacaggtgctggaaggtttcatcacccgcaaggtggtgaagcagacggtgatgacggtggtgtacggggtcacgcgctatggcgggcgcctgcagattgagaagcgcctccgggagctgagcgactttccccaggtgcgccaggcatgtcgcgctgcgaacacgttggtttcactgccatttaaaactcagacgggtgtgcccccgacccagcctcccagcaggagccctggctgctcccccagaccactggctgcctgttagggcttgggcgtttcttgctgactgcgtgggcgccaggccccattcccctgcccccctgctaggccctaggccttgggcctcagtggctctgaaataggggggagtagggcgtgaacgggccagcccctgaggacctgcacctgctgcccgccctctgcaggagttcgtgtgggaggcctctcactatctcgtacgccaggtcttcaagagtctacaggagatgttctcggggacccgggccatccaggtacgtcctgtcctgtccccgctcggccaggactccctaagcaagtggacgggatccccggcctgagccccctgccctgagacggcctgtgtcccacagcactggctgaccgagagtgcccgcctcatctcccacatgggctctgtggtggagtgggtcacacccctgggcgtccccgtcatccagccctatcgcctggactccaaggtcaaggtcagtgtaccaccccatccctccccagtgtgccaccatcccagtgtaacagcgtcccagtgtaccagtgccccagcgtaccaccaccccagtgtaccaccgccccagtgtgccaccccatcccgcgccagtgtgccaccatcctagcgtgccactaccccagtgtaccaccacctcaatgtaccaccaccgtgctgggcctcgggtgggactcaccggccccttcccctttttagcaaataggaggtggaattcagagcatcacctacacccacaacggagacatcagccggtgagtggggggcccgggctggggcctggccgggggtctggagagcagccacggtggagacagcggccagttaggggtgtccctgggccgcccccaccggcacctgttccgtcctcagaaagcccaacacacgtaagcagaagaacggcttcccgcccaacttcatccactcgctggactcctcccacatgatgctcaccgccctgcactgctacaggtgggcgtctccggacggcgtgggtgccgctcgcctcccaggggcgtcttctggctagcagggcacagctggcaaggctgggtctagctgggggcagggactgggcagagtggatctgctgtgtctgtggggcctgtgtagaggccctgaggctgtgtgccccgaatgtaggtggtcctggcagctcccctcctgtggcccgacagggagagccgagcatgggcgctgccccttcctgctgggcatttcttggcaccgcgatgtgtggttagatctggagcacgtggaggtcgttccagcagaatctaccggccacgggagctgggaatgctcaggatgcggggtggtggccctggcaggaggggacctcgtagccgccctcggagctgaggctcccacagcagcgtggggggcggcaggggtggggcgaggaggcttctgggaacatttcctccccctgtactgatacctaacccaggatgtttggggaggggcggggcgaggggtggggcgaggcctgcaggagggtgggcttccctggacctcaagtggagggggctgagcgcagggccaggatgctggtcactgggtggccctgtgagctcccctcccttcaggagtcctccgctctgccacaggaagggcctgaccttcgtctctgtgcacgactgttactggactcacgcagctgatgtctccgtcatgaaccaggtgcccccgtagtctgagcctcagtccacccatgggtggggcccccacactggggcgttgggtaggggtggtgcccagagcctgcctgatcacccctggggatcctctgactcctgcccaggtgtgccgggagcagtttgtccgcttgcacagcgagcccatcctgcaggacctgtccagattcctggtcaagcggttctgctctgagtaaggcgctccctgaccccaactacctggattccccccccaccccaaaacctggcctcagcctcaccccaccctgcttccaaccccaggccccagaagatcttggaggccagccagctgaaggagacactgcaggcggtgcccaagccaggtaggcgggtgggcagcctcgcaagggtggctcgaactgcgggccagggcggggctttcccgccacccacgccgtccgctctgcttcctccgcaggggccttcgacctggagcaggtgaagcgttccacctacttcttcagctgacaccccgtgagccttgtcagtgtgtaaataaagctcttttgccacccccaggagccactgtcttcaggaagggtgcacgccctgcgggtctcgggcaatcacacgcggccaggcttggcgccaatgctgtcgtttattgcgcggaatgggggtgtgggggttaatggggcgtggggggccacggtgggggcactgctgcctcggctcgtcagtacattcatcacggcggcgggaccccagcctcccccccgcgccctgcgcagccaggcctgcctctcggtgccagtgctggagggaggcggggtgctgctccccgaggtcaccgggggacgcgcgcggacgggggccgggccggttattgcgtgagcgcgatgggggcagcgggaagccggcgggccaagtattgcacttagaaaacgatcctcctcggacgggggccacctagagggtggggggcgggcggggctccacagccggctcctctcagccactgggccgccccgtccctgtcttacagctgggggaactgaggcaccgaggtgaagggagccccctcgcacgcgaggccgccgccgggggcaggggcgatgggggtgggcgcggggcgatgaggggggacggccgggggcgcggagggggctgccccgccggccctgcccgtccgtccaactacggctacctacgtctcgtctatggcttctgggcggactggcggccggggcagcgcaatggcatggctttggtctggatgacggccccgcccccggcccgcctgggcccgcggggcggtcggcgagggtcacaagttggacgagaggcgcgagcgcgcggagtcctgggggtccaggccgccggcggcgccgggtgaggccgagtccctgcggtccgggctgggcgcggcggcccgggcagcgggcgtgggccccaagcgcggtgtggagctgctggccgggcgtgtggaggccgcggggccgggggcgccgtgaggcagcgagggctgcgaggcggacagtgggcgcgacgcgcggctcaggcggcgcgcgggcagggcgggcccagcaaggggggcggcgggcaggccgccgtagggcgaggtccgcggtgcccgggggctggcgggcgcgcccggggggctggcggggggcgggggcccgggtgaggcggcggcaggtgcgggccccgggggcgggcggcgcacgaggcgcggcgagccgagcgccagcggccccacgagcggccgcgccacctgcgggcagaagctcatggccgccgcctgctgcagcgtggcgatggccgaggtgacctgcggcggcggcggcggcggcgggaagaggcccacgcgctgacccagctcggcctgctgcaccatctcgcggtcgtacttgacgatctcctggatgatggcgttctcctggttgttgaatacgcccgagttgaggtcatgctgcaccttgtgcaggaggatggaattcttcttgcctgcgggagagggggcgttagcgtgtgcacagggagcgcctgctgcgtacagcgggcaccgagcacctactctgcaccgcgggtgcgcacacagtgcttgccgtgtagagcaagcatgcatttaccacctactgtatacaataagcacatatgtagcatctctggcctatagtatggatattgagtacctgccatatacaggctgagcacctgctatgcacagccagcatgcatgaagcacctgcatacagaaagcagggcttttgttttgctttgtttctgagacggagtcttgctctgtcgcccgggctggagtgcagtggcgcgatctcggcccactgcaaggtccacctcccgggttcacaccattctcctgcctcatcctcccgagtagctgggactacaggcgcccgccaccacacccggctaattttttgtattttttttttagtagagacggggtttcaccatgttagccaggatggtctcaatctcctgacctcgtgatccacccaccttggcctccgaaagtgctgggactacaggcgtgagccaccgcgcctggcagtaaa

SEQ ID NO: 2 - protein, 1230 aa, Homo sapiens
MSALCWGRGAAGLKRALRPCGRPGLPGKEGTAGGVCGPRRSSSASPQEQDQDRRKDWGHVELLEVLQARVRQLQAESVSEVVVNRVDVARLPECGSGDGSLQPPRKVQMGAKDATPVPCGRWAKILEKDKRTQQMRMQRLKAKLQMPFQSGEFKALTRRLQVEPRLLSKQMAGCLEDCTRQAPESPWEEQLARLLQEAPGKLSLDVEQAPSGQHSQAQLSGQQQRLLAFFKCCLLTDQLPLAHHLLVVHHGQRQKRKLLTLDMYNAVMLGWARQGAFKELVYVLFMVKDAGLTPDLLSYAAALQCMGRQDQDAGTIERCLEQMSQEGLKLQALFTAVLLSEEDRATVLKAVHKVKPTFSLPPQLPPPVNTSKLLRDVYAKDGRVSYPKLHLPLKTLQCLFEKQLHMELASRVCVVSVEKPTLPSKEVKHARKTLKTLRDQWEKALCRALRETKNRLEREVYEGRFSLYPFLCLLDEREVVRMLLQVLQALPAQGESFTTLARELSARTFSRHVVQRQRVSGQVQALQNHYRKYLCLLASDAEVPEPCLPRQYWEELGAPEALREQPWPLPVQMELGKLLAEMLVQATQMPCSLDKPHRSSRLVPVLYHVYSFRNVQQIGILKPHPAYVQLLEKAAEPTLTFEAVDVPMLCPPLPWTSPHSGAFLLSPTKLMRTVEGATQHQELLETCPPTALHGALDALTQLGNCAWRVNGRVLDLVLQLFQAKGCPQLGVPAPPSEAPQPPEAHLPHSAAPARKAELRRELAHCQKVAREMHSLRAEALYRLSLAQHLRDRVFWLPHNMDFRGRTYPCPPHFNHLGSDVARALLEFAQGRPLGPHGLDWLKIHLVNLTGLKKREPLRKRLAFAEEVMDDILDSADQPLTGRKWWMGAEEPWQTLACCMEVANAVRASDPAAYVSHLPVHQDGSCNGLQHYAALGRDSVGAASVNLEPSDVPQDVYSGVAAQVEVFRRQDAQRGMRVAQVLEGFITRKVVKQTVMTVVYGVTRYGGRLQIEKRLRELSDFPQEFVWEASHYLVRQVFKSLQEMFSGTRAIQHWLTESARLISHMGSVVEWVTPLGVPVIQPYRLDSKVKQIGGGIQSITYTHNGDISRKPNTRKQKNGFPPNFIHSLDSSHMMLTALHCYRKGLTFVSVHDCYWTHAADVSVMNQVCREQFVRLHSEPILQDLSRFLVKRFCSEPQKILEASQLKETLQAVPKPGAFDLEQVKRSTYFFS

SEQ ID NO: 3 - DNA, 20 bp, synthetic construct
  source 1..20: mol_type = other DNA
ccccagtccttcctgcggtc

SEQ ID NO: 4 - DNA, 20 bp, synthetic construct
  source 1..20: mol_type = other DNA
tccacatcgaggctcagctt

SEQ ID NO: 5 - DNA, 20 bp, synthetic construct
  source 1..20: mol_type = other DNA
ctccacatcgaggctcagct

SEQ ID NO: 6 - DNA, 20 bp, synthetic construct
  source 1..20: mol_type = other DNA
tgctccacatcgaggctcag

SEQ ID NO: 7 - DNA, 20 bp, synthetic construct
  source 1..20: mol_type = other DNA
ccccagctcctcccagtact

SEQ ID NO: 8 - DNA, 20 bp, synthetic construct
  source 1..20: mol_type = other DNA
ggccaccttctggcagtgcg

SEQ ID NO: 9 - DNA, 20 bp, synthetic construct
  source 1..20: mol_type = other DNA
gccacgtcgctgcccaggtg

SEQ ID NO: 10 - DNA, 20 bp, synthetic construct
  source 1..20: mol_type = other DNA
cgccacgtcgctgcccaggt

SEQ ID NO: 11 - DNA, 20 bp, synthetic construct
  source 1..20: mol_type = other DNA
cccatccaccactttcggcc

SEQ ID NO: 12 - DNA, 20 bp, synthetic construct
  source 1..20: mol_type = other DNA
gcccatccaccactttcggc

SEQ ID NO: 13 - DNA, 20 bp, synthetic construct
  source 1..20: mol_type = other DNA
cgcccatccaccactttcgg

SEQ ID NO: 14 - DNA, 20 bp, synthetic construct
  source 1..20: mol_type = other DNA
ttcgccacctccatacagca

SEQ ID NO: 15 - DNA, 20 bp, Homo sapiens
  source 1..20: mol_type = genomic DNA
gaccgcaggaaggactgggg

SEQ ID NO: 16 - DNA, 20 bp, Homo sapiens
  source 1..20: mol_type = genomic DNA
aagctgagcctcgatgtgga

SEQ ID NO: 17 - DNA, 20 bp, Homo sapiens
  source 1..20: mol_type = genomic DNA
agctgagcctcgatgtggag

SEQ ID NO: 18 - DNA, 20 bp, Homo sapiens
  source 1..20: mol_type = genomic DNA
ctgagcctcgatgtggagca

SEQ ID NO: 19 - DNA, 20 bp, Homo sapiens
  source 1..20: mol_type = genomic DNA
agtactgggaggagctgggg

SEQ ID NO: 20 - DNA, 20 bp, Homo sapiens
  source 1..20: mol_type = genomic DNA
cgcactgccagaaggtggcc

SEQ ID NO: 21 - DNA, 20 bp, Homo sapiens
  source 1..20: mol_type = genomic DNA
cacctgggcagcgacgtggc

SEQ ID NO: 22 - DNA, 20 bp, Homo sapiens
  source 1..20: mol_type = genomic DNA
acctgggcagcgacgtggcg

SEQ ID NO: 23 - DNA, 20 bp, Homo sapiens
  source 1..20: mol_type = genomic DNA
ggccgaaagtggtggatggg

SEQ ID NO: 24 - DNA, 20 bp, Homo sapiens
  source 1..20: mol_type = genomic DNA
gccgaaagtggtggatgggc

SEQ ID NO: 25 - DNA, 20 bp, Homo sapiens
  source 1..20: mol_type = genomic DNA
ccgaaagtggtggatgggcg

SEQ ID NO: 26 - DNA, 20 bp, Homo sapiens
  source 1..20: mol_type = genomic DNA
tgctgtatggaggtggcgaa

SEQ ID NO: 27 - DNA, 20 bp, synthetic construct
  source 1..20: mol_type = other DNA
ggtcttggtcttgctcctgg

SEQ ID NO: 28 - DNA, 20 bp, synthetic construct
  source 1..20: mol_type = other DNA
gaggctaccatctccactgc

SEQ ID NO: 29 - DNA, 20 bp, synthetic construct
  source 1..20: mol_type = other DNA
gcatccttggcccccatctg

SEQ ID NO: 30 - DNA, 20 bp, synthetic construct
  source 1..20: mol_type = other DNA
ctccagtatctttgcccagc

SEQ ID NO: 31 - DNA, 20 bp, synthetic construct
  source 1..20: mol_type = other DNA
tctccagtatctttgcccag

SEQ ID NO: 32 - DNA, 20 bp, synthetic construct
  source 1..20: mol_type = other DNA
ttctccagtatctttgccca

SEQ ID NO: 33 - DNA, 20 bp, synthetic construct
  source 1..20: mol_type = other DNA
cttctccagtatctttgccc

SEQ ID NO: 34 - DNA, 20 bp, synthetic construct
  source 1..20: mol_type = other DNA
ccttctccagtatctttgcc

SEQ ID NO: 35 - DNA, 20 bp, synthetic construct
  source 1..20: mol_type = other DNA
tccttctccagtatctttgc

SEQ ID NO: 36 - DNA, 20 bp, synthetic construct
  source 1..20: mol_type = other DNA
cacatataccagctccttga

SEQ ID NO: 37 - DNA, 20 bp, synthetic construct
  source 1..20: mol_type = other DNA
tcctcctcagacagcagaac

SEQ ID NO: 38 - DNA, 20 bp, synthetic construct
  source 1..20: mol_type = other DNA
cactgcagggtcttcaaggg

SEQ ID NO: 39 - DNA, 20 bp, synthetic construct
  source 1..20: mol_type = other DNA
tgcttgacctccttgcttgg

SEQ ID NO: 40 - DNA, 20 bp, synthetic construct
  source 1..20: mol_type = other DNA
gtgcttgacctccttgcttg

SEQ ID NO: 41 - DNA, 20 bp, synthetic construct
  source 1..20: mol_type = other DNA
tgcccaggtggttgaagtgc

SEQ ID NO: 42 - DNA, 20 bp, synthetic construct
  source 1..20: mol_type = other DNA
tgtagactcttgaagacctg

SEQ ID NO: 43 - DNA, 20 bp, synthetic construct
  source 1..20: mol_type = other DNA
agcatcatgtgggaggagtc

SEQ ID NO: 44 - DNA, 20 bp, synthetic construct
  source 1..20: mol_type = other DNA
gagcatcatgtgggaggagt

SEQ ID NO: 45 - DNA, 20 bp, synthetic construct
  source 1..20: mol_type = other DNA
ttcagctggctggcctccaa

SEQ ID NO: 46 - DNA, 20 bp, Homo sapiens
  source 1..20: mol_type = genomic DNA
ccaggagcaagaccaagacc

SEQ ID NO: 47 - DNA, 20 bp, Homo sapiens
  source 1..20: mol_type = genomic DNA
gcagtggagatggtagcctc

SEQ ID NO: 48 - DNA, 20 bp, Homo sapiens
  source 1..20: mol_type = genomic DNA
cagatgggggccaaggatgc

SEQ ID NO: 49 - DNA, 20 bp, Homo sapiens
  source 1..20: mol_type = genomic DNA
gctgggcaaagatactggag

SEQ ID NO: 50 - DNA, 20 bp, Homo sapiens
  source 1..20: mol_type = genomic DNA
ctgggcaaagatactggaga

SEQ ID NO: 51 - DNA, 20 bp, Homo sapiens
  source 1..20: mol_type = genomic DNA
tgggcaaagatactggagaa

SEQ ID NO: 52 - DNA, 20 bp, Homo sapiens
  source 1..20: mol_type = genomic DNA
gggcaaagatactggagaag

SEQ ID NO: 53 - DNA, 20 bp, Homo sapiens
  source 1..20: mol_type = genomic DNA
ggcaaagatactggagaagg

SEQ ID NO: 54 - DNA, 20 bp, Homo sapiens
  source 1..20: mol_type = genomic DNA
gcaaagatactggagaagga

SEQ ID NO: 55 - DNA, 20 bp, Homo sapiens
  source 1..20: mol_type = genomic DNA
tcaaggagctggtatatgtg

SEQ ID NO: 56 - DNA, 20 bp, Homo sapiens
  source 1..20: mol_type = genomic DNA
gttctgctgtctgaggagga

SEQ ID NO: 57 - DNA, 20 bp, Homo sapiens
  source 1..20: mol_type = genomic DNA
cccttgaagaccctgcagtg

SEQ ID NO: 58 - DNA, 20 bp, Homo sapiens
  source 1..20: mol_type = genomic DNA
ccaagcaaggaggtcaagca

SEQ ID NO: 59 - DNA, 20 bp, Homo sapiens
  source 1..20: mol_type = genomic DNA
caagcaaggaggtcaagcac

SEQ ID NO: 60 - DNA, 20 bp, Homo sapiens
  source 1..20: mol_type = genomic DNA
gcacttcaaccacctgggca

SEQ ID NO: 61 - DNA, 20 bp, Homo sapiens
  source 1..20: mol_type = genomic DNA
caggtcttcaagagtctaca

SEQ ID NO: 62 - DNA, 20 bp, Homo sapiens
  source 1..20: mol_type = genomic DNA
gactcctcccacatgatgct

SEQ ID NO: 63 - DNA, 20 bp, Homo sapiens
  source 1..20: mol_type = genomic DNA
actcctcccacatgatgctc

SEQ ID NO: 64 - DNA, 20 bp, Homo sapiens
  source 1..20: mol_type = genomic DNA
ttggaggccagccagctgaa

SEQ ID NO: 65 - DNA, 20 bp, Homo sapiens
  source 1..20: mol_type = genomic DNA
agaaacggctaccacatcca

SEQ ID NO: 66 - DNA, 20 bp, Homo sapiens
  source 1..20: mol_type = genomic DNA
ccctccaatggatcctcgtt

SEQ ID NO: 67 - DNA, 18 bp, Homo sapiens
  source 1..18: mol_type = genomic DNA
actgttctgaaggccgtg

SEQ ID NO: 68 - DNA, 18 bp, Homo sapiens
  source 1..18: mol_type = genomic DNA
ggtcttcaagggcaggtg

SEQ ID NO: 69 - DNA, 20 bp, Homo sapiens
  source 1..20: mol_type = genomic DNA
acaattctccgatccgtccc

SEQ ID NO: 70 - DNA, 20 bp, Homo sapiens
  source 1..20: mol_type = genomic DNA
gtgattggcttagtgggcga

SEQ ID NO: 71 - DNA, 19 bp, synthetic construct
  source 1..19: mol_type = other DNA
gagatagtgagaggcctcc

SEQ ID NO: 72 - DNA, 19 bp, synthetic construct
  source 1..19: mol_type = other DNA
aggaatacacgtggtagag

SEQ ID NO: 73 - DNA, 19 bp, synthetic construct
  source 1..19: mol_type = other DNA
ccagtactgccgcggcagg

SEQ ID NO: 74 - DNA, 19 bp, synthetic construct
  source 1..19: mol_type = other DNA
agtccaggatgtcatccat

SEQ ID NO: 75 - DNA, 19 bp, synthetic construct
  source 1..19: mol_type = other DNA
tcttctgcttacgtgtgtt

SEQ ID NO: 76 - DNA, 19 bp, synthetic construct
  source 1..19: mol_type = other DNA
atcatgtgggaggagtcca

SEQ ID NO: 77 - DNA, 19 bp, synthetic construct
  source 1..19: mol_type = other DNA
gcctgcaggacctgcagga

SEQ ID NO: 78 - DNA, 19 bp, synthetic construct
  source 1..19: mol_type = other DNA
cagctggtcagtgagcagg

SEQ ID NO: 79 - DNA, 19 bp, synthetic construct
  source 1..19: mol_type = other DNA
ctatttgcttgaccttgga

SEQ ID NO: 80 - DNA, 19 bp, synthetic construct
  source 1..19: mol_type = other DNA
caaagcatgggtacatcca

SEQ ID NO: 81 - DNA, 19 bp, synthetic construct
  source 1..19: mol_type = other DNA
aaggggtaaagtgagaacc

SEQ ID NO: 82 - DNA, 19 bp, synthetic construct
  source 1..19: mol_type = other DNA
agaggtacttcctgtagtg

SEQ ID NO: 83 - DNA, 19 bp, synthetic construct
  source 1..19: mol_type = other DNA
aacacatataccagctcct

SEQ ID NO: 84 - DNA, 19 bp, synthetic construct
  source 1..19: mol_type = other DNA
ttcatgacggagacatcag

SEQ ID NO: 85 - DNA, 19 bp, synthetic construct
  source 1..19: mol_type = other DNA
gtagggctcgtttgagccc

SEQ ID NO: 86 - DNA, 19 bp, synthetic construct
  source 1..19: mol_type = other DNA
cccatgtgggagatgaggc

SEQ ID NO: 87 - DNA, 19 bp, synthetic construct
  source 1..19: mol_type = other DNA
gagtcaagccggcatcctt

SEQ ID NO: 88 - DNA, 19 bp, synthetic construct
  source 1..19: mol_type = other DNA
ccaagatcttctggggctc

SEQ ID NO: 89 - DNA, 19 bp, synthetic construct
  source 1..19: mol_type = other DNA
cactgcagggtcttcaagg

SEQ ID NO: 90 - DNA, 19 bp, synthetic construct
  source 1..19: mol_type = other DNA
cacaccctgctggccagct

SEQ ID NO: 91 - DNA, 20 bp, synthetic construct
  source 1..20: mol_type = other DNA
acgccgctccaggccacccc

SEQ ID NO: 92 - DNA, 20 bp, synthetic construct
  source 1..20: mol_type = other DNA
gcacgccgctccaggccacc

SEQ ID NO: 93 - DNA, 20 bp, synthetic construct
  source 1..20: mol_type = other DNA
acgcacgccgctccaggcca

SEQ ID NO: 94 - DNA, 20 bp, synthetic construct
  source 1..20: mol_type = other DNA
ggccccagcaaagtgccgac

SEQ ID NO: 95 - DNA, 20 bp, synthetic construct
  source 1..20: mol_type = other DNA
gtccttcctgcggtcttggt

SEQ ID NO: 96 - DNA, 20 bp, synthetic construct
  source 1..20: mol_type = other DNA
agtccttcctgcggtcttgg

SEQ ID NO: 97 - DNA, 20 bp, synthetic construct
  source 1..20: mol_type = other DNA
ccccagtccttcctgcggtc

SEQ ID NO: 98 - DNA, 20 bp, synthetic construct
  source 1..20: mol_type = other DNA
cacgtggccccagtccttcc

SEQ ID NO: 99 - DNA, 20 bp, synthetic construct
  source 1..20: mol_type = other DNA
catccaccctgttcaccacc

SEQ ID NO: 100 - DNA, 20 bp, synthetic construct
  source 1..20: mol_type = other DNA
cacatccaccctgttcacca

SEQ ID NO: 101 - DNA, 20 bp, synthetic construct
  source 1..20: mol_type = other DNA
gcgccacatccaccctgttc

SEQ ID NO: 102 - DNA, 20 bp, synthetic construct
  source 1..20: mol_type = other DNA
gcccccatctggaccttcct

SEQ ID NO: 103 - DNA, 20 bp, synthetic construct
  source 1..20: mol_type = other DNA
gaactccccgctctggaatg

SEQ ID NO: 104 - DNA, 20 bp, synthetic construct
  source 1..20: mol_type = other DNA
cttgaactccccgctctgga

SEQ ID NO: 105 - DNA, 20 bp, synthetic construct
  source 1..20: mol_type = other DNA
cggccatctgcttgctcagg

SEQ ID NO: 106 - DNA, 20 bp, synthetic construct
  source 1..20: mol_type = other DNA
ccggccatctgcttgctcag

SEQ ID NO: 107 - DNA, 20 bp, synthetic construct
  source 1..20: mol_type = other DNA
acccggccatctgcttgctc

SEQ ID NO: 108 - DNA, 20 bp, synthetic construct
  source 1..20: mol_type = other DNA
ctccaggcacccggccatct

SEQ ID NO: 109 - DNA, 20 bp, synthetic construct
  source 1..20: mol_type = other DNA
cctccaggcacccggccatc

SEQ ID NO: 110 - DNA, 20 bp, synthetic construct
  source 1..20: mol_type = other DNA
atcgaggctcagcttcccag

SEQ ID NO: 111 - DNA, 20 bp, synthetic construct
  source 1..20: mol_type = other DNA
cctgctccacatcgaggctc

SEQ ID NO: 112 - DNA, 20 bp, synthetic construct
  source 1..20: mol_type = other DNA
ggcgttgtacatgtccagcg

SEQ ID NO: 113 - DNA, 20 bp, synthetic construct
  source 1..20: mol_type = other DNA
gcatcacggcgttgtacatg

SEQ ID NO: 114 - DNA, 20 bp, synthetic construct
  source 1..20: mol_type = other DNA
agcatcacggcgttgtacat

SEQ ID NO: 115 - DNA, 20 bp, synthetic construct
  source 1..20: mol_type = other DNA
aagcatcacggcgttgtaca

SEQ ID NO: 116 - DNA, 20 bp, synthetic construct
  source 1..20: mol_type = other DNA
tccagacacctttcgatggt

SEQ ID NO: 117 - DNA, 20 bp, synthetic construct
  source 1..20: mol_type = other DNA
cagaacggcggtgaagagtg

SEQ ID NO: 118 - DNA, 20 bp, synthetic construct
  source 1..20: mol_type = other DNA
gcagaacggcggtgaagagt

SEQ ID NO: 119 - DNA, 20 bp, synthetic construct
  source 1..20: mol_type = other DNA
gacagcagaacggcggtgaa

SEQ ID NO: 120 - DNA, 20 bp, synthetic construct
  source 1..20: mol_type = other DNA
acggccttcagaacagtggc

SEQ ID NO: 121 - DNA, 20 bp, synthetic construct
  source 1..20: mol_type = other DNA
caccttgtgcacggccttca

SEQ ID NO: 122 - DNA, 20 bp, synthetic construct
  source 1..20: mol_type = other DNA
acacgcccatccttggcata

SEQ ID NO: 123 - DNA, 20 bp, synthetic construct
  source 1..20: mol_type = other DNA
ggacacacgcccatccttgg

SEQ ID NO: 124 - DNA, 20 bp, synthetic construct
  source 1..20: mol_type = other DNA
aggacacacgcccatccttg

SEQ ID NO: 125 - DNA, 20 bp, synthetic construct
  source 1..20: mol_type = other DNA
gtaggacacacgcccatcct

SEQ ID NO: 126 - DNA, 20 bp, synthetic construct
  source 1..20: mol_type = other DNA
ggtaggacacacgcccatcc

SEQ ID NO: 127 - DNA, 20 bp, synthetic construct
  source 1..20: mol_type = other DNA
gacctccttgcttggcaacg

SEQ ID NO: 128 - DNA, 20 bp, synthetic construct
  source 1..20: mol_type = other DNA
tttctcccattggtcccgca

SEQ ID NO: 129 - DNA, 20 bp, synthetic construct
  source 1..20: mol_type = other DNA
gtgctttctcccattggtcc

SEQ ID NO: 130 - DNA, 20 bp, synthetic construct
  source 1..20: mol_type = other DNA
ccggcacagtgctttctccc

SEQ ID NO: 131 - DNA, 20 bp, synthetic construct
  source 1..20: mol_type = other DNA
ccactgacccgctgcctctg

SEQ ID NO: 132 - DNA, 20 bp, synthetic construct
  source 1..20: mol_type = other DNA
ctcctcccagtactgccgcg

SEQ ID NO: 133 - DNA, 20 bp, synthetic construct
  source 1..20: mol_type = other DNA
ttgtccaggctgcatggcat

SEQ ID NO: 134 - DNA, 20 bp, synthetic construct
  source 1..20: mol_type = other DNA
tccaccgcctcgaaggtcag

SEQ ID NO: 135 - DNA, 20 bp, synthetic construct
  source 1..20: mol_type = other DNA
atccaccgcctcgaaggtca

SEQ ID NO: 136 - DNA, 20 bp, synthetic construct
  source 1..20: mol_type = other DNA
catccaccgcctcgaaggtc

SEQ ID NO: 137 - DNA, 20 bp, synthetic construct
  source 1..20: mol_type = other DNA
acatccaccgcctcgaaggt

SEQ ID NO: 138 - DNA, 20 bp, synthetic construct
  source 1..20: mol_type = other DNA
ggtacatccaccgcctcgaa

SEQ ID NO: 139 - DNA, 20 bp, synthetic construct
  source 1..20: mol_type = other DNA
gtgcggcgatgtccagggca

SEQ ID NO: 140 - DNA, 20 bp, synthetic construct
  source 1..20: mol_type = other DNA
agagtgcggcgatgtccagg

SEQ ID NO: 141 - DNA, 20 bp, synthetic construct
  source 1..20: mol_type = other DNA
cagagtgcggcgatgtccag

SEQ ID NO: 142 - DNA, 20 bp, synthetic construct
  source 1..20: mol_type = other DNA
ccagagtgcggcgatgtcca

SEQ ID NO: 143 - DNA, 20 bp, synthetic construct
  source 1..20: mol_type = other DNA
caccagagtgcggcgatgtc

SEQ ID NO: 144 - DNA, 20 bp, synthetic construct
  source 1..20: mol_type = other DNA
agcaccagagtgcggcgatg

SEQ ID NO: 145 - DNA, 20 bp, synthetic construct
  source 1..20: mol_type = other DNA
ggtccagcacgcgcccgttg

SEQ ID NO: 146 - DNA, 20 bp, synthetic construct
  source 1..20: mol_type = other DNA
aggtccagcacgcgcccgtt

SEQ ID NO: 147 - DNA, 20 bp, synthetic construct
  source 1..20: mol_type = other DNA
gccagctcacggcgcagctc

SEQ ID NO: 148 - DNA, 20 bp, Homo sapiens
  source 1..20: mol_type = genomic DNA
ggggtggcctggagcggcgt

SEQ ID NO: 149 - DNA, 20 bp, Homo sapiens
  source 1..20: mol_type = genomic DNA
ggtggcctggagcggcgtgc

SEQ ID NO: 150 - DNA, 20 bp, Homo sapiens
  source 1..20: mol_type = genomic DNA
tggcctggagcggcgtgcgt

SEQ ID NO: 151 - DNA, 20 bp, Homo sapiens
  source 1..20: mol_type = genomic DNA
gtcggcactttgctggggcc

SEQ ID NO: 152 - DNA, 20 bp, Homo sapiens
  source 1..20: mol_type = genomic DNA
accaagaccgcaggaaggac

SEQ ID NO: 153 - DNA, 20 bp, Homo sapiens
  source 1..20: mol_type = genomic DNA
ccaagaccgcaggaaggact

SEQ ID NO: 154 - DNA, 20 bp, Homo sapiens
  source 1..20: mol_type = genomic DNA
gaccgcaggaaggactgggg

SEQ ID NO: 155 - DNA, 20 bp, Homo sapiens
  source 1..20: mol_type = genomic DNA
ggaaggactggggccacgtg

SEQ ID NO: 156 - DNA, 20 bp, Homo sapiens
  source 1..20: mol_type = genomic DNA
ggtggtgaacagggtggatg

SEQ ID NO: 157 - DNA, 20 bp, Homo sapiens
  source 1..20: mol_type = genomic DNA
tggtgaacagggtggatgtg

SEQ ID NO: 158 - DNA, 20 bp, Homo sapiens
  source 1..20: mol_type = genomic DNA
gaacagggtggatgtggcgc

SEQ ID NO: 159 - DNA, 20 bp, Homo sapiens
  source 1..20: mol_type = genomic DNA
aggaaggtccagatgggggc

SEQ ID NO: 160 - DNA, 20 bp, Homo sapiens
  source 1..20: mol_type = genomic DNA
cattccagagcggggagttc

SEQ ID NO: 161 - DNA, 20 bp, Homo sapiens
  source 1..20: mol_type = genomic DNA
tccagagcggggagttcaag

SEQ ID NO: 162 - DNA, 20 bp, Homo sapiens
  source 1..20: mol_type = genomic DNA
cctgagcaagcagatggccg

SEQ ID NO: 163 - DNA, 20 bp, Homo sapiens
  source 1..20: mol_type = genomic DNA
ctgagcaagcagatggccgg

SEQ ID NO: 164 - DNA, 20 bp, Homo sapiens
  source 1..20: mol_type = genomic DNA
gagcaagcagatggccgggt

SEQ ID NO: 165 - DNA, 20 bp, Homo sapiens
  source 1..20: mol_type = genomic DNA
agatggccgggtgcctggag

SEQ ID NO: 166 - DNA, 20 bp, Homo sapiens
  source 1..20: mol_type = genomic DNA
gatggccgggtgcctggagg

SEQ ID NO: 167 - DNA, 20 bp, Homo sapiens
  source 1..20: mol_type = genomic DNA
ctgggaagctgagcctcgat

SEQ ID NO: 168 - DNA, 20 bp, Homo sapiens
  source 1..20: mol_type = genomic DNA
gagcctcgatgtggagcagg

SEQ ID NO: 169 - DNA, 20 bp, Homo sapiens
  source 1..20: mol_type = genomic DNA
cgctggacatgtacaacgcc

SEQ ID NO: 170 - DNA, 20 bp, Homo sapiens
  source 1..20: mol_type = genomic DNA
catgtacaacgccgtgatgc

SEQ ID NO: 171 - DNA, 20 bp, Homo sapiens
  source 1..20: mol_type = genomic DNA
atgtacaacgccgtgatgct

SEQ ID NO: 172 - DNA, 20 bp, Homo sapiens
  source 1..20: mol_type = genomic DNA
tgtacaacgccgtgatgctt

SEQ ID NO: 173 - DNA, 20 bp, Homo sapiens
  source 1..20: mol_type = genomic DNA
accatcgaaaggtgtctgga

SEQ ID NO: 174 - DNA, 20 bp, Homo sapiens
  source 1..20: mol_type = genomic DNA
cactcttcaccgccgttctg

SEQ ID NO: 175 - DNA, 20 bp, Homo sapiens
  source 1..20: mol_type = genomic DNA
actcttcaccgccgttctgc

SEQ ID NO: 176 - DNA, 20 bp, Homo sapiens
  source 1..20: mol_type = genomic DNA
ttcaccgccgttctgctgtc

SEQ ID NO: 177 - DNA, 20 bp, Homo sapiens
  source 1..20: mol_type = genomic DNA
gccactgttctgaaggccgt

SEQ ID NO: 178 - DNA, 20 bp, Homo sapiens
  source 1..20: mol_type = genomic DNA
tgaaggccgtgcacaaggtg

SEQ ID NO: 179 - DNA, 20 bp, Homo sapiens
  source 1..20: mol_type = genomic DNA
tatgccaaggatgggcgtgt

SEQ ID NO: 180 - DNA, 23 bp, Homo sapiens
  source 1..23: mol_type = genomic DNA
cctgcaaggatgggcgtgtgtcc

SEQ ID NO: 181 - DNA, 20 bp, Homo sapiens
  source 1..20: mol_type = genomic DNA
caaggatgggcgtgtgtcct

SEQ ID NO: 182 - DNA, 20 bp, Homo sapiens
  source 1..20: mol_type = genomic DNA
aggatgggcgtgtgtcctac

SEQ ID NO: 183 - DNA, 20 bp, Homo sapiens
  source 1..20: mol_type = genomic DNA
ggatgggcgtgtgtcctacc

SEQ ID NO: 184 - DNA, 20 bp, Homo sapiens
  source 1..20: mol_type = genomic DNA
cgttgccaagcaaggaggtc

SEQ ID NO: 185 - DNA, 20 bp, Homo sapiens
  source 1..20: mol_type = genomic DNA
tgcgggaccaatgggagaaa

SEQ ID NO: 186 - DNA, 20 bp, Homo sapiens
  source 1..20: mol_type = genomic DNA
ggaccaatgggagaaagcac

SEQ ID NO: 187 - DNA, 20 bp, Homo sapiens
  source 1..20: mol_type = genomic DNA
gggagaaagcactgtgccgg

SEQ ID NO: 188 - DNA, 20 bp, Homo sapiens
  source 1..20: mol_type = genomic DNA
cagaggcagcgggtcagtgg

SEQ ID NO: 189 - DNA, 20 bp, Homo sapiens
  source 1..20: mol_type = genomic DNA
cgcggcagtactgggaggag

SEQ ID NO: 190 - DNA, 20 bp, Homo sapiens
  source 1..20: mol_type = genomic DNA
atgccatgcagcctggacaa

SEQ ID NO: 191 - DNA, 20 bp, Homo sapiens
  source 1..20: mol_type = genomic DNA
ctgaccttcgaggcggtgga

SEQ ID NO: 192 - DNA, 20 bp, Homo sapiens
  source 1..20: mol_type = genomic DNA
tgaccttcgaggcggtggat

SEQ ID NO: 193 - DNA, 20 bp, Homo sapiens
  source 1..20: mol_type = genomic DNA
gaccttcgaggcggtggatg

SEQ ID NO: 194 - DNA, 20 bp, Homo sapiens
  source 1..20: mol_type = genomic DNA
accttcgaggcggtggatgt

SEQ ID NO: 195 - DNA, 20 bp, Homo sapiens
  source 1..20: mol_type = genomic DNA
ttcgaggcggtggatgtacc

SEQ ID NO: 196 - DNA, 20 bp, Homo sapiens
  source 1..20: mol_type = genomic DNA
tgccctggacatcgccgcac

SEQ ID NO: 197 - DNA, 20 bp, Homo sapiens
  source 1..20: mol_type = genomic DNA
cctggacatcgccgcactct

SEQ ID NO: 198 - DNA, 20 bp, Homo sapiens
  source 1..20: mol_type = genomic DNA
ctggacatcgccgcactctg

SEQ ID NO: 199 - DNA, 20 bp, Homo sapiens
  source 1..20: mol_type = genomic DNA
tggacatcgccgcactctgg

SEQ ID NO: 200 - DNA, 20 bp, Homo sapiens
  source 1..20: mol_type = genomic DNA
gacatcgccgcactctggtg

SEQ ID NO: 201 - DNA, 20 bp, Homo sapiens
  source 1..20: mol_type = genomic DNA
catcgccgcactctggtgct

SEQ ID NO: 202 - DNA, 20 bp, Homo sapiens
  source 1..20: mol_type = genomic DNA
caacgggcgcgtgctggacc

SEQ ID NO: 203 - DNA, 20 bp, Homo sapiens
  source 1..20: mol_type = genomic DNA
aacgggcgcgtgctggacct

SEQ ID NO: 204 - DNA, 20 bp, Homo sapiens
  source 1..20: mol_type = genomic DNA
gagctgcgccgtgagctggc

SEQ ID NO: 205 - DNA, 3771 bp, Homo sapiens
  source 1..3771: mol_type = genomic DNA
ggggtggcctggagcggcgtgcgtaatgtcggcactttgctggggccgcggagcggcggggctcaaacgagccctacggccttgcggccgcccgggactccccggcaaagaagggaccgccggtggcgtctgcggccccaggaggagctcgtccgccagcccccaggagcaagaccaagaccgcaggaaggactggggccacgtggagctgctggaggtgctccaggcgcgggtgcggcagctgcaggctgagagcgtgtcggaggtggtggtgaacagggtggatgtggcgcggctcccagaatgtggcagtggagatggtagcctccagccacccaggaaggtccagatgggggccaaggatgccaccccggtgccctgtggccgctgggcaaagatactggagaaggataagcggacccagcagatgcgtatgcagcggttgaaggcgaagctgcagatgccattccagagcggggagttcaaggcgctgaccaggcgcctgcaggtggagccccggctcctgagcaagcagatggccgggtgcctggaggactgcacgcgccaggcccccgagagcccctgggaggagcagctggcccggctgctgcaggaggcccctgggaagctgagcctcgatgtggagcaggccccgtcggggcagcactcgcaggcccagctctcaggtcagcagcagaggctcctggccttcttcaagtgctgcctgctcactgaccagctgcccctcgcccaccacctgctggtcgtccaccacggccagcggcagaagcggaagctgctcacgctggacatgtacaacgccgtgatgcttggctgggcgcggcagggtgccttcaaggagctggtatatgtgttattcatggtgaaggatgccggcttgactccggacctgctgtcctatgcggctgccctccagtgcatggggaggcaggaccaggacgccgggaccatcgaaaggtgtctggaacagatgagccaggaggggctgaagctgcaggcactcttcaccgccgttctgctgtctgaggaggatcgggccactgttctgaaggccgtgcacaaggtgaagcccaccttcagcctcccgccgcagctgccgcccccggtcaacacctccaagctgctcagggacgtgtatgccaaggatgggcgtgtgtcctacccgaagctgcacctgcccttgaagaccctgcagtgcctctttgagaagcagctccacatggagctggccagcagggtgtgcgtggtgtccgtggagaagcccacgttgccaagcaaggaggtcaagcacgcgcggaagaccctgaagaccctgcgggaccaatgggagaaagcactgtgccgggcgctgcgggagaccaagaaccgcctagagcgcgaggtgtacgagggccggttctcactttaccccttcctgtgcctgctggacgagcgcgaggtggtgcggatgctcctgcaggtcctgcaggcgctgcccgcccaaggtgagtccttcaccaccctggcccgggagctgagtgcgcgcactttcagccggcacgtggtgcagaggcagcgggtcagtggccaggtgcaggcgctgcagaaccactacaggaagtacctctgcttgctggcctccgacgccgaggtgcccgagccctgcctgccgcggcagtactgggaggagctgggggcgcccgaggccctgcgggagcagccctggcccctgccagtgcagatggagctgggcaagctgctggcggagatgctggtgcaggctacgcagatgccatgcagcctggacaagccgcatcgttcctctcggcttgtccccgtgctctaccacgtgtattccttccgcaacgtccagcagatcggcatcctgaagccgcacccggcctacgtgcagctgctggagaaggccgcggagcccacgctgaccttcgaggcggtggatgtacccatgctttgccccccgctgccctggacatcgccgcactctggtgctttcctgctcagccccaccaagctgatgcgcacggtggaaggcgccacgcagcaccaggagctgctggaaacctgcccgcccaccgcgctgcatggcgcactggacgccctcacccaactgggcaactgcgcctggcgcgtcaacgggcgcgtgctggacctggtgctgcagctcttccaggccaagggctgcccccagctaggcgtgccggccccgccctccgaggcgccccagccgcccgaggcccacctgccgcacagcgccgcgcccgcccgcaaggccgagctgcgccgtgagctggcgcactgccagaaggtggcccgggagatgcacagcctgcgggcggaggcgctgtaccgcctctcgctggcgcagcacctgcgggaccgcgtcttctggctgccgcacaacatggacttccgcggccgcacctacccctgcccgccgcacttcaaccacctgggcagcgacgtggcgcgggccctgctggagttcgcccagggccgcccgctcggcccgcacggcctggattggctcaagatccacctggtcaatctcacggggttgaagaagcgggagccgctgcggaagcgcctggcctttgcggaggaggtgatggatgacatcctggactccgcggaccaacccttgacgggccgaaagtggtggatgggcgcggaggaaccctggcagacgctggcctgctgtatggaggtggcgaacgctgtgcgcgcctccgaccctgccgcctatgtctcccacctccccgtccatcaggacggctcttgcaacggcctgcagcattatgctgctctgggccgcgacagcgtgggcgccgcctccgtcaacctggagccctcggatgtgccgcaggacgtgtacagcggcgtggccgcgcaggtggaggtgttccgtaggcaggacgcccagcggggcatgcgggtggcacaggtgctggaaggtttcatcacccgcaaggtggtgaagcagacggtgatgacggtggtgtacggggtcacgcgctatggcgggcgcctgcagattgagaagcgcctccgggagctgagcgactttccccaggagttcgtgtgggaggcctctcactatctcgtacgccaggtcttcaagagtctacaggagatgttctcggggacccgggccatccagcactggctgaccgagagtgcccgcctcatctcccacatgggctctgtggtggagtgggtcacacccctgggcgtccccgtcatccagccctatcgcctggactccaaggtcaagcaaataggaggtggaattcagagcatcacctacacccacaacggagacatcagccgaaagcccaacacacgtaagcagaagaacggcttcccgcccaacttcatccactcgctggactcctcccacatgatgctcaccgccctgcactgctacaggaagggcctgaccttcgtctctgtgcacgactgttactggactcacgcagctgatgtctccgtcatgaaccaggtgtgccgggagcagtttgtccgcttgcacagcgagcccatcctgcaggacctgtccagattcctggtcaagcggttctgctctgagccccagaagatcttggaggccagccagctgaaggagacactgcaggcggtgcccaagccaggggccttcgacctggagcaggtgaagcgttccacctacttcttcagctgacaccccgtgagccttgtcagtgtgtaaataaagctcttttgccacccccagga

SEQ ID NO: 206 - DNA, 20 bp, synthetic construct
  source 1..20: mol_type = other DNA
gcacgccgctccaggccacc

SEQ ID NO: 207 - DNA, 20 bp, synthetic construct
  source 1..20: mol_type = other DNA
acgcacgccgctccaggcca

SEQ ID NO: 208 - DNA, 20 bp, synthetic construct
  source 1..20: mol_type = other DNA
ggccccagcaaagtgccgac

SEQ ID NO: 209 - DNA, 20 bp, synthetic construct
  source 1..20: mol_type = other DNA
gtccttcctgcggtcttggt

SEQ ID NO: 210 - DNA, 20 bp, synthetic construct
  source 1..20: mol_type = other DNA
agtccttcctgcggtcttgg

SEQ ID NO: 211 - DNA, 20 bp, synthetic construct
  source 1..20: mol_type = other DNA
ccccagtccttcctgcggtc

SEQ ID NO: 212 - DNA, 20 bp, synthetic construct
  source 1..20: mol_type = other DNA
cacgtggccccagtccttcc

SEQ ID NO: 213 - DNA, 20 bp, synthetic construct
  source 1..20: mol_type = other DNA
catccaccctgttcaccacc

SEQ ID NO: 214 - DNA, 20 bp, synthetic construct
  source 1..20: mol_type = other DNA
cacatccaccctgttcacca

SEQ ID NO: 215 - DNA, 20 bp, synthetic construct
  source 1..20: mol_type = other DNA
gcgccacatccaccctgttc

SEQ ID NO: 216 - DNA, 20 bp, synthetic construct
  source 1..20: mol_type = other DNA
gcccccatctggaccttcct

SEQ ID NO: 217 - DNA, 20 bp, synthetic construct
  source 1..20: mol_type = other DNA
gaactccccgctctggaatg

SEQ ID NO: 218 - DNA, 20 bp, synthetic construct
  source 1..20: mol_type = other DNA
cttgaactccccgctctgga

SEQ ID NO: 219 - DNA, 20 bp, synthetic construct
  source 1..20: mol_type = other DNA
cggccatctgcttgctcagg

SEQ ID NO: 220 - DNA, 20 bp, synthetic construct
  source 1..20: mol_type = other DNA
ccggccatctgcttgctcag

SEQ ID NO: 221 - DNA, 20 bp, synthetic construct
  source 1..20: mol_type = other DNA
acccggccatctgcttgctc

SEQ ID NO: 222 - DNA, 20 bp, synthetic construct
  source 1..20: mol_type = other DNA
ctccaggcacccggccatct

SEQ ID NO: 223 - DNA, 20 bp, synthetic construct
  source 1..20: mol_type = other DNA
cctccaggcacccggccatc

SEQ ID NO: 224 - DNA, 20 bp, synthetic construct
  source 1..20: mol_type = other DNA
atcgaggctcagcttcccag

SEQ ID NO: 225 - DNA, 20 bp, synthetic construct
  source 1..20: mol_type = other DNA
cctgctccacatcgaggctc

SEQ ID NO: 226 - DNA, 20 bp, synthetic construct
  source 1..20: mol_type = other DNA
ggcgttgtacatgtccagcg

SEQ ID NO: 227 - DNA, 20 bp, synthetic construct
  source 1..20: mol_type = other DNA
gcatcacggcgttgtacatg

SEQ ID NO: 228 - DNA, 20 bp, synthetic construct
  source 1..20: mol_type = other DNA
agcatcacggcgttgtacat

SEQ ID NO: 229 - DNA, 20 bp, synthetic construct
  source 1..20: mol_type = other DNA
aagcatcacggcgttgtaca

SEQ ID NO: 230 - DNA, 20 bp, synthetic construct
  source 1..20: mol_type = other DNA
tccagacacctttcgatggt

SEQ ID NO: 231 - DNA, 20 bp, synthetic construct
  source 1..20: mol_type = other DNA
cagaacggcggtgaagagtg

SEQ ID NO: 232 - DNA, 20 bp, synthetic construct
  source 1..20: mol_type = other DNA
gcagaacggcggtgaagagt

SEQ ID NO: 233 - DNA, 20 bp, synthetic construct
  source 1..20: mol_type = other DNA
gacagcagaacggcggtgaa

SEQ ID NO: 234 - DNA, 20 bp, synthetic construct
  source 1..20: mol_type = other DNA
acggccttcagaacagtggc

SEQ ID NO: 235 - DNA, 20 bp, synthetic construct
  source 1..20: mol_type = other DNA
caccttgtgcacggccttca

SEQ ID NO: 236 - DNA, 20 bp, synthetic construct
  source 1..20: mol_type = other DNA
acacgcccatccttggcata

SEQ ID NO: 237 - DNA, 20 bp, synthetic construct
  source 1..20: mol_type = other DNA
ggacacacgcccatccttgg

SEQ ID NO: 238 - DNA, 20 bp, synthetic construct
  source 1..20: mol_type = other DNA
aggacacacgcccatccttg

SEQ ID NO: 239 - DNA, 20 bp, synthetic construct
  source 1..20: mol_type = other DNA
gtaggacacacgcccatcct

SEQ ID NO: 240 - DNA, 20 bp, synthetic construct
  source 1..20: mol_type = other DNA
ggtaggacacacgcccatcc

SEQ ID NO: 241 - DNA, 20 bp, synthetic construct
  source 1..20: mol_type = other DNA
gacctccttgcttggcaacg

SEQ ID NO: 242 - DNA, 20 bp, synthetic construct
  source 1..20: mol_type = other DNA
tttctcccattggtcccgca

SEQ ID NO: 243 - DNA, 20 bp, synthetic construct
  source 1..20: mol_type = other DNA
gtgctttctcccattggtcc

SEQ ID NO: 244 - DNA, 20 bp, synthetic construct
  source 1..20: mol_type = other DNA
ccggcacagtgctttctccc

SEQ ID NO: 245 - DNA, 20 bp, synthetic construct
  source 1..20: mol_type = other DNA
ccactgacccgctgcctctg

SEQ ID NO: 246 - DNA, 20 bp, synthetic construct
  source 1..20: mol_type = other DNA
ctcctcccagtactgccgcg

SEQ ID NO: 247 - DNA, 20 bp, synthetic construct
  source 1..20: mol_type = other DNA
ttgtccaggctgcatggcat

SEQ ID NO: 248 - DNA, 20 bp, synthetic construct
  source 1..20: mol_type = other DNA
tccaccgcctcgaaggtcag

SEQ ID NO: 249 - DNA, 20 bp, synthetic construct
  source 1..20: mol_type = other DNA
atccaccgcctcgaaggtca

SEQ ID NO: 250 - DNA, 20 bp, synthetic construct
  source 1..20: mol_type = other DNA
catccaccgcctcgaaggtc

SEQ ID NO: 251 - DNA, 20 bp, synthetic construct
  source 1..20: mol_type = other DNA
acatccaccgcctcgaaggt

SEQ ID NO: 252 - DNA, 20 bp, synthetic construct
  source 1..20: mol_type = other DNA
ggtacatccaccgcctcgaa

SEQ ID NO: 253 - DNA, 20 bp, synthetic construct
  source 1..20: mol_type = other DNA
gtgcggcgatgtccagggca

SEQ ID NO: 254 - DNA, 20 bp, synthetic construct
  source 1..20: mol_type = other DNA
agagtgcggcgatgtccagg

SEQ ID NO: 255 - DNA, 20 bp, synthetic construct
  source 1..20: mol_type = other DNA
cagagtgcggcgatgtccag

SEQ ID NO: 256 - DNA, 20 bp, synthetic construct
  source 1..20: mol_type = other DNA
ccagagtgcggcgatgtcca

SEQ ID NO: 257 - DNA, 20 bp, synthetic construct
  source 1..20: mol_type = other DNA
caccagagtgcggcgatgtc

SEQ ID NO: 258 - DNA, 20 bp, synthetic construct
  source 1..20: mol_type = other DNA
agcaccagagtgcggcgatg

SEQ ID NO: 259 - DNA, 20 bp, synthetic construct
  source 1..20: mol_type = other DNA
ggtccagcacgcgcccgttg

SEQ ID NO: 260 - DNA, 20 bp, synthetic construct
  source 1..20: mol_type = other DNA
aggtccagcacgcgcccgtt

SEQ ID NO: 261 - DNA, 20 bp, synthetic construct
  source 1..20: mol_type = other DNA
gccagctcacggcgcagctc

SEQ ID NO: 262 - DNA, 20 bp, synthetic construct
  source 1..20: mol_type = other DNA
cgccagctcacggcgcagct

SEQ ID NO: 263 - DNA, 20 bp, synthetic construct
  source 1..20: mol_type = other DNA
gcagtgcgccagctcacggc

SEQ ID NO: 264 - DNA, 20 bp, synthetic construct
  source 1..20: mol_type = other DNA
cttctggcagtgcgccagct

SEQ ID NO: 265 - DNA, 20 bp, synthetic construct
  source 1..20: mol_type = other DNA
accttctggcagtgcgccag

SEQ ID NO: 266 - DNA, 20 bp, synthetic construct
  source 1..20: mol_type = other DNA
ccaccttctggcagtgcgcc

SEQ ID NO: 267 - DNA, 20 bp, synthetic construct
  source 1..20: mol_type = other DNA
tcccgggccaccttctggca

SEQ ID NO: 268 - DNA, 20 bp, synthetic construct
  source 1..20: mol_type = other DNA
gacgcggtcccgcaggtgct

SEQ ID NO: 269 - DNA, 20 bp, synthetic construct
  source 1..20: mol_type = other DNA
ggaagtccatgttgtgcggc

SEQ ID NO: 270 - DNA, 20 bp, synthetic construct
  source 1..20: mol_type = other DNA
gcggaagtccatgttgtgcg

SEQ ID NO: 271 - DNA, 20 bp, synthetic construct
  source 1..20: mol_type = other DNA
gtcgctgcccaggtggttga

SEQ ID NO: 272 - DNA, 20 bp, synthetic construct
  source 1..20: mol_type = other DNA
acgtcgctgcccaggtggtt

SEQ ID NO: 273 - DNA, 20 bp, synthetic construct
  source 1..20: mol_type = other DNA
ccacgtcgctgcccaggtgg

SEQ ID NO: 274 - DNA, 20 bp, synthetic construct
  source 1..20: mol_type = other DNA
cccgcgccacgtcgctgccc

SEQ ID NO: 275 - DNA, 20 bp, synthetic construct
  source 1..20: mol_type = other DNA
ggcccgcgccacgtcgctgc

SEQ ID NO: 276 - DNA, 20 bp, synthetic construct
  source 1..20: mol_type = other DNA
gcggccctgggcgaactcca

SEQ ID NO: 277 - DNA, 20 bp, synthetic construct
  source 1..20: mol_type = other DNA
caccactttcggcccgtcaa

SEQ ID NO: 278 - DNA, 20 bp, synthetic construct
  source 1..20: mol_type = other DNA
cctccgcgcccatccaccac

SEQ ID NO: 279 - DNA, 20 bp, synthetic construct
  source 1..20: mol_type = other DNA
tcctccgcgcccatccacca

SEQ ID NO: 280 - DNA, 20 bp, synthetic construct
  source 1..20: mol_type = other DNA
gttcctccgcgcccatccac

SEQ ID NO: 281 - DNA, 20 bp, synthetic construct
  source 1..20: mol_type = other DNA
gggttcctccgcgcccatcc

SEQ ID NO: 282 - DNA, 20 bp, synthetic construct
  source 1..20: mol_type = other DNA
aggccagcgtctgccagggt

SEQ ID NO: 283 - DNA, 20 bp, synthetic construct
  source 1..20: mol_type = other DNA
cggcacatccgagggctcca

SEQ ID NO: 284 - DNA, 20 bp, synthetic construct
  source 1..20: mol_type = other DNA
cacgccgctgtacacgtcct

SEQ ID NO: 285 - DNA, 20 bp, synthetic construct
  source 1..20: mol_type = other DNA
ccacgccgctgtacacgtcc

SEQ ID NO: 286 - DNA, 20 bp, synthetic construct
  source 1..20: mol_type = other DNA
gccacgccgctgtacacgtc

SEQ ID NO: 287 - DNA, 20 bp, synthetic construct
  source 1..20: mol_type = other DNA
ggccacgccgctgtacacgt

SEQ ID NO: 288 - DNA, 20 bp, synthetic construct
  source 1..20: mol_type = other DNA
tgcgcggccacgccgctgta

SEQ ID NO: 289 - DNA, 20 bp, synthetic construct
  source 1..20: mol_type = other DNA
ctgcctacggaacacctcca

SEQ ID NO: 290 - DNA, 20 bp, synthetic construct
  source 1..20: mol_type = other DNA
ttccagcacctgtgccaccc

SEQ ID NO: 291 - DNA, 20 bp, synthetic construct
  source 1..20: mol_type = other DNA
gaggcctcccacacgaactc

SEQ ID NO: 292 - DNA, 20 bp, synthetic construct
  source 1..20: mol_type = other DNA
agaggcctcccacacgaact

SEQ ID NO: 293 - DNA, 20 bp, synthetic construct
  source 1..20: mol_type = other DNA
tgaattccacctcctatttg

SEQ ID NO: 294 - DNA, 20 bp, synthetic construct
  source 1..20: mol_type = other DNA
cgaaggtcaggcccttcctg

SEQ ID NO: 295 - DNA, 20 bp, synthetic construct
  source 1..20: mol_type = other DNA
agacgaaggtcaggcccttc

SEQ ID NO: 296 - DNA, 20 bp, synthetic construct
  source 1..20: mol_type = other DNA
gagacgaaggtcaggccctt

SEQ ID NO: 297 - DNA, 20 bp, synthetic construct
  source 1..20: mol_type = other DNA
agagacgaaggtcaggccct

SEQ ID NO: 298 - DNA, 20 bp, synthetic construct
  source 1..20: mol_type = other DNA
agtaacagtcgtgcacagag

SEQ ID NO: 299 - DNA, 20 bp, Homo sapiens
  source 1..20: mol_type = genomic DNA
ggggtggcctggagcggcgg

SEQ ID NO: 300 - DNA, 20 bp, Homo sapiens
  source 1..20: mol_type = genomic DNA
ggtggcctggagcggcgtgc

SEQ ID NO: 301 - DNA, 20 bp, Homo sapiens
  source 1..20: mol_type = genomic DNA
tggcctggagcggcgtgcgt

SEQ ID NO: 302 - DNA, 20 bp, Homo sapiens
  source 1..20: mol_type = genomic DNA
gtcggcactttgctggggcc

SEQ ID NO: 303 - DNA, 20 bp, Homo sapiens
  source 1..20: mol_type = genomic DNA
accaagaccgcaggaaggac

SEQ ID NO: 304 - DNA, 20 bp, Homo sapiens
  source 1..20: mol_type = genomic DNA
ccaagaccgcaggaaggact

SEQ ID NO: 305 - DNA, 20 bp, Homo sapiens
  source 1..20: mol_type = genomic DNA
gaccgcaggaaggactgggg

SEQ ID NO: 306 - DNA, 20 bp, Homo sapiens
  source 1..20: mol_type = genomic DNA
ggaaggactggggccacgtg

SEQ ID NO: 307 - DNA, 20 bp, Homo sapiens
  source 1..20: mol_type = genomic DNA
ggtggtgaacagggtggatg

SEQ ID NO: 308 - DNA, 20 bp, Homo sapiens
  source 1..20: mol_type = genomic DNA
tggtgaacagggtggatgtg

SEQ ID NO: 309 - DNA, 20 bp, Homo sapiens
  source 1..20: mol_type = genomic DNA
gaacagggtggatgtggcgc

SEQ ID NO: 310 - DNA, 20 bp, Homo sapiens
  source 1..20: mol_type = genomic DNA
aggaaggtccagatgggggc

SEQ ID NO: 311 - DNA, 20 bp, Homo sapiens
  source 1..20: mol_type = genomic DNA
cattccagagcggggagttc

SEQ ID NO: 312 - DNA, 20 bp, Homo sapiens
  source 1..20: mol_type = genomic DNA
tccagagcggggagttcaag

SEQ ID NO: 313 - DNA, 20 bp, Homo sapiens
  source 1..20: mol_type = genomic DNA
cctgagcaagcagatggccg

SEQ ID NO: 314 - DNA, 20 bp, Homo sapiens
  source 1..20: mol_type = genomic DNA
ctgagcaagcagatggccgg

SEQ ID NO: 315 - DNA, 20 bp, Homo sapiens
  source 1..20: mol_type = genomic DNA
gagcaagcagatggccgggt

SEQ ID NO: 316 - DNA, 20 bp, Homo sapiens
  source 1..20: mol_type = genomic DNA
agatggccgggtgcctggag

SEQ ID NO: 317 - DNA, 20 bp, Homo sapiens
  source 1..20: mol_type = genomic DNA
gatggccgggtgcctggagg

SEQ ID NO: 318 - DNA, 20 bp, Homo sapiens
  source 1..20: mol_type = genomic DNA
ctgggaagctgagcctcgat

SEQ ID NO: 319 - DNA, 20 bp, Homo sapiens
  source 1..20: mol_type = genomic DNA
gagcctcgatgtggagcagg

SEQ ID NO: 320 - DNA, 20 bp, Homo sapiens
  source 1..20: mol_type = genomic DNA
cgctggacatgtacaacgcc

SEQ ID NO: 321 - DNA, 20 bp, Homo sapiens
  source 1..20: mol_type = genomic DNA
catgtacaacgccgtgatgc

SEQ ID NO: 322 - DNA, 20 bp, Homo sapiens
  source 1..20: mol_type = genomic DNA
atgtacaacgccgtgatgct

SEQ ID NO: 323 - DNA, 20 bp, Homo sapiens
  source 1..20: mol_type = genomic DNA
tgtacaacgccgtgatgctt

SEQ ID NO: 324 - DNA, 20 bp, Homo sapiens
  source 1..20: mol_type = genomic DNA
accatcgaaaggtgtctgga

SEQ ID NO: 325 - DNA, 20 bp, Homo sapiens
  source 1..20: mol_type = genomic DNA
cactcttcaccgccgttctg

SEQ ID NO: 326 - DNA, 20 bp, Homo sapiens
  source 1..20: mol_type = genomic DNA
actcttcaccgccgttctgc

SEQ ID NO: 327 - DNA, 20 bp, Homo sapiens
  source 1..20: mol_type = genomic DNA
ttcaccgccgttctgctgtc

SEQ ID NO: 328 - DNA, 20 bp, Homo sapiens
  source 1..20: mol_type = genomic DNA
gccactgttctgaaggccgt

SEQ ID NO: 329 - DNA, 20 bp, Homo sapiens
  source 1..20: mol_type = genomic DNA
tgaaggccgtgcacaaggtg

SEQ ID NO: 330 - DNA, 20 bp, Homo sapiens
  source 1..20: mol_type = genomic DNA
tatgccaaggatgggcgtgt

SEQ ID NO: 331 - DNA, 20 bp, Homo sapiens
  source 1..20: mol_type = genomic DNA
ccaaggatgggcgtgtgtcc

SEQ ID NO: 332 - DNA, 20 bp, Homo sapiens
  source 1..20: mol_type = genomic DNA
caaggatgggcgtgtgtcct

SEQ ID NO: 333 - DNA, 20 bp, Homo sapiens
  source 1..20: mol_type = genomic DNA
aggatgggcgtgtgtcctac

SEQ ID NO: 334 - DNA, 20 bp, Homo sapiens
  source 1..20: mol_type = genomic DNA
ggatgggcgtgtgtcctacc

SEQ ID NO: 335 - DNA, 20 bp, Homo sapiens
  source 1..20: mol_type = genomic DNA
cgttgccaagcaaggaggtc

SEQ ID NO: 336 - DNA, 20 bp, Homo sapiens
  source 1..20: mol_type = genomic DNA
tgcgggaccaatgggagaaa

SEQ ID NO: 337 - DNA, 20 bp, Homo sapiens
  source 1..20: mol_type = genomic DNA
ggaccaatgggagaaagcac

SEQ ID NO: 338 - DNA, 20 bp, Homo sapiens
  source 1..20: mol_type = genomic DNA
gggagaaagcactgtgccgg

SEQ ID NO: 339 - DNA, 20 bp, Homo sapiens
  source 1..20: mol_type = genomic DNA
cagaggcagcgggtcagtgg

SEQ ID NO: 340 - DNA, 20 bp, Homo sapiens
  source 1..20: mol_type = genomic DNA
cgcggcagtactgggaggag

SEQ ID NO: 341 - DNA, 20 bp, Homo sapiens
  source 1..20: mol_type = genomic DNA
atgccatgcagcctggacaa

SEQ ID NO: 342 - DNA, 20 bp, Homo sapiens
  source 1..20: mol_type = genomic DNA
ctgaccttcgaggcggtgga

SEQ ID NO: 343 - DNA, 20 bp, Homo sapiens
  source 1..20: mol_type = genomic DNA
tgaccttcgaggcggtggat

SEQ ID NO: 344 - DNA, 20 bp, Homo sapiens
  source 1..20: mol_type = genomic DNA
gaccttcgaggcggtggatg

SEQ ID NO: 345 - DNA, 20 bp, Homo sapiens
  source 1..20: mol_type = genomic DNA
accttcgaggcggtggatgt

SEQ ID NO: 346 - DNA, 20 bp, Homo sapiens
  source 1..20: mol_type = genomic DNA
ttcgaggcggtggatgtacc

SEQ ID NO: 347 - DNA, 20 bp, Homo sapiens
  source 1..20: mol_type = genomic DNA
tgccctggacatcgccgcac

SEQ ID NO: 348 - DNA, 20 bp, Homo sapiens
  source 1..20: mol_type = genomic DNA
cctggacatcgccgcactct

SEQ ID NO: 349 - DNA, 20 bp, Homo sapiens
  source 1..20: mol_type = genomic DNA
ctggacatcgccgcactctg

SEQ ID NO: 350 - DNA, 20 bp, Homo sapiens
  source 1..20: mol_type = genomic DNA
tggacatcgccgcactctgg

SEQ ID NO: 351 - DNA, 20 bp, Homo sapiens
  source 1..20: mol_type = genomic DNA
gacatcgccgcactctggtg

SEQ ID NO: 352 - DNA, 20 bp, Homo sapiens
  source 1..20: mol_type = genomic DNA
catcgccgcactctggtgct

SEQ ID NO: 353 - DNA, 20 bp, Homo sapiens
  source 1..20: mol_type = genomic DNA
caacgggcgcgtgctggacc

SEQ ID NO: 354 - DNA, 20 bp, Homo sapiens
  source 1..20: mol_type = genomic DNA
aacgggcgcgtgctggacct

SEQ ID NO: 355 - DNA, 20 bp, Homo sapiens
  source 1..20: mol_type = genomic DNA
gagctgcgccgtgagctggc

SEQ ID NO: 356 - DNA, 20 bp, Homo sapiens
  source 1..20: mol_type = genomic DNA
agctgcgccgtgagctggcg

SEQ ID NO: 357 - DNA, 20 bp, Homo sapiens
  source 1..20: mol_type = genomic DNA
gccgtgagctggcgcactgc

SEQ ID NO: 358 - DNA, 20 bp, Homo sapiens
  source 1..20: mol_type = genomic DNA
agctggcgcactgccagaag

SEQ ID NO: 359 - DNA, 20 bp, Homo sapiens
  source 1..20: mol_type = genomic DNA
ctggcgcactgccagaaggt

SEQ ID NO: 360 - DNA, 20 bp, Homo sapiens
  source 1..20: mol_type = genomic DNA
ggcgcactgccagaaggtgg

SEQ ID NO: 361 - DNA, 20 bp, Homo sapiens
  source 1..20: mol_type = genomic DNA
tgccagaaggtggcccggga

SEQ ID NO: 362 - DNA, 20 bp, Homo sapiens
  source 1..20: mol_type = genomic DNA
agcacctgcgggaccgcgtc

SEQ ID NO: 363 - DNA, 20 bp, Homo sapiens
  source 1..20: mol_type = genomic DNA
gccgcacaacatggacttcc

SEQ ID NO: 364 - DNA, 20 bp, Homo sapiens
  source 1..20: mol_type = genomic DNA
cgcacaacatggacttccgc

SEQ ID NO: 365 - DNA, 20 bp, Homo sapiens
  source 1..20: mol_type = genomic DNA
tcaaccacctgggcagcgac

SEQ ID NO: 366 - DNA, 20 bp, Homo sapiens
  source 1..20: mol_type = genomic DNA
aaccacctgggcagcgacgt

SEQ ID NO: 367 - DNA, 20 bp, Homo sapiens
  source 1..20: mol_type = genomic DNA
ccacctgggcagcgacgtgg

SEQ ID NO: 368 - DNA, 20 bp, Homo sapiens
  source 1..20: mol_type = genomic DNA
gggcagcgacgtggcgcggg

SEQ ID NO: 369 - DNA, 20 bp, Homo sapiens
  source 1..20: mol_type = genomic DNA
gcagcgacgtggcgcgggcc

SEQ ID NO: 370 - DNA, 20 bp, Homo sapiens
  source 1..20: mol_type = genomic DNA
tggagttcgcccagggccgc

SEQ ID NO: 371 - DNA, 20 bp, Homo sapiens
  source 1..20: mol_type = genomic DNA
ttgacgggccgaaagtggtg

SEQ ID NO: 372 - DNA, 20 bp, Homo sapiens
  source 1..20: mol_type = genomic DNA
gtggtggatgggcgcggagg

SEQ ID NO: 373 - DNA, 20 bp, Homo sapiens
  source 1..20: mol_type = genomic DNA
tggtggatgggcgcggagga

SEQ ID NO: 374 - DNA, 20 bp, Homo sapiens
  source 1..20: mol_type = genomic DNA
gtggatgggcgcggaggaac

SEQ ID NO: 375 - DNA, 20 bp, Homo sapiens
  source 1..20: mol_type = genomic DNA
ggatgggcgcggaggaaccc

SEQ ID NO: 376 - DNA, 20 bp, Homo sapiens
  source 1..20: mol_type = genomic DNA
accctggcagacgctggcct

SEQ ID NO: 377 - DNA, 20 bp, Homo sapiens
  source 1..20: mol_type = genomic DNA
tggagccctcggatgtgccg

SEQ ID NO: 378 - DNA, 20 bp, Homo sapiens
  source 1..20: mol_type = genomic DNA
aggacgtgtacagcggcgtg

SEQ ID NO: 379 - DNA, 20 bp, Homo sapiens
  source 1..20: mol_type = genomic DNA
ggacgtgtacagcggcgtgg

SEQ ID NO: 380 - DNA, 20 bp, Homo sapiens
  source 1..20: mol_type = genomic DNA
gacgtgtacagcggcgtggc

SEQ ID NO: 381 - DNA, 20 bp, Homo sapiens
  source 1..20: mol_type = genomic DNA
acgtgtacagcggcgtggcc

SEQ ID NO: 382 - DNA, 20 bp, Homo sapiens
  source 1..20: mol_type = genomic DNA
tacagcggcgtggccgcgca

SEQ ID NO: 383 - DNA, 20 bp, Homo sapiens
  source 1..20: mol_type = genomic DNA
tggaggtgttccgtaggcag

SEQ ID NO: 384 - DNA, 20 bp, Homo sapiens
  source 1..20: mol_type = genomic DNA
gggtggcacaggtgctggaa

SEQ ID NO: 385 - DNA, 20 bp, Homo sapiens
  source 1..20: mol_type = genomic DNA
gagttcgtgtgggaggcctc

SEQ ID NO: 386 - DNA, 20 bp, Homo sapiens
  source 1..20: mol_type = genomic DNA
agttcgtgtgggaggcctct

SEQ ID NO: 387 - DNA, 20 bp, Homo sapiens
  source 1..20: mol_type = genomic DNA
caaataggaggtggaattca

SEQ ID NO: 388 - DNA, 20 bp, Homo sapiens
  source 1..20: mol_type = genomic DNA
caggaagggcctgaccttcg

SEQ ID NO: 389 - DNA, 20 bp, Homo sapiens
  source 1..20: mol_type = genomic DNA
gaagggcctgaccttcgtct

SEQ ID NO: 390 - DNA, 20 bp, Homo sapiens
  source 1..20: mol_type = genomic DNA
aagggcctgaccttcgtctc

SEQ ID NO: 391 - DNA, 20 bp, Homo sapiens
  source 1..20: mol_type = genomic DNA
agggcctgaccttcgtctct

SEQ ID NO: 392 - DNA, 20 bp, Homo sapiens
  source 1..20: mol_type = genomic DNA
ctctgtgcacgactgttact

SEQ ID NO: 393 - DNA, 20 bp, synthetic construct
  source 1..20: mol_type = other DNA
cacggtgttgtacatgtgca

SEQ ID NO: 394 - DNA, 20 bp, synthetic construct
  source 1..20: mol_type = other DNA
atcacggtgttgtacatgtg

SEQ ID NO: 395 - DNA, 20 bp, synthetic construct
  source 1..20: mol_type = other DNA
catcacggtgttgtacatgt

SEQ ID NO: 396 - DNA, 20 bp, synthetic construct
  source 1..20: mol_type = other DNA
gtgcatctctcgagccacct

SEQ ID NO: 397 - DNA, 20 bp, synthetic construct
  source 1..20: mol_type = other DNA
tgtgcatctctcgagccacc

SEQ ID NO: 398 - DNA, 20 bp, synthetic construct
  source 1..20: mol_type = other DNA
ctgtgcatctctcgagccac

SEQ ID NO: 399 - DNA, 20 bp, synthetic construct
  source 1..20: mol_type = other DNA
gtgctgtgccagcgacaggc

SEQ ID NO: 400 - DNA, 20 bp, synthetic construct
  source 1..20: mol_type = other DNA
gtccatgttgtgcggcaacc

SEQ ID NO: 401 - DNA, 20 bp, synthetic construct
  source 1..20: mol_type = other DNA
aggtcactgcccaggtggtt

SEQ ID NO: 402 - DNA, 20 bp, synthetic construct
  source 1..20: mol_type = other DNA
tccatccaccacttccggcc

SEQ ID NO: 403 - DNA, 20 bp, synthetic construct
  source 1..20: mol_type = other DNA
acctccatgcagcaggccag

SEQ ID NO: 404 - DNA, 20 bp, synthetic construct
  source 1..20: mol_type = other DNA
cacctccatgcagcaggcca

SEQ ID NO: 405 - DNA, 20 bp, synthetic construct
  source 1..20: mol_type = other DNA
ccacctccatgcagcaggcc

SEQ ID NO: 406 - DNA, 20 bp, synthetic construct
  source 1..20: mol_type = other DNA
gccacctccatgcagcaggc

SEQ ID NO: 407 - DNA, 20 bp, synthetic construct
  source 1..20: mol_type = other DNA
tgccacctccatgcagcagg

SEQ ID NO: 408 - DNA, 20 bp, synthetic construct
  source 1..20: mol_type = other DNA
caccaccttgcggctgatga

SEQ ID NO: 409 - DNA, 20 bp, synthetic construct
  source 1..20: mol_type = other DNA
ttcaccaccttgcggctgat

SEQ ID NO: 410 - DNA, 20 bp, synthetic construct
  source 1..20: mol_type = other DNA
ctgctgtctgtctccgttgt

SEQ ID NO: 411 - DNA, 20 bp, synthetic construct
  source 1..20: mol_type = other DNA
acctgctgtctgtctccgtt

SEQ ID NO: 412 - DNA, 20 bp, synthetic construct
  source 1..20: mol_type = other DNA
gcacctgctgtctgtctccg

SEQ ID NO: 413 - DNA, 20 bp, synthetic construct
  source 1..20: mol_type = other DNA
tgttgtacatgtgcagtgtg

SEQ ID NO: 414 - DNA, 20 bp, synthetic construct
  source 1..20: mol_type = other DNA
cggtgttgtacatgtgcagt

SEQ ID NO: 415 - DNA, 20 bp, synthetic construct
  source 1..20: mol_type = other DNA
gaccaggtcagtgaagagca

SEQ ID NO: 416 - DNA, 20 bp, synthetic construct
  source 1..20: mol_type = other DNA
tccaggaccaggtcagtgaa

SEQ ID NO: 417 - DNA, 20 bp, synthetic construct
  source 1..20: mol_type = other DNA
cttccaggaccaggtcagtg

SEQ ID NO: 418 - DNA, 20 bp, synthetic construct
  source 1..20: mol_type = other DNA
ctccccttcgctcagcaggc

SEQ ID NO: 419 - DNA, 20 bp, synthetic construct
  source 1..20: mol_type = other DNA
actccccttcgctcagcagg

SEQ ID NO: 420 - DNA, 20 bp, synthetic construct
  source 1..20: mol_type = other DNA
aactccccttcgctcagcag

SEQ ID NO: 421 - DNA, 20 bp, synthetic construct
  source 1..20: mol_type = other DNA
gcccaggttatgggccaact

SEQ ID NO: 422 - DNA, 20 bp, synthetic construct
  source 1..20: mol_type = other DNA
ctgttgtgcaggggcctcca

SEQ ID NO: 423 - DNA, 20 bp, synthetic construct
  source 1..20: mol_type = other DNA
caattgcttgcccagctgta

SEQ ID NO: 424 - DNA, 20 bp, synthetic construct
  source 1..20: mol_type = other DNA
ccaattgcttgcccagctgt

SEQ ID NO: 425 - DNA, 20 bp, synthetic construct
  source 1..20: mol_type = other DNA
cgccaattgcttgcccagct

SEQ ID NO: 426 - DNA, 20 bp, synthetic construct
  source 1..20: mol_type = other DNA
ccgccaattgcttgcccagc

SEQ ID NO: 427 - DNA, 20 bp, synthetic construct
  source 1..20: mol_type = other DNA
tccgccaattgcttgcccag

SEQ ID NO: 428 - DNA, 20 bp, synthetic construct
  source 1..20: mol_type = other DNA
ctccgccaattgcttgccca

SEQ ID NO: 429 - DNA, 20 bp, synthetic construct
  source 1..20: mol_type = other DNA
gctccgccaattgcttgccc

SEQ ID NO: 430 - DNA, 20 bp, synthetic construct
  source 1..20: mol_type = other DNA
agctccgccaattgcttgcc

SEQ ID NO: 431 - DNA, 20 bp, synthetic construct
  source 1..20: mol_type = other DNA
agggcattgctccagcagac

SEQ ID NO: 432 - DNA, 20 bp, synthetic construct
  source 1..20: mol_type = other DNA
cgcagttccccaactgtgtg

SEQ ID NO: 433 - DNA, 20 bp, synthetic construct
  source 1..20: mol_type = other DNA
gcgcagttccccaactgtgt

SEQ ID NO: 434 - DNA, 20 bp, synthetic construct
  source 1..20: mol_type = other DNA
ctctcgagccaccttgaggc

SEQ ID NO: 435 - DNA, 20 bp, synthetic construct
  source 1..20: mol_type = other DNA
actgtgcatctctcgagcca

SEQ ID NO: 436 - DNA, 20 bp, synthetic construct
  source 1..20: mol_type = other DNA
gactgtgcatctctcgagcc

SEQ ID NO: 437 - DNA, 20 bp, synthetic construct
  source 1..20: mol_type = other DNA
agactgtgcatctctcgagc

SEQ ID NO: 438 - DNA, 20 bp, synthetic construct
  source 1..20: mol_type = other DNA
cagactgtgcatctctcgag

SEQ ID NO: 439 - DNA, 20 bp, synthetic construct
  source 1..20: mol_type = other DNA
gcagactgtgcatctctcga

SEQ ID NO: 440 - DNA, 20 bp, synthetic construct
  source 1..20: mol_type = other DNA
gcggaagtccatgttgtgcg

SEQ ID NO: 441 - DNA, 20 bp, synthetic construct
  source 1..20: mol_type = other DNA
ccgcggaagtccatgttgtg

SEQ ID NO: 442 - DNA, 20 bp, synthetic construct
  source 1..20: mol_type = other DNA
cgctaggtcactgcccaggt

SEQ ID NO: 443 - DNA, 20 bp, synthetic construct
  source 1..20: mol_type = other DNA
gcgctaggtcactgcccagg

SEQ ID NO: 444 - DNA, 20 bp, synthetic construct
  source 1..20: mol_type = other DNA
cgcgctaggtcactgcccag

SEQ ID NO: 445 - DNA, 20 bp, synthetic construct
  source 1..20: mol_type = other DNA
ccggccctcagcaaactcca

SEQ ID NO: 446 - DNA, 20 bp, synthetic construct
  source 1..20: mol_type = other DNA
ttccatccaccacttccggc

SEQ ID NO: 447 - DNA, 20 bp, synthetic construct
  source 1..20: mol_type = other DNA
gcttccatccaccacttccg

SEQ ID NO: 448 - DNA, 20 bp, synthetic construct
  source 1..20: mol_type = other DNA
agcttccatccaccacttcc

SEQ ID NO: 449 - DNA, 20 bp, synthetic construct
  source 1..20: mol_type = other DNA
cagcttccatccaccacttc

SEQ ID NO: 450 - DNA, 20 bp, synthetic construct
  source 1..20: mol_type = other DNA
tcagcttccatccaccactt

SEQ ID NO: 451 - DNA, 20 bp, synthetic construct
  source 1..20: mol_type = other DNA
atcagcttccatccaccact

SEQ ID NO: 452 - DNA, 20 bp, synthetic construct
  source 1..20: mol_type = other DNA
catcagcttccatccaccac

SEQ ID NO: 453 - DNA, 20 bp, synthetic construct
  source 1..20: mol_type = other DNA
tcatcagcttccatccacca

SEQ ID NO: 454 - DNA, 20 bp, synthetic construct
  source 1..20: mol_type = other DNA
gctcatcagcttccatccac

SEQ ID NO: 455 - DNA, 20 bp, synthetic construct
  source 1..20: mol_type = other DNA
ggctcatcagcttccatcca

SEQ ID NO: 456 - DNA, 20 bp, synthetic construct
  source 1..20: mol_type = other DNA
gtgccacctccatgcagcag

SEQ ID NO: 457 - DNA, 20 bp, synthetic construct
  source 1..20: mol_type = other DNA
tgtgccacctccatgcagca

SEQ ID NO: 458 - DNA, 20 bp, synthetic construct
  source 1..20: mol_type = other DNA
gtgtgccacctccatgcagc

SEQ ID NO: 459 - DNA, 20 bp, synthetic construct
  source 1..20: mol_type = other DNA
cgtgtgccacctccatgcag

SEQ ID NO: 460 - DNA, 20 bp, synthetic construct
  source 1..20: mol_type = other DNA
gcgtgtgccacctccatgca

SEQ ID NO: 461 - DNA, 20 bp, synthetic construct
  source 1..20: mol_type = other DNA
tgcgtgtgccacctccatgc

SEQ ID NO: 462 - DNA, 20 bp, synthetic construct
  source 1..20: mol_type = other DNA
cagtccacttgcgtgcccac

SEQ ID NO: 463 - DNA, 20 bp, synthetic construct
  source 1..20: mol_type = other DNA
gggcagtccacttgcgtgcc

SEQ ID NO: 464 - DNA, 20 bp, synthetic construct
  source 1..20: mol_type = other DNA
gccatgggcagtccacttgc

SEQ ID NO: 465 - DNA, 20 bp, synthetic construct
  source 1..20: mol_type = other DNA
cacaggtcaccccagaaact

SEQ ID NO: 466 - DNA, 20 bp, synthetic construct
  source 1..20: mol_type = other DNA
cacccacactgtctcggcct

SEQ ID NO: 467 - DNA, 20 bp, synthetic construct
  source 1..20: mol_type = other DNA
gcacccacactgtctcggcc

SEQ ID NO: 468 - DNA, 20 bp, synthetic construct
  source 1..20: mol_type = other DNA
tgttgccacctccctgtaca

SEQ ID NO: 469 - DNA, 20 bp, synthetic construct
  source 1..20: mol_type = other DNA
tgtgttgccacctccctgta

SEQ ID NO: 470 - DNA, 20 bp, synthetic construct
  source 1..20: mol_type = other DNA
ctgtgttgccacctccctgt

SEQ ID NO: 471 - DNA, 20 bp, synthetic construct
  source 1..20: mol_type = other DNA
ctggcacccacctgtgttgc

SEQ ID NO: 472 - DNA, 20 bp, synthetic construct
  source 1..20: mol_type = other DNA
cactccacaggccatccggc

SEQ ID NO: 473 - DNA, 20 bp, synthetic construct
  source 1..20: mol_type = other DNA
cccactccacaggccatccg

SEQ ID NO: 474 - DNA, 20 bp, synthetic construct
  source 1..20: mol_type = other DNA
gacccactccacaggccatc

SEQ ID NO: 475 - DNA, 20 bp, synthetic construct
  source 1..20: mol_type = other DNA
ccaccttttacctgcagacc

SEQ ID NO: 476 - DNA, 20 bp, synthetic construct
  source 1..20: mol_type = other DNA
gccaccttttacctgcagac

SEQ ID NO: 477 - DNA, 20 bp, synthetic construct
  source 1..20: mol_type = other DNA
ggccaccttttacctgcaga

SEQ ID NO: 478 - DNA, 20 bp, synthetic construct
  source 1..20: mol_type = other DNA
ctccccgcagtacaggactc

SEQ ID NO: 479 - DNA, 20 bp, synthetic construct
  source 1..20: mol_type = other DNA
cctccccgcagtacaggact

SEQ ID NO: 480 - DNA, 20 bp, synthetic construct
  source 1..20: mol_type = other DNA
ccctccccgcagtacaggac

SEQ ID NO: 481 - DNA, 20 bp, synthetic construct
  source 1..20: mol_type = other DNA
ctcccctccccgcagtacag

SEQ ID NO: 482 - DNA, 20 bp, synthetic construct
  source 1..20: mol_type = other DNA
tcctgcccatccctccatcc

SEQ ID NO: 483 - DNA, 20 bp, synthetic construct
  source 1..20: mol_type = other DNA
ttcctgcccatccctccatc

SEQ ID NO: 484 - DNA, 20 bp, synthetic construct
  source 1..20: mol_type = other DNA
cctttcctgcccatccctcc

SEQ ID NO: 485 - DNA, 20 bp, synthetic construct
  source 1..20: mol_type = other DNA
gcatgtgtccagaagcagtc

SEQ ID NO: 486 - DNA, 20 bp, synthetic construct
  source 1..20: mol_type = other DNA
ttgcgcaccgccagaaagtg

SEQ ID NO: 487 - DNA, 20 bp, Mus sp.
  source 1..20: mol_type = genomic DNA
tgcacatgtacaacaccgtg

SEQ ID NO: 488 - DNA, 20 bp, Mus sp.
  source 1..20: mol_type = genomic DNA
cacatgtacaacaccgtgat

SEQ ID NO: 489 - DNA, 20 bp, Mus sp.
  source 1..20: mol_type = genomic DNA
acatgtacaacaccgtgatg

SEQ ID NO: 490 - DNA, 20 bp, Mus sp.
  source 1..20: mol_type = genomic DNA
aggtggctcgagagatgcac

SEQ ID NO: 491 - DNA, 20 bp, Mus sp.
  source 1..20: mol_type = genomic DNA
ggtggctcgagagatgcaca

SEQ ID NO: 492 - DNA, 20 bp, Mus sp.
  source 1..20: mol_type = genomic DNA
gtggctcgagagatgcacag

SEQ ID NO: 493 - DNA, 20 bp, Mus sp.
  source 1..20: mol_type = genomic DNA
gcctgtcgctggcacagcac

SEQ ID NO: 494 - DNA, 20 bp, Mus sp.
  source 1..20: mol_type = genomic DNA
ggttgccgcacaacatggac

SEQ ID NO: 495 - DNA, 20 bp, Mus sp.
  source 1..20: mol_type = genomic DNA
aaccacctgggcagtgacct

SEQ ID NO: 496 - DNA, 20 bp, Mus sp.
  source 1..20: mol_type = genomic DNA
ggccggaagtggtggatgga

SEQ ID NO: 497 - DNA, 20 bp, Mus sp.
  source 1..20: mol_type = genomic DNA
ctggcctgctgcatggaggt

SEQ ID NO: 498 - DNA, 20 bp, Mus sp.
  source 1..20: mol_type = genomic DNA
tggcctgctgcatggaggtg

SEQ ID NO: 499 - DNA, 20 bp, Mus sp.
  source 1..20: mol_type = genomic DNA
ggcctgctgcatggaggtgg

SEQ ID NO: 500 - DNA, 20 bp, Mus sp.
  source 1..20: mol_type = genomic DNA
gcctgctgcatggaggtggc

SEQ ID NO: 501 - DNA, 20 bp, Mus sp.
  source 1..20: mol_type = genomic DNA
cctgctgcatggaggtggca

SEQ ID NO: 502 - DNA, 20 bp, Mus sp.
  source 1..20: mol_type = genomic DNA
tcatcagccgcaaggtggtg

SEQ ID NO: 503 - DNA, 20 bp, Mus sp.
  source 1..20: mol_type = genomic DNA
atcagccgcaaggtggtgaa

SEQ ID NO: 504 - DNA, 20 bp, Mus sp.
  source 1..20: mol_type = genomic DNA
acaacggagacagacagcag

SEQ ID NO: 505 - DNA, 20 bp, Mus sp.
  source 1..20: mol_type = genomic DNA
aacggagacagacagcaggt

SEQ ID NO: 506 - DNA, 20 bp, Mus sp.
  source 1..20: mol_type = genomic DNA
cggagacagacagcaggtgc

SEQ ID NO: 507 - DNA, 20 bp, Mus sp.
  source 1..20: mol_type = genomic DNA
cacactgcacatgtacaaca

SEQ ID NO: 508 - DNA, 20 bp, Mus sp.
  source 1..20: mol_type = genomic DNA
actgcacatgtacaacaccg

SEQ ID NO: 509 - DNA, 20 bp, Mus sp.
  source 1..20: mol_type = genomic DNA
tgctcttcactgacctggtc

SEQ ID NO: 510 - DNA, 20 bp, Mus sp.
  source 1..20: mol_type = genomic DNA
ttcactgacctggtcctgga

SEQ ID NO: 511 - DNA, 20 bp, Mus sp.
  source 1..20: mol_type = genomic DNA
cactgacctggtcctggaag

SEQ ID NO: 512 - DNA, 20 bp, Mus sp.
  source 1..20: mol_type = genomic DNA
gcctgctgagcgaaggggag

SEQ ID NO: 513 - DNA, 20 bp, Mus sp.
  source 1..20: mol_type = genomic DNA
cctgctgagcgaaggggagt

SEQ ID NO: 514 - DNA, 20 bp, Mus sp.
  source 1..20: mol_type = genomic DNA
ctgctgagcgaaggggagtt

SEQ ID NO: 515 - DNA, 20 bp, Mus sp.
  source 1..20: mol_type = genomic DNA
agttggcccataacctgggc

SEQ ID NO: 516 - DNA, 20 bp, Mus sp.
  source 1..20: mol_type = genomic DNA
tggaggcccctgcacaacag

SEQ ID NO: 517 - DNA, 20 bp, Mus sp.
  source 1..20: mol_type = genomic DNA
tacagctgggcaagcaattg

SEQ ID NO: 518 - DNA, 20 bp, Mus sp.
  source 1..20: mol_type = genomic DNA
acagctgggcaagcaattgg

SEQ ID NO: 519 - DNA, 20 bp, Mus sp.
  source 1..20: mol_type = genomic DNA
agctgggcaagcaattggcg

SEQ ID NO: 520 - DNA, 20 bp, Mus sp.
  source 1..20: mol_type = genomic DNA
gctgggcaagcaattggcgg

SEQ ID NO: 521 - DNA, 20 bp, Mus sp.
  source 1..20: mol_type = genomic DNA
ctgggcaagcaattggcgga

SEQ ID NO: 522 - DNA, 20 bp, Mus sp.
  source 1..20: mol_type = genomic DNA
tgggcaagcaattggcggag

SEQ ID NO: 523 - DNA, 20 bp, Mus sp.
  source 1..20: mol_type = genomic DNA
gggcaagcaattggcggagc

SEQ ID NO: 524 - DNA, 20 bp, Mus sp.
  source 1..20: mol_type = genomic DNA
ggcaagcaattggcggagct

SEQ ID NO: 525 - DNA, 20 bp, Mus sp.
  source 1..20: mol_type = genomic DNA
gtctgctggagcaatgccct

SEQ ID NO: 526 - DNA, 20 bp, Mus sp.
  source 1..20: mol_type = genomic DNA
cacacagttggggaactgcg

SEQ ID NO: 527 - DNA, 20 bp, Mus sp.
  source 1..20: mol_type = genomic DNA
acacagttggggaactgcgc

SEQ ID NO: 528 - DNA, 20 bp, Mus sp.
  source 1..20: mol_type = genomic DNA
gcctcaaggtggctcgagag

SEQ ID NO: 529 - DNA, 20 bp, Mus sp.
  source 1..20: mol_type = genomic DNA
tggctcgagagatgcacagt

SEQ ID NO: 530 - DNA, 20 bp, Mus sp.
  source 1..20: mol_type = genomic DNA
ggctcgagagatgcacagtc

SEQ ID NO: 531 - DNA, 20 bp, Mus sp.
  source 1..20: mol_type = genomic DNA
gctcgagagatgcacagtct

SEQ ID NO: 532 - DNA, 20 bp, Mus sp.
  source 1..20: mol_type = genomic DNA
ctcgagagatgcacagtctg

SEQ ID NO: 533 - DNA, 20 bp, Mus sp.
  source 1..20: mol_type = genomic DNA
tcgagagatgcacagtctgc

SEQ ID NO: 534 - DNA, 20 bp, Mus sp.
  source 1..20: mol_type = genomic DNA
cgcacaacatggacttccgc

SEQ ID NO: 535 - DNA, 20 bp, Mus sp.
  source 1..20: mol_type = genomic DNA
cacaacatggacttccgcgg

SEQ ID NO: 536 - DNA, 20 bp, Mus sp.
  source 1..20: mol_type = genomic DNA
acctgggcagtgacctagcg

SEQ ID NO: 537 - DNA, 20 bp, Mus sp.
  source 1..20: mol_type = genomic DNA
cctgggcagtgacctagcgc

SEQ ID NO: 538 - DNA, 20 bp, Mus sp.
  source 1..20: mol_type = genomic DNA
ctgggcagtgacctagcgcg

SEQ ID NO: 539 - DNA, 20 bp, Mus sp.
  source 1..20: mol_type = genomic DNA
tggagtttgctgagggccgg

SEQ ID NO: 540 - DNA, 20 bp, Mus sp.
  source 1..20: mol_type = genomic DNA
gccggaagtggtggatggaa

SEQ ID NO: 541 - DNA, 20 bp, Mus sp.
  source 1..20: mol_type = genomic DNA
cggaagtggtggatggaagc

SEQ ID NO: 542 - DNA, 20 bp, Mus sp.
  source 1..20: mol_type = genomic DNA
ggaagtggtggatggaagct

SEQ ID NO: 543 - DNA, 20 bp, Mus sp.
  source 1..20: mol_type = genomic DNA
gaagtggtggatggaagctg

SEQ ID NO: 544 - DNA, 20 bp, Mus sp.
  source 1..20: mol_type = genomic DNA
aagtggtggatggaagctga

SEQ ID NO: 545 - DNA, 20 bp, Mus sp.
  source 1..20: mol_type = genomic DNA
agtggtggatggaagctgat

SEQ ID NO: 546 - DNA, 20 bp, Mus sp.
  source 1..20: mol_type = genomic DNA
gtggtggatggaagctgatg

SEQ ID NO: 547 - DNA, 20 bp, Mus sp.
  source 1..20: mol_type = genomic DNA
tggtggatggaagctgatga

SEQ ID NO: 548 - DNA, 20 bp, Mus sp.
  source 1..20: mol_type = genomic DNA
gtggatggaagctgatgagc

SEQ ID NO: 549 - DNA, 20 bp, Mus sp.
  source 1..20: mol_type = genomic DNA
tggatggaagctgatgagcc

SEQ ID NO: 550 - DNA, 20 bp, Mus sp.
  source 1..20: mol_type = genomic DNA
ctgctgcatggaggtggcac

SEQ ID NO: 551 - DNA, 20 bp, Mus sp.
  source 1..20: mol_type = genomic DNA
tgctgcatggaggtggcaca

SEQ ID NO: 552 - DNA, 20 bp, Mus sp.
  source 1..20: mol_type = genomic DNA
gctgcatggaggtggcacac

SEQ ID NO: 553 - DNA, 20 bp, Mus sp.
  source 1..20: mol_type = genomic DNA
ctgcatggaggtggcacacg

SEQ ID NO: 554 - DNA, 20 bp, Mus sp.
  source 1..20: mol_type = genomic DNA
tgcatggaggtggcacacgc

SEQ ID NO: 555 - DNA, 20 bp, Mus sp.
  source 1..20: mol_type = genomic DNA
gcatggaggtggcacacgca

SEQ ID NO: 556 - DNA, 20 bp, Mus sp.
  source 1..20: mol_type = genomic DNA
gtgggcacgcaagtggactg

SEQ ID NO: 557 - DNA, 20 bp, Mus sp.
  source 1..20: mol_type = genomic DNA
ggcacgcaagtggactgccc

SEQ ID NO: 558 - DNA, 20 bp, Mus sp.
  source 1..20: mol_type = genomic DNA
gcaagtggactgcccatggc

SEQ ID NO: 559 - DNA, 20 bp, Mus sp.
  source 1..20: mol_type = genomic DNA
agtttctggggtgacctgtg

SEQ ID NO: 560 - DNA, 20 bp, Mus sp.
  source 1..20: mol_type = genomic DNA
aggccgagacagtgtgggtg

SEQ ID NO: 561 - DNA, 20 bp, Mus sp.
  source 1..20: mol_type = genomic DNA
ggccgagacagtgtgggtgc

SEQ ID NO: 562 - DNA, 20 bp, Mus sp.
  source 1..20: mol_type = genomic DNA
tgtacagggaggtggcaaca

SEQ ID NO: 563 - DNA, 20 bp, Mus sp.
  source 1..20: mol_type = genomic DNA
tacagggaggtggcaacaca

SEQ ID NO: 564 - DNA, 20 bp, Mus sp.
  source 1..20: mol_type = genomic DNA
acagggaggtggcaacacag

SEQ ID NO: 565 - DNA, 20 bp, Mus sp.
  source 1..20: mol_type = genomic DNA
gcaacacaggtgggtgccag

SEQ ID NO: 566 - DNA, 20 bp, Mus sp.
  source 1..20: mol_type = genomic DNA
gccggatggcctgtggagtg

SEQ ID NO: 567 - DNA, 20 bp, Mus sp.
  source 1..20: mol_type = genomic DNA
cggatggcctgtggagtggg

SEQ ID NO: 568 - DNA, 20 bp, Mus sp.
  source 1..20: mol_type = genomic DNA
gatggcctgtggagtgggtc

SEQ ID NO: 569 - DNA, 20 bp, Mus sp.
  source 1..20: mol_type = genomic DNA
ggtctgcaggtaaaaggtgg

SEQ ID NO: 570 - DNA, 20 bp, Mus sp.
  source 1..20: mol_type = genomic DNA
gtctgcaggtaaaaggtggc

SEQ ID NO: 571 - DNA, 20 bp, Mus sp.
  source 1..20: mol_type = genomic DNA
tctgcaggtaaaaggtggcc

SEQ ID NO: 572 - DNA, 20 bp, Mus sp.
  source 1..20: mol_type = genomic DNA
gagtcctgtactgcggggag

SEQ ID NO: 573 - DNA, 20 bp, Mus sp.
  source 1..20: mol_type = genomic DNA
agtcctgtactgcggggagg

SEQ ID NO: 574 - DNA, 20 bp, Mus sp.
  source 1..20: mol_type = genomic DNA
gtcctgtactgcggggaggg

SEQ ID NO: 575 - DNA, 20 bp, Mus sp.
  source 1..20: mol_type = genomic DNA
ctgtactgcggggaggggag

SEQ ID NO: 576 - DNA, 20 bp, Mus sp.
  source 1..20: mol_type = genomic DNA
ggatggagggatgggcagga

SEQ ID NO: 577 - DNA, 20 bp, Mus sp.
  source 1..20: mol_type = genomic DNA
gatggagggatgggcaggaa

SEQ ID NO: 578 - DNA, 20 bp, Mus sp.
  source 1..20: mol_type = genomic DNA
ggagggatgggcaggaaagg

SEQ ID NO: 579 - DNA, 20 bp, Mus sp.
  source 1..20: mol_type = genomic DNA
gactgcttctggacacatgc

SEQ ID NO: 580 - DNA, 20 bp, Mus sp.
  source 1..20: mol_type = genomic DNA
cactttctggcggtgcgcaa

SEQ ID NO: 581 - DNA, 10459 bp, Mus sp.
  source 1..10459: mol_type = genomic DNA
ctcattactttggtgggtttgcgcaccgccagaaagtggcggctggcataggtagacacaaactttcatttatttacattatgcacaggtttagggaccgcatggcgtgggtcagctgaaaaagtaggtggatcttatcacctgtcctagatcgaaggtacctgtaaagccaagagtgcagaactaggctcgggccactgattcatcctcaaggccagcgtgcatgtcccctttaaatccttacctgtctttggcaaggactgtagtgtctcctgcagcttggtgaccagggcgcgctccgaggactttagggacttgatgctgcaaacaggttagggtagacatcagtgcggcagcaggtggcaggcagggcaccaggccacaagagggggtatctcacctggacacggagcagaaacgcttcttcaggaacttggccaggtcttccaggatgggctggctgtgcaggcgcacgaattgctcgcggcatacctgaggggttgcaggctcggagtcagccatggtacaaattcccaggatcaaggccccattttctacttgtggggtcacgggggggggggggggggcgggacgggacggacgggacacacctcgttcatcgtggggatgtcagcggcatgtgtccagaagcagtcgtgcacggagacgaaaatcaggcccttcctgagcagggagatcccgagtgagatagctagttgcttggccagctactgcctgaacctcgtctgacagatatcctgtcatctgccccagcactgggcactctgccagggttccctggacacctttggttccccagtgctctaggtagtagatgttcaagaagatggcttacattccctcaaacagctgcggggtcttggagtaaagggaagcccctgtctgcaccaaggactcccgcaactttccagcagtgcctccatcctgtcctgcccctcatcatgggggtacctaccatgtccatgtccccagaaagcagaacaaggctggcatgccccaggcagctaaataccacagactgaaacctagacccagcgctcacctgaagcaagagcctgcctgcctgcccgccctgctgggcccacactgtgtcctggtcacacttcgtcctgggacccacctgtagcagtgtagggcggtcagcatcatgtgggaggagtccagggagtggatgaagttgggcgggaagccatttttctgcttcagagtgttgggcttcctgtggacagaaattgtaggctggtaccctcttagcccagagcctttcctgcccatccctccatcctctcccctccccgcagtacaggactcactgactctcatctaccgagctggtgagggtgatgctctggaggccaccttttacctgcagaccggggttggaggaggggtgagtccagcccttggcagggaggaggggagatgagggaaaaggagaagggcatttcccaagtacctggaccttggactcgcggtgatagggctgtatgatggggatgcccaggggcgtgacccactccacaggccatccggcgtgagagatgaggttggcactctcagtcagccagtgctgtgggtgtgacagtacaccaggttgggtgggtgagctgtggggtggaggggcccccagatgggagagggctggcagggacgcacctgaatggcccgcgtgctggtgaacatctcctgtagacttttgaagacctggcgcacgaggtagtgtgaggcttcccagacaaactcctgtggggacattgcgatatgaatggcaggtgggcggggccagaaccgcggctttacaggactagaacggttcagaagagcccggctcagagtccctgtgctgaagggagtcacacttgggtgaggacagggcaggctgcacacctgagggaagtcgctgagttcgcgcaggcgcttctctatctgcaggcgccctccgtagcgtgtgaccccatacaccaccgtcatcactgtctgcttcaccaccttgcggctgatgaagccctccagcacctgagccacccgtagaccctccttggcgtcctgctggcggaactcctccacctggatgggattaggggtcagggaagccattacgtgacctcccctccccactaaggaccccatcacttgtagaaaggctaccccgagtggggacaggaggggtttcctggcacccacctgtgttgccacctccctgtacacatcttggggcaggtcggacggcgttaggttgactgaggcagcacccacactgtctcggcctagtgcggcgtaatgctgtaagccattgcaggagccatcctgaccacaaggggtgcccatgagctgaggccaaatgacccaatagggacacacaggtcaccccagaaacttattccaccacctggtgcaaggagacccgggagcaacctccctcataaatcaagagactagtcccacagggcaatcccagtttacggtaagacccaactgcaaatgccagttcctaaggcacctcgggctcagccatgggcagtccacttgcgtgcccacctggtgaactggcaggtgggaaatgtaggcagcagggtctggggaccggactgcgtgtgccacctccatgcagcaggccagggtctgccagggctcatcagcttccatccaccacttccggccctttggagggtaggatccatgggaatcgagtcagcacaagccagcctgggcagctcagtggaacacagggacgtagggacactaggcaagcatgtgtgatgtagggggatgaggaaggctgctcgggtgatagctggcagctgctgctgggctagggagccactagtggcactaagaggggacaccgtgggaaggaaggatgcagatagctgtggtgaagggttgaaagcatatgagggaggtggcccaagcctgagtggatgacaggctggcagagccagagaaagacaggaggcagagatgagggctgccagcgagaggagctcgtgactccataccgtcaaggggttgtctgcagagtccaagatctcctccatgacctcatctgcgaaggctaggcgcatgcgcagcgagtctcccttcttgaggccagtcaggttgatcaggtggatcttaagccagtccagaccacgtggtcccagtggccggccctcagcaaactccaatagcgcacgcgctaggtcactgcccaggtggttgaagtgtggcgggcaggggtaagtgcggccgcggaagtccatgttgtgcggcaaccagaagacacggtggcgtaggtgctgtgccagcgacaggcgatacagggcctcgctgcgcagactgtgcatctctcgagccaccttgaggcaacgcgccagctccttccgcagctcagacttgtgcactggtgtggagccgggtggcagctgataccgggccggccgcggtgcttctgagcgcgggggaggcacgcccaagggcatacagcccttgtctctaaagatctgcagcaccaagtccagcagatgcccgtttacacgccaggcgcagttccccaactgtgtgagcgcatccagggggccgtgcagctgggcaggagggcattgctccagcagacgctggtgctgtgtggtaccctccgtggcacgcattagtttggtggagctcagcaagtaggcaccagaatgcagcgacgtccagggcagtggtgggcacagcatgggcacttccgtggtctcgaaggtcagtgtgggctctgctgccgtctctagcaggtgcgtgaaggcagggtgaggcttaaggatgcccacctgggagaggaagcagacacaaatggtatgcatcagaggccgagtgccagtgcctctgtgcaacggaccacgcaggcagggagaacagaaaactccaagccctagactgatgtaacccctcctgctctgtacacacatcccacacggaggggatggcagacacctgctgccctatgtcccatccaggcgccggccggccctgggagcctcacctggcggtagcttcggaaggaatacacatggtaaagcactgggatggagcgctgagcaccctgccgggccgccaggctgcgcggcatctgcactgcctgcaccagcagctccgccaattgcttgcccagctgtagcagcactggcacggaccagggctgttgtgcaggggcctccagtgggcccagcgattcccagtactcccgaggaaggcagggcgccacctgtaaggacagctggtcaggtgagctgtggggctgcccacgttttccagagggaaccgaggctcagacggactgatgcagctggcaaggggcgcctgaggagtgtggtggggagggacctgggtggaacctggagcaagtggggtgaagtgaactgggaggggatgtctccaagggagccacctgggtcaccagaagtccatcccacgttacctgggtgtcagacgccagcagctggaggtactgcgagtaccgctgccccagcttctgcacatggttagtgacctgcttctgcttcaccaaatgccggtttaacacccgaaggcccaggttatgggccaactggataaggggctcaccctgcgcaggcaggaccttcagaacctgaggtgcaagaggatcgcgagctgtgactgcctgaacacagccatctgccctgtgcccaggaacctccagacccagcccacctgcatcagtatgctcacaaactccccttcgctcagcaggcacaggaacgggtagagggtgggctggccttcataggcctggcggcccatagtggcctttgtctcccgcagcacgcgcaacagctccacttcccactgctcccgcagggcctgcagggtcttccgctgtaggagagcagctgtcaacacgctgagcttaaagccccctgccaagacctcccaaggccccaactggggtcatagctccgacccctactggccatgatgggtacagcctcaggatgagacccttaccgcctcaatgacctccttggacattactggagccttctccaccgactggacacagacactggagctcagctccacatgcagctgctggtagaagaggtcctgcagggtgtccaggggcaggtgcagctttgggtaggacacagggccctcctgctgggcacaggaggcagtgagggtgggcactacaccctgtcaagaagccccctgccaaacgcatgtccactcaaagtaaaaacccagagtactaagagttcagacacctgactcccagtaaaaacagcccaatgccccgaacagatgatggatacacaggaggccccatctacacactggaatacaactgagccatgagtaggagcgagcttccaacatattccgcagcgtgaagggaccttgaggacactgtaatcgatgagccagatacaaaaggccacagggactagggctctatgatagcaaacgcccagaacaggagaatccagaggaagtggatttgggggtgctcctctgaggggaccaacatgtgcctgatgcaaggagggaatgaaacaggctgtcatgacttcttgcctgggactcaccttgctatatatgtccttgagcagcgtggatgtgttaactgggcttggggcctgtggcggtggacgaaaggcaggctcagccttgaccactgctctcaggagcgcggcccggtcctcctcttccaggaccaggtcagtgaagagcagctggggctggaagccttcttccatcatctgcttcagacacctgaggcaatacaggggtgggggtgggggtgtgcaaacttagcatgggagcatggggtggggggagcagggccccagcacattcctgacccacctctggatggtgcgaacatcctggtccctgcgtcccatgcactggagtgcagctgcataggagcacaggtctggggagaggccagcatccttcagcatgaggaacacatagaccagctctctgaaggagccctggggacaaggtggggtcaagaacaacacctggaccctgaccaatctgttccctaaaatcctcagtaatttgggaggggggatctggcagtaggtgagacacagggcatctttctgagggacactgcgtcattggggactgaactcgggcctcttgcaggccaggcatatgctctaccactaagctacagcttcaggacttgtgttttatagctggcttggtttttgttgtggttctttgtttgtttgttgagaacttgtctcactgtgtagccctggctggcctgaaactctctaagtaggtcagactggccttgaactcacagacctgtctcctgtctactgctctgtcctttccttgtttttattttgcttttctgtctttgaactcctagactgacactctcctacactgctaagatgttagggaacaaactgaggacacacagctctcatagcaccaaagcgacacacagaaagagaagcgccgggcgtggtagcgcacgcctttgatcccagcactcgggaggcagaagcaggcggatttctgagttcgaggccagcctggtctacagagtgagttccaggacagccagggctacacagagaaaccctgtcttgaaaaaccaaaaaaaaaaataaaaataaaaaaaaaataaaaataaaaaaaaaataaagagaagcaagcgctcccatcctggagcgtggaatgtgtcccacaaggcttagcagccactgggacagcagcggcagcccctgcacaggagactcagaagtcctgcacagaactacactcaggccctcccaatctcctccttcattttaggggatagcaggattttaatgtgttgaccaggctgccctctaactctcagagatccacctgcctctgcctcccaagtgctgggactaaaggcttgcgccaccatgcctggatattccaatcggtttttgttttgtttcctgtttgttcgtttttaaggaaagagcgttttctctctgaaaaacctaaaggaatttaccccatgggaaggaagcagcccttgggccgggtctcaccttgcgggcccagccaagcatcacggtgttgtacatgtgcagtgtgagcacctgctgtctgtctccgttgttatggtgagtgaccagcacgtggtgagcgaggggcacttggccagtgcagacgcagcactcaaagaaagccaggaacttctgctggaggaccaggatctgcgcctccaccgccttggctttcttcctggccagggcctctgcctcacgggagctgagcctccccagagcagcctgaagggcctgggccagctgcttttcctctgagttcgtgggtgttcccttcttgcttggctgcaggtagccagccagcttcttgttccagatcttgggctccttgtgaagggtccagaactcctgtgtgagggcttgcttctgctggtcaacttccttttggcgcctctgcttcacccttttctcagcctctagcttttgcgcccagcggctgctgtggccctgtggaggccggtccacctgcaccttcttcaccctcatctcaggtgtgccctctgcccggagctgccgcacccgagcctccagcactgcagagagcagagaagggtatgaaccactcctgcagctcaggatgctctgagtctaccccaggatcatcccaaacacagtctcctggctgtcaccatgccctcggctctgcctgccaacccctcctatcacctcctgccggctgtcacctgcttctgcttctccctccttcactccgccagccttaagggctccgacctcactggccaatatatagtagcctgcagtttgatacaggcacatacttgatacagcttggtagaaaccgcaaacatccgtgactcagtgccctaaggaccaaaaggcaatcactggcaccatacaaggaggcccctgctgtatggctttcagcagtaaccaagtttgcacataaaaaggccaaagactggccagggtcttggcaagcagacagccatggttagtggtgagcgcagaaacaaggcaggctgagccataaggctacaactcaggcgctggttgccaagaccagctgcttgtctcccctcccggtactccagtccctggcccaaggtcagtgtgaggcaggggcctggctcacaggtgtacaaaacgaaagttgggaggagtgcagtgcatgcgagaggatcagagttctggggagatcctgcctcagtgaacatctcgtggcactcagggcacatcagaggcagaggacacatctgtgtgaggccacctgcaaagcaaacaggtggcagacggggacaccgacttctttctgaagggacccaagagacaaaagggtgcaaaaaggccctggggcagaactggttggtctgtcaggagtagaagagttacatagctgctgtaagctccatggcccacaaagcactgccttctgtccctgacagaaaggagcctcataagagggagcaggcgccacacactaagtctgcagatccctctgcccaaatgacagattccagcatgacataccctggaacgtgagtcaggaagcagttgctgctaagggtagacaggagccacagttagaccattgtctttacctcctaacccaccctctaagcgtctccaccttctgcagctgccatgcagacctgacataggatcaggagggaaatctggacatcccgtctactagacttggtagtaaaacacttaaaattgggttccaaagtcagtggtcgtgcatgcctttattttgttttatttttgtttgtttggtttttttttttttttttttttttttggttttttctttttttcaagacagggtttctctgtgtagccctggctgtcctggaactcattttgtagaccaggctggcctcgaactcagaaatccgcctgcctctgcctccagagtgctgggatcaaaggcgtgcgccaccatacccggctcatgcacacctttaatcccagcactccaggggcagaggcaggcagacctgagttcaaggccagcctggtctacaaagcagccaggacttcctacacagagaaaccctgtctcgaaaaaccaagaaagaaaaaagagagaaagagagaagggaggaatgaggagagaaagaaagaaagaggaaggaaggaaggaagacagacaggcaggcctggcagacaggctggattccagctgcacctcgactctgcaggactaaaactggagacttctatgtcagcattccaacaatgcaaatccggtattgttcaggtgtatttggggcctccactgtcttcctagaagtgagagccccaggtgggaatctggaactcagggaatggaattacagcagctcccagaaccagcacaagaggacccacaggaccacagacaggccagccagacaacactgctcagtggactctggaccccattttacatactcccttggagtggtcaggtacaacgcagagcacactccctcaaggcttcactgtggagaaggacttaggacaccaacttaccttccagcagttcagcatggccccactccctcaggacatgttgctcacggggactggcagcggagctccttcttgagctgcagaagccaccaaaagtccctagaaagagacatgaagaataagaacacagaaaccatcctatcaagtggacaccgtggcatgcacacttattaccccaggtctctggaggctgaggtgggagggtctccaattggattccagcctgggctacaaaggaagttctaggttaagctagtgtgagttcctgcctcaaacttacgtcatgcccagaaagaaatggatcaaaagacaaggactgccaggcatgggatctcagaactcaggaggttgaagcagtaggacctgagttcaaataaagcatccagcatgaaccacatggaaacaccatggctcaaaaaaaaaaaaaaaaaaaaaaaaaaaacacgccccgcccacaaataaatccagacccagaaaaagaccaacttcaagggcagaacaggccaaagctataaggatcggggtgcaggatccagcaagaagtactcctaactaaaagtagccaaaggtcaatgccaatatccgtaagtccccaacagtgggtcagagacatggaaaagcgagcaacaatcaaaagtcaaagtgccctgcggacctagagagacgttgccaactagtgcccagctaaatgggtccccagaaactcctccataggtcaaagaacaaaggttgaaggtgaccgggatcctaaggaagggaagattatagtcctggatccgacactcctttaccttcctccgaaggcgggcggtggggcccaggggagcgcagaacccggccaagcccggctgcgcttcgggtccaccggagcgccgacatgccgcctgctgcccgcagtgcgcacgcgc

SEQ ID NO: 582 - DNA, 4288 bp, Mus sp.
  source 1..4288: mol_type = genomic DNA
gcactgcgggcagcaggcggcatgtcggcgctccggtggacccgaagcgcagccgggcttggccgggttctgcgctcccctgggccccaccgcccgccttcggaggaagggacttttggtggcttctgcagctcaagaaggagctccgctgccagtccccgtgagcaacatgtcctgagggagtggggccatgctgaactgctggaagtgctggaggctcgggtgcggcagctccgggcagagggcacacctgagatgagggtgaagaaggtgcaggtggaccggcctccacagggccacagcagccgctgggcgcaaaagctagaggctgagaaaagggtgaagcagaggcgccaaaaggaagttgaccagcagaagcaagccctcacacaggagttctggacccttcacaaggagcccaagatctggaacaagaagctggctggctacctgcagccaagcaagaagggaacacccacgaactcagaggaaaagcagctggcccaggcccttcaggctgctctggggaggctcagctcccgtgaggcagaggccctggccaggaagaaagccaaggcggtggaggcgcagatcctggtcctccagcagaagttcctggctttctttgagtgctgcgtctgcactggccaagtgcccctcgctcaccacgtgctggtcactcaccataacaacggagacagacagcaggtgctcacactgcacatgtacaacaccgtgatgcttggctgggcccgcaagggctccttcagagagctggtctatgtgttcctcatgctgaaggatgctggcctctccccagacctgtgctcctatgcagctgcactccagtgcatgggacgcagggaccaggatgttcgcaccatccagaggtgtctgaagcagatgatggaagaaggcttccagccccagctgctcttcactgacctggtcctggaagaggaggaccgggccgcgctcctgagagcagtggtcaaggctgagcctgcctttcgtccaccgccacaggccccaagcccagttaacacatccacgctgctcaaggacatatatagcaaggagggccctgtgtcctacccaaagctgcacctgcccctggacaccctgcaggacctcttctaccagcagctgcatgtggagctgagctccagtgtctgtgtccagtcggtggagaaggctccagtaatgtccaaggaggtcattgaggcgcggaagaccctgcaggccctgcgggagcagtgggaagtggagctgttgcgcgtgctgcgggagacaaaggccactatgggccgccaggcctatgaaggccagcccaccctctacccgttcctgtgcctgctgagcgaaggggagtttgtgagcatactgatgcaggttctgaaggtcctgcctgcgcagggtgagccccttatccagttggcccataacctgggccttcgggtgttaaaccggcatttggtgaagcagaagcaggtcactaaccatgtgcagaagctggggcagcggtactcgcagtacctccagctgctggcgtctgacacccaggtggcgccctgccttcctcgggagtactgggaatcgctgggcccactggaggcccctgcacaacagccctggtccgtgccagtgctgctacagctgggcaagcaattggcggagctgctggtgcaggcagtgcagatgccgcgcagcctggcggcccggcagggtgctcagcgctccatcccagtgctttaccatgtgtattccttccgaagctaccgccaggtgggcatccttaagcctcaccctgccttcacgcacctgctagagacggcagcagagcccacactgaccttcgagaccacggaagtgcccatgctgtgcccaccactgccctggacgtcgctgcattctggtgcctacttgctgagctccaccaaactaatgcgtgccacggagggtaccacacagcaccagcgtctgctggagcaatgccctcctgcccagctgcacggccccctggatgcgctcacacagttggggaactgcgcctggcgtgtaaacgggcatctgctggacttggtgctgcagatctttagagacaagggctgtatgcccttgggcgtgcctcccccgcgctcagaagcaccgcggccggcccggtatcagctgccacccggctccacaccagtgcacaagtctgagctgcggaaggagctggcgcgttgcctcaaggtggctcgagagatgcacagtctgcgcagcgaggccctgtatcgcctgtcgctggcacagcacctacgccaccgtgtcttctggttgccgcacaacatggacttccgcggccgcacttacccctgcccgccacacttcaaccacctgggcagtgacctagcgcgtgcgctattggagtttgctgagggccggccactgggaccacgtggtctggactggcttaagatccacctgatcaacctgactggcctcaagaagggagactcgctgcgcatgcgcctagccttcgcagatgaggtcatggaggagatcttggactctgcagacaaccccttgacgggccggaagtggtggatggaagctgatgagccctggcagaccctggcctgctgcatggaggtggcacacgcagtccggtccccagaccctgctgcctacatttcccacctgccagttcaccaggtgggcacgcaagtggactgcccatggctgagcccgaggtgccttaggaactggcatttgcagttgggtcttaccgtaaactgggattgccctgtgggactagtctcttgatttatgagggaggttgctcccgggtctccttgcaccaggtggtggaataagtttctggggtgacctgtgtgtccctattgggtcatttggcctcagctcatgggcaccccttgtggtcaggatggctcctgcaatggcttacagcattacgccgcactaggccgagacagtgtgggtgctgcctcagtcaacctaacgccgtccgacctgccccaagatgtgtacagggaggtggcaacacaggtgggtgccaggaaacccctcctgtccccactcggggtagcctttctacaagtgatggggtccttagtggggaggggaggtcacgtaatggcttccctgacccctaatcccatccaggtggaggagttccgccagcaggacgccaaggagggtctacgggtggctcaggtgctggagggcttcatcagccgcaaggtggtgaagcagacagtgatgacggtggtgtatggggtcacacgctacggagggcgcctgcagatagagaagcgcctgcgcgaactcagcgacttccctcaggagtttgtctgggaagcctcacactacctcgtgcgccaggtcttcaaaagtctacaggagatgttcaccagcacgcgggccattcagcactggctgactgagagtgccaacctcatctctcacgccggatggcctgtggagtgggtcacgcccctgggcatccccatcatacagccctatcaccgcgagtccaaggtccaggtacttgggaaatgcccttctccttttccctcatctcccctcctccctgccaagggctggactcacccctcctccaaccccggtctgcaggtaaaaggtggcctccagagcatcaccctcaccagctcggtagatgagagtcagtgagtcctgtactgcggggaggggagaggatggagggatgggcaggaaaggctctgggctaagagggtaccagcctacaatttctgtccacaggaagcccaacactctgaagcagaaaaatggcttcccgcccaacttcatccactccctggactcctcccacatgatgctgaccgccctacactgctacaggaagggcctgattttcgtctccgtgcacgactgcttctggacacatgccgctgacatccccacgatgaacgaggtatgccgcgagcaattcgtgcgcctgcacagccagcccatcctggaagacctggccaagttcctgaagaagcgtttctgctccgtgtccagcatcaagtccctaaagtcctcggagcgcgccctggtcaccaagctgcaggagacactacagtccttgccaaagacaggtaccttcgatctaggacaggtgataagatccacctactttttcagctgacccacgccatgcggtccctaaacctgtgcataatgtaaataaatgaaagtttgtgtctacctatgccagccgccactttctggcggtgcgcaaacccaccaaagtaatgag

SEQ ID NO: 583 - DNA, 20 bp, Mus sp.
  source 1..20: mol_type = genomic DNA
cccacgatgaacgaggtatg

SEQ ID NO: 584 - DNA, 22 bp, Mus sp.
  source 1..22: mol_type = genomic DNA
cgaggactttagggacttgatg

SEQ ID NO: 585 - DNA, 20 bp, Mus sp.
  source 1..20: mol_type = genomic DNA
gccgctagaggtgaaattct

SEQ ID NO: 586 - DNA, 20 bp, Mus sp.
  source 1..20: mol_type = genomic DNA
tcggaactacgacggtatct

SEQ ID NO: 587 - DNA, 20 bp, synthetic construct
  source 1..20: mol_type = other DNA
gaagtccatgttgtgcggca

SEQ ID NO: 588 - DNA, 20 bp, synthetic construct
  source 1..20: mol_type = other DNA
cggaagtccatgttgtgcgg

SEQ ID NO: 589 - DNA, 20 bp, synthetic construct
  source 1..20: mol_type = other DNA
cgcggaagtccatgttgtgc

SEQ ID NO: 590 - DNA, 20 bp, synthetic construct
  source 1..20: mol_type = other DNA
gccgcggaagtccatgttgt

SEQ ID NO: 591 - DNA, 20 bp, synthetic construct
  source 1..20: mol_type = other DNA
ggccgcggaagtccatgttg

SEQ ID NO: 592 - DNA, 20 bp, synthetic construct
  source 1..20: mol_type = other DNA
cggccgcggaagtccatgtt

SEQ ID NO: 593 - DNA, 20 bp, synthetic construct
  source 1..20: mol_type = other DNA
gcggccgcggaagtccatgt

SEQ ID NO: 594 - DNA, 20 bp, synthetic construct
  source 1..20: mol_type = other DNA
tgcggccgcggaagtccatg

SEQ ID NO: 595 - DNA, 20 bp, synthetic construct
  source 1..20: mol_type = other DNA
gtgcggccgcggaagtccat

SEQ ID NO: 596 - DNA, 20 bp, synthetic construct
  source 1..20: mol_type = other DNA
ctgcccaggtggttgaagtg

SEQ ID NO: 597 - DNA, 20 bp, synthetic construct
  source 1..20: mol_type = other DNA
tctgcttcaccaccttgcgg

SEQ ID NO: 598 - DNA, 20 bp, synthetic construct
  source 1..20: mol_type = other DNA
gtctgcttcaccaccttgcg

SEQ ID NO: 599 - DNA, 20 bp, synthetic construct
  source 1..20: mol_type = other DNA
atgaagttgggcgggaagcc

SEQ ID NO: 600 - DNA, 20 bp, synthetic construct
  source 1..20: mol_type = other DNA
gatgaagttgggcgggaagc

SEQ ID NO: 601 - DNA, 20 bp, synthetic construct
  source 1..20: mol_type = other DNA
ggatgaagttgggcgggaag

SEQ ID NO: 602 - DNA, 20 bp, synthetic construct
  source 1..20: mol_type = other DNA
tggatgaagttgggcgggaa

SEQ ID NO: 603 - DNA, 20 bp, synthetic construct
  source 1..20: mol_type = other DNA
gtggatgaagttgggcggga

SEQ ID NO: 604 - DNA, 20 bp, synthetic construct
  source 1..20: mol_type = other DNA
gagtggatgaagttgggcgg

SEQ ID NO: 605 - DNA, 20 bp, synthetic construct
  source 1..20: mol_type = other DNA
gagtggatgaagttgggcgg

SEQ ID NO: 606 - DNA, 20 bp, synthetic construct
  source 1..20: mol_type = other DNA
atcatgtgggaggagtccag

SEQ ID NO: 607 - DNA, 20 bp, synthetic construct
  source 1..20: mol_type = other DNA
catcatgtgggaggagtcca

SEQ ID NO: 608 - DNA, 20 bp, synthetic construct
  source 1..20: mol_type = other DNA
gcatcatgtgggaggagtcc

SEQ ID NO: 609 - DNA, 20 bp, synthetic construct
  source 1..20: mol_type = other DNA
aggcccttcctgtagcagtg

SEQ ID NO: 610 - DNA, 20 bp, synthetic construct
  source 1..20: mol_type = other DNA
caggcccttcctgtagcagt

SEQ ID NO: 611 - DNA, 20 bp, synthetic construct
  source 1..20: mol_type = other DNA
tcaggcccttcctgtagcag

SEQ ID NO: 612 - DNA, 18 bp, synthetic construct
  source 1..18: mol_type = other DNA
cccagccaagcatcacgg

SEQ ID NO: 613 - DNA, 18 bp, synthetic construct
  source 1..18: mol_type = other DNA
gcccagccaagcatcacg

SEQ ID NO: 614 - DNA, 18 bp, synthetic construct
  source 1..18: mol_type = other DNA
agtccatgttgtgcggca

SEQ ID NO: 615 - DNA, 18 bp, synthetic construct
  source 1..18: mol_type = other DNA
aagtccatgttgtgcggc

SEQ ID NO: 616 - DNA, 18 bp, synthetic construct
  source 1..18: mol_type = other DNA
gaagtccatgttgtgcgg

SEQ ID NO: 617 - DNA, 18 bp, synthetic construct
  source 1..18: mol_type = other DNA
ggaagtccatgttgtgcg

SEQ ID NO: 618 - DNA, 18 bp, synthetic construct
  source 1..18: mol_type = other DNA
cggaagtccatgttgtgc

SEQ ID NO: 619 - DNA, 18 bp, synthetic construct
  source 1..18: mol_type = other DNA
gcggaagtccatgttgtg

SEQ ID NO: 620 - DNA, 18 bp, synthetic construct
  source 1..18: mol_type = other DNA
cgcggaagtccatgttgt

SEQ ID NO: 621 - DNA, 18 bp, synthetic construct
  source 1..18: mol_type = other DNA
ccgcggaagtccatgttg

SEQ ID NO: 622 - DNA, 18 bp, synthetic construct
  source 1..18: mol_type = other DNA
gccgcggaagtccatgtt

SEQ ID NO: 623 - DNA, 18 bp, synthetic construct
  source 1..18: mol_type = other DNA
ggccgcggaagtccatgt

SEQ ID NO: 624 - DNA, 18 bp, synthetic construct
  source 1..18: mol_type = other DNA
cggccgcggaagtccatg

SEQ ID NO: 625 - DNA, 18 bp, synthetic construct
  source 1..18: mol_type = other DNA
gcggccgcggaagtccat

SEQ ID NO: 626 - DNA, 18 bp, synthetic construct
  source 1..18: mol_type = other DNA
tgcggccgcggaagtcca

SEQ ID NO: 627 - DNA, 18 bp, synthetic construct
  source 1..18: mol_type = other DNA
gtgcggccgcggaagtcc

SEQ ID NO: 628 - DNA, 18 bp, synthetic construct
  source 1..18: mol_type = other DNA
gcccaggtggttgaagtg

SEQ ID NO: 629 - DNA, 18 bp, synthetic construct
  source 1..18: mol_type = other DNA
tgcccaggtggttgaagt

SEQ ID NO: 630 - DNA, 18 bp, synthetic construct
  source 1..18: mol_type = other DNA
ctgcccaggtggttgaag

SEQ ID NO: 631 - DNA, 18 bp, synthetic construct
  source 1..18: mol_type = other DNA
tgcttcaccaccttgcgg

SEQ ID NO: 632 - DNA, 18 bp, synthetic construct
  source 1..18: mol_type = other DNA
ctgcttcaccaccttgcg

SEQ ID NO: 633 - DNA, 18 bp, synthetic construct
  source 1..18: mol_type = other DNA
tctgcttcaccaccttgc

SEQ ID NO: 634 - DNA, 18 bp, synthetic construct
  source 1..18: mol_type = other DNA
gtctgcttcaccaccttg

SEQ ID NO: 635 - DNA, 18 bp, synthetic construct
  source 1..18: mol_type = other DNA
gaacatctcctgtagact

SEQ ID NO: 636 - DNA, 18 bp, synthetic construct
  source 1..18: mol_type = other DNA
gaagttgggcgggaagcc

SEQ ID NO: 637 - DNA, 18 bp, synthetic construct
  source 1..18: mol_type = other DNA
tgaagttgggcgggaagc

SEQ ID NO: 638 - DNA, 18 bp, synthetic construct
  source 1..18: mol_type = other DNA
atgaagttgggcgggaag

SEQ ID NO: 639 - DNA, 18 bp, synthetic construct
  source 1..18: mol_type = other DNA
gatgaagttgggcgggaa

SEQ ID NO: 640 - DNA, 18 bp, synthetic construct
  source 1..18: mol_type = other DNA
ggatgaagttgggcggga

SEQ ID NO: 641 - DNA, 18 bp, synthetic construct
  source 1..18: mol_type = other DNA
tggatgaagttgggcggg

SEQ ID NO: 642 - DNA, 18 bp, synthetic construct
  source 1..18: mol_type = other DNA
gtggatgaagttgggcgg

SEQ ID NO: 643 - DNA, 18 bp, synthetic construct
  source 1..18: mol_type = other DNA
agtggatgaagttgggcg

SEQ ID NO: 644 - DNA, 18 bp, synthetic construct
  source 1..18: mol_type = other DNA
gagtggatgaagttgggc

SEQ ID NO: 645 - DNA, 18 bp, synthetic construct
  source 1..18: mol_type = other DNA
catgtgggaggagtccag

SEQ ID NO: 646 - DNA, 18 bp, synthetic construct
  source 1..18: mol_type = other DNA
tcatgtgggaggagtcca

SEQ ID NO: 647 - DNA, 18 bp, synthetic construct
  source 1..18: mol_type = other DNA
atcatgtgggaggagtcc

SEQ ID NO: 648 - DNA, 18 bp, synthetic construct
  source 1..18: mol_type = other DNA
catcatgtgggaggagtc

SEQ ID NO: 649 - DNA, 18 bp, synthetic construct
  source 1..18: mol_type = other DNA
gcatcatgtgggaggagt

SEQ ID NO: 650 - DNA, 18 bp, synthetic construct
  source 1..18: mol_type = other DNA
agcatcatgtgggaggag

SEQ ID NO: 651 - DNA, 18 bp, synthetic construct
  source 1..18: mol_type = other DNA
gcccttcctgtagcagtg

SEQ ID NO: 652 - DNA, 18 bp, synthetic construct
  source 1..18: mol_type = other DNA
ggcccttcctgtagcagt

SEQ ID NO: 653 - DNA, 18 bp, synthetic construct
  source 1..18: mol_type = other DNA
aggcccttcctgtagcag

SEQ ID NO: 654 - DNA, 18 bp, synthetic construct
  source 1..18: mol_type = other DNA
caggcccttcctgtagca

SEQ ID NO: 655 - DNA, 18 bp, synthetic construct
  source 1..18: mol_type = other DNA
tcaggcccttcctgtagc

SEQ ID NO: 656 - DNA, 20 bp, Homo sapiens
  source 1..20: mol_type = genomic DNA
tgccgcacaacatggacttc

SEQ ID NO: 657 - DNA, 20 bp, Homo sapiens
  source 1..20: mol_type = genomic DNA
ccgcacaacatggacttccg

SEQ ID NO: 658 - DNA, 20 bp, Homo sapiens
  source 1..20: mol_type = genomic DNA
gcacaacatggacttccgcg

SEQ ID NO: 659 - DNA, 20 bp, Homo sapiens
  source 1..20: mol_type = genomic DNA
acaacatggacttccgcggc

SEQ ID NO: 660 - DNA, 20 bp, Homo sapiens
  source 1..20: mol_type = genomic DNA
caacatggacttccgcggcc

SEQ ID NO: 661 - DNA, 20 bp, unidentified
  source 1..20: mol_type = unassigned DNA
aacatggacttccgcggccg

SEQ ID NO: 662 - DNA, 20 bp, Homo sapiens
  source 1..20: mol_type = genomic DNA
acatggacttccgcggccgc

SEQ ID NO: 663 - DNA, 20 bp, unidentified
  source 1..20: mol_type = unassigned DNA
catggacttccgcggccgca

SEQ ID NO: 664 - DNA, 20 bp, Homo sapiens
  source 1..20: mol_type = genomic DNA
atggacttccgcggccgcac

SEQ ID NO: 665 - DNA, 20 bp, Homo sapiens
  source 1..20: mol_type = genomic DNA
cacttcaaccacctgggcag

SEQ ID NO: 666 - DNA, 20 bp, unidentified
  source 1..20: mol_type = unassigned DNA
ccgcaaggtggtgaagcaga

SEQ ID NO: 667 - DNA, 20 bp, unidentified
  source 1..20: mol_type = unassigned DNA
cgcaaggtggtgaagcagac

SEQ ID NO: 668 - DNA, 20 bp, Homo sapiens
  source 1..20: mol_type = genomic DNA
ggcttcccgcccaacttcat

SEQ ID NO: 669 - DNA, 20 bp, Homo sapiens
  source 1..20: mol_type = genomic DNA
gcttcccgcccaacttcatc

SEQ ID NO: 670 - DNA, 20 bp, Homo sapiens
  source 1..20: mol_type = genomic DNA
cttcccgcccaacttcatcc

SEQ ID NO: 671 - DNA, 20 bp, Homo sapiens
  source 1..20: mol_type = genomic DNA
ttcccgcccaacttcatcca

SEQ ID NO: 672 - DNA, 20 bp, Homo sapiens
  source 1..20: mol_type = genomic DNA
tcccgcccaacttcatccac

SEQ ID NO: 673 - DNA, 20 bp, Homo sapiens
  source 1..20: mol_type = genomic DNA
ccgcccaacttcatccactc

SEQ ID NO: 674 - DNA, 20 bp, Homo sapiens
  source 1..20: mol_type = genomic DNA
ccgcccaacttcatccactc

SEQ ID NO: 675 - DNA, 20 bp, Homo sapiens
  source 1..20: mol_type = genomic DNA
ctggactcctcccacatgat

SEQ ID NO: 676 - DNA, 20 bp, Homo sapiens
  source 1..20: mol_type = genomic DNA
tggactcctcccacatgatg

SEQ ID NO: 677 - DNA, 20 bp, Homo sapiens
  source 1..20: mol_type = genomic DNA
ggactcctcccacatgatgc

SEQ ID NO: 678 - DNA, 20 bp, Homo sapiens
  source 1..20: mol_type = genomic DNA
cactgctacaggaagggcct

SEQ ID NO: 679 - DNA, 20 bp, Homo sapiens
  source 1..20: mol_type = genomic DNA
actgctacaggaagggcctg

SEQ ID NO: 680 - DNA, 20 bp, Homo sapiens
  source 1..20: mol_type = genomic DNA
ctgctacaggaagggcctga

SEQ ID NO: 681 - DNA, 18 bp, unidentified
  source 1..18: mol_type = unassigned DNA
ccgtgatgcttggctggg

SEQ ID NO: 682 - DNA, 18 bp, unidentified
  source 1..18: mol_type = unassigned DNA
cgtgatgcttggctgggc

SEQ ID NO: 683 - DNA, 18 bp, Homo sapiens
  source 1..18: mol_type = genomic DNA
tgccgcacaacatggact

SEQ ID NO: 684 - DNA, 18 bp, Homo sapiens
  source 1..18: mol_type = genomic DNA
gccgcacaacatggactt

SEQ ID NO: 685 - DNA, 18 bp, Homo sapiens
  source 1..18: mol_type = genomic DNA
ccgcacaacatggacttc

SEQ ID NO: 686 - DNA, 18 bp, Homo sapiens
  source 1..18: mol_type = genomic DNA
cgcacaacatggacttcc

SEQ ID NO: 687 - DNA, 18 bp, Homo sapiens
  source 1..18: mol_type = genomic DNA
gcacaacatggacttccg

SEQ ID NO: 688 - DNA, 18 bp, Homo sapiens
  source 1..18: mol_type = genomic DNA
cacaacatggacttccgc

SEQ ID NO: 689 - DNA, 18 bp, Homo sapiens
  source 1..18: mol_type = genomic DNA
acaacatggacttccgcg

SEQ ID NO: 690 - DNA, 18 bp, Homo sapiens
  source 1..18: mol_type = genomic DNA
caacatggacttccgcgg

SEQ ID NO: 691 - DNA, 18 bp, Homo sapiens
  source 1..18: mol_type = genomic DNA
aacatggacttccgcggc

SEQ ID NO: 692 - DNA, 18 bp, unidentified
  source 1..18: mol_type = unassigned DNA
acatggacttccgcggcc

SEQ ID NO: 693 - DNA, 18 bp, unidentified
  source 1..18: mol_type = unassigned DNA
catggacttccgcggccg

SEQ ID NO: 694 - DNA, 18 bp, unidentified
  source 1..18: mol_type = unassigned DNA
atggacttccgcggccgc

SEQ ID NO: 695 - DNA, 18 bp, unidentified
  source 1..18: mol_type = unassigned DNA
tggacttccgcggccgca

SEQ ID NO: 696 - DNA, 18 bp, Homo sapiens
  source 1..18: mol_type = genomic DNA
ggacttccgcggccgcac

SEQ ID NO: 697 - DNA, 18 bp, Homo sapiens
  source 1..18: mol_type = genomic DNA
cacttcaaccacctgggc

SEQ ID NO: 698 - DNA, 18 bp, Homo sapiens
  source 1..18: mol_type = genomic DNA
acttcaaccacctgggca

SEQ ID NO: 699 - DNA, 18 bp, Homo sapiens
  source 1..18: mol_type = genomic DNA
cttcaaccacctgggcag

SEQ ID NO: 700 - DNA, 18 bp, Homo sapiens
  source 1..18: mol_type = genomic DNA
ccgcaaggtggtgaagca

SEQ ID NO: 701 - DNA, 18 bp, unidentified
  source 1..18: mol_type = unassigned DNA
cgcaaggtggtgaagcag

SEQ ID NO: 702 - DNA, 18 bp, unidentified
  source 1..18: mol_type = unassigned DNA
gcaaggtggtgaagcaga

SEQ ID NO: 703 - DNA, 18 bp, unidentified
  source 1..18: mol_type = unassigned DNA
caaggtggtgaagcagac

SEQ ID NO: 704 - DNA, 18 bp, Homo sapiens
  source 1..18: mol_type = genomic DNA
agtctacaggagatgttc

SEQ ID NO: 705 - DNA, 18 bp, Homo sapiens
  source 1..18: mol_type = genomic DNA
ggcttcccgcccaacttc

SEQ ID NO: 706 - DNA, 18 bp, Homo sapiens
  source 1..18: mol_type = genomic DNA
gcttcccgcccaacttca

SEQ ID NO: 707 - DNA, 18 bp, Homo sapiens
  source 1..18: mol_type = genomic DNA
cttcccgcccaacttcat

SEQ ID NO: 708 - DNA, 18 bp, Homo sapiens
  source 1..18: mol_type = genomic DNA
ttcccgcccaacttcatc

SEQ ID NO: 709 - DNA, 18 bp, Homo sapiens
  source 1..18: mol_type = genomic DNA
tcccgcccaacttcatcc

SEQ ID NO: 710 - DNA, 18 bp, Homo sapiens
  source 1..18: mol_type = genomic DNA
cccgcccaacttcatcca

SEQ ID NO: 711 - DNA, 18 bp, Homo sapiens
  source 1..18: mol_type = genomic DNA
ccgcccaacttcatccac

SEQ ID NO: 712 - DNA, 18 bp, Homo sapiens
  source 1..18: mol_type = genomic DNA
cgcccaacttcatccact

SEQ ID NO: 713 - DNA, 18 bp, Homo sapiens
  source 1..18: mol_type = genomic DNA
gcccaacttcatccactc

SEQ ID NO: 714 - DNA, 18 bp, Homo sapiens
  source 1..18: mol_type = genomic DNA
ctggactcctcccacatg

SEQ ID NO: 715 - DNA, 18 bp, Homo sapiens
  source 1..18: mol_type = genomic DNA
tggactcctcccacatga

SEQ ID NO: 716 - DNA, 18 bp, Homo sapiens
  source 1..18: mol_type = genomic DNA
ggactcctcccacatgat

SEQ ID NO: 717 - DNA, 18 bp, Homo sapiens
  source 1..18: mol_type = genomic DNA
gactcctcccacatgatg

SEQ ID NO: 718 - DNA, 18 bp, Homo sapiens
  source 1..18: mol_type = genomic DNA
actcctcccacatgatgc

SEQ ID NO: 719 - DNA, 18 bp, Homo sapiens
  source 1..18: mol_type = genomic DNA
ctcctcccacatgatgct

SEQ ID NO: 720 - DNA, 18 bp, Homo sapiens
  source 1..18: mol_type = genomic DNA
cactgctacaggaagggc

SEQ ID NO: 721 - DNA, 18 bp, Homo sapiens
  source 1..18: mol_type = genomic DNA
actgctacaggaagggcc

SEQ ID NO: 722 - DNA, 18 bp, Homo sapiens
  source 1..18: mol_type = genomic DNA
ctgctacaggaagggcct

SEQ ID NO: 723 - DNA, 18 bp, Homo sapiens
  source 1..18: mol_type = genomic DNA
tgctacaggaagggcctg

SEQ ID NO: 724 - DNA, 18 bp, Homo sapiens
  source 1..18: mol_type = genomic DNA
gctacaggaagggcctga

SEQ ID NO: 725 - DNA, 29 bp, Homo sapiens
  source 1..29: mol_type = genomic DNA
caacgccgtgatgcttggctgggcgcggc

SEQ ID NO: 726 - DNA, 32 bp, Homo sapiens
  source 1..32: mol_type = genomic DNA
cgcacaacatggacttccgcggccgcacctac

SEQ ID NO: 727 - DNA, 31 bp, Homo sapiens
  source 1..31: mol_type = genomic DNA
atcacccgcaaggtggtgaagcagacggtga

SEQ ID NO: 728 - DNA, 20 bp, synthetic construct
  source 1..20: mol_type = other DNA
  modified_base 1: mod_base = OTHER; note = 2'-O-methoxyethyl modified nucleotide with phosphorothioate linkage
  modified_base 2: mod_base = OTHER; note = 2'-O-methoxyethyl modified nucleotide with phosphorothioate linkage
  modified_base 3: mod_base = OTHER; note = 2'-O-methoxyethyl modified nucleotide with phosphorothioate linkage
  modified_base 4: mod_base = OTHER; note = 2'-O-methoxyethyl modified nucleotide with phosphorothioate linkage
  modified_base 5: mod_base = OTHER; note = 2'-O-methoxyethyl modified nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Thymine with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl modified nucleotide with phosphorothioate linkage
  modified_base 17: mod_base = OTHER; note = 2'-O-methoxyethyl thymine with phosphorothioate linkage
  modified_base 18: mod_base = OTHER; note = 2'-O-methoxyethyl modified nucleotide with phosphorothioate linkage
  modified_base 19: mod_base = OTHER; note = 2'-O-methoxyethyl thymine with phosphorothioate linkage
  modified_base 20: mod_base = OTHER; note = 2'-O-methoxyethyl thymine
  misc_feature 1..5: note = RNA
  misc_feature 6..15: note = DNA
  misc_feature 16..20: note = RNA
cggccgcggaagtccatgtt

SEQ ID NO: 729 - DNA, 20 bp, synthetic construct
  source 1..20: mol_type = other DNA
  modified_base 1: mod_base = OTHER; note = 2'-O-methoxyethyl thymine with phosphorothioate linkage
  modified_base 2: mod_base = OTHER; note = 2'-O-methoxyethyl modified nucleotide with phosphorothioate linkage
  modified_base 3: mod_base = OTHER; note = 2'-O-methoxyethyl modified nucleotide with phosphorothioate linkage
  modified_base 4: mod_base = OTHER; note = 2'-O-methoxyethyl modified nucleotide with phosphorothioate linkage
  modified_base 5: mod_base = OTHER; note = 2'-O-methoxyethyl modified nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Thymine with phosphorothioate linkage
  modified_base 16: mod_base = OTHER; note = 2'-O-methoxyethyl modified nucleotide with phosphorothioate linkage
  modified_base 17: mod_base = OTHER; note = 2'-O-methoxyethyl modified nucleotide with phosphorothioate linkage
  modified_base 18: mod_base = OTHER; note = 2'-O-methoxyethyl modified nucleotide with phosphorothioate linkage
  modified_base 19: mod_base = OTHER; note = 2'-O-methoxyethyl thymine with phosphorothioate linkage
  modified_base 20: mod_base = OTHER; note = 2'-O-methoxyethyl modified nucleotide
  misc_feature 1..5: note = RNA
  misc_feature 6..15: note = DNA
  misc_feature 16..20: note = RNA
tgcggccgcggaagtccatg

SEQ ID NO: 730 - DNA, 20 bp, synthetic construct
  source 1..20: mol_type = other DNA
  modified_base 1: mod_base = OTHER; note = 2'-O-methoxyethyl thymine with phosphorothioate linkage
  modified_base 2: mod_base = OTHER; note = 2'-O-methoxyethyl modified nucleotide with phosphorothioate linkage
  modified_base 3: mod_base = OTHER; note = 2'-O-methoxyethyl thymine with phosphorothioate linkage
  modified_base 4: mod_base = OTHER; note = 2'-O-methoxyethyl modified nucleotide with phosphorothioate linkage
  modified_base 5: mod_base = OTHER; note = 2'-O-methoxyethyl modified nucleotide with phosphorothioate linkage
  modified_base 6: mod_base = OTHER; note = Thymine with phosphorothioate linkage
  modified_base 7: mod_base = OTHER; note = Thymine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Thymine with phosphorothioate linkage
  modified_base 16: mod_base = OTHER; note = 2'-O-methoxyethyl thymine with phosphorothioate linkage
  modified_base 17: mod_base = OTHER; note = 2'-O-methoxyethyl modified nucleotide with phosphorothioate linkage
  modified_base 18: mod_base = OTHER; note = 2'-O-methoxyethyl modified nucleotide with phosphorothioate linkage
  modified_base 19: mod_base = OTHER; note = 2'-O-methoxyethyl modified nucleotide with phosphorothioate linkage
  modified_base 20: mod_base = OTHER; note = 2'-O-methoxyethyl modified nucleotide
  misc_feature 1..5: note = RNA
  misc_feature 6..15: note = DNA
  misc_feature 16..20: note = RNA
tctgcttcaccaccttgcgg

SEQ ID NO: 731 - DNA, 20 bp, synthetic construct
  source 1..20: mol_type = other DNA
  modified_base 1: mod_base = OTHER; note = 2'-O-methoxyethyl modified nucleotide with phosphorothioate linkage
  modified_base 2: mod_base = OTHER; note = 2'-O-methoxyethyl thymine with phosphorothioate linkage
  modified_base 3: mod_base = OTHER; note = 2'-O-methoxyethyl modified nucleotide with phosphorothioate linkage
  modified_base 4: mod_base = OTHER; note = 2'-O-methoxyethyl thymine with phosphorothioate linkage
  modified_base 5: mod_base = OTHER; note = 2'-O-methoxyethyl modified nucleotide with phosphorothioate linkage
  modified_base 6: mod_base = OTHER; note = Nucleotide with phosphorothioate linkage
  modified_base 7: mod_base = OTHER; note = Thymine with phosphorothioate linkage
  modified_base 8: mod_base = OTHER; note = Thymine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl thymine with phosphorothioate linkage
  modified_base 17: mod_base = OTHER; note = 2'-O-methoxyethyl thymine with phosphorothioate linkage
  modified_base 18: mod_base = OTHER; note = 2'-O-methoxyethyl modified nucleotide with phosphorothioate linkage
  modified_base 19: mod_base = OTHER; note = 2'-O-methoxyethyl modified nucleotide with phosphorothioate linkage
  modified_base 20: mod_base = OTHER; note = 2'-O-methoxyethyl modified nucleotide
  misc_feature 1..5: note = RNA
  misc_feature 6..15: note = DNA
  misc_feature 16..20: note = RNA
gtctgcttcaccaccttgcg

SEQ ID NO: 732 - DNA, 18 bp, synthetic construct
  source 1..18: mol_type = other DNA
  modified_base 1: mod_base = OTHER; note = 2'-O-methoxyethyl modified nucleotide with phosphorothioate linkage
  modified_base 2: mod_base = OTHER; note = 2'-O-methoxyethyl modified nucleotide with phosphorothioate linkage
  modified_base 3: mod_base = OTHER; note = 2'-O-methoxyethyl modified nucleotide with phosphorothioate linkage
  modified_base 4: mod_base = OTHER; note = 2'-O-methoxyethyl modified nucleotide with phosphorothioate linkage
  modified_base 5: mod_base = OTHER; note = 2'-O-methoxyethyl modified nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Thymine with phosphorothioate linkage
  modified_base 14: mod_base = OTHER; note = 2'-O-methoxyethyl modified nucleotide with phosphorothioate linkage
  modified_base 15: mod_base = OTHER; note = 2'-O-methoxyethyl modified nucleotide with phosphorothioate linkage
  modified_base 16: mod_base = OTHER; note = 2'-O-methoxyethyl modified nucleotide with phosphorothioate linkage
  modified_base 17: mod_base = OTHER; note = 2'-O-methoxyethyl modified nucleotide with phosphorothioate linkage
  modified_base 18: mod_base = OTHER; note = 2'-O-methoxyethyl modified nucleotide
  misc_feature 1..5: note = RNA
  misc_feature 6..13: note = DNA
  misc_feature 14..18: note = RNA
cccagccaagcatcacgg

SEQ ID NO: 733 - DNA, 18 bp, synthetic construct
  source 1..18: mol_type = other DNA
  modified_base 1: mod_base = OTHER; note = 2'-O-methoxyethyl modified nucleotide with phosphorothioate linkage
  modified_base 2: mod_base = OTHER; note = 2'-O-methoxyethyl modified nucleotide with phosphorothioate linkage
  modified_base 3: mod_base = OTHER; note = 2'-O-methoxyethyl modified nucleotide with phosphorothioate linkage
  modified_base 4: mod_base = OTHER; note = 2'-O-methoxyethyl modified nucleotide with phosphorothioate linkage
  modified_base 5: mod_base = OTHER; note = 2'-O-methoxyethyl modified nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 2'-O-methoxyethyl thymine with phosphorothioate linkage
  modified_base 15: mod_base = OTHER; note = 2'-O-methoxyethyl modified nucleotide with phosphorothioate linkage
  modified_base 16: mod_base = OTHER; note = 2'-O-methoxyethyl modified nucleotide with phosphorothioate linkage
  modified_base 17: mod_base = OTHER; note = 2'-O-methoxyethyl modified nucleotide with phosphorothioate linkage
  modified_base 18: mod_base = OTHER; note = 2'-O-methoxyethyl modified nucleotide
  misc_feature 1..5: note = RNA
  misc_feature 6..13: note = DNA
  misc_feature 14..18: note = RNA
gcccagccaagcatcacg

SEQ ID NO: 734 - DNA, 18 bp, synthetic construct
  source 1..18: mol_type = other DNA
  modified_base 1: mod_base = OTHER; note = 2'-O-methoxyethyl modified nucleotide with phosphorothioate linkage
  modified_base 2: mod_base = OTHER; note = 2'-O-methoxyethyl modified nucleotide with phosphorothioate linkage
  modified_base 3: mod_base = OTHER; note = 2'-O-methoxyethyl modified nucleotide with phosphorothioate linkage
  modified_base 4: mod_base = OTHER; note = 2'-O-methoxyethyl modified nucleotide with phosphorothioate linkage
  modified_base 5: mod_base = OTHER; note = 2'-O-methoxyethyl modified nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Thymine with phosphorothioate linkage
  modified_base 13: mod_base = OTHER; note = Nucleotide with phosphorothioate linkage
  modified_base 14: mod_base = OTHER; note = 2'-O-methoxyethyl modified nucleotide with phosphorothioate linkage
  modified_base 15: mod_base = OTHER; note = 2'-O-methoxyethyl modified nucleotide with phosphorothioate linkage
  modified_base 16: mod_base = OTHER; note = 2'-O-methoxyethyl thymine with phosphorothioate linkage
  modified_base 17: mod_base = OTHER; note = 2'-O-methoxyethyl modified nucleotide with phosphorothioate linkage
  modified_base 18: mod_base = OTHER; note = 2'-O-methoxyethyl thymine
  misc_feature 1..5: note = RNA
  misc_feature 6..13: note = DNA
  misc_feature 14..18: note = RNA
ggccgcggaagtccatgt

SEQ ID NO: 735 - DNA, 18 bp, synthetic construct
  source 1..18: mol_type = other DNA
  modified_base 1: mod_base = OTHER; note = 2'-O-methoxyethyl modified nucleotide with phosphorothioate linkage
  modified_base 2: mod_base = OTHER; note = 2'-O-methoxyethyl modified nucleotide with phosphorothioate linkage
  modified_base 3: mod_base = OTHER; note = 2'-O-methoxyethyl modified nucleotide with phosphorothioate linkage
  modified_base 4: mod_base = OTHER; note = 2'-O-methoxyethyl modified nucleotide with phosphorothioate linkage
  modified_base 5: mod_base = OTHER; note = 2'-O-methoxyethyl modified nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Thymine with phosphorothioate linkage
  modified_base 14: mod_base = OTHER; note = 2'-O-methoxyethyl modified nucleotide with phosphorothioate linkage
  modified_base 15: mod_base = OTHER; note = 2'-O-methoxyethyl modified nucleotide with phosphorothioate linkage
  modified_base 16: mod_base = OTHER; note = 2'-O-methoxyethyl modified nucleotide with phosphorothioate linkage
  modified_base 17: mod_base = OTHER; note = 2'-O-methoxyethyl thymine with phosphorothioate linkage
  modified_base 18: mod_base = OTHER; note = 2'-O-methoxyethyl modified nucleotide
  misc_feature 1..5: note = RNA
  misc_feature 6..13: note = DNA
  misc_feature 14..18: note = RNA
cggccgcggaagtccatg

SEQ ID NO: 736 - DNA, 18 bp, synthetic construct
  source 1..18: mol_type = other DNA
  modified_base 1: mod_base = OTHER; note = 2'-O-methoxyethyl modified nucleotide with phosphorothioate linkage
  modified_base 2: mod_base = OTHER; note = 2'-O-methoxyethyl modified nucleotide with phosphorothioate linkage
  modified_base 3: mod_base = OTHER; note = 2'-O-methoxyethyl modified nucleotide with phosphorothioate linkage
  modified_base 4: mod_base = OTHER; note = 2'-O-methoxyethyl modified nucleotide with phosphorothioate linkage
  modified_base 5: mod_base = OTHER; note = 2'-O-methoxyethyl modified nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 2'-O-methoxyethyl thymine with phosphorothioate linkage
  modified_base 15: mod_base = OTHER; note = 2'-O-methoxyethyl modified nucleotide with phosphorothioate linkage
  modified_base 16: mod_base = OTHER; note = 2'-O-methoxyethyl modified nucleotide with phosphorothioate linkage
  modified_base 17: mod_base = OTHER; note = 2'-O-methoxyethyl modified nucleotide with phosphorothioate linkage
  modified_base 18: mod_base = OTHER; note = 2'-O-methoxyethyl thymine
  misc_feature 1..5: note = RNA
  misc_feature 6..13: note = DNA
  misc_feature 14..18: note = RNA
gcggccgcggaagtccat

SEQ ID NO: 737 - DNA, 18 bp, synthetic construct
  source 1..18: mol_type = other DNA
  modified_base 1: mod_base = OTHER; note = 2'-O-methoxyethyl thymine with phosphorothioate linkage
  modified_base 2: mod_base = OTHER; note = 2'-O-methoxyethyl modified nucleotide with phosphorothioate linkage
  modified_base 3: mod_base = OTHER; note = 2'-O-methoxyethyl modified nucleotide with phosphorothioate linkage
  modified_base 4: mod_base = OTHER; note = 2'-O-methoxyethyl modified nucleotide with phosphorothioate linkage
  modified_base 5: mod_base = OTHER; note = 2'-O-methoxyethyl modified nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 2'-O-methoxyethyl modified nucleotide with phosphorothioate linkage
  modified_base 15: mod_base = OTHER; note = 2'-O-methoxyethyl thymine with phosphorothioate linkage
  modified_base 16: mod_base = OTHER; note = 2'-O-methoxyethyl modified nucleotide with phosphorothioate linkage
  modified_base 17: mod_base = OTHER; note = 2'-O-methoxyethyl modified nucleotide with phosphorothioate linkage
  modified_base 18: mod_base = OTHER; note = 2'-O-methoxyethyl modified nucleotide
  misc_feature 1..5: note = RNA
  misc_feature 6..13: note = DNA
  misc_feature 14..18: note = RNA
tgcggccgcggaagtcca

SEQ ID NO: 738 - DNA, 18 bp, synthetic construct
  source 1..18: mol_type = other DNA
  modified_base 1: mod_base = OTHER; note = 2'-O-methoxyethyl modified nucleotide with phosphorothioate linkage
  modified_base 2: mod_base = OTHER; note = 2'-O-methoxyethyl thymine with phosphorothioate linkage
  modified_base 3: mod_base = OTHER; note = 2'-O-methoxyethyl modified nucleotide with phosphorothioate linkage
  modified_base 4: mod_base = OTHER; note = 2'-O-methoxyethyl modified nucleotide with phosphorothioate linkage
  modified_base 5: mod_base = OTHER; note = 2'-O-methoxyethyl thymine with phosphorothioate linkage
  modified_base 6: mod_base = OTHER; note = Thymine with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 2'-O-methoxyethyl thymine with phosphorothioate linkage
  modified_base 15: mod_base = OTHER; note = 2'-O-methoxyethyl thymine with phosphorothioate linkage
  modified_base 16: mod_base = OTHER; note = 2'-O-methoxyethyl modified nucleotide with phosphorothioate linkage
  modified_base 17: mod_base = OTHER; note = 2'-O-methoxyethyl modified nucleotide with phosphorothioate linkage
  modified_base 18: mod_base = OTHER; note = 2'-O-methoxyethyl modified nucleotide
  misc_feature 1..5: note = RNA
  misc_feature 6..13: note = DNA
  misc_feature 14..18: note = RNA
ctgcttcaccaccttgcg

SEQ ID NO: 739 - DNA, 18 bp, synthetic construct
  source 1..18: mol_type = other DNA
  modified_base 1: mod_base = OTHER; note = 2'-O-methoxyethyl thymine with phosphorothioate linkage
  modified_base 2: mod_base = OTHER; note = 2'-O-methoxyethyl modified nucleotide with phosphorothioate linkage
  modified_base 3: mod_base = OTHER; note = 2'-O-methoxyethyl thymine with phosphorothioate linkage
  modified_base 4: mod_base = OTHER; note = 2'-O-methoxyethyl modified nucleotide with phosphorothioate linkage
  modified_base 5: mod_base = OTHER; note = 2'-O-methoxyethyl modified nucleotide with phosphorothioate linkage
  modified_base 6: mod_base = OTHER; note = Thymine with phosphorothioate linkage
  modified_base 7: mod_base = OTHER; note = Thymine with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 2'-O-methoxyethyl modified nucleotide with phosphorothioate linkage
  modified_base 15: mod_base = OTHER; note = 2'-O-methoxyethyl thymine with phosphorothioate linkage
  modified_base 16: mod_base = OTHER; note = 2'-O-methoxyethyl thymine with phosphorothioate linkage
  modified_base 17: mod_base = OTHER; note = 2'-O-methoxyethyl modified nucleotide with phosphorothioate linkage
  modified_base 18: mod_base = OTHER; note = 2'-O-methoxyethyl modified nucleotide
  misc_feature 1..5: note = RNA
  misc_feature 6..13: note = DNA
  misc_feature 14..18: note = RNA
tctgcttcaccaccttgc

SEQ ID NO: 740 - DNA, 18 bp, synthetic construct
  source 1..18: mol_type = other DNA
  modified_base 1: mod_base = OTHER; note = 2'-O-methoxyethyl modified nucleotide with phosphorothioate linkage
  modified_base 2: mod_base = OTHER; note = 2'-O-methoxyethyl thymine with phosphorothioate linkage
  modified_base 3: mod_base = OTHER; note = 2'-O-methoxyethyl modified nucleotide with phosphorothioate linkage
  modified_base 4: mod_base = OTHER; note = 2'-O-methoxyethyl thymine with phosphorothioate linkage
  modified_base 5: mod_base = OTHER; note = 2'-O-methoxyethyl modified nucleotide with phosphorothioate linkage
  modified_base 6: mod_base = OTHER; note = Nucleotide with phosphorothioate linkage
  modified_base 7: mod_base = OTHER; note = Thymine with phosphorothioate linkage
  modified_base 8: mod_base = OTHER; note = Thymine with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 2'-O-methoxyethyl modified nucleotide with phosphorothioate linkage
  modified_base 15: mod_base = OTHER; note = 2'-O-methoxyethyl modified nucleotide with phosphorothioate linkage
  modified_base 16: mod_base = OTHER; note = 2'-O-methoxyethyl thymine with phosphorothioate linkage
  modified_base 17: mod_base = OTHER; note = 2'-O-methoxyethyl thymine with phosphorothioate linkage
  modified_base 18: mod_base = OTHER; note = 2'-O-methoxyethyl modified nucleotide
  misc_feature 1..5: note = RNA
  misc_feature 6..13: note = DNA
  misc_feature 14..18: note = RNA
gtctgcttcaccaccttg

SEQ ID NO: 741 - DNA, 20 bp, synthetic construct
  source 1..20: mol_type = other DNA
ccgccgctccaggccacccc

SEQ ID NO: 742 - DNA, 12 bp, synthetic construct
  source 1..12: mol_type = other DNA
aatttgttataa

SEQ ID NO: 743 - DNA, 20 bp, synthetic construct
  source 1..20: mol_type = other DNA
  modified_base 1: mod_base = OTHER; note = 2'-O-methoxyethyl modified nucleotide with phosphorothioate linkage
  modified_base 2: mod_base = OTHER; note = 2'-O-methoxyethyl modified nucleotide with phosphorothioate linkage
  modified_base 3: mod_base = OTHER; note = 2'-O-methoxyethyl modified nucleotide with phosphorothioate linkage
  modified_base 4: mod_base = OTHER; note = 2'-O-methoxyethyl modified nucleotide with phosphorothioate linkage
  modified_base 5: mod_base = OTHER; note = 2'-O-methoxyethyl thymine with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Thymine with phosphorothioate linkage
  modified_base 14: mod_base = OTHER; note = Thymine with phosphorothioate linkage
  modified_base 15: mod_base = OTHER; note = Thymine with phosphorothioate linkage
  modified_base 16: mod_base = OTHER; note = 2'-O-methoxyethyl modified nucleotide with phosphorothioate linkage
  modified_base 17: mod_base = OTHER; note = 2'-O-methoxyethyl modified nucleotide with phosphorothioate linkage
  modified_base 18: mod_base = OTHER; note = 2'-O-methoxyethyl modified nucleotide with phosphorothioate linkage
  modified_base 19: mod_base = OTHER; note = 2'-O-methoxyethyl modified nucleotide with phosphorothioate linkage
  modified_base 20: mod_base = OTHER; note = 2'-O-methoxyethyl modified nucleotide
  misc_feature 1..5: note = RNA
  misc_feature 6..15: note = DNA
  misc_feature 16..20: note = RNA
cccatccaccactttcggcc

SEQ ID NO: 744 - DNA, 20 bp, synthetic construct
  source 1..20: mol_type = other DNA
  modified_base 1: mod_base = OTHER; note = 2'-O-methoxyethyl modified nucleotide with phosphorothioate linkage
  modified_base 2: mod_base = OTHER; note = 2'-O-methoxyethyl modified nucleotide with phosphorothioate linkage
  modified_base 3: mod_base = OTHER; note = 2'-O-methoxyethyl modified nucleotide with phosphorothioate linkage
  modified_base 4: mod_base = OTHER; note = 2'-O-methoxyethyl modified nucleotide with phosphorothioate linkage
  modified_base 5: mod_base = OTHER; note = 2'-O-methoxyethyl modified nucleotide with phosphorothioate linkage
  modified_base 6: mod_base = OTHER; note = Thymine with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Thymine with phosphorothioate linkage
  modified_base 15: mod_base = OTHER; note = Thymine with phosphorothioate linkage
  modified_base 16: mod_base = OTHER; note = 2'-O-methoxyethyl thymine with phosphorothioate linkage
  modified_base 17: mod_base = OTHER; note = 2'-O-methoxyethyl modified nucleotide with phosphorothioate linkage
  modified_base 18: mod_base = OTHER; note = 2'-O-methoxyethyl modified nucleotide with phosphorothioate linkage
  modified_base 19: mod_base = OTHER; note = 2'-O-methoxyethyl modified nucleotide with phosphorothioate linkage
  modified_base 20: mod_base = OTHER; note = 2'-O-methoxyethyl modified nucleotide
  misc_feature 1..5: note = RNA
  misc_feature 6..15: note = DNA
  misc_feature 16..20: note = RNA
gcccatccaccactttcggc

(19) United States
(12) Patent Application Publication
(10) Pub. No.: US 2026/0185103 A1
(43) Pub. Date: Jul. 2, 2026

(54) COMPOSITIONS AND METHODS FOR TREATMENT OF CANCER AND METABOLIC DISEASE

(71) Applicant: Pretzel Therapeutics, Inc., Waltham, MA (US)

(72) Inventors: Yonghong Shi, Mölndal, Västergötland (SE); Xie Xie, Mölndal, Västergötland (SE); Xuefeng Zhu, Mölndal, Västergötland (SE)

(21) Appl. No.: 19/132,263

(22) PCT Filed: Nov. 22, 2023
(86) PCT No.: PCT/US2023/080992

§ 371 Date: May 22, 2025

Related U.S. Application Data
(60) Provisional application No. 63/427,685, filed on Nov. 23, 2022.
(60) Provisional application No. 63/495,479, filed on Apr. 11, 2023.

(51) Int. Cl. C12N 15/113

(52) U.S. Cl. CPC C12N 15/1137; C12Y 207/07006; C12N 2310/11; C12N 2310/313; C12N 2310/315; C12N 2310/321; C12N 2310/322; C12N 2310/3231; C12N 2310/3233; C12N 2310/3341; C12N 2310/351; C12N 2310/3525

(57) ABSTRACT

Oligonucleotide compositions and their use in treating various diseases such as cancer and metabolic disorders are described.

### CROSS REFERENCE TO RELATED APPLICATIONS

This application claims the benefit of U.S. Provisional Application Nos. 63/495,479, filed Apr. 11, 2023, and 63/427,685, filed Nov. 23, 2022, the contents of each of which are herein incorporated by reference in their entirety.

### BACKGROUND

Human mitochondrial RNA polymerase, POLRMT (also referred to as hmtRNAP), is a nuclear-encoded single-subunit DNA-dependent RNA polymerase. A primary biological role of POLRMT is to transcribe the mitochondrial genome to produce the RNAs needed for expression of mitochondrial DNA (mtDNA). The mitochondrial genome encodes the various subunits of the electron transport chain (see, e.g., Shokolenko, I. N., et al., Annu. Rev. Biochem., 85, 133-160, 2016). Specifically, transcription of the mitochondrial genome is necessary for the expression of 13 subunits of the oxidative phosphorylation (OXPHOS) system, as well as two rRNAs and 22 tRNAs (see, e.g., Shokolenko, I. N., et al., Frontiers in Bioscience, Landmark, 22, 835-853, 2017). Thus, POLRMT is essential for biogenesis of the OXPHOS system, resulting in ATP production. This, in turn, is vital for energy homeostasis in the cell.

Dysregulation of POLRMT and the OXPHOS system have been implicated in various disease states including cancer and metabolic disease. High rates of OXPHOS have been shown to support growth in cancer cell lines, including in a subset of diffuse large B cell lymphoma cells (see, e.g., DeBeradinis, R. J., Cancer Cell, 22, 423-24, 2012).

Cancer is now the second leading cause of death in the United States, with projections indicating that almost two million new cases will be diagnosed in 2022 and over 600,000 deaths will be the result of cancer (see Siegel, R. L. et al., CA Cancer J. Clin. (72) 7-33, 2022). Accordingly, there exists a need to develop new therapeutic strategies for treatment and prevention.

### SUMMARY

The present disclosure is based, at least in part, on the insight that, dysregulation of POLRMT and the OXPHOS system have been implicated in various disease states including cancer and metabolic disease. The present disclosure provides, among other things, the recognition that oligonucleotides that inhibit POLRMT are particularly beneficial as a treatment for cancer and metabolic diseases associated with mitochondrial dysfunction.

In one aspect, the present disclosure provides an oligonucleotide comprising a sequence that is substantially complementary to 8 to 30 contiguous nucleotides of a POLRMT RNA transcript. In some embodiments, the oligonucleotide comprises a sequence that is at least 85%, at least 90%, or at least 95% complementary to 8 to 30 contiguous nucleotides of a POLRMT RNA transcript. In some embodiments, the oligonucleotide comprises a sequence that is perfectly complementary to 8 to 30 contiguous nucleotides of a POLRMT RNA transcript. In some embodiments, the 8 to 30 contiguous nucleotides is 15 to 25 contiguous nucleotides. In some embodiments, the oligonucleotide is 8 to 30 nucleotides in length. In some embodiments, the oligonucleotide is 18 to 22 nucleotides in length. In some embodiments, the oligonucleotide is 20 nucleotides in length.

In some embodiments, the POLRMT RNA transcript is a human PORLMT RNA transcript. In some embodiments, the human POLRMT RNA transcript comprises SEQ ID NO: 205. In some embodiments, the 8 to 30 contiguous nucleotides is within or includes an exon region of the POLRMT RNA transcript. In some embodiments, the exon comprises an exon identified in any one of Ensemble ID Nos: ENSE00000655271, ENSE00000655279, and ENSE00000655283. In some embodiments, the oligonucleotide is complementary to 16-20 contiguous nucleotides of a sequence that corresponds to nucleotides 817-845, 2415-2446, or 2978-3008 of SEQ ID NO: 205 (i.e., the nucleotide sequences represented in SEQ ID NOs: 725, 726, or 727).

In some embodiments, the 8 to 30 contiguous nucleotides comprises a sequence that corresponds to nucleotides 2420-2439, 2422-2441, 2983-3002, 2984-3003, 822-839, 823-840, 2421-2438, 2422-2439, 2423-2440, 2424-2441, 2984-3001, 2985-3002, or 2986-3003 of SEQ ID NO: 205.

In another aspect, the present disclosure provides, an oligonucleotide comprising a sequence having at least 80% identity to a sequence selected from a group consisting of SEQ ID NOs: 592, 594, 597, 598, 612, 613, 623, 624, 625, 626, 632, 633, and 634. In some embodiments, the oligonucleotide comprises a sequence having at least 90% identity to a sequence selected from a group consisting of SEQ ID NOs: 592, 594, 597, 598, 612, 613, 623, 624, 625, 626, 632, 633, and 634. In some embodiments, the oligonucleotide comprises a sequence selected from a group consisting of SEQ ID NOs: 592, 594, 597, 598, 612, 613, 623, 624, 625, 626, 632, 633, and 634. In some embodiments, the oligonucleotide comprises SEQ ID NO: 594. In some embodiments, the oligonucleotide comprises SEQ ID NO: 612. In some embodiments, the oligonucleotide comprises SEQ ID NO: 632.

In another aspect, the present disclosure provides, an oligonucleotide comprising a sequence that is substantially complementary to a sequence selected from a group consisting of SEQ ID NOs: 661, 663, 666, 667, 681, 682, 692, 693, 694, 695, 701, 702, and 703. In some embodiments, the oligonucleotide is at least 85%, at least 90%, or at least 95% complementary to a sequence selected from a group consisting of SEQ ID NOs: 661, 663, 666, 667, 681, 682, 692, 693, 694, 695, 701, 702, and 703. In some embodiments, the oligonucleotide is perfectly complementary to a sequence selected from a group consisting of SEQ ID NOs: 661, 663, 666, 667, 681, 682, 692, 693, 694, 695, 701, 702, and 703. In some embodiments, the oligonucleotide comprises a sequence that is complementary to SEQ ID NO: 663. In some embodiments, the oligonucleotide comprises a sequence that is complementary to SEQ ID NO: 681. In some embodiments, the oligonucleotide comprises a sequence that is complementary to SEQ ID NO: 701.

In some embodiments, the oligonucleotide is a chirally pure oligonucleotide.

In some embodiments, the oligonucleotide comprises at least one modified nucleotide. In some embodiments, the modified nucleotide comprises a base modification, a sugar modification, a sugar phosphate modification, an internucleotidic linkage modification, or a combination thereof.

In some embodiments, the internucleotidic linkage modification comprises a phosphorothioate or phosphodithioate linkage modification.

In some embodiments, the sugar modification comprises a 2′-O-methoxyethyl (2′-MOE) modification, a 2′-Fluoro (2′-F) modification, a 2′-O-methyl (2′-O-Me) modification, an unlocked nucleic acid (UNA), or a locked nucleic acid (LNA).

In some embodiments, the sugar phosphate modification comprises a phosphorodiamidate morpholino (PMO) modification and/or a peptide nucleic acid (PNA) modification.

In some embodiments, the base modification comprises a 5′-methylcytosine modification or a G-clamp modification.

In some embodiments, each nucleotide comprises a phosphorothioate (PS) internucleotide linkage.

In some embodiments, the oligonucleotide comprises five nucleotides at the 5′-end and five nucleotides at the 3′-end of the oligonucleotide sequence which contain a 2′-MOE modification. In some embodiments, the oligonucleotide comprises any one of SEQ ID NOs: 728-740.

In some embodiments, each nucleotide contains a 2′-MOE modification.

In some embodiments, the oligonucleotide further comprises at least at least one ligand attached to the 5′ end and/or the 3′ end. In some embodiments, the ligand comprises at least one lipid, peptide, and/or sugar. In some embodiments, the sugar comprises one or more N-acetylgalactosamine (GalNAc) moieties.

In some embodiments, the GalNAc moiety comprises a structural formula comprising:

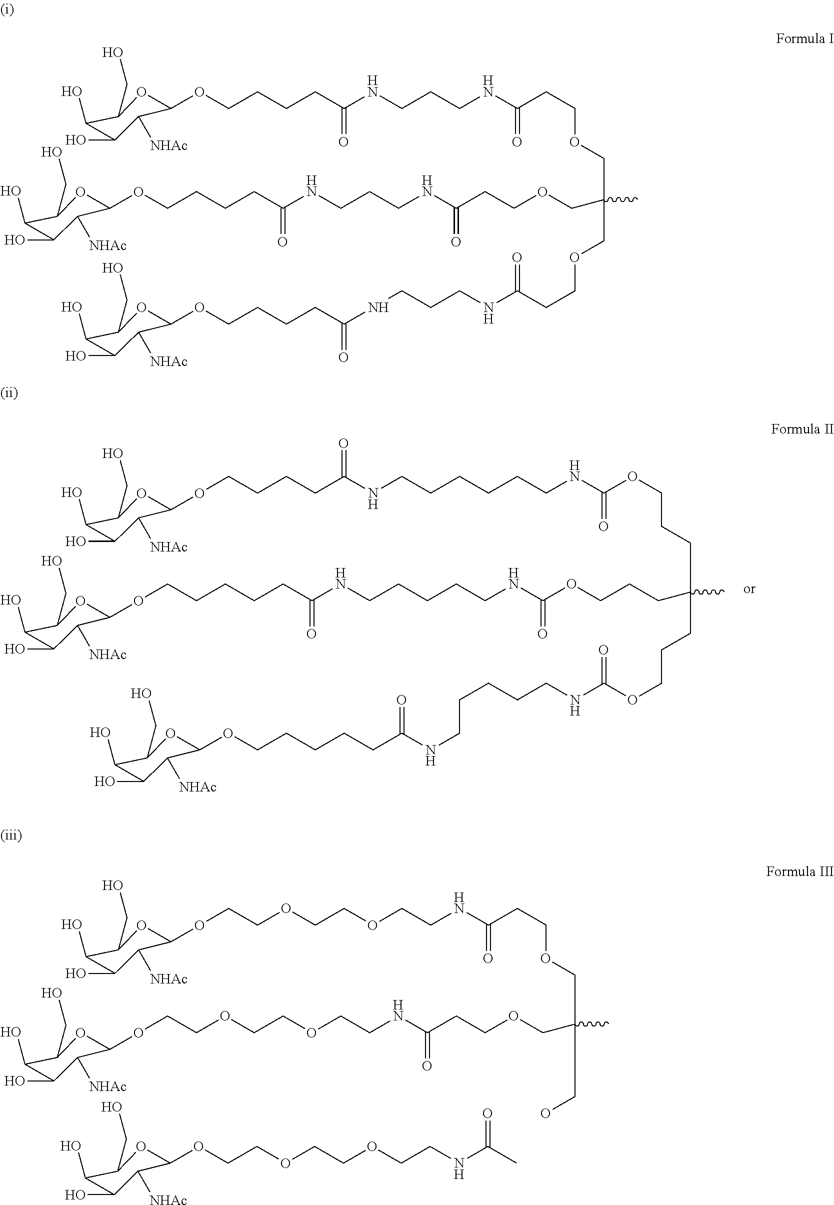

In some embodiments, the GalNAc moiety is conjugated to the oligonucleotide via a linker. In some embodiments, the linker comprises Formula A as follows:

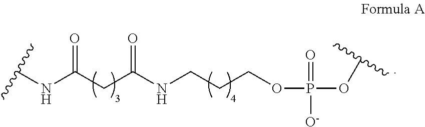

In some embodiments, the GalNAc moiety is conjugated to the oligonucleotide via a linker. In some embodiments, a 2′ deoxyadenosine phosphodiester is inserted between the oligonucleotide and the one or more GalNAc moieties.

In some embodiments, the oligonucleotide, when administered to a cell, is capable of reducing the level of POLRMT mRNA expression, POLRMT protein, and/or PORLMT activity in a cell by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% relative to a level before the administration. In some embodiments, the cell is a human cell.

In another aspect, the disclosure features an oligonucleotide sequence comprising a sequence that is complementary to a sequence that differs by no more than 1, 2, 3, or 4 nucleotides from a target region that spans between 8 to 30 contiguous nucleotides of SEQ ID NO: 1. In some embodiments, the oligonucleotide comprises a sequence that is complementary to a target region that spans between 8 to 30 contiguous nucleotides of SEQ ID NO: 1. In some embodiments, the target region spans between 15 to 25 contiguous nucleotides of SEQ ID NO: 1. In some embodiments, the target region spans 20 contiguous nucleotides of SEQ ID NO: 1. In some embodiments, the target region comprises an exon region of POLRMT. In some embodiments, the target region comprises a region that corresponds to nucleotides 5696-5715, 8808-8827, 8809-8828, 8811-8830, 16221-16240, 17159-17178, 17314-17333, 17315-17334, 18082-18101, 18083-18102, 18084-18103, 18130-18149, 5680-5699, 8491-8510, 8529-8548, 8569-8588, 8570-8589, 8571-8590, 8572-8591, 8573-8592, 8574-8593, 13322-13341, 13719-13738, 14999-15018, 15092-15111, 15093-15112, 17304-17323, 19309-19328,20041-20060, 20042-20061, or 21102-21121 of SEQ ID NO: 1.

In some embodiments, the oligonucleotide comprises a sequence that is substantially complementary to 8 to 30 contiguous nucleotides of a POLRMT RNA transcript.

In another aspect, the disclosure provides an oligonucleotide comprising a sequence having at least 80% identity to a sequence selected from a group consisting of SEQ ID NOs: 3-14. In some embodiments, the oligonucleotide comprises a sequence having at least 90% identity to a sequence selected from a group consisting of SEQ ID NOs: 3-14. In some embodiments, the oligonucleotide comprises a sequence selected from a group consisting of SEQ ID NOs: 3-14. In some embodiments, the oligonucleotide comprises SEQ ID NO: 11. In some embodiments, the oligonucleotide comprises SEQ ID NO: 12.

In another aspect, the disclosure provides an oligonucleotide comprising a sequence that is complementary to a sequence that is at least 80% identical to a sequence selected from a group consisting of SEQ ID NOs: 15-26. In some embodiments, the oligonucleotide comprises a sequence that is complementary to a sequence that is at least 90% identical to any one of SEQ ID NOs: 15-26. In some embodiments, the oligonucleotide comprises a sequence that is complementary to a sequence selected from a group consisting of SEQ ID NOs: 15-26. In some embodiments, the oligonucleotide comprises a sequence that is complementary to SEQ ID NO: 23. In some embodiments, the oligonucleotide comprises a sequence that is complementary to SEQ ID NO: 24.

In another aspect, the disclosure provides an oligonucleotide comprising a sequence that differs by no more than 1, 2, 3, or 4 nucleotides from any one of SEQ ID NOs: 3-14 and/or is complementary to a nucleotide sequence that differs by no more than 1, 2, 3, or 4 nucleotides from any one of SEQ ID NO: 15-26.

In another aspect, the disclosure provides an oligonucleotide sequence comprising a sequence that is complementary to a sequence that differs by no more than 1, 2, 3, or 4 nucleotides from a target region that spans between 8 to 30 contiguous nucleotides of SEQ ID NO: 581. In some embodiments, an oligonucleotide comprises a sequence that is complementary to a target region that spans between 8 to 30 contiguous nucleotides of SEQ ID NO: 581. In some embodiments, a target region spans between 15 to 25 contiguous nucleotides of SEQ ID NO: 581. In some embodiments, a target region spans 20 contiguous nucleotides of SEQ ID NO: 581. In some embodiments, a target region comprises an exon region of POLRMT. In some embodiments, a target region comprises a region that corresponds to nucleotides 3348-3367 or 3198-3217 of SEQ ID NO: 581.

In another aspect, the disclosure provides an oligonucleotide comprising a sequence having at least 80% identity to a sequence selected from a group consisting of SEQ ID NOs: 393-486. In some embodiments, an oligonucleotide comprises a sequence having at least 90% identity to a sequence selected from a group consisting of SEQ ID NOs: 393-486. In some embodiments, an oligonucleotide comprises a sequence selected from a group consisting of SEQ ID NOs: 393-486. In some embodiments, an oligonucleotide comprises SEQ ID NO: 434. In some embodiments, an oligonucleotide comprises SEQ ID NO: 442.

In another aspect, the disclosure provides an oligonucleotide comprising a sequence that is complementary to a sequence that is at least 80% identical to a sequence selected from a group consisting of SEQ ID NOs: 487-580. In some embodiments, an oligonucleotide comprises a sequence that is complementary to a sequence that is at least 90% identical to any one of SEQ ID NOs: 487-580. In some embodiments, an oligonucleotide comprises a sequence that is complementary to a sequence selected from a group consisting of SEQ ID NOs: 487-580. In some embodiments, an oligonucleotide comprises a sequence that is complementary to SEQ ID NO: 528. In some embodiments, an oligonucleotide comprises a sequence that is complementary to SEQ ID NO: 536.

In another aspect, the disclosure provides an oligonucleotide comprising a sequence that differs by no more than 1, 2, 3, or 4 nucleotides from any one of SEQ ID NOs: 393-486 and/or is complementary to a nucleotide sequence that differs by no more than 1, 2, 3, or 4 nucleotides from any one of SEQ ID NO: 487-580.

In some embodiments, oligonucleotides according to various aspects of the disclosure are chirally pure oligonucleotides.

In some embodiments, an oligonucleotide comprises at least one modified nucleotide. In some embodiments, the modified nucleotide comprises a base modification, a sugar or sugar phosphate modification, an internucleotidic linkage modification, or a combination thereof. In some embodiments, the internucleotidic linkage modification comprises a phosphorothioate or phosphodithioate linkage modification. In some embodiments, the sugar or sugar phosphate modification comprises a 2′-O-methoxyethyl (2′-MOE) modification, a 2′-fluoro (2′-F) modification, a 2′-O-methyl (2′-O-Me) modification, a phosphorodiamidate morpholino (PMO) modification, a peptide nucleic acid (PNA) modification, an unlocked nucleic acid (UNA), or a locked nucleic acid (LNA). In some embodiments, the base modification comprises a 5′-methylcytosine modification or a G-clamp modification. In some embodiments, each nucleotide comprises a phosphorothioate (PS) internucleotide linkage. In some embodiments, the oligonucleotide comprises five nucleotides at the 5′-end and five nucleotides at the 3′-end of the oligonucleotide sequence which contain a 2′-MOE modification. In some embodiments, each nucleotide contains a 2′-MOE modification.

In some embodiments, an oligonucleotide further comprises at least at least one ligand attached to the 5′ end and/or the 3′ end. In some embodiments, the ligand comprises at least one lipid, peptide, and/or sugar. In some embodiments, the sugar comprises N-acetylgalactosamine (GalNAc) moiety.

In another aspect, the disclosure provides a composition comprising an oligonucleotide described herein and a carrier and/or excipient.

In another aspect, the disclosure provides an expression vector comprising one or more sequences encoding one of more oligonucleotides described herein.

In another aspect, the disclosure provides a method of treating a subject having or at risk of cancer or metabolic disease, the method comprising administering to the subject a composition comprising an effective amount of an oligonucleotide described herein.

In some embodiments, a level of mitochondrial RNA polymerase (POLRMT) mRNA expression or POLRMT protein in the subject or in a biological sample from the subject after the administration of the composition is reduced relative to a level before the administration of the composition. In some embodiments, the level of POLRMT mRNA expression or POLRMT protein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% relative to a level before the administration. In some embodiments, the composition is administered intravenously, intrathecally, intramuscularly, orally, intranasaly, or subcutaneously to the subject. In some embodiments, the subject is a human.

In another aspect, the disclosure provides a method of treating and/or preventing a cancer or a metabolic disease in a subject comprising: administering to the subject an oligonucleotide that is complementary to a target region of a nucleic acid sequence encoding POLRMT.

In another aspect, the disclosure provides a method of decreasing mitochondrial transcription in a subject that is susceptible to or suffering from cancer or metabolic disease, the method comprising: administering to the subject an oligonucleotide that is complementary to a target region of a nucleic acid sequence encoding POLRMT.

In some embodiments, the nucleic acid sequence encoding POLRMT comprises SEQ ID NO: 1. In some embodiments, the target region comprises a region that spans between 8 to 30 contiguous nucleotides within SEQ ID NO: 1. In some embodiments, the target region comprises a region that corresponds to nucleotides 5696-5715, 8808-8827, 8809-8828, 8811-8830, 16221-16240, 17159-17178, 17314-17333, 17315-17334, 18082-18101, 18083-18102, 18084-18103, 18130-18149, 5680-5699, 8491-8510, 8529-8548, 8569-8588, 8570-8589, 8571-8590, 8572-8591, 8573-8592, 8574-8593, 13322-13341, 13719-13738, 14999-15018, 15092-15111, 15093-15112, 17304-17323, 19309-19328, 20041-20060, 20042-20061, or 21102-21121 of SEQ ID NO: 1. In some embodiments, the oligonucleotide comprises a sequence having at least 80% identity to a sequence selected from a group consisting of SEQ ID NOs: 3-14. In some embodiments, the oligonucleotide comprises a sequence having at least 90% identity to a sequence selected from a group consisting of SEQ ID NOs: 3-14. In some embodiments, the oligonucleotide comprises a sequence selected from a group consisting of SEQ ID NOs: 3-14.

In some embodiments, upon administration of the oligonucleotide to the subject, the level of POLRMT mRNA expression in the subject is decreased. In some embodiments, upon administration of the oligonucleotide to the subject, the level of POLRMT protein or activity in the subject is decreased. In some embodiments, the level of POLRMT mRNA expression, POLRMT protein, or POLRMT activity is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% relative to a level before the administration.

In some embodiments, the subject is a human. In some embodiments, the metabolic disease comprises include obesity, diabetes, non-alcoholic steatohepatitis (NASH), a disorder of amino acid metabolism (amino acidemias), a disorder of organic acid metabolism (organic acidurias, organic acidemias), a disorder of lipid metabolism (lipid storage disorders), a lysosomal storage disorder, a peroxisomal disorder, phenylketonuria (PKU), a glycogen storage disease, or a urea cycle disorder.

In some embodiments, the composition is delivered to the liver. In some embodiments, the composition is delivered to the muscle. In some embodiments, the composition is delivered to the CNS. In some embodiments, the composition is delivered to the cerebrospinal fluid.

In another aspect, the disclosure provides a pharmaceutical composition comprising an oligonucleotide described herein. In some embodiments, the pharmaceutical composition comprises a pharmaceutically acceptable carrier. In some embodiments, the oligonucleotide is formulated in a nanocarrier. In some embodiments, the oligonucleotide is formulated in a lipid nanoparticle (LNP). In some embodiments, the oligonucleotide is conjugated to at least one GalNAc moiety.

In some embodiments, the composition is formulated for systemic or localized administration. In some embodiments, the composition is formulated for delivery route selected from intrathecal, intramuscular, or intravenous administration.

In another aspect, the disclosure provides a method of reducing or inhibiting POLRMT expression in a cell, the method comprising contacting the cell with the oligonucleotide described herein. In some embodiments, the level of POLRMT mRNA expression, POLRMT protein, or POLRMT activity is reduced by at least 10%, at least 20%, at least 30%, at least 40%, at least 50%, at least 60%, at least 70%, at least 80%, or at least 90% relative to the level before the cell is contacted with the oligonucleotide.

In some embodiments, the cell is in a subject. In some embodiments, the subject is a human. In some embodiments, the human is suffering from or susceptible to cancer or a metabolic disorder.

### Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Acyl: As used herein, the term “acyl” means —C(O)R, wherein R is C₁₋₂₀ aliphatic.

Aliphatic: The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkyl: As used herein, the term “alkyl” means a straight-chain (i.e., unbranched) or branched, substituted or unsubstituted hydrocarbon chain that is completely saturated and that has a single point of attachment to the rest of the molecule.

Alkenyl: As used herein, the term “alkenyl” means a straight-chain (i.e., unbranched) or branched, substituted or unsubstituted hydrocarbon chain that comprises at least one carbon-carbon double bond and that has a single point of attachment to the rest of the molecule.

Alkynyl: As used herein, the term “alkynyl” means a straight-chain (i.e., unbranched) or branched, substituted or unsubstituted hydrocarbon chain that comprises at least one carbon-carbon triple bond and that has a single point of attachment to the rest of the molecule.

Alkylene: As used herein, the term “alkylene” refers to a bivalent alkyl group. An “alkylene chain” is a polymethylene group, i.e., —(CH₂)₄—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms in the chain are replaced with a substituent. Suitable substituents include those described below for a substituted aliphatic group.

Alkenylene: As used herein, the term “alkenylene” refers to a bivalent alkenyl group. A substituted alkenylene chain is a polymethylene group containing at least one carbon-carbon double bond in which one or more hydrogen atoms in the chain are replaced with a substituent. Suitable substituents include those described below for a substituted aliphatic group.

Alkynylene: As used herein, the term “alkynylene” refers to a bivalent alkynyl group. A substituted alkynylene chain is a polymethylene group containing at least one carbon-carbon triple bond in which one or more hydrogen atoms in the chain are replaced with a substituent. Suitable substituents include those described below for a substituted aliphatic group.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Cancer: As used herein, the term “cancer” refers to a disease, disorder, or condition in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer may be characterized by one or more tumors. Those skilled in the art are aware of a variety of types of cancer including, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, myeloid leukemia), lymphoma (e.g., Burkitt lymphoma [non-Hodgkin lymphoma], cutaneous T cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

Aryl: As used herein, the term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like. In certain preferred embodiments, the term aryl refers to phenyl.

Carbocyclic: As used herein, the terms “cycloaliphatic”, “carbocycle” or “cycloalkyl” refer to a monocyclic C₃-C₆ hydrocarbon that is completely saturated or that contains one or more units of unsaturation, but which is not aromatic, that has a single point of attachment to the rest of the molecule.

Complementary: As used herein, in accordance with its art-accepted meaning, “complementary” refers to the capacity for pairing between particular bases, nucleosides, nucleotides or nucleic acids. For example, adenine (A) and uracil (U) are complementary; adenine (A) and thymine (T) are complementary; and guanine (G) and cytosine (C) are complementary and are referred to in the art as Watson-Crick base pairings. If an oligonucleotide, i.e., a molecule comprising nucleotides, at a certain position (at a certain nucleotide of the oligonucleotide) within its sequence is complementary to a nucleotide in a second oligonucleotide when the oligonucleotides are aligned in anti-parallel orientation, the nucleotides of each oligonucleotide form a complementary base pairing and the oligonucleotides are said to complementary at that certain position. Thus, two oligonucleotides can be characterized by their percent of complimentary base pairing of their nucleotides. For example, the percent complementarity of a first oligonucleotide having a first nucleic acid sequence to a second oligonucleotide having a longer nucleic acid sequence may be evaluated by aligning them in antiparallel orientation and maximizing their complimentary base pairing. When an oligonucleotide is engineered to a target gene, the oligonucleotide may be evaluated for its complementarity to the pre-RNA or mRNA sequence of the target gene, and the alignment is said to be done over a window of evaluation along the RNA sequence. In this example, the percent complementarity of the base pairs in the oligonucleotide to the RNA sequence window is determined by the total number of nucleotides in the oligonucleotide and RNA sequence window that form base pairings, divided by the total number of nucleotides within the RNA sequence window, and multiplying by 100. For example, if the RNA sequence is AATTTGTTATAA, the window of evaluation (“RNA sequence window”) may be from nucleic acid at position #3, i.e., T (counting from left to right) to the nucleotide in position number #10, which is also a T. The RNA sequence window of this exemplary RNA sequence is 8 contiguous nucleotides in length. Aligning an oligonucleotide of AAAAAAAA along the aforementioned RNA sequence window would have an optimized alignment resulting in a maximum 75% complementary base parings since there are 6 nucleotides in Watson-Crick base pairings out of a total of 8 nucleotides in the RNA sequence window. A position occupied by two, non-complementary nucleotides constitutes a mismatch, i.e., the position is occupied by a non-complementary base pair. In the above example, 2 of the 8 nucleotides within the RNA sequence window are mismatched. When each nucleotide of an oligonucleotide is base pairing with each nucleotide of a second sequence of equal length (be it another oligonucleotide or RNA sequence window), such sequences can be referred to as “perfectly complementary” (100% complementarity) with respect to each other. Two nucleic acid sequences that are at least 80% complementary over a window of evaluation are considered “substantially complementary” over that window. In certain embodiments, two nucleic acid sequences are at least 75%, at least 80%, at least 85%, at least 90%, or at least 95% complementary over a window of evaluation. Where a first nucleic acid sequence is referred to as “substantially complementary” with respect to a second nucleic acid sequence herein, they may comprise one or more unmatched bases upon hybridization, e.g., up to about 5%, 10%, 15%, or 20% unmatched bases upon hybridization, e.g., 1, 2, 3, 4, 5, or 6 mismatched base pairs upon hybridization for a duplex up to 30 base pairs. It should be understood that where two oligonucleotides are designed to form, upon hybridization, one or more single stranded overhangs, such overhangs are not regarded as mismatches or unpaired nucleotides with regard to the determination of percent complementarity. “Complementary” sequences, as used herein may include one or more non-Watson-Crick base pairs and/or base pairs formed from non-natural nucleobases, in so far as the requirements with respect to their ability to hybridize are fulfilled. Such non-Watson-Crick base pairs include, but are not limited to, G:U Wobble or Hoogsteen base pairing. Those of ordinary skill in the art are aware that guanine, cytosine, adenine, thymine, and uracil can be replaced by other bases without substantially altering the base pairing properties of a polynucleotide comprising a nucleotide bearing such bases, according to the so-called “wobble” rules (see, e.g., Murphy, FV IV & V Ramakrishnan, V., Nature Structural and Molecular Biology 11: 1251-1252 (2004)). For example, a nucleotide comprising inosine as its base can base pair with nucleotides containing adenine, cytosine, thymine, or uracil. Thus, nucleotides containing uracil, guanine, thymine, or adenine can be replaced in the nucleic acid sequence of an oligonucleotide described herein by a nucleotide containing, for example, inosine, without decreasing the % complementarity. If a pair of bases is able to base pair (e.g., through Watson-Crick or Wobble base pairing), then such base pairs are considered to be complementary for purposes of determining % complementarity.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleotide residue in an oligonucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids and “corresponding” nucleotides. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/Hhsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides, oligonucleotides and/or nucleic acid sequences in accordance with the present disclosure.

Halogen: As used herein, the term “halogen” means F, Cl, Br, or I.

Heteroaryl: As used herein, the terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

Heteroatom: As used herein, the term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

Heterocycle: As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

Host cell: As used herein, the term “host cell” refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-1 1 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, and a cell line derived from an aforementioned cell. In some embodiments, the cell comprises one or more viral genes.

Identity: As used herein, the term “identity” refers to the overall relatedness between polymeric molecules, e.g., between nucleic acid molecules (e.g., DNA molecules and/or RNA molecules, such as oligonucleotides) and/or between polypeptide molecules. In some embodiments, polymeric molecules are considered to be “substantially identical” to one another if their sequences are at least 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can alternatively be determined using the GAP program in the GCG software package using an NWSgapdna.CMP matrix.

Linked: As used herein, the term “linked”, when used with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the invention the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linker” or “linking moiety” or “linking portion”, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which may be protected as appropriate, depending upon the conditions), and in sufficient amount, to produce a reasonable yield.

Operably linked: As used herein, the term “operably linked” refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

Optionally substituted or substituted: As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂, —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂, —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, (haloR^{•}), —OH, —OR^{•}, —O(haloR^{•}), —CN, —C(O)OH, —C(O)OR^{•}, —NH₂, —NHR^{•}, —NR^{•}₂, or NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Recombinant: As used herein, the term “recombinant” is intended to refer to polypeptides, polynucleotides, or oligonucleotides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Subject: As used herein, the term “subject” or “test subject” refers to any organism to which a provided compound or composition is administered in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder, and/or condition has been diagnosed with and/or displays one or more symptoms of a disease, disorder, and/or condition.

Target gene: A “target gene”, as used herein, refers to a gene whose expression is to be modulated, e.g., inhibited.

Target Region: As used herein, the term “target region” refers to a region within the RNA transcript of the target gene where the RNA is to be degraded or translationally repressed or otherwise inhibited using one or more oligonucleotides. In some embodiments, an oligonucleotide described herein is complementary to a target region (e.g., substantially or perfectly complementary), such that the oligonucleotide is capable of hybridizing to the target region. A target region, as described herein, may be described by its position (i.e., the coordinates of the nucleotides of the target region) within a target RNA sequence or the corresponding region within the target gene sequence. The RNA may be a primary RNA transcript transcribed from the target gene (e.g., a pre-mRNA) or a processed transcript, e.g., mRNA encoding a polypeptide. In some embodiments, a target region of an mRNA is at least long enough to serve as a substrate for RNAase-mediated degradation within that portion in the presence of a suitable oligonucleotides. A target region may be from about 8-36 nucleotides in length, e.g., about 8-30, 10-20, or about 15-30 nucleotides in length. A target region length may have specific value or subrange within the afore-mentioned ranges.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition. In some embodiments, a therapeutic agent is an oligonucleotide designed to target a certain region of target gene.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or signs of the disease, disorder, and/or condition. In some embodiments, a therapeutically effective amount is administered in a single dose; in some embodiments, multiple unit doses are required to deliver a therapeutically effective amount.

Treating: As used herein, the term “treating” refers to providing treatment, i.e., providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a cancer or metabolic-related condition, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of the disclosure can be administered to a subject who has developed cancer or a metabolic-related disorder or is at increased risk of developing such a disorder relative to a member of the general population. A composition of the disclosure can be administered prophylactically, i.e., before development of any symptom or manifestation of the condition. Typically, in this case the subject will be at risk of developing the condition.

Nucleic acid: The term “nucleic acid” includes any nucleotides, analogs thereof, and polymers thereof. The term “polynucleotide” or “oligonucleotide” as used herein refers to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogs of either RNA or DNA made from nucleotide analogs and modified polynucleotides such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges (also referred to herein as “internucleotide linkages”). The terms further encompass nucleic acids containing any combinations of nucleobases, modified nucleobases, sugars, modified sugars, phosphate bridges or modified phosphorus atom bridges. Examples include, and are not limited to, nucleic acids containing ribose moieties, the nucleic acids containing deoxy-ribose moieties and nucleic acids containing both ribose and deoxyribose moieties, nucleic acids containing ribose and modified ribose moieties. In some embodiments, the prefix poly- refers to a nucleic acid containing 2 to about 10,000, 2 to about 50,000, or 2 to about 100,000 nucleotide monomer units. In some embodiments, the prefix oligo- refers to a nucleic acid containing 2 to about 200 nucleotide monomer units.

Unsaturated: The term “unsaturated”, as used herein, means that a moiety has one or more units of unsaturation.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### BRIEF DESCRIPTION OF THE DRAWING

FIG. 1 shows a schematic of mitochondria in a cell, including the oxidative phosphorylation system (OXPHOS) and the mitochondrial DNA (mtDNA), which is an exemplary target of the oligonucleotides described herein.

FIG. 2 shows a schematic of the various POLRMT RNA transcripts targeted by exemplary oligonucleotides described herein.

FIG. 3 shows two exemplary oligonucleotides described herein, including their nucleotide sequence and particular modification pattern. In this figure, a blue shaded circle indicates a 2′-O-MOE group and a red line between nucleotides represents a phosphorothioate (PS) bond.

FIG. 4 shows relative POLRMT mRNA expression in HeLa cells transfected with exemplary oligonucleotides.

FIG. 5 shows relative CytB mRNA expression in HeLa cells transfected with exemplary oligonucleotides.

FIG. 6 shows relative POLRMT mRNA expression in 3T3 cells transfected with exemplary oligonucleotides at 100 nM.

FIG. 7 shows relative POLRMT mRNA expression in 3T3 cells transfected with exemplary oligonucleotides at 100 nM.

FIG. 8 shows relative POLRMT mRNA expression in 3T3 cells transfected with exemplary oligonucleotides at 100 nM.

FIG. 9 shows relative POLRMT mRNA expression in 3T3 cells transfected with exemplary oligonucleotides at 100 nM.

FIG. 10 shows relative POLRMT mRNA expression in 3T3 cells transfected with exemplary oligonucleotides at 30 nM.

FIG. 11 shows relative POLRMT mRNA expression in 3T3 cells transfected with exemplary oligonucleotides at 30 nM.

FIG. 12 shows a schematic of the human POLRMT transcript and exemplary human-mouse matched oligonucleotides arranged based on their target region on the POLRMT transcript. This schematic also identifies three “hotspot” regions identified and described herein.

FIG. 13 shows POLRMT expression and cell viability of human 143B and mouse 3T3 cells transfected with exemplary oligonucleotides (corresponding to nucleotide sequences shown in SEQ ID NOs: 612, 613, 623, 624, 632, 633, and 634) at various concentrations. Panel (A) shows POLRMT expression in human 143B cells transfected with exemplary oligonucleotides at various concentrations. Panel (B) shows viability of human 143B cells transfected with exemplary oligonucleotides at various concentrations. Panel (C) shows POLRMT expression in mouse 3T3 cells transfected with exemplary oligonucleotides at various concentrations. Panel (D) shows viability of mouse 3T3 cells transfected with exemplary oligonucleotides at various concentrations.

FIG. 14 shows POLRMT expression and cell viability of human 143B and mouse 3T3 cells transfected with exemplary oligonucleotides (corresponding to nucleotide sequences shown in SEQ ID NOs: 592, 594, 597, 598, 625, and 626) at various concentrations. Panel (A) shows POLRMT expression in human 143B cells transfected with exemplary oligonucleotides at various concentrations. Panel (B) shows viability of human 143B cells transfected with exemplary oligonucleotides at various concentrations. Panel (C) shows POLRMT expression in mouse 3T3 cells transfected with exemplary oligonucleotides at various concentrations. Panel (D) shows viability of mouse 3T3 cells transfected with exemplary oligonucleotides at various concentrations.

FIG. 15 shows in vitro toxicity of HepG2 cells and 3T3 cells transfected with various exemplary oligonucleotides described herein at 100 nM, expressed as a ratio relative to a vehicle control.

FIG. 16 shows expression of POLRMT in HepG2 cells transfected with exemplary oligonucleotides described herein at a concentration of 100 nM relative to a vehicle control (Panel A) and viability of HepG2 cells transfected with exemplary oligonucleotides expressed as a percentage (%) relative to a vehicle control (Panel B).

### DETAILED DESCRIPTION OF CERTAIN EMBODIMENTS

### Mitochondrial RNA Polymerase (POLRMT), Cancer and Metabolic Disease

The present disclosure provides, among other things, compositions and methods for treating cancer and metabolic diseases through inhibition of POLRMT.

Human mitochondrial RNA polymerase, POLRMT (also referred to as hmtRNAP), is a nuclear-encoded single-subunit DNA-dependent RNA polymerase. POLRMT is 1230 amino acids in length and consists of three distinct regions: (1) a C-terminal polymerase domain (CTD) (residues 648-1230); (2) an N-terminal domain (NTD) (residues 369-647); and (3) an N-terminal extension (NTE) (residues 1-368) (see, e.g., Arnold, J. J., et al., Biochim. Biophys. Acta, 1819, 948-960, 2012). It is structurally related to the single-subunit RNA polymerase encoded by bacteriophage T7. The CTD is also known as the catalytic domain due to its function of catalyzing nucleotide incorporation into a growing RNA molecule during transcription. This domain is highly conserved across species, whereas by contrast the NTE demonstrates significant sequence variability, suggesting organism-specific roles for this domain of POLRMT. Structurally, the NTD of POLRMT resembles the N-terminal domain (also called the promoter-binding domain) of T7 RNA polymerase. However, for promoter-specific transcription initiation, POLRMT requires assistance from additional transcription factors, whereas T7 RNA polymerase does not.

The protein sequence of wildtype human POLRMT is as follows (1230 amino acids):

(SEQ ID NO: 2, transit peptide)
MSALCWGRGAAGLKRALRPCGRPGLPGKEGTAGGVCGPRRSSSASPQEQDQDRRKDW
GHVELLEVLQARVRQLQAESVSEVVVNRVDVARLPECGSGDGSLQPPRKVQMGAKDA
TPVPCGRWAKILEKDKRTQQMRMQRLKAKLQMPFQSGEFKALTRRLQVEPRLLSKQM
AGCLEDCTRQAPESPWEEQLARLLQEAPGKLSLDVEQAPSGQHSQAQLSGQQQRLLAFF
KCCLLTDQLPLAHHLLVVHHGQRQKRKLLTLDMYNAVMLGWARQGAFKELVYVLFM
VKDAGLTPDLLSYAAALQCMGRQDQDAGTIERCLEQMSQEGLKLQALFTAVLLSEEDR
ATVLKAVHKVKPTFSLPPQLPPPVNTSKLLRDVYAKDGRVSYPKLHLPLKTLQCLFEKQ
LHMELASRVCVVSVEKPTLPSKEVKHARKTLKTLRDQWEKALCRALRETKNRLEREVY
EGRFSLYPFLCLLDEREVVRMLLQVLQALPAQGESFTTLARELSARTFSRHVVQRQRVS
GQVQALQNHYRKYLCLLASDAEVPEPCLPRQYWEELGAPEALREQPWPLPVQMELGKL
LAEMLVQATQMPCSLDKPHRSSRLVPVLYHVYSFRNVQQIGILKPHPAYVQLLEKAAEP
TLTFEAVDVPMLCPPLPWTSPHSGAFLLSPTKLMRTVEGATQHQELLETCPPTALHGAL
DALTQLGNCAWRVNGRVLDLVLQLFQAKGCPQLGVPAPPSEAPQPPEAHLPHSAAPAR
KAELRRELAHCQKVAREMHSLRAEALYRLSLAQHLRDRVFWLPHNMDFRGRTYPCPP
HFNHLGSDVARALLEFAQGRPLGPHGLDWLKIHLVNLTGLKKREPLRKRLAFAEEVMD
DILDSADQPLTGRKWWMGAEEPWQTLACCMEVANAVRASDPAAYVSHLPVHQDGSC
NGLQHYAALGRDSVGAASVNLEPSDVPQDVYSGVAAQVEVFRRQDAQRGMRVAQVL
EGFITRKVVKQTVMTVVYGVTRYGGRLQIEKRLRELSDFPQEFVWEASHYLVRQVFKS
LQEMFSGTRAIQHWLTESARLISHMGSVVEWVTPLGVPVIQPYRLDSKVKQIGGGIQSIT
YTHNGDISRKPNTRKQKNGFPPNFIHSLDSSHMMLTALHCYRKGLTFVSVHDCYWTHA
ADVSVMNQVCREQFVRLHSEPILQDLSRFLVKRFCSEPQKILEASQLKETLQAVPKPGAF
DLEQVKRSTYFFS

There are 8 known mRNA splice variants of the POLRMT gene, which are publicly available, and identified in ENSEMBL IDs: ENST00000588649.7, ENST00000590573.4, ENST00000590336.2, ENST00000592863.2, ENST00000587057.5, ENST00000590709.3, ENST00000589961.2, and ENST00000592633.5. The POLRMT mRNA transcript that encodes the full-length POLRMT protein (identified above in SEQ ID NO: 2) is identified in ENSEMBL ID: ENST00000588649.7 (corresponding to SEQ ID NO: 205).

A primary biological role of POLRMT is to transcribe the mitochondrial genome to produce the RNAs needed for expression of mitochondrial DNA (mtDNA). Initiation, elongation, and termination are the three steps of mitochondrial transcription. Each of a light-strand promoter (LSP) and two heavy-strand promoters (HSP-1 and HSP-2) on the mtDNA contains a transcription initiation site (see, e.g., Basu, U. et al., J. Biol. Chem., 295(52), 18406-425, 2020). For promoter-specific transcription initiation, POLRMT requires two transcription factors, TFAM (transcription factor A mitochondrial) and TFB2M (transcription factor B mitochondrial). See id. Various models suggest different mechanisms by which the initiation complex structure with POLRMT, TFAM, and TFB2M comes together to cover the promoter DNA for initiation of transcription. In one current model TFAM recruits POLRMT to the promoter site to form a protein-protein pre-initiation complex, to which TFB2M binds to form the initiation complex, which covers the promoter DNA. See id. During initiation, the RNA is elongated to about 8-10 nucleotides in length. Conformational changes occur at that point, including promoter release and displacement of the initiation factors, converting the initiation complex into an elongation complex at which time transcription occurs. See id.

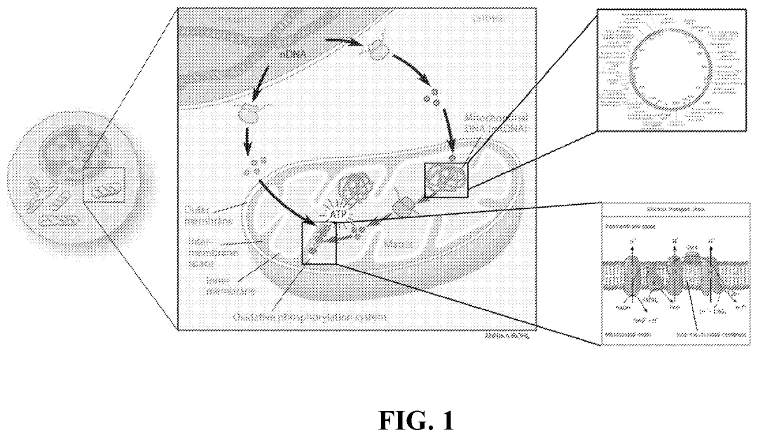

The mitochondrial genome encodes the various subunits of the electron transport chain (see, e.g., Shokolenko, I. N., et al., Annu. Rev. Biochem., 85, 133-160, 2016). Specifically, transcription of the mitochondrial genome is necessary for the expression of 13 subunits of the oxidative phosphorylation (OXPHOS) system, as well as two rRNAs and 22 tRNAs (see, e.g., Shokolenko, I. N., et al., Frontiers in Bioscience, Landmark, 22, 835-853, 2017). Thus, POLRMT is essential for biogenesis of the OXPHOS system, resulting in ATP production. This, in turn, is vital for energy homeostasis in the cell. FIG. 1 shows a schematic of the mitochondria, including the OXPHOS system and the mtDNA genome.

Dysregulation of POLRMT and the OXPHOS system have been implicated in various disease states, in particular cancer. Cancer is now the second leading cause of death in the United States, with projections indicating that almost two million new cases will be diagnosed in 2022 and over 600,000 deaths will be the result of cancer (see Siegel, R. L. et al., CA Cancer J. Clin. (72) 7-33, 2022). High rates of OXPHOS have been shown to support growth in cancer cell lines, including in a subset of diffuse large B cell lymphoma cells (see, e.g., DeBeradinis, R. J., Cancer Cell, 22, 423-24, 2012). Noteworthy is the observation that metabolic heterogeneity exists not only between different types of cancer, but also among tumors of the same type. Similarly, in a study using melanoma cell lines representative of various stages of tumor progression and that collectively mimic the mixture of cells found in a tumor, it was found that metastatic cells demonstrated a high OXPHOS capacity (Rodrigues, M. F., et al., Biochem. J. 473: 703-715, 2016). These data suggest mitochondria play a role as cells progress toward metastasis, possibly to provide the energy needed for tumor cell migration and invasion.

Relatedly, overexpression of POLRMT has been linked to multiple types of cancers, suggesting that it plays a role in tumor growth. Supporting this hypothesis is, for example, a study involving acute myeloid leukemia (AML) cells, which are known to have high oxidative phosphorylation and mitochondrial mass, as well as low respiratory chain spare reserve capacity. POLRMT knockdown in AML cells demonstrated a reduction in POLRMT levels, decreased oxidative phosphorylation, and increased cell death as compared to control AML cells (see Bralha, F. N., et al., Oncotarget, 6(35), 37216-228, 2015). In other work, injection into nude mice of a human breast cancer cell line that overexpresses POLRMT resulted in increased tumor growth, independent of tumor angiogenesis, suggesting that POLRMT should be considered a tumor promoter or metabolic oncogene (Salem, A. F., et al. Cell Cycle, 11(22), 4174-80, 2012). Recently, the expression of POLRMT in non-small cell lung cancer (NSCLC) has been examined (see Zhou, T. et al., Cell Death and Disease, 12, 751, 2021).

The development of multidrug resistance (MDR) to numerous cancers is associated with poor prognosis and presents significant challenges in the treatment of this disease. Because such resistance encompasses drugs having different structures and mechanisms of action, identifying and targeting a single biochemical pathway that could re-sensitize MDR cancer cells to established chemotherapy would provide a promising treatment strategy (see Yu, H.-J., Front. Chem., 9:775226, 2021). A main reason for the development of MDR is enhanced drug efflux from and decreased drug accumulation in MDR cells due to ATP-dependent protein transporters that pump drugs out of cells. Inhibiting POLRMT and consequently the production of the proteins essential for the OXPHOS system could compromise ATP production and, in turn, the ATP-dependent efflux of chemotherapeutic agents from cancer cells.

Consistent with the findings that the OXPHOS system and POLRMT may be involved in the etiology of and in some cases overexpressed in some cancers, small molecule inhibitors of POLRMT have been developed (see, e.g., EP 3 598 972 A1; WO 2019/057821 A1; and WO 2020/188049 A1, which are herein incorporated by reference in their entirety). Some of these inhibitors have been shown to be useful in inhibiting cancer cell proliferation without affecting control cells (see Bonekamp, N. A., et al., Nature, 588, 712-716, 2020). The cancer cell toxicity was correlated to a considerable increase in the levels of mono- and diphosphate nucleotides with a concomitant decrease in nucleotide triphosphate levels, all the result of a debilitated OXPHOS system. Similarly, treatment with POLRMT inhibitors caused a decrease in citric-acid cycle intermediates and ultimately cellular amino acid levels, the result of which is a state of severe energy and nutrient depletion. See id. Such inhibitors also produced a decrease in tumor volume in mice with no significant toxicity in control animals, Specifically, mtDNA transcript levels in tumor cells were decreased as compared to transcript levels in differentiated tissue. These data highlight the importance of mtDNA expression in rapidly dividing cells as opposed to post-mitotic tissue, a distinction that may be capitalized on using POLRMT inhibitors that are capable of modulating mtDNA transcription and ultimately the OXPHOS system.

While mitochondria are an emerging target for cancer treatment, the resistance mechanisms induced by chronic inhibition of mitochondrial function are poorly understood. In view of the challenges presented by drug resistance in cancer chemotherapy, the development of such resistance to small molecule inhibitors of POLRMT has been investigated (see Mennuni, M. et al., EMBO reports, 23: e53054 1-18, 2022). Using a CRISPR-Cas9 whole-genome screen, loss of genes belonging to von Hippel-Lindau (VHL) and mammalian target of rapamycin complex 1 (mTORC1) were the pathways that caused resistance to acute treatment with a POLRMT inhibitor. See id. at pp. 1-2. Moreover, dose-escalated chronic treatment of cells with this molecule resulted in drug-resistant cells that had increased levels of mtDNA, thereby giving rise to increased levels of mitochondrial transcripts and proteins. See id. at p. 5. The drug-resistant cells maintained higher levels of nucleotide levels, tricarboxylic acid cycle intermediates, and amino acids. See id. at p. 7. Notably, the drug-resistant cells did not have mutations in POLRMT that compromise inhibitor binding to the polymerase. See id. The development of resistance to POLRMT inhibitors underscores the importance and need for the development of other POLRMT inhibitors to understand and treat cancers of varying types.

Alterations in the OXPHOS system also have been implicated in the development of metabolic diseases such as insulin resistance and ultimately Type-2 diabetes. In studies involving apoptosis inducing factor (AIF) knockout mice, a primary OXPHOS defect that produced OXPHOS deficiency revealed an increase in insulin sensitivity and resistance to diabetes and obesity (see Pospisilik, J. A., et al., Cell, 131, 476-91, 2007). Correlated with these phenotypic changes were the metabolic alterations of increased glucose uptake and enhanced fuel utilization, Manipulation of the OXPHOS system with POLRMT modulators affords the potential for further understanding the physiological mechanisms involved in diseases such as diabetes and for the development of novel treatments for intervention of such metabolic disorders.

In addition to its critical role in transcription, POLRMT acts as the primase for mtDNA replication, thus playing a part in the regulation of mtDNA levels. Human mtDNA is a circular double-stranded DNA that is packaged in DNA-protein structures called mitochondrial nucleoids, for which TFAM is the most abundant structural component (see, e.g., Filograna, R., et al., FEBS Letters, 595, 976-1002, 2021). TFAM facilitates mtDNA compaction, which results in regulating the accessibility of the DNA to cellular replication and transcription components. With respect to mtDNA replication, POLRMT is part of the mtDNA replisome along with the hexameric helicase TWINKLE, the heterotrimeric DNA polymerase gamma (POL7) and the tetrameric mitochondrial single-stranded DNA-binding protein (mtSSB). See id. Its function in this replisome is to synthesize the RNA primers required for the initiation of the synthesis of both strands of mtDNA. While there may be many mechanisms by which mtDNA levels may be regulated, including modulation of POLRMT, what is known to date is that mtDNA copy number can be manipulated through modulation of TFAM expression.

While the correlation is not completely straightforward, changed levels of mtDNA have been implicated in neurodegenerative disorders, cancer, and aging (see e.g., Filograna, R., et al., FEBS Letters, 595, 976-1002, 2021). Particularly challenging is the attempt to understand the relationship between mtDNA copy number and cancer. It appears that such copy number can correlate with both increased and decreased disease burden. As such, tumor type and stage of disease may be important factors in determining the role of mtDNA copy number in the diagnosis and/or prognosis of cancer. With respect to aging, most data show a reduction in mtDNA levels in the older population. That being said, other study data are inconsistent as to the relationship between mtDNA copy number and longevity. By contrast, there appears to be a clearer correlation between neurodegeneration in Alzheimer's disease and reduction in mtDNA levels. Complicating the understanding of the relationship between mtDNA levels and disease is the role that mtDNA mutations have on various disorders. While accumulation of mtDNA mutations appears to occur in almost all types of cancer, it is unclear whether such mutations are causative of the cancer or merely a by-product of rapid replication in fast-dividing cells. Nonetheless, since POLRMT plays a key role in mtDNA replication, POLMRT modulation may provide an effective mechanism by which to understand various disease states and how to slow or alter the progression of disease.

Mutations affecting POLRMT may also cause human disease (see Oláhová, M., et al., Nat. Commun., 12, 1135, 2021). POLRMT variants have been identified in a number of unrelated families. Patients present with multiple phenotypes, including global developmental delay, hypotonia, short stature, and speech/intellectual disability in childhood. POLRMT modulation may provide a mechanism to slow or alter the progression of disease.

POLRMT is of fundamental importance for both expression and replication of the human mitochondrial genome. While aspects of POLRMT biochemistry are known, its full physiological role in mitochondrial gene expression and homeostasis, as well as its underlying impact in the etiology of various disease states, remains unclear. Its dysfunction and/or deregulation impacts mitochondrial metabolism, sometimes through the OXPHOS system, which ultimately contributes to many metabolic, degenerative and age-related diseases such as cancer, diabetes, obesity, and Alzheimer's disease. Inhibition of POLRMT is one means by which to gain a further understanding of the role of this polymerase in cell physiology and the development of disease. Regulation of metabolic mechanisms, including oxidative phosphorylation, with POLRMT modulators affords an opportunity for intervention in complex disorders. In view of the numerous and varied roles of POLRMT, the need exists for potent and specific modulators of POLRMT.

### POLRMT Oligonucleotides

In some embodiments, the present disclosure provides oligonucleotides that bind to and inhibit expression of messenger RNA (mRNA) produced by a target gene (e.g., POLRMT). As used herein, the terms “oligonucleotides” and “antisense oligonucleotides” are used interchangeably.

In some embodiments, administration of an oligonucleotide can decrease or inhibit mRNA expression of POLRMT in a subject or in a biological sample compared to a level before administration. In some embodiments, administration of an oligonucleotide can decrease level of POLRMT protein in a subject or in a biological sample compared to a level before administration. In some embodiments, administration of an oligonucleotide can decrease POLRMT activity (thereby decreasing mitochondrial transcription) in a subject or in a biological sample compared to a level before administration.

Indications of decreased POLRMT activity include decreased level of mitochondrial transcription. In some embodiments, decreased level of mitochondrial transcription can be measured by total mtDNA. Additionally, decreased mitochondrial transcription can also be indicated by a decrease in mRNA expression of various mitochondrial proteins, for example, decreased mRNA expression of Cytochrome B. Other mitochondrial proteins include various subunits of the electron transport chain (see, e.g., Shokolenko, I. N., et al., Annu. Rev. Biochem., 85, 133-160, 2016), and more specifically, the 13 subunits of the oxidative phosphorylation (OXPHOS) system (see, e.g., Shokolenko, I. N., et al., Frontiers in Bioscience, Landmark, 22, 835-853, 2017). Indications of decreased POLRMT activity also include decreased level ATP production.

In some embodiments, when oligonucleotides described herein are administered to a cell, level of POLRMT mRNA expression, POLRMT protein, and/or POLRMT activity is reduced in the cell by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% relative to a level before the administration. In some embodiments, administration of an oligonucleotide can lead to complete or substantially complete inhibition of POLRMT mRNA expression.

In some embodiments, an oligonucleotide described herein is an RNase H-dependent oligonucleotide, wherein the oligonucleotide induces the degradation of mRNA by RNase H. In some embodiments, an oligonucleotide inhibits expression of a target gene through steric-blocking, wherein the oligonucleotide physically prevents or inhibits the progression of splicing or translational machinery. Oligonucleotides, as described herein, are capable of hybridizing to a target region of a target nucleic acid, resulting in at least one antisense activity. In some embodiments, antisense activity comprises degradation of a target nucleic acid by RNase H. In some embodiments, antisense activity comprises an oligonucleotide physically preventing or inhibiting the progression of splicing or translational machinery.

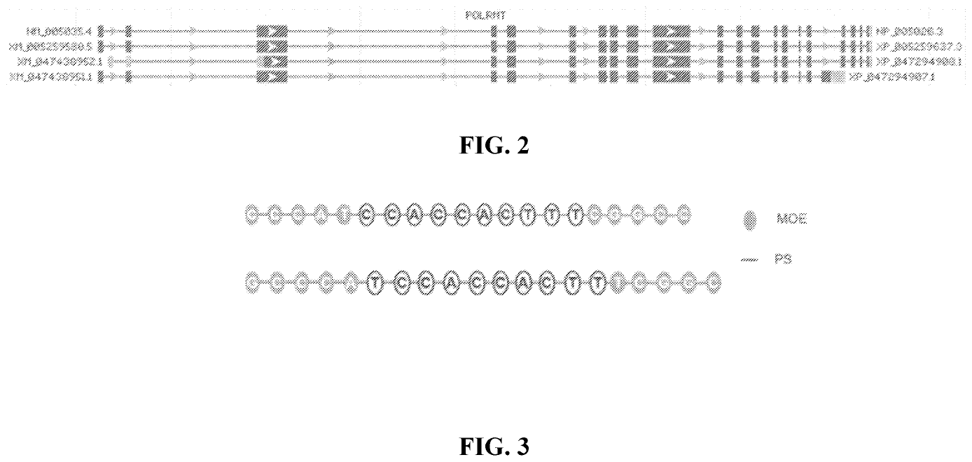

In some embodiments, oligonucleotides described herein specifically hybridize to one or more target regions on an RNA transcript of a target gene. In some embodiments, a target region comprises a region of an mRNA (e.g., a region within SEQ ID NO: 205). In some embodiments, a target region comprises a region of a pre-mRNA. In some embodiments, a target region comprises a region of pre-mRNA that spans an exon/intron junction. In some embodiments, a target region comprises a region of pre-mRNA spanning or including an intron region. In some embodiments, a target region corresponds to a region of a DNA sequence, i.e., a target gene sequence. In some embodiments, a target region comprises a region near to, that includes or is within a 5′-UTR region. In some embodiments, a target region comprises a region near to, that includes, or is within a 3′-UTR region. In some embodiments, a target region comprises a region near to, that includes, or is within an exon region (e.g., as shown in the transcripts of FIG. 2).

Exemplary target regions as described herein are shown in several POLRMT transcripts as shown in FIG. 2. The amino acid and nucleotide sequences of human POLRMT are known in the art and can be found in publicly available databases. For example, POLRMT transcript sequences are identified in Accession Numbers NM_005035.4, XM_005259580.5, XM_047438952.1, and XM_047438951.1 and ENSEMBL IDs of the 8 known mRNA transcripts are identified in: ENST00000588649.7, ENST00000590573.4, ENST00000590336.2, ENST00000592863.2, ENST00000587057.5, ENST00000590709.3, ENST00000589961.2, and ENST00000592633.5. The POLRMT mRNA transcript that encodes the full-length POLRMT protein (SEQ ID NO: 2) is identified in ENSEMBL ID: ENST00000588649.7 (corresponding to SEQ ID NO: 205). Additionally, the full human POLRMT gene sequence is represented in Reference No. NG_023049.1 (SEQ ID NO: 1).

A POLRMT mRNA transcript sequence is presented herein in SEQ ID NO: 205 (ENSEMBL ID: ENST00000588649.7), where U residues are represented by T residues in the provided sequence. One of ordinary skill in the art will appreciate that where one refers to a sequence as “RNA” or “mRNA” or “pre-mRNA” or “transcript” the actual sequence contains U rather than T, but may be presented either way in the present disclosure.

Strategies for targeting particular regions of the POLRMT transcript, corresponding to regions with the gene sequence (e.g., SEQ ID NO: 1, NCBI Reference No. NG_023049.1), may be utilized in targeting a region within one or more POLRMT transcripts. FIG. 2 provides several exemplary POLRMT transcript sequences that may be targeted by oligonucleotides described herein (e.g., as identified in Accession Numbers NM_005035.4 (ENST00000588649.7 corresponding to SEQ ID NO: 205), XM_005259580.5, XM_047438952.1, and XM_047438951.1). One of skill in the art understands that an oligonucleotide targeting a region within SEQ ID NO: 205 may also target the corresponding region in other POLRMT RNA transcripts, although they may vary slightly in the exact coordinates within the nucleic acid sequence. Additionally, one of skill in the art will understand that a target region within a POLRMT transcript (e.g., SEQ ID NO: 205) may be characterized by the corresponding coordinates within the full POLRMT gene sequence (SEQ ID NO: 1).

In some embodiments, an oligonucleotide is capable of targeting a POLRMT sequence of one or more non-human species, e.g., a non-human primate POLRMT, e.g., Macaca fascicularis POLRMT, or e.g., Chlorocebus sabaeus in addition to human POLRMT. Such sequences are known in the art and publicly available. In some embodiments, an oligonucleotide is complementary to a target region that is identical in the human and Macaca fascicularis POLRMT transcripts. In some embodiments, an oligonucleotide is complementary to a target region of a human POLRMT transcript that differs by 1, 2, or 3 nucleotides from a sequence in a Macaca fascicularis POLRMT transcript. It will be appreciated that an oligonucleotide that targets human POLRMT and inhibits or decreases POLRMT expression level may also have such an effect on non-primate POLRMT e.g., rat or mouse POLRMT, particularly if conserved regions of POLRMT transcript are targeted. One of skill in the art understands that a target region of an oligonucleotide within a mouse POLRMT RNA transcript (e.g., SEQ ID NO: 582) may target a corresponding region within a human POLRMT RNA transcript (e.g., SEQ ID NO: 205), particularly if the target region is within a conserved region of POLRMT RNA transcript.

In some embodiments, an oligonucleotide has a nucleotide sequence comprising a region having sufficient complementarity to a target nucleic acid sequence to allow hybridization and insufficient complementarity to any non-target nucleic acid sequences so as to avoid non-specific hybridization to any non-target nucleic acid sequences under conditions in which specific hybridization is desired (e.g., under physiological conditions for in vivo or therapeutic uses, and under conditions in which assays are performed in the case of in vitro assays).

In some embodiments, the present disclosure provides oligonucleotides that are perfectly complementary to a target nucleotide sequence over the entire length of the oligonucleotide. In some embodiments, an oligonucleotide is at least 95% complementary to a PORLMT nucleotide sequence over the entire length of the oligonucleotide. In some embodiments, an oligonucleotide is at least 90% complementary to a PORLMT nucleotide sequence over the entire length of the oligonucleotide. In some embodiments, an oligonucleotide is at least 85% complementary to a POLRMT nucleotide sequence over the entire length of the oligonucleotide. In some embodiments, an oligonucleotide is at least 80% complementary to a POLRMT nucleotide sequence over the entire length of the oligonucleotide. In some embodiments, an oligonucleotide is between 80% and 100% complementary to a POLRMT nucleotide sequence over the entire length of the oligonucleotide (i.e., substantially complementary). In some embodiments, an oligonucleotide comprises a region that is perfectly complementary to a POLRMT nucleotide sequence and is at least 80% complementary to the POLRMT nucleotide sequence over the entire length of the oligonucleotide. In some embodiments, the region of perfect complementarity is from 6 to 30 nucleotides in length.

In some embodiments, an oligonucleotide comprises DNA. In some embodiments, an oligonucleotide comprises RNA. In some embodiments, an oligonucleotide comprises both RNA and DNA. In some embodiments, an oligonucleotide is between 5 and 100 nucleotides in length. In some embodiments, an oligonucleotide is between 5 and 90 nucleotides in length. In some embodiments, an oligonucleotide is between 5 and 80 nucleotides in length. In some embodiments, an oligonucleotide is between 5 and 70 nucleotides in length. In some embodiments, an oligonucleotide is between 5 and 60 nucleotides in length. In some embodiments, an oligonucleotide is 5 to 50 nucleotides in length. In some embodiments, an oligonucleotide is 5 to 40 nucleotides in length. In some embodiments, an oligonucleotide is 5 to 30 nucleotides in length. In some embodiments, an oligonucleotide is 5 to 25 nucleotides in length. In some embodiments, an oligonucleotide is 5 to 20 nucleotides in length. In some embodiments, an oligonucleotide is 5 to 15 nucleotides in length. In some embodiments, an oligonucleotide is 5 to 10 nucleotides in length. In some embodiments, an oligonucleotide is 10 to 100 nucleotides in length. In some embodiments, an oligonucleotide is 15 to 100 nucleotides in length. In some embodiments, an oligonucleotide is 20 to 100 nucleotides in length. In some embodiments, an oligonucleotide is 25 to 100 nucleotides in length. In some embodiments, an oligonucleotide is 30 to 100 nucleotides in length. In some embodiments, an oligonucleotide is 40 to 100 nucleotides in length. In some embodiments, an oligonucleotide is 50 to 100 nucleotides in length. In some embodiments, an oligonucleotide is 60 to 100 nucleotides in length. In some embodiments, an oligonucleotide is 70 to 100 nucleotides in length. In some embodiments, an oligonucleotide is 90 to 100 nucleotides in length. In some embodiments, an oligonucleotide is between 8 and 30 nucleotides in length. In some embodiments, an oligonucleotide is 15 to 25 nucleotides in length. In some embodiments, an oligonucleotide is 16 to 22 nucleotides in length. In some embodiments, an oligonucleotide is 18 to 20 nucleotides in length. In some embodiments, an oligonucleotide is 8, 9, 10, 11, 12, 13, 14, 15, 16, 17, 18, 19, 20, 21, 22, 23, 24, 25, 26, 27, 28, 29, or 30 nucleotides in length. In some embodiments, an oligonucleotide is 18 nucleotides in length. In some embodiments, an oligonucleotide is 20 nucleotides in length. In some embodiments, an oligonucleotide is 19 nucleotides in length.

In some embodiments, an oligonucleotide comprises a sequence having at least 80% identity to a sequence selected from a group consisting of SEQ ID NOs: 3-14, 27-45, 71-147, 205-298, and 587-655. In some embodiments, an oligonucleotide comprises a sequence having at least 85% identity to a sequence selected from a group consisting of SEQ ID NOs: 3-14, 27-45, 71-147, 205-298, and 587-655. In some embodiments, an oligonucleotide comprises a sequence having at least 90% identity to a sequence selected from a group consisting of SEQ ID NOs: 3-14, 27-45, 71-147, 205-298, and 587-655. In some embodiments, an oligonucleotide comprises a sequence having at least 95% identity to a sequence selected from a group consisting of SEQ ID NOs: 3-14, 27-45, 71-147, 205-298, and 587-655. In some embodiments, an oligonucleotide comprises a sequence selected from a group consisting of SEQ ID NOs: 3-14, 27-45, 71-147, 205-298, and 587-655.

In some embodiments, an oligonucleotide comprises a sequence having at least 80% identity to a sequence selected from a group consisting of SEQ ID NOs: 592, 594, 597, 598, 612, 613, 623, 624, 625, 626, 632, 633, 634, and 728-740. In some embodiments, an oligonucleotide comprises a sequence having at least 85% identity to a sequence selected from a group consisting of SEQ ID NOs: 592, 594, 597, 598, 612, 613, 623, 624, 625, 626, 632, 633, 634, and 728-740. In some embodiments, an oligonucleotide comprises a sequence having at least 90% identity to a sequence selected from a group consisting of SEQ ID NOs: 592, 594, 597, 598, 612, 613, 623, 624, 625, 626, 632, 633, 634, and 728-740. In some embodiments, an oligonucleotide comprises a sequence having at least 95% identity to a sequence selected from a group consisting of SEQ ID NOs: 592, 594, 597, 598, 612, 613, 623, 624, 625, 626, 632, 633, 634, and 728-740. In some embodiments, an oligonucleotide comprises a sequence selected from a group consisting of SEQ ID NOs: 592, 594, 597, 598, 612, 613, 623, 624, 625, 626, 632, 633, 634, and 728-740.

In some embodiments, an oligonucleotide comprises a sequence having at least 80% identity to SEQ ID NO: 11. In some embodiments, an oligonucleotide comprises a sequence having at least 85% to SEQ ID NO: 11. In some embodiments, an oligonucleotide comprises a sequence having at least 90% identity to SEQ ID NO: 11. In some embodiments, an oligonucleotide comprises a sequence having at least 95% identity to SEQ ID NO: 11. In some embodiments, an oligonucleotide comprises SEQ ID NO: 11.

In some embodiments, an oligonucleotide comprises a sequence having at least 80% identity to SEQ ID NO: 12. In some embodiments, an oligonucleotide comprises a sequence having at least 85% to SEQ ID NO: 12. In some embodiments, an oligonucleotide comprises a sequence having at least 90% identity to SEQ ID NO: 12. In some embodiments, an oligonucleotide comprises a sequence having at least 95% identity to SEQ ID NO: 12. In some embodiments, an oligonucleotide comprises SEQ ID NO: 12.

In some embodiments, an oligonucleotide comprises a sequence having at least 80% identity to SEQ ID NO: 594. In some embodiments, an oligonucleotide comprises a sequence having at least 85% to SEQ ID NO: 594. In some embodiments, an oligonucleotide comprises a sequence having at least 90% identity to SEQ ID NO: 594. In some embodiments, an oligonucleotide comprises a sequence having at least 95% identity to SEQ ID NO: 594. In some embodiments, an oligonucleotide comprises SEQ ID NO: 594.

In some embodiments, an oligonucleotide comprises a sequence having at least 80% identity to SEQ ID NO: 612. In some embodiments, an oligonucleotide comprises a sequence having at least 85% to SEQ ID NO: 612. In some embodiments, an oligonucleotide comprises a sequence having at least 90% identity to SEQ ID NO: 612. In some embodiments, an oligonucleotide comprises a sequence having at least 95% identity to SEQ ID NO: 612. In some embodiments, an oligonucleotide comprises SEQ ID NO: 612.

In some embodiments, an oligonucleotide comprises a sequence having at least 80% identity to SEQ ID NO: 632. In some embodiments, an oligonucleotide comprises a sequence having at least 85% to SEQ ID NO: 632. In some embodiments, an oligonucleotide comprises a sequence having at least 90% identity to SEQ ID NO: 632. In some embodiments, an oligonucleotide comprises a sequence having at least 95% identity to SEQ ID NO: 632. In some embodiments, an oligonucleotide comprises SEQ ID NO: 632.

In some embodiments, an oligonucleotide comprises a nucleotide sequence that is at least 80%, 85%, 90%, 95%, or 100% identical to any one of SEQ ID NOs: 3-14, 27-45, 71-147, 205-298, and 587-655 in the following Table 1.

In some embodiments, an oligonucleotide comprises a sequence that differs by no more than 1, 2, 3, or 4 nucleotides from any one of the sequences listed below in Table 1. Table 1 includes exemplary POLRMT oligonucleotide sequences, the target region of complementarity in the POLRMT mRNA transcript, and the corresponding coordinates of the target region within the POLRMT gene sequence as shown in SEQ ID NO: 1.

TABLE 1
Oligonucleotide Sequences, Target Region on PORLMT transcript, and Corresponding
Coordinates of the Target Region on POLRMT gene sequence (SEQ ID NO: 1)

| SEQ ID | Oligonucleotide Sequence | Target | Corresponding Coordinates |
|---|---|---|---|
| NO: | (5′ to 3′) | Region | within SEQ ID NO: 1 |
| 3 | CCCCAGTCCTTCCTGCGGTC | Exon | 5696-5715 |
| 4 | TCCACATCGAGGCTCAGCTT | Exon | 8808-8827 |
| 5 | CTCCACATCGAGGCTCAGCT | Exon | 8809-8828 |
| 6 | TGCTCCACATCGAGGCTCAG | Exon | 8811-8830 |
| 7 | CCCCAGCTCCTCCCAGTACT | Exon | 16221-16240 |
| 8 | GGCCACCTTCTGGCAGTGCG | Exon | 17159-17178 |
| 9 | GCCACGTCGCTGCCCAGGTG | Exon | 17314-17333 |
| 10 | CGCCACGTCGCTGCCCAGGT | Exon | 17315-17334 |
| 11 | CCCATCCACCACTTTCGGCC | Exon | 18082-18101 |
| 12 | GCCCATCCACCACTTTCGGC | Exon | 18083-18102 |
| 13 | CGCCCATCCACCACTTTCGG | Exon | 18084-18103 |
| 14 | TTCGCCACCTCCATACAGCA | Exon | 18130-18149 |
| 27 | GGTCTTGGTCTTGCTCCTGG | Exon | 5680-5699 |
| 28 | GAGGCTACCATCTCCACTGC | Exon | 8491-8510 |
| 29 | GCATCCTTGGCCCCCATCTG | Exon | 8529-8548 |
| 30 | CTCCAGTATCTTTGCCCAGC | Exon | 8569-8588 |
| 31 | TCTCCAGTATCTTTGCCCAG | Exon | 8570-8589 |
| 32 | TTCTCCAGTATCTTTGCCCA | Exon | 8571-8590 |
| 33 | CTTCTCCAGTATCTTTGCCC | Exon | 8572-8591 |
| 34 | CCTTCTCCAGTATCTTTGCC | Exon | 8573-8592 |
| 35 | TCCTTCTCCAGTATCTTTGC | Exon | 8574-8593 |
| 36 | CACATATACCAGCTCCTTGA | Exon | 13322-13341 |
| 37 | TCCTCCTCAGACAGCAGAAC | Exon | 13719-13738 |
| 38 | CACTGCAGGGTCTTCAAGGG | Exon | 14999-15018 |
| 39 | TGCTTGACCTCCTTGCTTGG | Exon | 15092-15111 |
| 40 | GTGCTTGACCTCCTTGCTTG | Exon | 15093-15112 |
| 41 | TGCCCAGGTGGTTGAAGTGC | Exon | 17304-17323 |
| 42 | TGTAGACTCTTGAAGACCTG | Exon | 19309-19328 |
| 43 | AGCATCATGTGGGAGGAGTC | Exon | 20041-20060 |
| 44 | GAGCATCATGTGGGAGGAGT | Exon | 20042-20061 |
| 45 | TTCAGCTGGCTGGCCTCCAA | Exon | 21102-21121 |
| 71 | GAGATAGTGAGAGGCCTCC | Exon | 19284-19302 |
| 72 | AGGAATACACGTGGTAGAG | Exon | 16385-16403 |
| 73 | CCAGTACTGCCGCGGCAGG | Exon | 16210-16228 |
| 74 | AGTCCAGGATGTCATCCAT | Exon | 17473-17491 |
| 75 | TCTTCTGCTTACGTGTGTT | Exon | 19990-20008 |
| 76 | ATCATGTGGGAGGAGTCCA | Exon | 20039-20057 |
| 77 | GCCTGCAGGACCTGCAGGA | Exon | 15742-15748 and 15817-15827 |
| 78 | CAGCTGGTCAGTGAGCAGG | Exon | 8096-8924 |
| 79 | CTATTTGCTTGACCTTGGA | Exon | 19561-19572 and 19809-19815 |
| 80 | CAAAGCATGGGTACATCCA | Exon | 16802-16820 |
| 81 | AAGGGGTAAAGTGAGAACC | Exon | 15684-15702 |
| 82 | AGAGGTACTTCCTGTAGTG | Exon | 15946-15964 |
| 83 | AACACATATACCAGCTCCT | Exon | 13325-13343 |
| 84 | TTCATGACGGAGACATCAG | Exon | 20770-20788 |
| 85 | GTAGGGCTCGTTTGAGCCC | Exon | 5090-5108 |
| 86 | CCCATGTGGGAGATGAGGC | Exon | 19481-19499 |
| 87 | GAGTCAAGCCGGCATCCTT | Exon | 13354-13372 |
| 88 | CCAAGATCTTCTGGGGCTC | Exon | 20998-20999 and 21089-21105 |
| 89 | CACTGCAGGGTCTTCAAGG | Exon | 15000-15018 |
| 90 | CACACCCTGCTGGCCAGCT | Exon | 15045-15063 |
| 91 | ACGCCGCTCCAGGCCACCCC | 5′UTR | 5032-5051 |
| 92 | GCACGCCGCTCCAGGCCACC | 5′UTR | 5034-5053 |
| 93 | ACGCACGCCGCTCCAGGCCA | 5′UTR | 5036-5055 |
| 94 | GGCCCCAGCAAAGTGCCGAC | exon | 5059-5078 |
| 95 | GTCCTTCCTGCGGTCTTGGT | exon | 5691-5710 |
| 96 | AGTCCTTCCTGCGGTCTTGG | exon | 5692-5711 |
| 97 | CCCCAGTCCTTCCTGCGGTC | exon | 5696-5715 |
| 98 | CACGTGGCCCCAGTCCTTCC | exon | 5703-5722 |
| 99 | CATCCACCCTGTTCACCACC | exon | 8450-8469 |
| 100 | CACATCCACCCTGTTCACCA | exon | 8452-8471 |
| 101 | GCGCCACATCCACCCTGTTC | exon | 8456-8475 |
| 102 | GCCCCCATCTGGACCTTCCT | exon | 8520-8539 |
| 103 | GAACTCCCCGCTCTGGAATG | exon | 8647-8666 |
| 104 | CTTGAACTCCCCGCTCTGGA | exon | 8650-8669 |
| 105 | CGGCCATCTGCTTGCTCAGG | exon | 8705-8724 |
| 106 | CCGGCCATCTGCTTGCTCAG | exon | 8706-8725 |
| 107 | ACCCGGCCATCTGCTTGCTC | exon | 8708-8727 |
| 108 | CTCCAGGCACCCGGCCATCT | exon | 8716-8735 |
| 109 | CCTCCAGGCACCCGGCCATC | exon | 8717-8736 |
| 110 | ATCGAGGCTCAGCTTCCCAG | exon | 8803-8822 |
| 111 | CCTGCTCCACATCGAGGCTC | exon | 8813-8832 |
| 112 | GGCGTTGTACATGTCCAGCG | exon | 8986-9005 |
| 113 | GCATCACGGCGTTGTACATG | exon | 8993-9012 |
| 114 | AGCATCACGGCGTTGTACAT | exon | 8995-9014 |
| 115 | AAGCATCACGGCGTTGTACA | exon | 8994-9013 |
| 116 | TCCAGACACCTTTCGATGGT | exon | 13435-13447 and 13666-13672 |
| 117 | CAGAACGGCGGTGAAGAGTG | exon | 13705-13724 |
| 118 | GCAGAACGGCGGTGAAGAGT | exon | 13706-13725 |
| 119 | GACAGCAGAACGGCGGTGAA | exon | 13710-13729 |
| 120 | ACGGCCTTCAGAACAGTGGC | exon | 13743-13762 |
| 121 | CACCTTGTGCACGGCCTTCA | exon | 13753-13772 |
| 122 | ACACGCCCATCCTTGGCATA | exon | 13842-13851 and 14967-14976 |
| 123 | GGACACACGCCCATCCTTGG | exon | 14959-14980 |
| 124 | AGGACACACGCCCATCCTTG | exon | 14963-14981 |
| 125 | GTAGGACACACGCCCATCCT | exon | 14964-14983 |
| 126 | GGTAGGACACACGCCCATCC | exon | 14965-14984 |
| 127 | GACCTCCTTGCTTGGCAACG | exon | 15087-15106 |
| 128 | TTTCTCCCATTGGTCCCGCA | exon | 15603-15622 |
| 129 | GTGCTTTCTCCCATTGGTCC | exon | 15607-15626 |
| 130 | CCGGCACAGTGCTTTCTCCC | exon | 15615-15634 |
| 131 | CCACTGACCCGCTGCCTCTG | exon | 15904-15923 |
| 132 | CTCCTCCCAGTACTGCCGCG | exon | 16215-16234 |
| 133 | TTGTCCAGGCTGCATGGCAT | exon | 16334-16353 |
| 134 | TCCACCGCCTCGAAGGTCAG | exon | 16786-16805 |
| 135 | ATCCACCGCCTCGAAGGTCA | exon | 16787-16806 |
| 136 | CATCCACCGCCTCGAAGGTC | exon | 16788-16807 |
| 137 | ACATCCACCGCCTCGAAGGT | exon | 16789-16808 |
| 138 | GGTACATCCACCGCCTCGAA | exon | 16792-16811 |
| 139 | GTGCGGCGATGTCCAGGGCA | exon | 16829-16848 |
| 140 | AGAGTGCGGCGATGTCCAGG | exon | 16832-16851 |
| 141 | CAGAGTGCGGCGATGTCCAG | exon | 16833-16852 |
| 142 | CCAGAGTGCGGCGATGTCCA | exon | 16834-16853 |
| 143 | CACCAGAGTGCGGCGATGTC | exon | 16836-16855 |
| 144 | AGCACCAGAGTGCGGCGATG | exon | 16838-16857 |
| 145 | GGTCCAGCACGCGCCCGTTG | exon | 16998-17017 |
| 146 | AGGTCCAGCACGCGCCCGTT | exon | 16999-17018 |
| 147 | GCCAGCTCACGGCGCAGCTC | exon | 17140-17159 |
| 205 | CCGCCGCTCCAGGCCACCCC | exon | 5032-5051 |
| 206 | GCACGCCGCTCCAGGCCACC | exon | 5034-5053 |
| 207 | ACGCACGCCGCTCCAGGCCA | exon | 5036-5055 |
| 208 | GGCCCCAGCAAAGTGCCGAC | exon | 5059-5078 |
| 209 | GTCCTTCCTGCGGTCTTGGT | exon | 5691-5710 |
| 210 | AGTCCTTCCTGCGGTCTTGG | exon | 5692-5711 |
| 211 | CCCCAGTCCTTCCTGCGGTC | exon | 5696-5715 |
| 212 | CACGTGGCCCCAGTCCTTCC | exon | 5703-5722 |
| 213 | CATCCACCCTGTTCACCACC | exon | 8450-8469 |
| 214 | CACATCCACCCTGTTCACCA | exon | 8452-8471 |
| 215 | GCGCCACATCCACCCTGTTC | exon | 8456-8475 |
| 216 | GCCCCCATCTGGACCTTCCT | exon | 8520-8539 |
| 217 | GAACTCCCCGCTCTGGAATG | exon | 8647-8666 |
| 218 | CTTGAACTCCCCGCTCTGGA | exon | 8650-8669 |
| 219 | CGGCCATCTGCTTGCTCAGG | exon | 8705-8724 |
| 220 | CCGGCCATCTGCTTGCTCAG | exon | 8706-8725 |
| 221 | ACCCGGCCATCTGCTTGCTC | exon | 8708-8727 |
| 222 | CTCCAGGCACCCGGCCATCT | exon | 8716-8735 |
| 223 | CCTCCAGGCACCCGGCCATC | exon | 8717-8736 |
| 224 | ATCGAGGCTCAGCTTCCCAG | exon | 8803-8822 |
| 225 | CCTGCTCCACATCGAGGCTC | exon | 8813-8832 |
| 226 | GGCGTTGTACATGTCCAGCG | exon | 8986-9005 |
| 227 | GCATCACGGCGTTGTACATG | exon | 8993-9012 |
| 228 | AGCATCACGGCGTTGTACAT | exon | 8994-9013 |
| 229 | AAGCATCACGGCGTTGTACA | exon | 8995-9014 |
| 230 | TCCAGACACCTTTCGATGGT | exon | 13435-13447 and 13666-13672 |
| 231 | CAGAACGGCGGTGAAGAGTG | exon | 13705-13724 |
| 232 | GCAGAACGGCGGTGAAGAGT | exon | 13706-13725 |
| 233 | GACAGCAGAACGGCGGTGAA | exon | 13710-13729 |
| 234 | ACGGCCTTCAGAACAGTGGC | exon | 13743-13762 |
| 235 | CACCTTGTGCACGGCCTTCA | exon | 13753-13772 |
| 236 | ACACGCCCATCCTTGGCATA | exon | 13842-13850 and 14966-14976 |
| 237 | GGACACACGCCCATCCTTGG | exon | 13846-13850 and 14966-14980 |
| 238 | AGGACACACGCCCATCCTTG | exon | 13847-13850 and 14966-14981 |
| 239 | GTAGGACACACGCCCATCCT | exon | 13849-13850 and 14966-14983 |
| 240 | GGTAGGACACACGCCCATCC | exon | 13850-13850 and 14966-14984 |
| 241 | GACCTCCTTGCTTGGCAACG | exon | 15087-15106 |
| 242 | TTTCTCCCATTGGTCCCGCA | exon | 15603-15622 |
| 243 | GTGCTTTCTCCCATTGGTCC | exon | 15607-15626 |
| 244 | CCGGCACAGTGCTTTCTCCC | exon | 15615-15634 |
| 245 | CCACTGACCCGCTGCCTCTG | exon | 15904-15923 |
| 246 | CTCCTCCCAGTACTGCCGCG | exon | 16215-16234 |
| 247 | TTGTCCAGGCTGCATGGCAT | exon | 16334-16353 |
| 248 | TCCACCGCCTCGAAGGTCAG | exon | 16786-16805 |
| 249 | ATCCACCGCCTCGAAGGTCA | exon | 16787-16806 |
| 250 | CATCCACCGCCTCGAAGGTC | exon | 16788-16807 |
| 251 | ACATCCACCGCCTCGAAGGT | exon | 16789-16808 |
| 252 | GGTACATCCACCGCCTCGAA | exon | 16792-16811 |
| 253 | GTGCGGCGATGTCCAGGGCA | exon | 16829-16848 |
| 254 | AGAGTGCGGCGATGTCCAGG | exon | 16832-16851 |
| 255 | CAGAGTGCGGCGATGTCCAG | exon | 16833-16852 |
| 256 | CCAGAGTGCGGCGATGTCCA | exon | 16834-16853 |
| 257 | CACCAGAGTGCGGCGATGTC | exon | 16836-16855 |
| 258 | AGCACCAGAGTGCGGCGATG | exon | 16838-16857 |
| 259 | GGTCCAGCACGCGCCCGTTG | exon | 16998-17017 |
| 260 | AGGTCCAGCACGCGCCCGTT | exon | 16999-17018 |
| 261 | GCCAGCTCACGGCGCAGCTC | exon | 17140-17159 |
| 262 | CGCCAGCTCACGGCGCAGCT | exon | 17141-17160 |
| 263 | GCAGTGCGCCAGCTCACGGC | exon | 17147-17166 |
| 264 | CTTCTGGCAGTGCGCCAGCT | exon | 17153-17172 |
| 265 | ACCTTCTGGCAGTGCGCCAG | exon | 17155-17174 |
| 266 | CCACCTTCTGGCAGTGCGCC | exon | 17157-17176 |
| 267 | TCCCGGGCCACCTTCTGGCA | exon | 17164-17183 |
| 268 | GACGCGGTCCCGCAGGTGCT | exon | 17231-17250 |
| 269 | GGAAGTCCATGTTGTGCGGC | exon | 17259-17278 |
| 270 | GCGGAAGTCCATGTTGTGCG | exon | 17261-17280 |
| 271 | GTCGCTGCCCAGGTGGTTGA | exon | 17309-17328 |
| 272 | ACGTCGCTGCCCAGGTGGTT | exon | 17311-17330 |
| 273 | CCACGTCGCTGCCCAGGTGG | exon | 17313-17332 |
| 274 | CCCGCGCCACGTCGCTGCCC | exon | 17319-17338 |
| 275 | GGCCCGCGCCACGTCGCTGC | exon | 17321-17340 |
| 276 | GCGGCCCTGGGCGAACTCCA | exon | 17345-17364 |
| 277 | CACCACTTTCGGCCCGTCAA | exon | 17506-17512 and 18083-18095 |
| 278 | CCTCCGCGCCCATCCACCAC | exon | 18090-18109 |
| 279 | TCCTCCGCGCCCATCCACCA | exon | 18091-18110 |
| 280 | GTTCCTCCGCGCCCATCCAC | exon | 18093-18112 |
| 281 | GGGTTCCTCCGCGCCCATCC | exon | 18095-18114 |
| 282 | AGGCCAGCGTCTGCCAGGGT | exon | 18111-18130 |
| 283 | CGGCACATCCGAGGGCTCCA | exon | 18562-18581 |
| 284 | CACGCCGCTGTACACGTCCT | exon | 18583-18602 |
| 285 | CCACGCCGCTGTACACGTCC | exon | 18584-18603 |
| 286 | GCCACGCCGCTGTACACGTC | exon | 18585-18604 |
| 287 | GGCCACGCCGCTGTACACGT | exon | 18586-18605 |
| 288 | TGCGCGGCCACGCCGCTGTA | exon | 18591-18610 |
| 289 | CTGCCTACGGAACACCTCCA | exon | 18805-18824 |
| 290 | TTCCAGCACCTGTGCCACCC | exon | 18844-18863 |
| 291 | GAGGCCTCCCACACGAACTC | exon | 19273-19292 |
| 292 | AGAGGCCTCCCACACGAACT | exon | 19274-19293 |
| 293 | TGAATTCCACCTCCTATTTG | exon | 19809-19828 |
| 294 | CGAAGGTCAGGCCCTTCCTG | exon | 20717-20736 |
| 295 | AGACGAAGGTCAGGCCCTTC | exon | 20720-20739 |
| 296 | GAGACGAAGGTCAGGCCCTT | exon | 20721-20740 |
| 297 | AGAGACGAAGGTCAGGCCCT | exon | 20722-20741 |
| 298 | AGTAACAGTCGTGCACAGAG | exon | 20738-20757 |
| 587 | GAAGTCCATGTTGTGCGGCA | Exon | 17258-17277 |
| 588 | CGGAAGTCCATGTTGTGCGG | Exon | 17260-17279 |
| 589 | CGCGGAAGTCCATGTTGTGC | Exon | 17262-17281 |
| 590 | GCCGCGGAAGTCCATGTTGT | Exon | 17264-17283 |
| 591 | GGCCGCGGAAGTCCATGTTG | Exon | 17265-17285 |
| 592 | CGGCCGCGGAAGTCCATGTT | Exon | 17266-17285 |
| 593 | GCGGCCGCGGAAGTCCATGT | Exon | 17267-17286 |
| 594 | TGCGGCCGCGGAAGTCCATG | Exon | 17268-17287 |
| 595 | GTGCGGCCGCGGAAGTCCAT | Exon | 17269-17288 |
| 596 | CTGCCCAGGTGGTTGAAGTG | Exon | 17305-17324 |
| 597 | TCTGCTTCACCACCTTGCGG | exon | 18875-18894 |
| 598 | GTCTGCTTCACCACCTTGCG | exon | 18876-18895 |
| 599 | ATGAAGTTGGGCGGGAAGCC | Exon | 20011-20030 |
| 600 | GATGAAGTTGGGCGGGAAGC | Exon | 20012-20031 |
| 601 | GGATGAAGTTGGGCGGGAAG | Exon | 20013-20032 |
| 602 | TGGATGAAGTTGGGCGGGAA | Exon | 20014-20033 |
| 603 | GTGGATGAAGTTGGGCGGGA | Exon | 20015-20034 |
| 604 | GAGTGGATGAAGTTGGGCGG | Exon | 20017-20036 |
| 605 | GAGTGGATGAAGTTGGGCGG | Exon | 20017-20036 |
| 606 | ATCATGTGGGAGGAGTCCAG | Exon | 20038-20057 |
| 607 | CATCATGTGGGAGGAGTCCA | Exon | 20039-20058 |
| 608 | GCATCATGTGGGAGGAGTCC | Exon | 20040-20059 |
| 609 | AGGCCCTTCCTGTAGCAGTG | Exon | 20071-20081 and 20720-20728 |
| 610 | CAGGCCCTTCCTGTAGCAGT | Exon | 20072-20081 and 20720-20729 |
| 611 | TCAGGCCCTTCCTGTAGCAG | Exon | 20073-20081 and 20720-20730 |
| 612 | CCCAGCCAAGCATCACGG | Exon | 9004-9021 |
| 613 | GCCCAGCCAAGCATCACG | Exon | 9005-9022 |
| 614 | AGTCCATGTTGTGCGGCA | Exon | 17258-17275 |
| 615 | AAGTCCATGTTGTGCGGC | Exon | 17259-17276 |
| 616 | GAAGTCCATGTTGTGCGG | Exon | 17260-17277 |
| 617 | GGAAGTCCATGTTGTGCG | Exon | 17261-17278 |
| 618 | CGGAAGTCCATGTTGTGC | Exon | 17262-17279 |
| 619 | GCGGAAGTCCATGTTGTG | Exon | 17263-17280 |
| 620 | CGCGGAAGTCCATGTTGT | Exon | 17264-17281 |
| 621 | CCGCGGAAGTCCATGTTG | Exon | 17265-17282 |
| 622 | GCCGCGGAAGTCCATGTT | Exon | 17266-17283 |
| 623 | GGCCGCGGAAGTCCATGT | Exon | 17267-17284 |
| 624 | CGGCCGCGGAAGTCCATG | Exon | 17268-17285 |
| 625 | GCGGCCGCGGAAGTCCAT | Exon | 17269-17286 |
| 626 | TGCGGCCGCGGAAGTCCA | Exon | 17270-17287 |
| 627 | GTGCGGCCGCGGAAGTCC | Exon | 17271-17288 |
| 628 | GCCCAGGTGGTTGAAGTG | Exon | 17305-17322 |
| 629 | TGCCCAGGTGGTTGAAGT | Exon | 17306-17323 |
| 630 | CTGCCCAGGTGGTTGAAG | Exon | 17307-17324 |
| 631 | TGCTTCACCACCTTGCGG | Exon | 18875-18892 |
| 632 | CTGCTTCACCACCTTGCG | Exon | 18876-18893 |
| 633 | TCTGCTTCACCACCTTGC | Exon | 18877-18894 |
| 634 | GTCTGCTTCACCACCTTG | Exon | 18878-18895 |
| 635 | GAACATCTCCTGTAGACT | Exon | 19321-19338 |
| 636 | GAAGTTGGGCGGGAAGCC | Exon | 20011-20028 |
| 637 | TGAAGTTGGGCGGGAAGC | Exon | 20012-20029 |
| 638 | ATGAAGTTGGGCGGGAAG | Exon | 20013-20030 |
| 639 | GATGAAGTTGGGCGGGAA | Exon | 20014-20031 |
| 640 | GGATGAAGTTGGGCGGGA | Exon | 20015-20032 |
| 641 | TGGATGAAGTTGGGCGGG | Exon | 20016-20033 |
| 642 | GTGGATGAAGTTGGGCGG | Exon | 20017-20034 |
| 643 | AGTGGATGAAGTTGGGCG | Exon | 20018-20035 |
| 644 | GAGTGGATGAAGTTGGGC | Exon | 20019-20036 |
| 645 | CATGTGGGAGGAGTCCAG | Exon | 20038-20055 |
| 646 | TCATGTGGGAGGAGTCCA | Exon | 20039-20056 |
| 647 | ATCATGTGGGAGGAGTCC | Exon | 20040-20057 |
| 648 | CATCATGTGGGAGGAGTC | Exon | 20041-20058 |
| 649 | GCATCATGTGGGAGGAGT | Exon | 20042-20059 |
| 650 | AGCATCATGTGGGAGGAG | Exon | 20043-20060 |
| 651 | GCCCTTCCTGTAGCAGTG | Exon | 20071-20081 and 20720-20726 |
| 652 | GGCCCTTCCTGTAGCAGT | Exon | 20072-20081 and 20720-20727 |
| 653 | AGGCCCTTCCTGTAGCAG | Exon | 20073-20081 and 20720-20728 |
| 654 | CAGGCCCTTCCTGTAGCA | Exon | 20074-20081 and 20720-20729 |
| 655 | TCAGGCCCTTCCTGTAGC | Exon | 20075-20081 and 20720-20730 |

In some embodiments, an oligonucleotide targets a region of a murine POLRMT transcript. In some embodiments, an oligonucleotide targets a region of a mouse POLRMT transcript. The amino acid and nucleotide sequences encoding the mouse POLRMT gene are known in the art and can be found in publicly available databases. For example, mouse POLRMT gene sequences are identified in the sequence span on mouse chromosome 10 corresponding to coordinates GRCm39 10_79571957_79582415 and the POLRMT transcript is represented in, e.g., Reference No. ENSMUST00000161765. In some embodiments, an oligonucleotide targets an exon region within a mouse POLRMT transcript (e.g., the longest exon in a mouse POLRMT transcript represented in Reference No. ENSMUST00000161765, SEQ ID NO: 582).

In some embodiments, an oligonucleotide comprises a sequence having at least 80% identity to a sequence selected from a group consisting of SEQ ID Nos: 393-486, 592, 594, 597, 598, 612, 613, 623, 624, 625, 626, 632, 633, and 634. In some embodiments, an oligonucleotide comprises a sequence having at least 85% identity to a sequence selected from a group consisting of SEQ ID Nos: 393-486, 592, 594, 597, 598, 612, 613, 623, 624, 625, 626, 632, 633, and 634. In some embodiments, an oligonucleotide comprises a sequence having at least 90% identity to a sequence selected from a group consisting of SEQ ID Nos: 393-486, 592, 594, 597, 598, 612, 613, 623, 624, 625, 626, 632, 633, and 634. In some embodiments, an oligonucleotide comprises a sequence selected from a group consisting of SEQ ID Nos: 393-486, 592, 594, 597, 598, 612, 613, 623, 624, 625, 626, 632, 633, and 634. In some embodiments, an oligonucleotide comprises a sequence selected from a group consisting of SEQ ID Nos: 393-486, 592, 594, 597, 598, 612, 613, 623, 624, 625, 626, 632, 633, and 634.

In some embodiments, an oligonucleotide comprises a sequence having at least 80% identity to SEQ ID NO: 434. In some embodiments, an oligonucleotide comprises a sequence having at least 85% to SEQ ID NO: 434. In some embodiments, an oligonucleotide comprises a sequence having at least 90% identity to SEQ ID NO: 434. In some embodiments, an oligonucleotide comprises a sequence having at least 95% identity to SEQ ID NO: 434. In some embodiments, an oligonucleotide comprises SEQ ID NO: 434.

In some embodiments, an oligonucleotide comprises a sequence having at least 80% identity to SEQ ID NO: 442. In some embodiments, an oligonucleotide comprises a sequence having at least 85% to SEQ ID NO: 442. In some embodiments, an oligonucleotide comprises a sequence having at least 90% identity to SEQ ID NO: 442. In some embodiments, an oligonucleotide comprises a sequence having at least 95% identity to SEQ ID NO: 442. In some embodiments, an oligonucleotide comprises SEQ ID NO: 442.

In some embodiments, an oligonucleotide comprises a nucleotide sequence that is at least 80%, 85%, 90%, 95%, or 100% identical to any one of SEQ ID Nos: 393-486 in the following Table 2.

In some embodiments, an oligonucleotide comprises a sequence that differs by no more than 1, 2, 3, or 4 nucleotides from any one of the sequences listed below in Table 2. Table 2 includes exemplary mouse POLRMT oligonucleotide sequences, the target region of complementarity in the mouse POLRMT mRNA transcript, and the corresponding coordinates of the target region within the mouse POLRMT gene sequence as shown in SEQ ID NO: 581.

TABLE 2
Oligonucleotide Sequences, Target Region, and Corresponding Coordinates
of the Target Region on mouse POLRMT gene sequence (SEQ ID NO: 581)

| | | | Corresponding |
|---|---|---|---|
| SEQ ID | Oligonucleotide Sequence | Target | Coordinates within |
| NO: | (5′ to 3′) | Region | SEQ ID NO: 581 |
| 393 | CACGGTGTTGTACATGTGCA | exon | 7077-7096 |
| 394 | ATCACGGTGTTGTACATGTG | exon | 7075-7094 |
| 395 | CATCACGGTGTTGTACATGT | exon | 7074-7093 |
| 396 | GTGCATCTCTCGAGCCACCT | exon | 3342-3361 |
| 397 | TGTGCATCTCTCGAGCCACC | exon | 3341-3360 |
| 398 | CTGTGCATCTCTCGAGCCAC | exon | 3340-3359 |
| 399 | GTGCTGTGCCAGCGACAGGC | exon | 3297-3316 |
| 400 | GTCCATGTTGTGCGGCAACC | exon | 3258-3277 |
| 401 | AGGTCACTGCCCAGGTGGTT | exon | 3202-3221 |
| 402 | TCCATCCACCACTTCCGGCC | exon | 2663-2682 |
| 403 | ACCTCCATGCAGCAGGCCAG | exon | 2621-2640 |
| 404 | CACCTCCATGCAGCAGGCCA | exon | 2620-2639 |
| 405 | CCACCTCCATGCAGCAGGCC | exon | 2619-2638 |
| 406 | GCCACCTCCATGCAGCAGGC | exon | 2618-2637 |
| 407 | TGCCACCTCCATGCAGCAGG | exon | 2617-2636 |
| 408 | CACCACCTTGCGGCTGATGA | exon | 2005-2024 |
| 409 | TTCACCACCTTGCGGCTGAT | exon | 2003-2022 |
| 410 | CTGCTGTCTGTCTCCGTTGT | exon | 7107-7126 |
| 411 | ACCTGCTGTCTGTCTCCGTT | exon | 7105-7124 |
| 412 | GCACCTGCTGTCTGTCTCCG | exon | 7103-7122 |
| 413 | TGTTGTACATGTGCAGTGTG | exon | 7082-7101 |
| 414 | CGGTGTTGTACATGTGCAGT | exon | 7079-7098 |
| 415 | GACCAGGTCAGTGAAGAGCA | exon | 5712-5731 |
| 416 | TCCAGGACCAGGTCAGTGAA | exon | 5707-5726 |
| 417 | CTTCCAGGACCAGGTCAGTG | exon | 5705-5724 |
| 418 | CTCCCCTTCGCTCAGCAGGC | exon | 4732-4751 |
| 419 | ACTCCCCTTCGCTCAGCAGG | exon | 4731-4750 |
| 420 | AACTCCCCTTCGCTCAGCAG | exon | 4730-4749 |
| 42 | GCCCAGGTTATGGGCCAACT | exon | 4568-4587 |
| 422 | CTGTTGTGCAGGGGCCTCCA | exon | 4178-4197 |
| 423 | CAATTGCTTGCCCAGCTGTA | exon | 4136-4155 |
| 424 | CCAATTGCTTGCCCAGCTGT | exon | 4135-4154 |
| 425 | CGCCAATTGCTTGCCCAGCT | exon | 4133-4152 |
| 426 | CCGCCAATTGCTTGCCCAGC | exon | 4132-4151 |
| 427 | TCCGCCAATTGCTTGCCCAG | exon | 4131-4150 |
| 428 | CTCCGCCAATTGCTTGCCCA | exon | 4130-4149 |
| 429 | GCTCCGCCAATTGCTTGCCC | exon | 4129-4148 |
| 430 | AGCTCCGCCAATTGCTTGCC | exon | 4128-4147 |
| 43 | AGGGCATTGCTCCAGCAGAC | exon | 3597-3616 |
| 432 | CGCAGTTCCCCAACTGTGTG | exon | 3545-3564 |
| 433 | GCGCAGTTCCCCAACTGTGT | exon | 3544-3563 |
| 434 | CTCTCGAGCCACCTTGAGGC | exon | 3348-3367 |
| 435 | ACTGTGCATCTCTCGAGCCA | exon | 3339-3358 |
| 436 | GACTGTGCATCTCTCGAGCC | exon | 3338-3357 |
| 437 | AGACTGTGCATCTCTCGAGC | exon | 3337-3356 |
| 438 | CAGACTGTGCATCTCTCGAG | exon | 3336-3355 |
| 439 | GCAGACTGTGCATCTCTCGA | exon | 3335-3354 |
| 440 | GCGGAAGTCCATGTTGTGCG | exon | 3252-3271 |
| 441 | CCGCGGAAGTCCATGTTGTG | exon | 3250-3269 |
| 442 | CGCTAGGTCACTGCCCAGGT | exon | 3198-3217 |
| 443 | GCGCTAGGTCACTGCCCAGG | exon | 3197-3216 |
| 444 | CGCGCTAGGTCACTGCCCAG | exon | 3196-3215 |
| 445 | CCGGCCCTCAGCAAACTCCA | exon | 3168-3187 |
| 446 | TTCCATCCACCACTTCCGGC | exon | 2662-2681 |
| 447 | GCTTCCATCCACCACTTCCG | exon | 2660-2679 |
| 448 | AGCTTCCATCCACCACTTCC | exon | 2659-2678 |
| 449 | CAGCTTCCATCCACCACTTC | exon | 2658-2677 |
| 450 | TCAGCTTCCATCCACCACTT | exon | 2657-2676 |
| 451 | ATCAGCTTCCATCCACCACT | exon | 2656-2675 |
| 452 | CATCAGCTTCCATCCACCAC | exon | 2655-2674 |
| 453 | TCATCAGCTTCCATCCACCA | exon | 2654-2673 |
| 454 | GCTCATCAGCTTCCATCCAC | exon | 2652-2671 |
| 455 | GGCTCATCAGCTTCCATCCA | exon | 2651-2670 |
| 456 | GTGCCACCTCCATGCAGCAG | exon | 2616-2635 |
| 457 | TGTGCCACCTCCATGCAGCA | exon | 2615-2634 |
| 458 | GTGTGCCACCTCCATGCAGC | exon | 2614-2633 |
| 459 | CGTGTGCCACCTCCATGCAG | exon | 2613-2632 |
| 460 | GCGTGTGCCACCTCCATGCA | exon | 2612-2631 |
| 461 | TGCGTGTGCCACCTCCATGC | exon | 2611-2630 |
| 462 | CAGTCCACTTGCGTGCCCAC | exon | 2540-2559 |
| 463 | GGGCAGTCCACTTGCGTGCC | exon | 2537-2556 |
| 464 | GCCATGGGCAGTCCACTTGC | exon | 2532-2551 |
| 465 | CACAGGTCACCCCAGAAACT | exon | 2380-2399 |
| 466 | CACCCACACTGTCTCGGCCT | exon | 2271-2290 |
| 467 | GCACCCACACTGTCTCGGCC | exon | 2270-2289 |
| 468 | TGTTGCCACCTCCCTGTACA | exon | 2209-2228 |
| 469 | TGTGTTGCCACCTCCCTGTA | exon | 2207-2226 |
| 470 | CTGTGTTGCCACCTCCCTGT | exon | 2206-2225 |
| 471 | CTGGCACCCACCTGTGTTGC | exon | 2195-2214 |
| 472 | CACTCCACAGGCCATCCGGC | exon | 1536-1555 |
| 473 | CCCACTCCACAGGCCATCCG | exon | 1534-1553 |
| 474 | GACCCACTCCACAGGCCATC | exon | 1532-1551 |
| 475 | CCACCTTTTACCTGCAGACC | exon | 1377-1396 |
| 476 | GCCACCTTTTACCTGCAGAC | exon | 1376-1395 |
| 477 | GGCCACCTTTTACCTGCAGA | exon | 1375-1394 |
| 478 | CTCCCCGCAGTACAGGACTC | exon | 1313-1332 |
| 479 | CCTCCCCGCAGTACAGGACT | exon | 1312-1331 |
| 480 | CCCTCCCCGCAGTACAGGAC | exon | 1311-1330 |
| 481 | CTCCCCTCCCCGCAGTACAG | exon | 1308-1327 |
| 482 | TCCTGCCCATCCCTCCATCC | exon | 1287-1306 |
| 483 | TTCCTGCCCATCCCTCCATC | exon | 1286-1305 |
| 484 | CCTTTCCTGCCCATCCCTCC | exon | 1283-1302 |
| 485 | GCATGTGTCCAGAAGCAGTC | exon | 637-656 |
| 486 | TTGCGCACCGCCAGAAAGTG | exon | 19-38 |

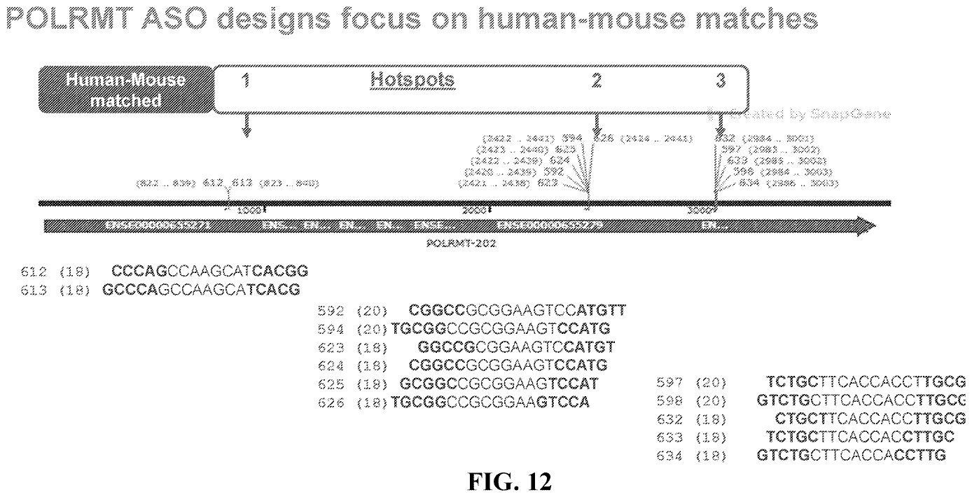

In some embodiments, an oligonucleotide of the present disclosure is complementary (e.g., substantially complementary or perfectly complementary) to a region of a POLRMT RNA transcript (e.g., SEQ ID NO: 205). In some embodiments, an oligonucleotide is complementary to a region of a 5′ untranslated region (UTR) of a POLRMT RNA transcript. In some embodiments, an oligonucleotide is complementary to a region that is within or includes an exon region of a POLRMT RNA transcript (e.g., within SEQ ID NO: 205). In some embodiments, an oligonucleotide is complementary to a region that is within or includes an intron region of a POLRMT pre-mRNA transcript. In some embodiments, an oligonucleotide is complementary to a region of a POLRMT pre-mRNA transcript that spans an exon/intron junction. In some embodiments, an oligonucleotide is complementary to a region that includes or is within a 3′ UTR region of a POLRMT RNA transcript. In some embodiments, an oligonucleotide is complementary to a region of a POLRMT RNA transcript as illustrated in FIG. 2, FIG. 12, and Table 3. In some embodiments, an oligonucleotide sequence is complementary to a region within the POLRMT gene sequence (SEQ ID NO: 1).

In some embodiments, an oligonucleotide is substantially complementary to 8 to 30 contiguous nucleotides of the POLRMT exon sequence identified in Ensemble ID ENSE00000655271. In some embodiments, an oligonucleotide is perfectly complementary to 8 to 30 contiguous nucleotides of the POLRMT exon sequence identified in Ensemble ID ENSE00000655271. In some embodiments, an oligonucleotide is complementary to 8 to 22 contiguous nucleotides of the POLRMT exon sequence identified in Ensemble ID ENSE00000655271. In some embodiments, an oligonucleotide is complementary to 8 to 19 contiguous nucleotides of SEQ ID NO: 725 (CAACGCCGTGATGCTTGGCTGGGCGCGGC), which corresponds to nucleotides 817-845 of the POLRMT transcript (SEQ ID NO: 205) and to nucleotides 8999-9027 on the POLRMT gene sequence (SEQ ID NO: 1). In some embodiments, an oligonucleotide is complementary to a sequence comprising SEQ ID NO: 681 or 682. In some embodiments an oligonucleotide targets a region within the POLRMT exon identified in Ensemble ID ENSE00000655271 and comprises a nucleotide sequence having at least 80%, at least 85%, at least 90%, at least 95% or 100% identity to SEQ ID NO: 612 or 613.

In some embodiments, an oligonucleotide is substantially complementary to 8 to 30 contiguous nucleotides of the POLRMT exon sequence identified in Ensemble ID ENSE00000655279. In some embodiments, an oligonucleotide is perfectly complementary to 8 to 30 contiguous nucleotides of the POLRMT exon sequence identified in Ensemble ID ENSE00000655279. In some embodiments, an oligonucleotide is complementary to 8 to 22 contiguous nucleotides of the POLRMT exon sequence identified in Ensemble ID ENSE00000655279. In some embodiments, an oligonucleotide is complementary 8 to 22 contiguous nucleotides of SEQ ID NO: 726 (CGCACAACATGGACTTCCGCGGCCGCACCTAC), which corresponds to nucleotides 2415-2446 of the POLRMT transcript (SEQ ID NO: 205) and nucleotides 117261-17292 of the POLRMT gene sequence (SEQ ID NO: 1). In some embodiments, an oligonucleotide is complementary to a sequence comprising SEQ ID NO: 661, 663, 692, 693, 694, or 695. In some embodiments an oligonucleotide targets a region within the POLRMT exon identified in Ensemble ID ENSE00000655279 and comprises a nucleotide sequence having at least 80%, at least 85%, at least 90%, at least 95% or 100% identity to SEQ ID NO: 592, 594, 623, 624, 625, or 626.

In some embodiments, an oligonucleotide is substantially complementary to 8 to 30 contiguous nucleotides of the POLRMT exon sequence identified in Ensemble ID ENSE00000655283. In some embodiments, an oligonucleotide is complementary to 8 to 30 contiguous nucleotides of the POLRMT exon sequence identified in Ensemble ID ENSE00000655283. In some embodiments, an oligonucleotide is complementary to 8 to 22 contiguous nucleotides of the POLRMT exon sequence identified in Ensemble ID ENSE00000655283. In some embodiments, an oligonucleotide is complementary to a sequence that spans 8 to 22 contiguous nucleotides of SEQ ID NO: 727 (ATCACCCGCAAGGTGGTGAAGCAGACGGTGA), which corresponds to nucleotides 2978-3008 of the POLRMT transcript (SEQ ID NO: 205) and nucleotides 18870-18900 of the POLRMT gene sequence (SEQ ID NO: 1). In some embodiments, an oligonucleotide is complementary to a sequence comprising SEQ ID NO: 661, 663, 692, 693, 694, or 695. In some embodiments an oligonucleotide targets a region within the POLRMT exon identified in Ensemble ID ENSE00000655283 and comprises a sequence that is at least 80%, 85%, 90%, 95%, or 100% identical to SEQ ID NO: 592, 594, 623, 624, 625, or 626.

In some embodiments, an oligonucleotide comprises a sequence that is complementary (e.g., substantially complementary or perfectly complementary) to a region within a POLRMT transcript, e.g., POLRMT mRNA or pre-mRNA transcript (e.g., complementary to a nucleotide sequence that is at least 80%, 85%, 90%, 95%, or 100% identical to a target region of SEQ ID NO: 205). In some embodiments, an oligonucleotide is complementary to 8 to 30 contiguous nucleotides of a POLRMT RNA transcript (i.e., the target region) e.g., 8, 9, 10, 11, 12, 13, 14, 15, 16, 17, 18, 19, 20, 21, 22, 23, 24, 25, 26, 27, 28, 29, or 30 nucleotides long, although shorter and longer target region are also contemplated.

In some embodiments, the 8 to 30 contiguous nucleotides on the POLRMT RNA transcript (i.e., the target region) comprises a sequence that is at least 80%, 85%, 90%, 95%, or 100% identical to any one of the sequences listed below in Table 3.

In some embodiments, an oligonucleotide is complementary to a target region on the POLRMT RNA transcript that comprises a sequence having at least 80% identity to a sequence selected from a group consisting of SEQ ID NOs: 15-26, 46-64, 299-392, and 656-724. In some embodiments, an oligonucleotide is complementary to a target region on the POLRMT RNA transcript that comprises a sequence having at least 85% identity to a sequence selected from a group consisting of SEQ ID NOs: 15-26, 46-64, 299-392, and 656-724. In some embodiments, an oligonucleotide is complementary to a target region on the POLRMT RNA transcript that comprises a sequence having at least 90% identity to a sequence selected from a group consisting of SEQ ID NOs: 15-26, 46-64, 299-392, and 656-724. In some embodiments, an oligonucleotide is complementary to a target region on the POLRMT RNA transcript that comprises a sequence having at least 95% identity to a sequence selected from a group consisting of SEQ ID NOs: 15-26, 46-64, 299-392, and 656-724. In some embodiments, an oligonucleotide is complementary to a target region on the POLRMT RNA transcript that comprises a sequence selected from a group consisting of SEQ ID NOs: 15-26, 46-64, 299-392, and 656-724.

In some embodiments, a target region comprises a sequence having at least 80% identity to SEQ ID NO: 23. In some embodiments, a target region comprises a sequence having at least 85% to SEQ ID NO: 23. In some embodiments, a target region comprises a sequence having at least 90% identity to SEQ ID NO: 23. In some embodiments, a target region comprises a sequence having at least 95% identity to SEQ ID NO: 23. In some embodiments, target region comprises SEQ ID NO: 23.

In some embodiments, a target region comprises a sequence having at least 80% identity to SEQ ID NO: 24. In some embodiments, a target region comprises a sequence having at least 85% to SEQ ID NO: 24. In some embodiments, a target region comprises a sequence having at least 90% identity to SEQ ID NO: 24. In some embodiments, a target region comprises a sequence having at least 95% identity to SEQ ID NO: 24. In some embodiments, target region comprises SEQ ID NO: 24.

In some embodiments, a target region comprises a sequence having at least 80% identity to SEQ ID NO: 663. In some embodiments, a target region comprises a sequence having at least 85% to SEQ ID NO: 663. In some embodiments, a target region comprises a sequence having at least 90% identity to SEQ ID NO: 663. In some embodiments, a target region comprises a sequence having at least 95% identity to SEQ ID NO: 663. In some embodiments, target region comprises SEQ ID NO: 663.

In some embodiments, a target region comprises a sequence having at least 80% identity to SEQ ID NO: 681. In some embodiments, a target region comprises a sequence having at least 85% to SEQ ID NO: 681. In some embodiments, a target region comprises a sequence having at least 90% identity to SEQ ID NO: 681. In some embodiments, a target region comprises a sequence having at least 95% identity to SEQ ID NO: 681. In some embodiments, target region comprises SEQ ID NO: 681.

In some embodiments, a target region comprises a sequence having at least 80% identity to SEQ ID NO: 701. In some embodiments, a target region comprises a sequence having at least 85% to SEQ ID NO: 701. In some embodiments, a target region comprises a sequence having at least 90% identity to SEQ ID NO: 701. In some embodiments, a target region comprises a sequence having at least 95% identity to SEQ ID NO: 701. In some embodiments, target region comprises SEQ ID NO: 701.

In some embodiments, an oligonucleotide comprises a sequence that is complementary (e.g., substantially complementary or perfectly complementary, and/or that includes no more than 1, 2, 3, or 4 nucleotide mismatches) to 8 to 30 contiguous nucleotides of a POLRMT transcript (e.g., SEQ ID NO: 205, and correspond to a region within gene sequence SEQ ID NO: 1). In some embodiments, an oligonucleotide comprises a sequence that is complementary to any one of the sequences listed below in Table 3.

In some embodiments, a target region on the POLRMT RNA transcript comprises a region that corresponds to nucleotides 5696-5715, 8808-8827, 8809-8828, 8811-8830, 16221-16240, 17159-17178, 17314-17333, 17315-17334, 18082-18101, 18083-18102, 18084-18103, 18130-18149, 5680-5699, 8491-8510, 8529-8548, 8569-8588, 8570-8589, 8571-8590, 8572-8591, 8573-8592, 8574-8593, 13322-13341, 13719-13738, 14999-15018, 15092-15111, 15093-15112, 17304-17323, 19309-19328, 20041-20060, 20042-20061, 21102-21121, 5032-5051, 5034-5053, 5036-5055, 5059-5078, 5691-5710, 5692-5711, 5696-5715, 5703-5722, 8450-8469, 8452-8471, 8456-8475, 8520-8539, 8647-8666, 8650-8669, 8705-8724, 8706-8725, 8708-8727, 8716-8735, 8717-8736, 8803-8822, 8813-8832, 8986-9005, 8993-9012, 8994-9013, 8995-9014, 13435-13447 and 13666-13672, 13705-13724, 13706-13725, 13710-13729, 13743-13762, 13753-13772, 13842-13850 and 14966-14976, 13846-13850 and 14966-14980, 13847-13850 and 14966-14981, 13849-13850 and 14966-14983, 13850-13850 and 14966-14984, 15087-15106, 15603-15622, 15607-15626, 15615-15634, 15904-15923, 16215-16234, 16334-16353, 16786-16805, 16787-16806, 16788-16807, 16789-16808, 16792-16811, 16829-16848, 16832-16851, 16833-16852, 16834-16853, 16836-16855, 16838-16857, 16998-17017, 16999-17018, 17140-17159, 17141-17160, 17147-17166, 17153-17172, 17155-17174, 17157-17176, 17164-17183, 17231-17250, 17259-17278, 17261-17280, 17309-17328, 17311-17330, 17313-17332, 17319-17338, 17321-17340, 17345-17364, 17506-17512 and 18083-18095, 18090-18109, 18091-18110, 18093-18112, 18095-18114, 18111-18130, 18562-18581, 18583-18602, 18584-18603, 18585-18604, 18586-18605, 18591-18610, 18805-18824, 18844-18863, 19273-19292, 19274-19293, 19809-19828, 20717-20736, 20720-20739, 20721-20740, 20722-20741, 20738-20757, 17266-17285, 17268-17287, 18875-18894, 18876-18895, 9004-9021, 9005-9022, 17267-17284, 17268-17285, 17269-17286, 17270-17287, 18876-18893, 18877-18894, or 18878-18895 of SEQ ID NO: 1. In some embodiments, a target region on the POLRMT RNA transcript comprises a region that corresponds to nucleotides 2420-2439, 2422-2441, 2983-3002, 2984-3003, 822-839, 823-840, 2421-2438, 2422-2439, 2423-2440, 2424-2441, 2984-3001, 2985-3002, or 2986-3003 of the POLRMT transcript sequence (SEQ ID NO: 205).

TABLE 3
Target Region Sequence of Human POLRMT

| SEQ ID | POLRMT RNA Sequence |
|---|---|
| NO: | (5′ to 3′) |
| 15 | GACCGCAGGAAGGACTGGGG |
| 16 | AAGCTGAGCCTCGATGTGGA |
| 17 | AGCTGAGCCTCGATGTGGAG |
| 18 | CTGAGCCTCGATGTGGAGCA |
| 19 | AGTACTGGGAGGAGCTGGGG |
| 20 | CGCACTGCCAGAAGGTGGCC |
| 21 | CACCTGGGCAGCGACGTGGC |
| 22 | ACCTGGGCAGCGACGTGGCG |
| 23 | GGCCGAAAGTGGTGGATGGG |
| 24 | GCCGAAAGTGGTGGATGGGC |
| 25 | CCGAAAGTGGTGGATGGGCG |
| 26 | TGCTGTATGGAGGTGGCGAA |
| 46 | CCAGGAGCAAGACCAAGACC |
| 47 | GCAGTGGAGATGGTAGCCTC |
| 48 | CAGATGGGGGCCAAGGATGC |
| 49 | GCTGGGCAAAGATACTGGAG |
| 50 | CTGGGCAAAGATACTGGAGA |
| 51 | TGGGCAAAGATACTGGAGAA |
| 52 | GGGCAAAGATACTGGAGAAG |
| 53 | GGCAAAGATACTGGAGAAGG |
| 54 | GCAAAGATACTGGAGAAGGA |
| 55 | TCAAGGAGCTGGTATATGTG |
| 56 | GTTCTGCTGTCTGAGGAGGA |
| 57 | CCCTTGAAGACCCTGCAGTG |
| 58 | CCAAGCAAGGAGGTCAAGCA |
| 59 | CAAGCAAGGAGGTCAAGCAC |
| 60 | GCACTTCAACCACCTGGGCA |
| 61 | CAGGTCTTCAAGAGTCTACA |
| 62 | GACTCCTCCCACATGATGCT |
| 63 | ACTCCTCCCACATGATGCTC |
| 64 | TTGGAGGCCAGCCAGCTGAA |
| 148 | GGGGTGGCCTGGAGCGGCGT |
| 149 | GGTGGCCTGGAGCGGCGTGC |
| 150 | TGGCCTGGAGCGGCGTGCGT |
| 151 | GTCGGCACTTTGCTGGGGCC |
| 152 | ACCAAGACCGCAGGAAGGAC |
| 153 | CCAAGACCGCAGGAAGGACT |
| 154 | GACCGCAGGAAGGACTGGGG |
| 155 | GGAAGGACTGGGGCCACGTG |
| 156 | GGTGGTGAACAGGGTGGATG |
| 157 | TGGTGAACAGGGTGGATGTG |
| 158 | GAACAGGGTGGATGTGGCGC |
| 159 | AGGAAGGTCCAGATGGGGGC |
| 160 | CATTCCAGAGCGGGGAGTTC |
| 161 | TCCAGAGCGGGGAGTTCAAG |
| 162 | CCTGAGCAAGCAGATGGCCG |
| 163 | CTGAGCAAGCAGATGGCCGG |
| 164 | GAGCAAGCAGATGGCCGGGT |
| 165 | AGATGGCCGGGTGCCTGGAG |
| 166 | GATGGCCGGGTGCCTGGAGG |
| 167 | CTGGGAAGCTGAGCCTCGAT |
| 168 | GAGCCTCGATGTGGAGCAGG |
| 169 | CGCTGGACATGTACAACGCC |
| 170 | CATGTACAACGCCGTGATGC |
| 171 | ATGTACAACGCCGTGATGCT |
| 172 | TGTACAACGCCGTGATGCTT |
| 173 | ACCATCGAAAGGTGTCTGGA |
| 174 | CACTCTTCACCGCCGTTCTG |
| 175 | ACTCTTCACCGCCGTTCTGC |
| 176 | TTCACCGCCGTTCTGCTGTC |
| 177 | GCCACTGTTCTGAAGGCCGT |
| 178 | TGAAGGCCGTGCACAAGGTG |
| 179 | TATGCCAAGGATGGGCGTGT |
| 180 | CCTGCAAGGATGGGCGTGTGTCC |
| 181 | CAAGGATGGGCGTGTGTCCT |
| 182 | AGGATGGGCGTGTGTCCTAC |
| 183 | GGATGGGCGTGTGTCCTACC |
| 184 | CGTTGCCAAGCAAGGAGGTC |
| 185 | TGCGGGACCAATGGGAGAAA |
| 186 | GGACCAATGGGAGAAAGCAC |
| 187 | GGGAGAAAGCACTGTGCCGG |
| 188 | CAGAGGCAGCGGGTCAGTGG |
| 189 | CGCGGCAGTACTGGGAGGAG |
| 190 | ATGCCATGCAGCCTGGACAA |
| 191 | CTGACCTTCGAGGCGGTGGA |
| 192 | TGACCTTCGAGGCGGTGGAT |
| 193 | GACCTTCGAGGCGGTGGATG |
| 194 | ACCTTCGAGGCGGTGGATGT |
| 195 | TTCGAGGCGGTGGATGTACC |
| 196 | TGCCCTGGACATCGCCGCAC |
| 197 | CCTGGACATCGCCGCACTCT |
| 198 | CTGGACATCGCCGCACTCTG |
| 199 | TGGACATCGCCGCACTCTGG |
| 200 | GACATCGCCGCACTCTGGTG |
| 201 | CATCGCCGCACTCTGGTGCT |
| 202 | CAACGGGCGCGTGCTGGACC |
| 203 | AACGGGCGCGTGCTGGACCT |
| 204 | GAGCTGCGCCGTGAGCTGGC |
| 299 | GGGGTGGCCTGGAGCGGCGG |
| 300 | GGTGGCCTGGAGCGGCGTGC |
| 301 | TGGCCTGGAGCGGCGTGCGT |
| 302 | GTCGGCACTTTGCTGGGGCC |
| 303 | ACCAAGACCGCAGGAAGGAC |
| 304 | CCAAGACCGCAGGAAGGACT |
| 305 | GACCGCAGGAAGGACTGGGG |
| 306 | GGAAGGACTGGGGCCACGTG |
| 307 | GGTGGTGAACAGGGTGGATG |
| 308 | TGGTGAACAGGGTGGATGTG |
| 309 | GAACAGGGTGGATGTGGCGC |
| 310 | AGGAAGGTCCAGATGGGGGC |
| 311 | CATTCCAGAGCGGGGAGTTC |
| 312 | TCCAGAGCGGGGAGTTCAAG |
| 313 | CCTGAGCAAGCAGATGGCCG |
| 314 | CTGAGCAAGCAGATGGCCGG |
| 315 | GAGCAAGCAGATGGCCGGGT |
| 316 | AGATGGCCGGGTGCCTGGAG |
| 317 | GATGGCCGGGTGCCTGGAGG |
| 318 | CTGGGAAGCTGAGCCTCGAT |
| 319 | GAGCCTCGATGTGGAGCAGG |
| 320 | CGCTGGACATGTACAACGCC |
| 321 | CATGTACAACGCCGTGATGC |
| 322 | ATGTACAACGCCGTGATGCT |
| 323 | TGTACAACGCCGTGATGCTT |
| 324 | ACCATCGAAAGGTGTCTGGA |
| 325 | CACTCTTCACCGCCGTTCTG |
| 326 | ACTCTTCACCGCCGTTCTGC |
| 327 | TTCACCGCCGTTCTGCTGTC |
| 328 | GCCACTGTTCTGAAGGCCGT |
| 329 | TGAAGGCCGTGCACAAGGTG |
| 330 | TATGCCAAGGATGGGCGTGT |
| 331 | CCAAGGATGGGCGTGTGTCC |
| 332 | CAAGGATGGGCGTGTGTCCT |
| 333 | AGGATGGGCGTGTGTCCTAC |
| 334 | GGATGGGCGTGTGTCCTACC |
| 335 | CGTTGCCAAGCAAGGAGGTC |
| 336 | TGCGGGACCAATGGGAGAAA |
| 337 | GGACCAATGGGAGAAAGCAC |
| 338 | GGGAGAAAGCACTGTGCCGG |
| 339 | CAGAGGCAGCGGGTCAGTGG |
| 340 | CGCGGCAGTACTGGGAGGAG |
| 341 | ATGCCATGCAGCCTGGACAA |
| 342 | CTGACCTTCGAGGCGGTGGA |
| 343 | TGACCTTCGAGGCGGTGGAT |
| 344 | GACCTTCGAGGCGGTGGATG |
| 345 | ACCTTCGAGGCGGTGGATGT |
| 346 | TTCGAGGCGGTGGATGTACC |
| 347 | TGCCCTGGACATCGCCGCAC |
| 348 | CCTGGACATCGCCGCACTCT |
| 349 | CTGGACATCGCCGCACTCTG |
| 350 | TGGACATCGCCGCACTCTGG |
| 351 | GACATCGCCGCACTCTGGTG |
| 352 | CATCGCCGCACTCTGGTGCT |
| 353 | CAACGGGCGCGTGCTGGACC |
| 354 | AACGGGCGCGTGCTGGACCT |
| 355 | GAGCTGCGCCGTGAGCTGGC |
| 356 | AGCTGCGCCGTGAGCTGGCG |
| 357 | GCCGTGAGCTGGCGCACTGC |
| 358 | AGCTGGCGCACTGCCAGAAG |
| 359 | CTGGCGCACTGCCAGAAGGT |
| 360 | GGCGCACTGCCAGAAGGTGG |
| 361 | TGCCAGAAGGTGGCCCGGGA |
| 362 | AGCACCTGCGGGACCGCGTC |
| 363 | GCCGCACAACATGGACTTCC |
| 364 | CGCACAACATGGACTTCCGC |
| 365 | TCAACCACCTGGGCAGCGAC |
| 366 | AACCACCTGGGCAGCGACGT |
| 367 | CCACCTGGGCAGCGACGTGG |
| 368 | GGGCAGCGACGTGGCGCGGG |
| 369 | GCAGCGACGTGGCGCGGGCC |
| 370 | TGGAGTTCGCCCAGGGCCGC |
| 371 | TTGACGGGCCGAAAGTGGTG |
| 372 | GTGGTGGATGGGCGCGGAGG |
| 373 | TGGTGGATGGGCGCGGAGGA |
| 374 | GTGGATGGGCGCGGAGGAAC |
| 375 | GGATGGGCGCGGAGGAACCC |
| 376 | ACCCTGGCAGACGCTGGCCT |
| 377 | TGGAGCCCTCGGATGTGCCG |
| 378 | AGGACGTGTACAGCGGCGTG |
| 379 | GGACGTGTACAGCGGCGTGG |
| 380 | GACGTGTACAGCGGCGTGGC |
| 381 | ACGTGTACAGCGGCGTGGCC |
| 382 | TACAGCGGCGTGGCCGCGCA |
| 383 | TGGAGGTGTTCCGTAGGCAG |
| 384 | GGGTGGCACAGGTGCTGGAA |
| 385 | GAGTTCGTGTGGGAGGCCTC |
| 386 | AGTTCGTGTGGGAGGCCTCT |
| 387 | CAAATAGGAGGTGGAATTCA |
| 388 | CAGGAAGGGCCTGACCTTCG |
| 389 | GAAGGGCCTGACCTTCGTCT |
| 390 | AAGGGCCTGACCTTCGTCTC |
| 391 | AGGGCCTGACCTTCGTCTCT |
| 392 | CTCTGTGCACGACTGTTACT |
| 656 | TGCCGCACAACATGGACTTC |
| 657 | CCGCACAACATGGACTTCCG |
| 658 | GCACAACATGGACTTCCGCG |
| 659 | ACAACATGGACTTCCGCGGC |
| 660 | CAACATGGACTTCCGCGGCC |
| 661 | AACATGGACTTCCGCGGCCG |
| 662 | ACATGGACTTCCGCGGCCGC |
| 663 | CATGGACTTCCGCGGCCGCA |
| 664 | ATGGACTTCCGCGGCCGCAC |
| 665 | CACTTCAACCACCTGGGCAG |
| 666 | CCGCAAGGTGGTGAAGCAGA |
| 667 | CGCAAGGTGGTGAAGCAGAC |
| 668 | GGCTTCCCGCCCAACTTCAT |
| 669 | GCTTCCCGCCCAACTTCATC |
| 670 | CTTCCCGCCCAACTTCATCC |
| 671 | TTCCCGCCCAACTTCATCCA |
| 672 | TCCCGCCCAACTTCATCCAC |
| 673 | CCGCCCAACTTCATCCACTC |
| 674 | CCGCCCAACTTCATCCACTC |
| 675 | CTGGACTCCTCCCACATGAT |
| 676 | TGGACTCCTCCCACATGATG |
| 677 | GGACTCCTCCCACATGATGC |
| 678 | CACTGCTACAGGAAGGGCCT |
| 679 | ACTGCTACAGGAAGGGCCTG |
| 680 | CTGCTACAGGAAGGGCCTGA |
| 681 | CCGTGATGCTTGGCTGGG |
| 682 | CGTGATGCTTGGCTGGGC |
| 683 | TGCCGCACAACATGGACT |
| 684 | GCCGCACAACATGGACTT |
| 685 | CCGCACAACATGGACTTC |
| 686 | CGCACAACATGGACTTCC |
| 687 | GCACAACATGGACTTCCG |
| 688 | CACAACATGGACTTCCGC |
| 689 | ACAACATGGACTTCCGCG |
| 690 | CAACATGGACTTCCGCGG |
| 691 | AACATGGACTTCCGCGGC |
| 692 | ACATGGACTTCCGCGGCC |
| 693 | CATGGACTTCCGCGGCCG |
| 694 | ATGGACTTCCGCGGCCGC |
| 695 | TGGACTTCCGCGGCCGCA |
| 696 | GGACTTCCGCGGCCGCAC |
| 697 | CACTTCAACCACCTGGGC |
| 698 | ACTTCAACCACCTGGGCA |
| 699 | CTTCAACCACCTGGGCAG |
| 700 | CCGCAAGGTGGTGAAGCA |
| 701 | CGCAAGGTGGTGAAGCAG |
| 702 | GCAAGGTGGTGAAGCAGA |
| 703 | CAAGGTGGTGAAGCAGAC |
| 704 | AGTCTACAGGAGATGTTC |
| 705 | GGCTTCCCGCCCAACTTC |
| 706 | GCTTCCCGCCCAACTTCA |
| 707 | CTTCCCGCCCAACTTCAT |
| 708 | TTCCCGCCCAACTTCATC |
| 709 | TCCCGCCCAACTTCATCC |
| 710 | CCCGCCCAACTTCATCCA |
| 711 | CCGCCCAACTTCATCCAC |
| 712 | CGCCCAACTTCATCCACT |
| 713 | GCCCAACTTCATCCACTC |
| 714 | CTGGACTCCTCCCACATG |
| 715 | TGGACTCCTCCCACATGA |
| 716 | GGACTCCTCCCACATGAT |
| 717 | GACTCCTCCCACATGATG |
| 718 | ACTCCTCCCACATGATGC |
| 719 | CTCCTCCCACATGATGCT |
| 720 | CACTGCTACAGGAAGGGC |
| 721 | ACTGCTACAGGAAGGGCC |
| 722 | CTGCTACAGGAAGGGCCT |
| 723 | TGCTACAGGAAGGGCCTG |
| 724 | GCTACAGGAAGGGCCTGA |

In some embodiments, an oligonucleotide comprises a sequence that differs by no more than 1, 2, 3, or 4 nucleotides from any one of SEQ ID NOs: 3-14, 592, 594, 597, 598, 612, 613, 623, 624, 625, 626, 632, 633, 634, or 728-740 and/or is complementary to a nucleotide sequence that differs by no more than 1, 2, 3, or 4 nucleotides from any one of SEQ ID NO: 15-26, 661, 663, 666, 667, 681, 682, 692, 693, 694, 695, 701, 702, or 703.

In some embodiments, an oligonucleotide comprises one or more mismatch(es) (e.g., 1, 2, 3, 4, or 5) with the target region (i.e., a nucleotide that is not complementary with the corresponding nucleotide in the target region sequence).

In some embodiments, an oligonucleotide is complementary to a target region within the mouse POLRMT RNA transcript (e.g., as shown in SEQ ID NO: 582). In some embodiments, an oligonucleotide is complementary to a target region within the mouse POLRMT transcript (e.g., as shown in SEQ ID NO: 582) and is also complementary to a corresponding target region within the human POLRMT transcript (e.g., as shown in SEQ ID NO: 205), particularly if the target region corresponds to a conserved region between the mouse and human POLRMT sequences. In some embodiments, an oligonucleotide is complementary to a particular target region within the mouse POLRMT RNA transcript that corresponds to a region within the mouse POLRMT gene sequence (e.g., as shown in SEQ ID NO: 581).

In some embodiments, an oligonucleotide of the present disclosure is complementary to 8 to 30 contiguous nucleotides (i.e., a target region) of a mouse POLRMT RNA transcript. In some embodiments, an oligonucleotide is complementary to a target region of a mouse POLRMT RNA transcript and comprises any one of the sequences as shown in Table 4.

In some embodiments, an oligonucleotide comprises a sequence that is complementary (e.g., substantially complementary or perfectly complementary) to a region of a mouse POLRMT transcript, e.g., mouse POLRMT mRNA or pre-mRNA RNA transcript (e.g., complementary to a nucleotide sequence that is at least 80%, 85%, 90%, 95%, or 100% identical to a target region of SEQ ID NO: 582). In some embodiments, an oligonucleotide is complementary to 8 to 30 contiguous nucleotides of a mouse PORLMT transcript (i.e., the target region) e.g., 8, 9, 10, 11, 12, 13, 14, 15, 16, 17, 18, 19, 20, 21, 22, 23, 24, 25, 26, 27, 28, 29, or 30 nucleotides long, although shorter and longer target region are also contemplated. In some embodiments, such an oligonucleotide sequence also targets a corresponding region within a human POLRMT transcript.

In some embodiments, the 8 to 30 contiguous nucleotides on the POLRMT RNA transcript (i.e., the target region) comprises a sequence that is at least 80%, 85%, 90%, 95%, or 100% identical to any one of the sequences listed below in Table 4.

In some embodiments, an oligonucleotide is complementary to a target region on the POLRMT RNA transcript that comprises a sequence having at least 80% identity to a sequence selected from a group consisting of SEQ ID NOs: 487-580, 661, 663, 666, 667, 681, 682, 692, 693, 694, 695, 701, 702, and 703. In some embodiments, an oligonucleotide is complementary to a target region on the POLRMT RNA transcript that comprises a sequence having at least 85% identity to a sequence selected from a group consisting of SEQ ID NOs: 487-580, 661, 663, 666, 667, 681, 682, 692, 693, 694, 695, 701, 702, and 703. In some embodiments, an oligonucleotide is complementary to a target region on the POLRMT RNA transcript that comprises a sequence having at least 90% identity to a sequence selected from a group consisting of SEQ ID NOs: 487-580, 661, 663, 666, 667, 681, 682, 692, 693, 694, 695, 701, 702, and 703. In some embodiments, an oligonucleotide is complementary to a target region on the POLRMT RNA transcript that comprises a sequence having at least 95% identity to a sequence selected from a group consisting of SEQ ID NOs: 487-580, 661, 663, 666, 667, 681, 682, 692, 693, 694, 695, 701, 702, and 703. In some embodiments, an oligonucleotide is complementary to a target region on the POLRMT RNA transcript that comprises a sequence selected from a group consisting of SEQ ID NOs: 487-580, 661, 663, 666, 667, 681, 682, 692, 693, 694, 695, 701, 702, and 703.

In some embodiments, a target region comprises a sequence having at least 80% identity to SEQ ID NO: 528. In some embodiments, a target region comprises a sequence having at least 85% to SEQ ID NO: 528. In some embodiments, a target region comprises a sequence having at least 90% identity to SEQ ID NO: 528. In some embodiments, a target region comprises a sequence having at least 95% identity to SEQ ID NO: 528. In some embodiments, target region comprises SEQ ID NO: 528.

In some embodiments, a target region comprises a sequence having at least 80% identity to SEQ ID NO: 536. In some embodiments, a target region comprises a sequence having at least 85% to SEQ ID NO: 536. In some embodiments, a target region comprises a sequence having at least 90% identity to SEQ ID NO: 536. In some embodiments, a target region comprises a sequence having at least 95% identity to SEQ ID NO: 536. In some embodiments, target region comprises SEQ ID NO: 536.

In some embodiments, an oligonucleotide comprises a sequence that is complementary (e.g., substantially complementary or perfectly complementary, and/or that includes no more than 1, 2, 3, or 4 nucleotide mismatches) to 8 to 30 contiguous nucleotides of a mouse POLRMT transcript (e.g., SEQ ID NO: 582) and corresponds to a region within the mouse gene sequence (e.g., SEQ ID NO: 581). In some embodiments, an oligonucleotide comprises a sequence that is complementary any one of the sequences listed below in Table 4. In some embodiments, an oligonucleotide may be complementary to any one of the sequences listed below in Table 4 but differs in one or more nucleotides in order to be complementary to the corresponding human POLRMT target region (e.g., within SEQ ID NO: 205, corresponding to a region within the human gene sequence SEQ ID NO: 1).

SEQ ID NO: 581 In some embodiments, a target region on the mouse POLRMT RNA transcript comprises a region that corresponds to nucleotides 7077-7096, 7075-7094, 7074-7093, 3342-3361, 3341-3360, 3340-3359, 3297-3316, 3258-3277, 3202-3221, 2663-2682, 2621-2640, 2620-2639, 2619-2638, 2618-2637, 2617-2636, 2005-2024, 2003-2022, 7107-7126, 7105-7124, 7103-7122, 7082-7101, 7079-7098, 5712-5731, 5707-5726, 5705-5724, 4732-4751, 4731-4750, 4730-4749, 4568-4587, 4178-4197, 4136-4155, 4135-4154, 4133-4152, 4132-4151, 4131-4150, 4130-4149, 4129-4148, 4128-4147, 3597-3616, 3545-3564, 3544-3563, 3348-3367, 3339-3358, 3338-3357, 3337-3356, 3336-3355, 3335-3354, 3252-3271, 3250-3269, 3198-3217, 3197-3216, 3196-3215, 3168-3187, 2662-2681, 2660-2679, 2659-2678, 2658-2677, 2657-2676, 2656-2675, 2655-2674, 2654-2673, 2652-2671, 2651-2670, 2616-2635, 2615-2634, 2614-2633, 2613-2632, 2612-2631, 2611-2630, 2540-2559, 2537-2556, 2532-2551, 2380-2399, 2271-2290, 2270-2289, 2209-2228, 2207-2226, 2206-2225, 2195-2214, 1536-1555, 1534-1553, 1532-1551, 1377-1396, 1376-1395, 1375-1394, 1313-1332, 1312-1331, 1311-1330, 1308-1327, 1287-1306, 1286-1305, 1283-1302, 637-656, or 19-38 of SEQ ID NO: 581, or a corresponding target region within human POLRMT sequence (as shown in SEQ ID NO: 1).

In some embodiments, a target region on the POLRMT RNA transcript comprises a region that corresponds to nucleotides 2329-2348, 2331-2350, 3240-3259, 3241-3260, 734-751, 735-752, 2330-2347, 2331-2348, 2332-2349, 2333-2350, 3241-3258, 3242-3259, or 3243-3260 of the mouse PORLMT transcript (SEQ ID NO: 582), or a corresponding region within a human POLRMT transcript sequence (e.g., as shown in SEQ ID NO: 205).

TABLE 4
Target Region Sequence of mouse POLRMT

| SEQ ID | POLRMT RNA sequence |
|---|---|
| NO: | (5′ to 3′) |
| 487 | TGCACATGTACAACACCGTG |
| 488 | CACATGTACAACACCGTGAT |
| 489 | ACATGTACAACACCGTGATG |
| 490 | AGGTGGCTCGAGAGATGCAC |
| 491 | GGTGGCTCGAGAGATGCACA |
| 492 | GTGGCTCGAGAGATGCACAG |
| 493 | GCCTGTCGCTGGCACAGCAC |
| 494 | GGTTGCCGCACAACATGGAC |
| 495 | AACCACCTGGGCAGTGACCT |
| 496 | GGCCGGAAGTGGTGGATGGA |
| 497 | CTGGCCTGCTGCATGGAGGT |
| 498 | TGGCCTGCTGCATGGAGGTG |
| 499 | GGCCTGCTGCATGGAGGTGG |
| 500 | GCCTGCTGCATGGAGGTGGC |
| 501 | CCTGCTGCATGGAGGTGGCA |
| 502 | TCATCAGCCGCAAGGTGGTG |
| 503 | ATCAGCCGCAAGGTGGTGAA |
| 504 | ACAACGGAGACAGACAGCAG |
| 505 | AACGGAGACAGACAGCAGGT |
| 506 | CGGAGACAGACAGCAGGTGC |
| 507 | CACACTGCACATGTACAACA |
| 508 | ACTGCACATGTACAACACCG |
| 509 | TGCTCTTCACTGACCTGGTC |
| 510 | TTCACTGACCTGGTCCTGGA |
| 511 | CACTGACCTGGTCCTGGAAG |
| 512 | GCCTGCTGAGCGAAGGGGAG |
| 513 | CCTGCTGAGCGAAGGGGAGT |
| 514 | CTGCTGAGCGAAGGGGAGTT |
| 515 | AGTTGGCCCATAACCTGGGC |
| 516 | TGGAGGCCCCTGCACAACAG |
| 517 | TACAGCTGGGCAAGCAATTG |
| 518 | ACAGCTGGGCAAGCAATTGG |
| 519 | AGCTGGGCAAGCAATTGGCG |
| 520 | GCTGGGCAAGCAATTGGCGG |
| 521 | CTGGGCAAGCAATTGGCGGA |
| 522 | TGGGCAAGCAATTGGCGGAG |
| 523 | GGGCAAGCAATTGGCGGAGC |
| 524 | GGCAAGCAATTGGCGGAGCT |
| 525 | GTCTGCTGGAGCAATGCCCT |
| 526 | CACACAGTTGGGGAACTGCG |
| 527 | ACACAGTTGGGGAACTGCGC |
| 528 | GCCTCAAGGTGGCTCGAGAG |
| 529 | TGGCTCGAGAGATGCACAGT |
| 530 | GGCTCGAGAGATGCACAGTC |
| 531 | GCTCGAGAGATGCACAGTCT |
| 532 | CTCGAGAGATGCACAGTCTG |
| 533 | TCGAGAGATGCACAGTCTGC |
| 534 | CGCACAACATGGACTTCCGC |
| 535 | CACAACATGGACTTCCGCGG |
| 536 | ACCTGGGCAGTGACCTAGCG |
| 537 | CCTGGGCAGTGACCTAGCGC |
| 538 | CTGGGCAGTGACCTAGCGCG |
| 539 | TGGAGTTTGCTGAGGGCCGG |
| 540 | GCCGGAAGTGGTGGATGGAA |
| 541 | CGGAAGTGGTGGATGGAAGC |
| 542 | GGAAGTGGTGGATGGAAGCT |
| 543 | GAAGTGGTGGATGGAAGCTG |
| 544 | AAGTGGTGGATGGAAGCTGA |
| 545 | AGTGGTGGATGGAAGCTGAT |
| 546 | GTGGTGGATGGAAGCTGATG |
| 547 | TGGTGGATGGAAGCTGATGA |
| 548 | GTGGATGGAAGCTGATGAGC |
| 549 | TGGATGGAAGCTGATGAGCC |
| 550 | CTGCTGCATGGAGGTGGCAC |
| 55 | TGCTGCATGGAGGTGGCACA |
| 552 | GCTGCATGGAGGTGGCACAC |
| 553 | CTGCATGGAGGTGGCACACG |
| 554 | TGCATGGAGGTGGCACACGC |
| 555 | GCATGGAGGTGGCACACGCA |
| 556 | GTGGGCACGCAAGTGGACTG |
| 557 | GGCACGCAAGTGGACTGCCC |
| 558 | GCAAGTGGACTGCCCATGGC |
| 559 | AGTTTCTGGGGTGACCTGTG |
| 560 | AGGCCGAGACAGTGTGGGTG |
| 561 | GGCCGAGACAGTGTGGGTGC |
| 562 | TGTACAGGGAGGTGGCAACA |
| 563 | TACAGGGAGGTGGCAACACA |
| 564 | ACAGGGAGGTGGCAACACAG |
| 565 | GCAACACAGGTGGGTGCCAG |
| 566 | GCCGGATGGCCTGTGGAGTG |
| 567 | CGGATGGCCTGTGGAGTGGG |
| 568 | GATGGCCTGTGGAGTGGGTC |
| 569 | GGTCTGCAGGTAAAAGGTGG |
| 570 | GTCTGCAGGTAAAAGGTGGC |
| 571 | TCTGCAGGTAAAAGGTGGCC |
| 572 | GAGTCCTGTACTGCGGGGAG |
| 573 | AGTCCTGTACTGCGGGGAGG |
| 574 | GTCCTGTACTGCGGGGAGGG |
| 575 | CTGTACTGCGGGGAGGGGAG |
| 576 | GGATGGAGGGATGGGCAGGA |
| 577 | GATGGAGGGATGGGCAGGAA |
| 578 | GGAGGGATGGGCAGGAAAGG |
| 579 | GACTGCTTCTGGACACATGC |
| 580 | CACTTTCTGGCGGTGCGCAA |
| 661 | AACATGGACTTCCGCGGCCG |
| 663 | CATGGACTTCCGCGGCCGCA |
| 666 | CCGCAAGGTGGTGAAGCAGA |
| 667 | CGCAAGGTGGTGAAGCAGAC |
| 681 | CCGTGATGCTTGGCTGGG |
| 682 | CGTGATGCTTGGCTGGGC |
| 692 | ACATGGACTTCCGCGGCC |
| 693 | CATGGACTTCCGCGGCCG |
| 694 | ATGGACTTCCGCGGCCGC |
| 695 | TGGACTTCCGCGGCCGCA |
| 701 | CGCAAGGTGGTGAAGCAG |
| 702 | GCAAGGTGGTGAAGCAGA |
| 703 | CAAGGTGGTGAAGCAGAC |

### Modifications

In some embodiments, an oligonucleotide of the disclosure comprises a sequence based on a phosphodiester backbone (i.e., an unmodified oligonucleotide sequence). In some embodiments, an oligonucleotide of the disclosure includes one or more modified nucleotides.

The use of naturally occurring nucleic acids (e.g., unmodified DNA or RNA) is limited, for example, by their susceptibility to endo- and exo-nucleases. As such, various synthetic counterparts have been developed to circumvent these shortcomings. These include synthetic oligonucleotides that contain chemical modification, e.g., base modifications, sugar modifications, backbone modifications, etc., which, among other things, render these molecules less susceptible to degradation and improve other properties of oligonucleotides. Chemical modifications may also lead to certain undesired effects, such as increased toxicities, etc.

Such as base sequence, chemical modifications (e.g., modifications of sugar, base, and/or internucleotidic linkages, and patterns thereof), and/or stereochemistry (e.g., stereochemistry of backbone chiral centers (chiral internucleotidic linkages), and/or patterns thereof) can have significant impact on properties, e.g., stability, splicing-altering capabilities, etc. In some embodiments, oligonucleotide properties can be adjusted by optimizing chemical modifications (modifications of base, sugar, and/or internucleotidic linkage) and/or stereochemistry (pattern of backbone chiral centers).

In some embodiments, a modified nucleotide comprises a base modification, a sugar or sugar phosphate modification, an internucleotidic linkage modification, or a combination thereof.

In some embodiments, an oligonucleotide of the disclosure includes one or more natural nucleobase and/or one or more modified nucleobases derived from a natural nucleobase. Examples include, but are not limited to, uracil, thymine, adenine, cytosine, and guanine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products).

Modified nucleobases also include expanded-size nucleobases in which one or more aryl rings, such as phenyl rings, have been added.

In some embodiments, modified nucleobases comprise any one of the following substituents, each of which is optionally substituted:

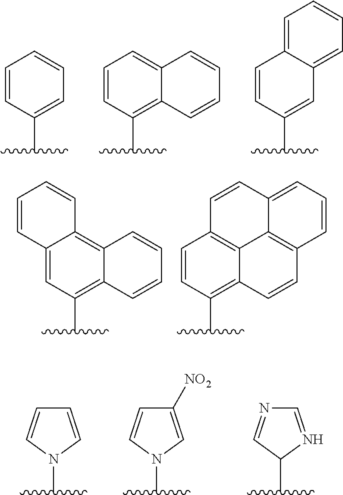

For example, a pyrene-modified guanine base can have the structure

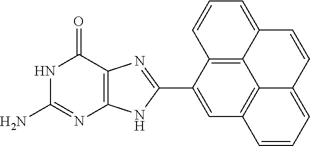 [SMILES: Nc1nc2[nH]c(-c3cc4cccc5ccc6cccc3c6c54)nc2c(=O)[nH]1]

A person skilled in the art would understand where and how a nucleobase can be modified with any of the foregoing groups.

In some embodiments, a modified nucleobase is unsubstituted. In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One representative example of such a universal base is 3-nitropyrrole.

In some embodiments, an oligonucleotide described herein includes nucleosides that incorporate modified nucleobases and/or nucleobases covalently bound to modified sugars (i.e., a “base modification”). Some examples of nucleosides that incorporate modified nucleobases include 4-acetylcytidine; 5-(carboxyhydroxylmethyl)uridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methylcytidine; 5-methylcytidine; 5-hydroxymethylcytidine; 5-methylcytosine, 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxyacetic acid; pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine. In some embodiments, an oligonucleotide described herein comprises at least one G-clamp modification.

In some embodiments, nucleosides include 6′-modified bicyclic nucleoside analogs that have either (R) or (S)-chirality at the 6′-position and include the analogs described in U.S. Pat. No. 7,399,845. In other embodiments, nucleosides include 5′-modified bicyclic nucleoside analogs that have either (R) or (S)-chirality at the 5′-position and include the analogs described in U.S. Publ. No. 20070287831. In some embodiments, a nucleobase or modified nucleobase is 5-bromouracil, 5-iodouracil, or 2,6-diaminopurine. In some embodiments, a nucleobase or modified nucleobase is modified by substitution with a fluorescent moiety.

In some embodiments, an oligonucleotide described herein includes one or more modified nucleotides wherein a phosphate group or linkage phosphorus in the nucleotides are linked to various positions of a sugar or modified sugar. As non-limiting examples, the phosphate group or linkage phosphorus can be linked to the 2′, 3′, 4′ or 5′ hydroxyl moiety of a sugar or modified sugar. Nucleotides that incorporate modified nucleobases as described herein are also contemplated in this context. In some embodiments, the sugar or sugar phosphate modification comprises a 2′-O-methoxyethyl (2′-MOE) modification, a 2′-fluoro (2′-F) modification, a 2′-O-methyl (2′-O-Me) modification, a phosphorodiamidate morpholino (PMO) modification, a peptide nucleic acid (PNA) modification, a glycol nucleic acid (GNA), an unlocked nucleic acid (UNA), or a locked nucleic acid (LNA).

Other modified sugars can also be incorporated within an oligonucleotide molecule. In some embodiments, a modified sugar contains one or more groups at the 2′ position selected from —F, —CF₃, —CN, —N₃, —NO, —NO₂, —OR′, —SR′, or —N(R′)₂, wherein each R′ is independently hydrogen or optionally substituted C₁-C₁₀ aliphatic. In some embodiments, a modified sugar contains one or more groups at the 2′ position selected from —F, —CF₃, —CN, —N₃, —NO, —NO₂, —O—(C₁-C₁₀ alkyl), —S—(C₁-C₁₀ alkyl), —NH—(C₁-C₁₀ alkyl), —N(C₁-C₁₀ alkyl)₂, —O—(C₂-C₁₀ alkenyl), —S—(C₂-C₁₀ alkenyl), —NH—(C₂-C₁₀ alkenyl), —N(C₂-C₁₀ alkenyl)₂, —O—(C₂-C₁₀ alkynyl), —S—(C₂-C₁₀ alkynyl), —NH—(C₂-C₁₀ alkynyl), —N(C₂-C₁₀ alkynyl)₂, —O—(C₁-C₁₀ alkylene)-O—(C₁-C₁₀ alkyl), —O—(C₁-C₁₀ alkylene)-NH—(C₁-C₁₀ alkyl), —O—(C₁-C₁₀ alkylene)-N(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each alkyl, alkylene, alkenyl and alkynyl may be substituted or unsubstituted. In some embodiments, an alkyl, alkenyl, or alkynyl is substituted by a group selected from —O(CH₂)ₙOCH₃ or —O(CH₂)ₙNH₂, wherein n is from 1 to about 10, MOE, DMAOE, and DMAEOE.

In some embodiments, the 2′-OH of a ribose is replaced with a group selected from —H, —F, —CF₃, —CN, —N₃, —NO, —NO₂, —OR′, —SR′, or —N(R′)₂, wherein each R′ is independently hydrogen or optionally substituted C₁-C₁₀ aliphatic. In some embodiments, a modified sugar contains one or more groups at the 2′ position selected from —F, —CF₃, —CN, —N₃, —NO, —NO₂, —O—(C₁-C₁₀ alkyl), —S—(C₁-C₁₀ alkyl), —NH—(C₁-C₁₀ alkyl), —N(C₁-C₁₀ alkyl)₂, —O—(C₂-C₁₀ alkenyl), —S—(C₂-C₁₀ alkenyl), —NH—(C₂-C₁₀ alkenyl), —N(C₂-C₁₀ alkenyl)₂, —O—(C₂-C₁₀ alkynyl), —S—(C₂-C₁₀ alkynyl), —NH—(C₂-C₁₀ alkynyl), —N(C₂-C₁₀ alkynyl)₂, —O—(C₁-C₁₀ alkylene)-O—(C₁-C₁₀ alkyl), —O—(C₁-C₁₀ alkylene)-NH—(C₁-C₁₀ alkyl), —O—(C₁-C₁₀ alkylene)-N(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each alkyl, alkylene, alkenyl and alkynyl may be substituted or unsubstituted. In some embodiments, the 2′-OH is replaced with —H (i.e., deoxyribose). In some embodiments, the 2′-OH is replaced with —F. In some embodiments, the 2′-OH is replaced with —OR′. In some embodiments, the 2′-OH is replaced with —OMe. In some embodiments, the 2′-OH is replaced with —OCH₂CH₂OMe (i.e., MOE).

Modified sugars also include locked nucleic acids (LNAs). In some embodiments, the locked nucleic acid has the structure indicated below. In some embodiments, a locked nucleic acid comprises the structure below, wherein Ba represents a nucleobase or modified nucleobase as described herein, and wherein R^{2s} is —OCH₂C4′-

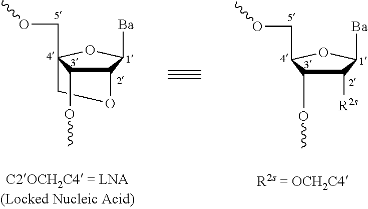

Modified sugars also include unlocked nucleic acids (UNAs). In some embodiments, an unlocked nucleic acid has the structure indicated below (see e.g., Fluiter, Kees, et al., Molecular BioSystems 5.8 (2009): 838-843, which is herein incorporated by reference in its entirety). In some embodiments, a locked nucleic acid comprises the structure below.

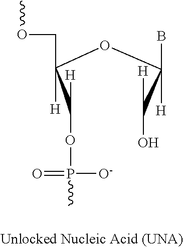

In some embodiments, an oligonucleotide described herein comprises at least one modified internucleotidic linkage. In some embodiments, an internucleotidic linkage modification comprises a phosphorothioate or phosphodithioate linkage modification.

In some embodiments, the present invention provides an oligonucleotide comprising one or more modified internucleotidic linkages independently having the structure of formula I:

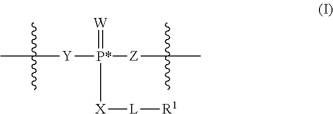

wherein:
P* is an asymmetric phosphorus atom and is either Rp or Sp;
W is O, S or Se;
each of X, Y and Z is independently —O—, —S—, —N(-L-R¹)—, or L;
L is a covalent bond or an optionally substituted, linear or branched, saturated or unsaturated C₁-C₁₀ aliphatic, wherein one or more methylene units of L are optionally and independently replaced by —C(R′)₂—, -Cy-, —O—, —S—, —S—S—, —N(R′)—, —C(O)—, —C(S)—, —C(NR′)—, —C(O)N(R′)—, —N(R′)C(O)N(R′), —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
R¹ is halogen, R, or an optionally substituted, linear or branched, saturated or unsaturated C₁-C₅₀ aliphatic wherein one or more methylene units are optionally and independently replaced by —C(R′)₂—, -Cy-, —O—, —S—, —S—S—, —N(R′)—, —C(O)—, —C(S)—, —C(NR′)—, —C(O)N(R′)—, —N(R′)C(O)N(R′), —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
each R′ is independently —R, C(O)R, CO₂R, or —SO₂R, or:
two R′ on the same nitrogen are taken together with their intervening atoms to form an optionally substituted heterocyclic or heteroaryl ring, or
two R′ on the same carbon are taken together with their intervening atoms to form an optionally substituted aryl, carbocyclic, heterocyclic, or heteroaryl ring;
-Cy- is an optionally substituted bivalent ring selected from carbocyclylene, arylene, heteroarylene, or heterocyclylene;
each R is independently hydrogen, or an optionally substituted group selected from C₁-C₆ aliphatic, carbocyclyl, aryl, heteroaryl, or heterocyclyl; and
each

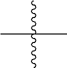

independently represents a connection to a nucleoside.

In some embodiments, the internucleotidic linkage having the structure of formula I

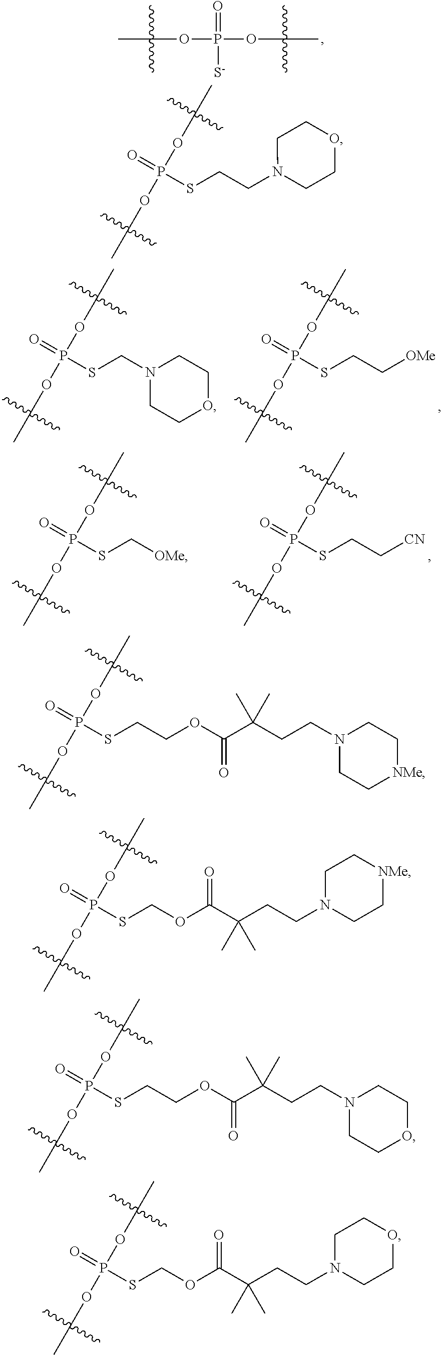

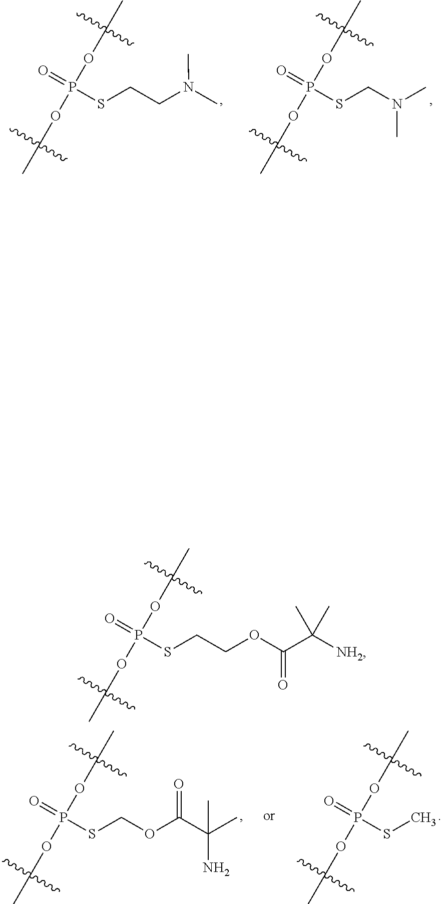

Among other things, the present disclosure provides oligonucleotides of various designs, which may comprise various nucleobases and patterns thereof, sugars and patterns thereof, internucleotidic linkages and patterns thereof, and/or additional chemical moieties and patterns thereof as described in the present disclosure. In some embodiments, provided oligonucleotides can decrease the level of POLRMT protein, POLRMT mRNA expression and/or POLRMT activity in a cell of a subject. In some embodiments, such an oligonucleotide has a base sequence which consists of, comprises, or comprises a portion (e.g., a span of 10, 11, 12, 13, 14, 15, 16, 17, 18, 19, 20 or more contiguous bases) of the base sequence of an oligonucleotide disclosed herein, wherein each T can be independently substituted with U and vice versa, and the oligonucleotide comprises at least one non-naturally-occurring modification of a base, sugar and/or internucleotidic linkage.

According to certain embodiments, various nucleotide modifications or nucleotide modification patterns may be in any of oligonucleotides described herein.

In some embodiments, an oligonucleotide comprises two or more chemically distinct regions, wherein the regions confer distinct properties on the compound. In some embodiments, at least one region is modified so as to confer upon the oligonucleotide increased resistance to nuclease degradation, increased cellular uptake, and/or increased binding affinity for the target nucleic acid and at least one additional region of the oligonucleotide can serve as a substrate for enzymes (e.g., RNase H) capable of cleaving RNA:DNA or RNA:RNA hybrids. In some embodiments, at least one region of the oligonucleotide can serve as a substrate for enzymes (e.g., RNase H) capable of cleaving RNA:DNA or RNA:RNA hybrids and at least one region can inhibit translation by steric blocking.

In some embodiments, an oligonucleotide comprises at least one (e.g., 1, 2, 3, 4, 5, 6, 7, 8, 9, 10, 11, 12, 13, 14, 15, 16, 17, 18, 19, 20 or more) phosphorothioate (PS) internucleotide bond. In some embodiments, an oligonucleotide comprises a sequence where each internucleotidic linkage comprises a phosphorothioate (PS) internucleotide bond. In some embodiments, an oligonucleotide comprises at least one (e.g., 1, 2, 3, 4, 5, 6, 7, 8, 9, 10, 11, 12, 13, 14, 15, 16, 17, 18, 19, 20 or more) phosphodithioate bond. In some embodiments, an oligonucleotide comprises a sequence where each internucleotidic linkage comprises a phosphodithioate bond.

In some embodiments, an oligonucleotide comprises at least one (e.g., 1, 2, 3, 4, 5, 6, 7, 8, 9, 10, 11, 12, 13, 14, 15, 16, 17, 18, 19, 20 or more) 2′-MOE modification.

In some embodiments, an oligonucleotide comprises five nucleotides at the 5′-end and five nucleotides at the 3′-end which contain a 2′-MOE modification.

In some embodiments, an oligonucleotide is modified so that each nucleotide comprises a 2′-MOE modification.

In some embodiments, an oligonucleotide comprises one of the following modification patterns or a portion thereof:

X_{MS}X_{MS}X_{MS}X_{MS}X_{S}X_{S}X_{S}X_{S}X_{S}X_{S}X_{S}X_{S}X_{MS}X_{MS}X_{MS}X_{MS} (“4-8-4” 16-mer)
X_{MS}X_{MS}X_{MS}X_{S}X_{S}X_{S}X_{S}X_{S}X_{S}X_{S}X_{S}X_{S}X_{S}X_{MS}X_{MS}X_{MS} (“3-10-3” 16-mer)
X_{MS}X_{MS}X_{MS}X_{MS}X_{MS}X_{S}X_{S}X_{S}X_{S}X_{S}X_{S}X_{S}X_{S}X_{MS}X_{MS}X_{MS}X_{MS}X_{MS} (“5-8-5” 18-mer)
X_{MS}X_{MS}X_{MS}X_{MS}X_{MS}X_{S}X_{S}X_{S}X_{S}X_{S}X_{S}X_{S}X_{S}X_{S}X_{MS}X_{MS}X_{MS}X_{MS} (“5-9-4” 18-mer)
X_{MS}X_{MS}X_{MS}X_{MS}X_{S}X_{S}X_{S}X_{S}X_{S}X_{S}X_{S}X_{S}X_{S}X_{MS}X_{MS}X_{MS}X_{MS}X_{MS} (“4-9-5” 18-mer)
X_{MS}X_{MS}X_{MS}X_{MS}X_{MS}X_{S}X_{S}X_{S}X_{S}X_{S}X_{S}X_{S}X_{S}X_{S}X_{S}X_{MS}X_{MS}X_{MS}X_{MS}X_{MS} (“5-10-5” 20-mer)
X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}

where “X” represents any nucleotide; a “M” represents a 2′-O-MOE group; and an “S” represents a phosphorothioate bond.

When DNA-based antisense oligonucleotides (ASO) bind to their cognate mRNA transcripts, the endogenous RNase H enzyme RNASEH1 recognizes RNA-DNA heteroduplex substrates that are formed and further cleaves at the site of ASO binding and results in degradation of the target RNA, thereby silencing target gene expression. Gapmer antisense oligonucleotides (ASOs), consisting of a DNA-based internal ‘gap’ and RNA-like flanking regions (often consisting of 2′-O-methyl (2′-OMe) or 2′-O-methoxyethyl (2′-OE) modified bases) bind to target transcripts with high affinity. In some embodiments, oligonucleotides comprise a Gapmer modification pattern.

In some embodiments, an oligonucleotide comprises any one of the sequences listed in Table 1 or Table 2, or a sequence that is at least 80%, at least 85%, at least 90%, or at least 95% identical to any one of the sequences listed in Table 1 or Table 2 and comprises the following modification pattern:

X_{MS}X_{MS}X_{MS}X_{MS}X_{MS}X_{S}X_{S}X_{S}X_{S}X_{S}X_{S}X_{S}X_{S}X_{S}X_{S}X_{MS}X_{MS}X_{MS}X_{MS}X_{MS}

where “X” represents any nucleotide; a “M” represents a 2′-O-MOE group; and an “S” represents a phosphorothioate bond.

In some embodiments, an oligonucleotide comprises any one of the sequences listed in Table 1 or Table 2, or a sequence that is at least 80%, at least 85%, at least 90%, at least 95% identical to any one of the sequences listed in Table 1 or Table 2 and comprises the following modification pattern:

X_{MS}X_{MS}X_{MS}X_{MS}X_{MS}X_{S}X_{S}X_{S}X_{S}X_{S}X_{S}X_{MS}X_{MS}X_{MS}X_{MS}X_{MS}
X_{MS}X_{MS}X_{MS}X_{MS}X_{MS}X_{S}X_{S}X_{S}X_{S}X_{S}X_{S}X_{S}X_{S}X_{MS}X_{MS}X_{MS}X_{MS}X_{MS},
X_{MS}X_{MS}X_{MS}X_{MS}X_{MS}X_{S}X_{S}X_{S}X_{S}X_{S}X_{S}X_{S}X_{S}X_{S}X_{S}X_{MS}X_{MS}X_{MS}X_{MS}X_{MS}
X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}
or
X_{MS}X_{MS}X_{MS}X_{MS}XMsX_{MS}X_{MS}X_{MS}X_{MS}X_{MS}X_{MS}X_{MS}X_{MS}X_{MS}X_{MS}X_{MS}X_{MS}X_{MS}X_{MS}X_{MS}

where “X” represents any nucleotide; a “M” represents a 2′-O-MOE group; and an “S” represents a phosphorothioate bond.

In some embodiments, oligonucleotides are provided and/or utilized in salt forms. In some embodiments, oligonucleotides are provided as salts comprising negatively-charged internucleotidic linkages (e.g., phosphorothioate internucleotidic linkages, natural phosphate linkages, etc.) existing as their salt forms. In some embodiments, oligonucleotides are provided as pharmaceutically acceptable salts. In some embodiments, oligonucleotides are provided as metal salts. In some embodiments, metal salts derived from appropriate bases include alkali metal, alkaline earth metal, ammonium, and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using the appropriate hydroxide or amine base. In some embodiments, oligonucleotides are provided as sodium salts. In some embodiments, oligonucleotides are provided as metal salts, e.g., sodium salts, wherein each negatively-charged internucleotidic linkage is independently in a salt form (e.g., for sodium salts, —O—P(O)(SNa)—O— for a phosphorothioate internucleotidic linkage, —O—P(O)(ONa)—O— for a natural phosphate linkage, etc.). In some embodiments, oligonucleotides are provided as ammonium salts.

In some embodiments, an oligonucleotide can be modified according to any one of the modifications and modification patterns described herein and can also be conjugated to a ligand, e.g., as described herein. In some such embodiments, a ligand can be attached to any of the 3′ or 5′ terminus of the oligonucleotide sequence.

In some embodiments, the ligand targets the nucleic acid molecule to hepatocytes. For example, in some embodiments the ligand binds to hepatocyte-specific asialoglycoprotein receptor (ASGPR). In some embodiments, the ligand comprises a galactose derivative, e.g., GalNAc.

In some embodiments, an oligonucleotide is conjugated to or otherwise physically associated with one or more moieties that modulate, e.g., enhance, the activity, stability, cellular distribution, and/or cellular uptake of the oligonucleotide and/or alter one or more physical properties of the oligonucleotide, such as charge or solubility. In some embodiments, a moiety may comprise an antibody or ligand. A ligand may be a carbohydrate, lectin, protein, glycoprotein, lipid, cholesterol, steroid, bile acid, nucleic acid hormone, growth factor, or receptor. In some embodiments a biologically inactive variant of a naturally occurring hormone, growth factor, or other ligand may be used. In some embodiments, the moiety comprises a targeting moiety that targets the oligonucleotide to a specified cell type, e.g., a hepatocyte. In some embodiments a targeting moiety binds to hepatocyte-specific asialoglycoprotein receptor (ASGPR).

In some embodiments, a moiety is attached to an oligonucleotide via a reversible linkage. A “reversible linkage” is a linkage that comprises a reversible bond. A “reversible bond” (also referred to as a labile bond or cleavable bond) is a covalent bond other than a covalent bond to a hydrogen atom that is capable of being selectively broken or cleaved more rapidly than other bonds in a molecule under selected conditions, the bond is capable of being selectively broken or cleaved under conditions that substantially will not break or cleave other covalent bonds in the same molecule. Cleavage or lability of a bond may be described in terms of the half-life (t_{1/2}) of bond cleavage (the time required for half of the bonds to cleave).

In some embodiments a moiety attached to an oligonucleotide comprises a carbohydrate. Representative carbohydrates include mono-, di-, tri- and oligosaccharides containing from about 4, 5, 6, 7, 8, or 9 monosaccharide units. In certain embodiments the carbohydrate comprises galactose or a galactose derivative such as galactosamine, N-formyl-galactosamine, N-acetylgalactosamine, N-propionyl-galactosamine, N-n-butanoyl-galactosamine, and N-iso-butanoylgalactos-amine. In certain embodiments of particular interest, a galactose derivative comprises N-acetylgalactosamine (GalNAc). In certain embodiments, a moiety comprises multiple instances of the galactose or galactose derivative, e.g., multiple N-acetylgalactosamine moieties, e.g., 3 GalNAc moieties (i.e., a triantennary GalNAc). A terminal galactose derivative may be attached to another moiety through the C-1 carbon of the galactose derivative. In some embodiments two or more, e.g., three, galactose derivatives are attached to a moiety that serves as a branch point and that can be attached to an oligonucleotide. In some embodiments, a galactose derivative is linked to the moiety that serves as a branch point via a linker or spacer. In some embodiments, the moiety that serves as a branch point may be attached to an oligonucleotide via a linker or spacer. For example, in some embodiments, a galactose derivative is attached to a branch point via a linker or spacer that comprises an amide, carbonyl, alkyl, oligoethylene glycol moiety, or combination thereof. In some embodiments, at least 3 nucleoside-GalNAc monomers or at least 3 non-nucleoside-GalNAc monomers are incorporated site-specifically into an oligonucleotide. In some embodiments, such incorporation may occur during solid-phase synthesis using phosphoramidite chemistry or via postsynthetic conjugation. In some embodiments, the galactose derivative-containing monomeric units are joined via phosphodiester bonds to each other and/or to nucleosides of the oligonucleotide that do not have a galactose derivative attached. One of ordinary skill in the art appreciates that the structure of the linking moieties that connect each GalNAc to a branch point may vary.

Exemplary galactose clusters are depicted below.

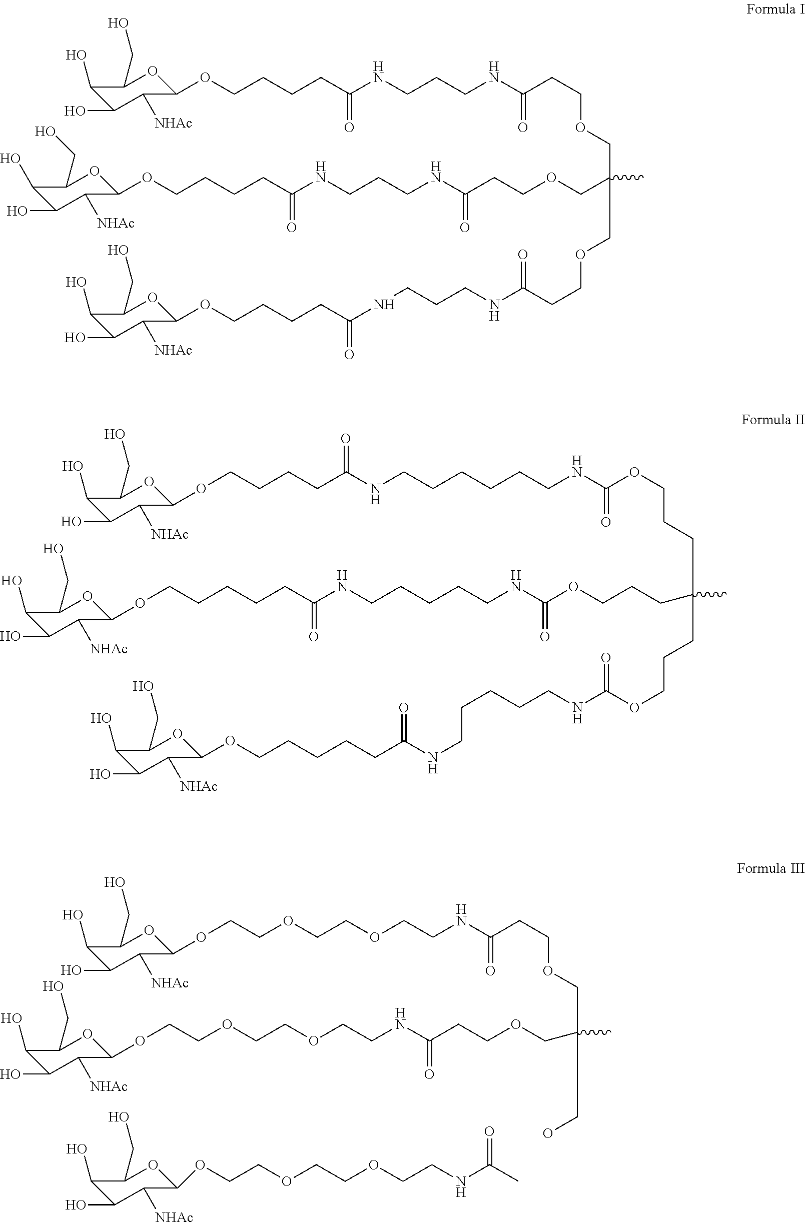

In some embodiments, a GalNAc moiety (e.g., a GalNAc moiety as represented in Formulas I-III) is conjugated to the 5′ end of an oligonucleotide described herein (e.g., an oligonucleotide represented in any one of SEQ ID NOs: 592, 594, 597, 598, 612, 613, 623, 624, 625, 626, 632, 633, 634, or 728-740).

In some embodiments, a GalNAc moiety (e.g., a GalNAc moiety as represented in Formulas I-III) is conjugated to the 3′ end of an oligonucleotide described herein (e.g., an oligonucleotide represented in any one of SEQ ID NOs: 592, 594, 597, 598, 612, 613, 623, 624, 625, 626, 632, 633, 634, or 728-740).

Methods of conjugating oligonucleotides to a GalNAc moiety are known in the art and exemplary methods are disclosed in Ostergaard, Michael E., et al., “Efficient synthesis and biological evaluation of 5′-GalNAc conjugated antisense oligonucleotides.” Bioconjugate chemistry 26.8 (2015): 1451-1455, which is herein incorporated by reference in its entirety.

In some embodiments, a 2′ deoxyadenosine phosphodiester is inserted between the oligonucleotide and the GalNAc conjugate to facilitate metabolic cleavage. Accordingly, in some embodiments, an oligonucleotide sequence contains an additional adenine (A) nucleotide residue at the 5′ or 3′ end where a GalNAc moiety is conjugated (see e.g., Ostergaard, Michael E., et al., 2015) and the additional A contains a phosphate bond between the A and the 5′ or 3′ nucleotide of the oligonucleotide. Exemplary modification patterns are shown below:

A_{O}X_{MS}X_{MS}X_{MS}X_{MS}X_{MS}X_{S}X_{S}X_{S}X_{S}X_{S}X_{S}X_{S}X_{S}X_{S}X_{S}X_{MS}X_{MS}X_{MS}X_{MS}X_{MS}
X_{MS}X_{MS}X_{MS}X_{MS}X_{MS}X_{S}X_{S}X_{S}X_{S}X_{S}X_{S}X_{S}X_{S}X_{S}X_{S}X_{MS}X_{MS}X_{MS}X_{MS}X_{MS}A_{O}
AoX_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}
X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}A_{O}
AoX_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}A_{O}

where “A” represents an adenine; an “X” represents any nucleotide; a “M” represents a 2′-O-MOE group; an “S” represents a phosphorothioate bond; and an “O” represents a phosphate linkage.

In some embodiments, an oligonucleotide comprises any one of the sequences listed in Table 1 or Table 2, or a sequence that is at least 80%, at least 85%, at least 90%, at least 95% identical to any one of the sequences listed in Table 1 or Table 2 and comprises the following modification pattern:

A_{O}X_{MS}X_{MS}X_{MS}X_{MS}X_{MS}X_{S}X_{S}X_{S}X_{S}X_{S}X_{S}X_{S}X_{S}X_{S}X_{S}X_{MS}X_{MS}X_{MS}X_{MS}X_{MS}
X_{MS}X_{MS}X_{MS}X_{MS}X_{MS}X_{S}X_{S}X_{S}X_{S}X_{S}X_{S}X_{S}X_{S}X_{S}X_{S}X_{MS}X_{MS}X_{MS}X_{MS}X_{MS}A_{O}
A_{O}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}
X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}A_{O}
A_{O}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}X_{MS}A_{O}

where “A” represents an adenine; an “X” represents any nucleotide; a “M” represents a 2′-O-MOE group; an “S” represents a phosphorothioate bond; and an “O” represents a phosphate linkage.

In some embodiments, a linking moiety connects an oligonucleotide described herein (e.g., an oligonucleotide represented in any one of SEQ ID NOs: 592, 594, 597, 598, 612, 613, 623, 624, 625, 626, 632, 633, 634, or 728-740) to a GalNAc moiety (e.g., as shown in Formulas I-III). In some embodiments, an oligonucleotide described herein is conjugated to GalNAc as depicted below:

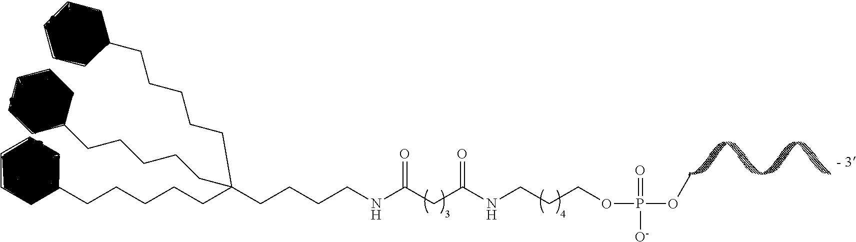

In some embodiments, a linking moiety comprises a structure as depicted below:

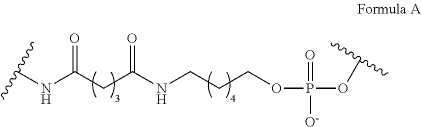

In some embodiments, an oligonucleotide (e.g., an oligonucleotide represented in any one of SEQ ID NOs: 592, 594, 597, 598, 612, 613, 623, 624, 625, 626, 632, 633, 634, or 728-740) is conjugated to a GalNAc moiety as shown in Formula I at its 5′ end via a linker as shown in Formula A. In some embodiments, an oligonucleotide (e.g., an oligonucleotide represented in any one of SEQ ID NOs: 592, 594, 597, 598, 612, 613, 623, 624, 625, 626, 632, 633, 634, or 728-740) is conjugated to a GalNAc moiety as shown in Formula I at its 3′ end via a linker as shown in Formula A. In some embodiments, an oligonucleotide (e.g., an oligonucleotide represented in any one of SEQ ID NOs: 592, 594, 597, 598, 612, 613, 623, 624, 625, 626, 632, 633, 634, or 728-740) additionally comprises a 2′ deoxyadenosine phosphodiester inserted between the oligonucleotide and the GalNAc/Linker moiety.

In some embodiments, an oligonucleotide (e.g., an oligonucleotide represented in any one of SEQ ID NOs: 592, 594, 597, 598, 612, 613, 623, 624, 625, 626, 632, 633, 634, or 728-740) is conjugated to a GalNAc moiety as shown in Formula II at its 5′ end via a linker as shown in Formula A. In some embodiments, an oligonucleotide (e.g., an oligonucleotide represented in any one of SEQ ID NOs: 592, 594, 597, 598, 612, 613, 623, 624, 625, 626, 632, 633, 634, or 728-740) is conjugated to a GalNAc moiety as shown in Formula II at its 3′ end via a linker as shown in Formula A. In some embodiments, an oligonucleotide (e.g., an oligonucleotide represented in any one of SEQ ID NOs: 592, 594, 597, 598, 612, 613, 623, 624, 625, 626, 632, 633, 634, or 728-740) additionally comprises a 2′ deoxyadenosine phosphodiester inserted between the oligonucleotide and the GalNAc/Linker moiety.

In some embodiments, an oligonucleotide (e.g., an oligonucleotide represented in any one of SEQ ID NOs: 592, 594, 597, 598, 612, 613, 623, 624, 625, 626, 632, 633, 634, or 728-740) is conjugated to a GalNAc moiety as shown in Formula III at its 5′ end via a linker as shown in Formula A. In some embodiments, an oligonucleotide (e.g., an oligonucleotide represented in any one of SEQ ID NOs: 592, 594, 597, 598, 612, 613, 623, 624, 625, 626, 632, 633, 634, or 728-740) is conjugated to a GalNAc moiety as shown in Formula III at its 3′ end via a linker as shown in Formula A. In some embodiments, an oligonucleotide (e.g., an oligonucleotide represented in any one of SEQ ID NOs: 592, 594, 597, 598, 612, 613, 623, 624, 625, 626, 632, 633, 634, or 728-740) additionally comprises a 2′ deoxyadenosine phosphodiester inserted between the oligonucleotide and the GalNAc/Linker moiety.

In certain embodiments, the moiety comprises a lipophilic moiety. In some embodiments, the lipophilic moiety comprises a tocopherol, e.g., alpha-tocopherol. In some embodiments, the lipophilic moiety comprises cholesterol. In some embodiments, the lipophilic compound comprises an alkyl or heteroalkyl group. In some embodiments the lipophilic compound comprises palmitoyl, hexadec-8-enoyl, oleyl, (9E,12E)-octadeca-9,12-dienoyl, dioctanoyl, or C₁₆-C₂₀ acyl. In some embodiments, the lipophilic moiety comprises at least 16 carbon atoms. In some embodiments the lipophilic moiety comprises —(CH_{y})ₙ—NH—(C═O)—(CHₓ)ₘ—CH₃, wherein each of m and n is independently 0-20; and each of x and y is independently 0-2. In some embodiments, n and m are each independently an integer from 1 to 20. In some embodiments n+m is at least 10, 12, 14, or 16.

Unless otherwise stated, structures depicted herein are also meant to include all isomeric (e.g., enantiomeric, diastereomeric, and geometric (or conformational)) forms of the structure; for example, the R and S configurations for each asymmetric center. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

### Compositions and Administration

In some embodiments, one or more oligonucleotides as described herein may be formulated in an oligonucleotide composition. In some embodiments, an oligonucleotide composition may comprise oligonucleotides comprising the same nucleotide sequence (e.g., any one of the sequences provided in Table 1 or Table 2). In some embodiments, an oligonucleotide composition may comprise oligonucleotides comprising more than one nucleotide sequence (e.g., more than one of the sequences provided in Table 1 or Table 2).

In some embodiments, provided oligonucleotide compositions may be or include pure preparations of individual stereochemically isomeric forms of a compound (e.g., comprising a chirally pure oligonucleotide). In some embodiments, provided oligonucleotide compositions may be or include mixtures of two or more stereochemically isomeric forms of the compound. In some embodiments, such mixtures contain equal amounts of different stereochemically isomeric forms. In some embodiments, such mixtures contain different amounts of at least two different stereochemically isomeric forms. In some embodiments, an oligonucleotide composition may contain all diastereomers and/or enantiomers of the compound. In some embodiments, an oligonucleotide composition may contain fewer than all diastereomers and/or enantiomers of a compound. In some embodiments, if a particular enantiomer of an oligonucleotide is desired, it may be prepared, for example, by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, diastereomeric salts are formed with an appropriate optically-active acid, and resolved, for example, by fractional crystallization.

### Pharmaceutical Compositions

In some embodiments, the present disclosure provides pharmaceutical compositions comprising one or more oligonucleotides. In some embodiments, a pharmaceutical composition comprises a pharmaceutically acceptable carrier.

In some embodiments, a pharmaceutical composition is formulated for systemic or localized administration. In some embodiments, a pharmaceutical composition is administered via a delivery route selected from intrathecal, oral, intramuscular, or intravenous administration.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. In some embodiments, an intranasal composition is an intranasal drop or spray (fine mist) in a liquid form such as, for example, a solution, emulsion or suspension.

Pharmaceutically acceptable compositions of this disclosure may also be adapted for pulmonary administration, such as an inhalation composition to be inhaled by the patient. The inhalation composition can be in the form of a dry powder inhalation composition, a pressurized aerosol inhalation composition or a nebulized inhalation composition (e.g., an aqueous suspension or solution).

In some embodiments an oligonucleotide is associated with a delivery agent. “Delivery agent” refers to a substance or entity that is non-covalently or covalently associated with an oligonucleotide or is co-administered with an oligonucleotide and serves one or more functions that increase the stability and/or efficacy of the biologically active agent beyond that which would result if the biologically active agent was delivered (e.g., administered to a subject) in the absence of the delivery agent. For example, a delivery agent may protect an oligonucleotide from degradation, may facilitate entry of an oligonucleotide into cells or into a cellular compartment of interest (e.g., the cytoplasm or mitochondria), and/or may enhance associations with particular cells containing the molecular target to be modulated. Those of ordinary skill in the art are aware of numerous delivery agents that may be used to deliver oligonucleotides. See Dhuri, Karishma, et al., “Antisense oligonucleotides: an emerging area in drug discovery and development.” Journal of clinical medicine 9.6 (2020), for review of some of these technologies. In some embodiments, e.g., for administering an oligonucleotide systemically, the oligonucleotide may be associated with a delivery agent such as a nanoparticle, a dendrimer, a polymer, liposomes, or a cationic delivery system. Lipids (e.g., cationic lipids, or neutral lipids), dendrimers, or polymers may be bound to an oligonucleotide or may form a vesicle or micelle that encapsulates an oligonucleotide.

In some embodiments an oligonucleotide is administered in association with a lipid or lipid-containing particle (e.g., a lipid nanoparticle (LNP)). In some embodiments an oligonucleotide is administered in association with a cationic polymer (which may be a polypeptide or a non-polypeptide polymer), a lipid, a peptide, PEG, cyclodextrin, or combination thereof, which may be in the form of a nanoparticle or microparticle. The lipid or peptide may be cationic. “Nanoparticle” refers to particles with lengths in two or three dimensions greater than 1 nanometer (nm) and smaller than about 150 nm e.g., 20 nm-50 nm or 50 nm-100 nm. “Microparticle” refers to particles with lengths in two or three dimensions greater than 150 nm and smaller than about 1000 nm. A nanoparticle may have a targeting moiety and/or cell-penetrating moiety or membrane active moiety covalently or noncovalently attached thereto. Nanoparticles, such as lipid nanoparticles, are described in, e.g., Tatiparti et al., Nanomaterials 7:77 (2017).

In some embodiments, a delivery agent comprises one or more amino acid lipids. Amino acid lipids are molecules containing an amino acid residue (e.g., arginine, homoarginine, norarginine, nor-norarginine, ornithine, lysine, homolysine, histidine, 1-methylhistidine, pyridylalanine, asparagine, N-ethylasparagine, glutamine, 4-aminophenylalanine, the N-methylated versions thereof, and side chain modified derivatives thereof) and one or more lipophilic tails. In some embodiments, a delivery agent comprises a lipopeptide compound comprising a central peptide and having lipophilic groups attached at each terminus. In some embodiments lipophilic groups can be derived from a naturally occurring lipid. In some embodiments a lipophilic group may comprise a C(1-22)alkyl, C(6-12)cycloalkyl, C(6-12)cycloalkyl-alkyl, C(3-18)alkenyl, C(3-18)alkynyl, C(1-5)alkoxy-C(1-5)alkyl, or a sphinganine, or (2R,3R)-2-amino-1,3-octadecanediol, icosasphinganine, sphingosine, phytosphingosine, or cis-4-sphingenine. The central peptide may comprise a cationic or amphipathic amino acid sequence. Examples of such lipopeptides and their use to deliver nucleic acids are described in, e.g., U.S. Pat. No. 9,220,785.

In some embodiments an oligonucleotide is conjugated to a delivery agent that is a polymer. Useful delivery polymers include, e.g., poly(acrylate) polymers (see., e.g., US Pat. Pub. No. 20150104408), poly(vinyl ester) polymers (see., e.g., US Pat. Pub. No. 20150110732) and certain polypeptides.

In some embodiments an oligonucleotide may be administered in “naked” form, i.e., administered in the absence of a delivery agent. The naked oligonucleotide may be in a suitable buffer solution. The buffer solution may, for example, comprise acetate, citrate, prolamine, carbonate, or phosphate, or any combination thereof. In some embodiments, the buffer solution is phosphate buffered saline (PBS). The pH and osmolarity of the buffer solution can be adjusted such that it is suitable for administering to a subject. In some embodiments, an oligonucleotide is administered not in physical association with a lipid or lipid-containing particle. In some embodiments, an oligonucleotide is administered not in physical association with a nanoparticle or microparticle. In some embodiments, an oligonucleotide is administered not in physical association with a cationic polymer.

Oligonucleotides described herein, can be incorporated into pharmaceutical compositions. Such pharmaceutical compositions are useful for, among other things, administration and delivery to a subject in vivo or ex vivo. In some embodiments, pharmaceutical compositions also contain a pharmaceutically acceptable carrier or excipient. Such excipients include any pharmaceutical agent, e.g., a pharmaceutical agent that does not itself induce an immune response harmful to the individual receiving the composition, and which may be administered without undue toxicity. As used herein the terms “pharmaceutically acceptable” and “physiologically acceptable” mean a biologically acceptable formulation, gaseous, liquid or solid, or mixture thereof, which is suitable for one or more routes of administration, in vivo delivery or contact. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

Pharmaceutical compositions may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In some embodiments, a pharmaceutical composition may be a lyophilized powder.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration can comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran. Additionally, suspensions of the active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility to allow for the preparation of highly concentrated solutions.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soy lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Pharmaceutical compositions and delivery systems appropriate for the compositions, methods and uses of the disclosure are known in the art (see, e.g., Remington: The Science and Practice of Pharmacy. 21st Edition. Philadelphia, PA. Lippincott Williams & Wilkins, 2005).

### Dosing and Administration

Oligonucleotides described herein, or a vector comprising a nucleotide sequence encoding an oligonucleotide described herein, can be used to treat cancer or a metabolic disease or disorder, e.g., subjects suffering from or susceptible to cancer or a metabolic disease or disorder described herein. The mode of administration of pharmaceutical compositions described herein can vary depending upon the desired results. One with skill in the art, i.e., a physician, is aware that dosage regimens can be adjusted to provide the desired response, e.g., a therapeutic response.

Methods of administration include, but are not limited to, intradermal, intramuscular, intraperitoneal, intravenous, subcutaneous, intranasal, epidural, oral, sublingual, intracerebral, intrathecal (e.g., intracisternal or via a lumbar puncture), intravaginal, transdermal, rectal, by inhalation, or topical, particularly to the ears, nose, eyes, or skin. In some embodiments, compositions of oligonucleotides are delivered to the central nervous system (CNS), e.g., delivered via intracerebroventricular administration.

In some embodiments, a pharmaceutical composition described herein is delivered to the liver. In some embodiments, a pharmaceutical composition described herein is delivered to the muscle. In some embodiments, a pharmaceutical composition described herein is delivered to the CNS (e.g., via intrathecal administration). In some embodiments, a pharmaceutical composition described herein is delivered to the cerebrospinal fluid.

Delivery of an oligonucleotide to a cell may be achieved in a number of different ways. In vivo delivery may be performed by administering a composition comprising an oligonucleotide to a subject, e.g., by parenteral administration route, e.g., subcutaneous or intravenous or intramuscular administration.

The disclosure also provides methods for administering an oligonucleotide, or a vector comprising a nucleotide sequence encoding an oligonucleotide described herein, into a cell or an animal. In some embodiments, such methods include contacting a subject (e.g., a cell or tissue of a subject) with, or administering to a subject (e.g., a subject such as a mammal), an oligonucleotide described herein (or a vector comprising a nucleotide sequence encoding an oligonucleotide described herein), such that the oligonucleotide is expressed in the subject (e.g., in a cell or tissue of a subject).

Compositions of oligonucleotides described herein (or a vector comprising a nucleotide sequence encoding an oligonucleotide described herein) can be administered in a sufficient or effective amount to a subject in need thereof. Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

In some embodiments, oligonucleotide compositions are administered to a subject in an amount that is between 0.01 mg/kg and 50 mg/kg. In some embodiments, the oligonucleotide composition is administered at a dose of about 0.01 mg/kg to about 10 mg/kg or about 0.5 mg/kg to about 15 mg/kg. In some embodiments, the oligonucleotide composition is administered at a dose of about 10 mg/kg to about 30 mg/kg. In some embodiments, the oligonucleotide composition is administered at a dose of about 0.5 mg/kg, about 1 mg/kg, about 1.5 mg/kg, about 2.0 mg/kg, about 2.5 mg/kg, about 3 mg/kg, about 3.5 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In some embodiments, the oligonucleotide composition is administered at a dose of between 0.01 mg/kg and 0.1 mg/kg, between 0.01 mg/kg and 0.1 mg/kg, between 0.1 mg/kg and 1.0 mg/kg, between 1.0 mg/kg and 2.5 mg/kg, between 2.5 mg/kg and 5.0 mg/kg, between 5.0 mg/kg and 10 mg/kg, between 10 mg/kg and 20 mg/kg, between 20 mg/kg and 30 mg/kg, between 30 mg/kg and 40 mg/kg or between 40 mg/kg and 50 mg/kg. In some embodiments, a fixed dose is administered. In some embodiments, the oligonucleotide composition is administered at a dose of between 5 mg and 1.0 g, e.g., between 5 mg and 10 mg, between 10 mg and 20 mg, between 20 mg and 40 mg, between 40 mg and 80 mg, between 80 mg and 160 mg, between 160 mg and 320 mg, between 320 mg and 640 mg, between 640 mg and 1 g. In some embodiments, the dose is about 1 mg, 5 mg, 10 mg, 25 mg, 50 mg, 100 mg, 150 mg, 200 mg, 250 mg, 300 mg, 350 mg, 400 mg, 450 mg, 500 mg, 600 mg, 700 mg, 800 mg, 900 mg, or 1000 mg.

In some embodiments, the dose is a daily dose. In some embodiments, the dose is administered according to a dosing regimen with a dosing interval of at least 2 days, e.g., at least 7 days, e.g., about 2, 3, 4, 6, or 8 weeks. For example, in some embodiments, an oligonucleotide composition is administered according to a dosing regimen with a dosing interval of at least 7 days. In some embodiments, an oligonucleotide composition is administered daily, weekly, monthly, or every 2, 3, 4, 5, or 6 months or longer. In some embodiments, any of the doses and/or dosing regimens described herein are administered subcutaneously. In some embodiments, an oligonucleotide composition is administered once and levels of inhibition are subsequently measured, and once the level of inhibition decreases to a certain level, a subsequent dose of the inhibitory composition is administered.

In some embodiments, a subject exhibits a sustained inhibition of POLRMT, e.g., measured by POLRMT mRNA expression (e.g., in a biological sample) for a period of time that is at least 2 days, e.g., at least 7 days, e.g., about 2, 3, 4, 6, 8, 10, 12, 16, or 20 weeks post-administration.

An effective amount or a sufficient amount can (but need not) be provided in a single administration, may require multiple administrations, and can (but need not) be, administered alone or in combination with another composition. For example, the amount may be proportionally increased as indicated by the need of the subject, type, status and severity of the disease treated or side effects (if any) of treatment. Amounts considered effective also include amounts that result in a reduction of the use of another treatment, therapeutic regimen or protocol, such as administration of another therapeutic described herein.

Accordingly, pharmaceutical compositions of the disclosure include compositions wherein the active ingredients are contained in an effective amount to achieve the intended therapeutic purpose. Determining a therapeutically effective dose is well within the capability of a skilled medical practitioner using the techniques and guidance provided in the disclosure. Therapeutic doses can depend on, among other factors, the age and general condition of the subject, the severity of the cancer or metabolic disease or disorder, and the strength of the control sequences regulating the expression levels of the oligonucleotide. Thus, a therapeutically effective amount in humans will fall in a relatively broad range that may be determined by a medical practitioner based on the response of an individual patient to vector-based treatment. Pharmaceutical compositions may be delivered to a subject, so as to allow production of an oligonucleotide described herein in vivo by gene- and or cell-based therapies or by ex-vivo modification of the patient's or donor's cells.

Methods and uses of the disclosure include delivery and administration systemically, regionally or locally, or by any route, for example, by injection or infusion. Delivery of a pharmaceutical composition in vivo may generally be accomplished via injection using a conventional syringe, although other delivery methods such as convection-enhanced delivery can also be used (see, e.g., U.S. Pat. No. 5,720,720). For example, compositions may be delivered subcutaneously, epidermally, intradermally, intrathecally, intraorbitally, intramucosally, intraperitoneally, intravenously, intra-pleurally, intraarterially, orally, intrahepatically, intracerebroventricularly (e.g., via intracerebroventricular injection), via the portal vein, or intramuscularly. Other modes of administration include oral and pulmonary administration, suppositories, and transdermal applications. A clinician specializing in the treatment of patients with cancer, or a metabolic disease or disorder may determine the optimal route for administration of an oligonucleotide composition or a vector comprising a nucleotide sequence encoding an oligonucleotide described herein.

In some embodiments, an oligonucleotide composition may be administered to a subject once daily, weekly, every 2, 3, or 4 weeks, or even at longer intervals. In some embodiments, an oligonucleotide composition described herein may be administered according to a dosing regimen that includes (i) an initial administration that is once daily, weekly, every 2, 3, or 4 weeks, or even at longer intervals; followed by (ii) a period of no administration of, e.g., 1, 2, 3, 4, 5, 6, 8, or 10 months, or 1, 2, 3, 4, 5, 6, 7, 8, 9, or 10 years. In some embodiments, a subject is monitored before and/or following treatment for level of POLRMT mRNA expression and/or activity or POLRMT protein level. In some embodiments, a subject is monitored before and/or following treatment for level of mtDNA, mRNA expression of other mitochondrial genes, or level of other mitochondrial proteins (e.g., indicating decrease in mitochondrial transcription). In some embodiments, a subject is treated, or is retreated, if a measured level of POLRMT mRNA expression and/or POLRMT activity or level of POLRMT protein is more than 10%, 20%, 30%, 40%, 50%, 100%, 200%, or more, relative to measured level in a control subject.

### Diseases, Disorders, and Conditions

The present disclosure provides, among other things, oligonucleotides and compositions comprising the same. In some embodiments, such compositions are used for treating cancer and metabolic diseases through inhibition of POLRMT.

### Cancer

In some embodiments, oligonucleotides described here may be used to treat cancer. Those skilled in the art are aware of a variety of types of cancer including, for example, adrenal gland cancer, anal cancer, adenocarcinoma, adrenocortical carcinoma, astrocytoma, angiosarcoma, basal cell carcinoma, bile duct cancer, bladder cancer, blastic plasmacytoid dendritic cell neoplasm, bone cancer, brain cancer, breast cancer, bronchogenic carcinoma, central nervous system (CNS) cancer, cervical cancer, carcinoid, cardiac, cholangiocarcinoma, chordoma, chronic myeloproliferative neoplasms, craniopharyngioma, cholangiocarcinoma, chondrosarcoma, colon cancer, choriocarcinoma, colorectal cancer, cancer of connective tissue, esophageal cancer, ductal carcinoma in situ, ependymoma, embryonal carcinoma, fibrosarcoma, gall bladder cancer, gastric cancer, glioblastomas, gastrointestinal carcinoid tumor, gastrointestinal stromal tumor (GIST), gestational trophoblastic disease, glioma, head and neck cancer, hematological cancer, histiocytosis, kidney cancer, intraocular melanoma, leukemias (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, myeloid leukemia, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoma (e.g., Burkitt lymphoma [non-Hodgkin lymphoma], cutaneous T cell lymphoma, Hodgkin lymphoma, mycosis fungoides, Sezary syndrome, AIDS-related lymphoma, follicular lymphoma, diffuse large B-cell lymphoma), melanoma, Merkel cell carcinoma, mesothelioma, myeloma (e.g., multiple myeloma), muscular cancer, myxosarcoma, myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), neuroblastomas, Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

In some embodiments, compositions of oligonucleotides described herein may be used to treat a tumor in a subject. In some embodiments, a tumor is or comprises a hematologic malignancy, including but not limited to, acute lymphoblastic leukemia, acute myeloid leukemia, chronic lymphocytic leukemia, chronic myelogenous leukemia, hairy cell leukemia, AIDS-related lymphoma, Hodgkin lymphoma, non-Hodgkin lymphoma, Langerhans cell histiocytosis, multiple myeloma, or myeloproliferative neoplasms.

In some embodiments, a tumor is or comprises a solid tumor, including but not limited to breast carcinoma, a squamous cell carcinoma, a colon cancer, a head and neck cancer, ovarian cancer, a lung cancer, mesothelioma, a genitourinary cancer, a bladder cancer, a rectal cancer, a gastric cancer, or an esophageal cancer.

In some particular embodiments, a tumor is or comprises an advanced tumor, and/or a refractory tumor. In some embodiments, a tumor is characterized as advanced when certain pathologies are observed in a tumor (e.g., in a tissue sample, such as a biopsy sample, obtained from a tumor) and/or when cancer patients with such tumors are typically considered not to be candidates for conventional chemotherapy. In some embodiments, pathologies characterizing tumors as advanced can include tumor size, altered expression of genetic markers, invasion of adjacent organs and/or lymph nodes by tumor cells. In some embodiments, a tumor is characterized as refractory when patients having such a tumor are resistant to one or more known therapeutic modalities (e.g., one or more conventional chemotherapy regimens) and/or when a particular patient has demonstrated resistance (e.g., lack of responsiveness) to one or more such known therapeutic modalities.

In some embodiments, compositions comprising one or more oligonucleotides described herein can be administered in combination with a cancer therapy. The present disclosure is not limited to any specific cancer therapy, and any known or developed cancer therapy is encompassed by the present disclosure. Known cancer therapies include, e.g., administration of therapeutic cancer vaccines, chemotherapeutic agents, radiation therapy, surgical excision, chemotherapy following surgical excision of tumor, adjuvant therapy, localized hypothermia or hyperthermia, anti-tumor antibodies, and anti-angiogenic agents. In some embodiments, cancer and/or adjuvant therapy includes a TLR agonist (e.g., CpG, Poly I:C, etc., see, e.g., Wittig et al., Crit. Rev. Oncol. Hematol. 94:31-44 (2015); Huen et al., Curr. Opin. Oncol. 26:237-44 (2014); Kaczanowska et al., J. Leukoc. Biol. 93:847-863 (2013)), a STING agonist (see, e.g., US20160362441; US20140329889; Fu et al., Sci. Transl. Med. 7:283ra52 (2015); and WO2014189805), a non-specific stimulus of innate immunity, and/or dendritic cells, or administration of GM-CSF, Interleukin-12, Interleukin-7, Flt-3, or other cytokines. In some embodiments, the cancer therapy is or comprises oncolytic virus therapy, e.g., talimogene leherparepvec. (See, e.g., Fukuhara et al., Cancer Sci. 107:1373-1379 (2016)). In some embodiments, the cancer therapy is or comprises bi-specific antibody therapy (e.g., Choi et al., 2011 Expert Opin Biol Ther; Huehls et al., 2015, Immunol and Cell Biol). In some embodiments, the cancer therapy is or comprises cellular therapy such as chimeric antigen receptor T (CAR-T) cells, TCR-transduced T cells, dendritic cells, tumor infiltrating lymphocytes (TIL), or natural killer (NK) cells (e.g., as reviewed in Sharpe and Mount, 2015, Dis Model Mech 8:337-50).

In some embodiments, a cancer therapy may include a chemotherapeutic agent. A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/anti-tumor antibiotics, topoisomerase inhibitors, antibodies, photosensitizers, and kinase inhibitors. Non-limiting examples of chemotherapeutic agents include erlotinib (TARCEVA®, Genentech/OSI Pharm.), docetaxel (TAXOTER®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine,dichloroplatinum(II), CAS No. 15663-27-1), carboplatin (CAS No. 41575-94-4), paclitaxel (TAXOL®, Bristol-Myers Squibb Oncology, Princeton, N.J.), temozolomide (4-methyl-5-oxo-2,3,4,6,8-pentazabicyclo [4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethyl-ethanamine, NOLVADEX®, ISTUBAL®, VALODEX®), and doxorubicin (ADRIAMYCIN®), Akti-1/2, HPPD, and rapamycin.

Additional examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (MEK inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, calicheamicin gamma1I, calicheamicin omegaI1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside (Ara-C); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

### Metabolic Disorders

In some embodiments, oligonucleotides described herein may be used to treat a metabolic disease associated with mitochondrial dysfunction. Types of metabolic diseases include obesity, diabetes, non-alcoholic steatohepatitis (NASH), and related metabolic syndromes such as non-alcoholic fatty liver disease (NAFLD), Familial hypercholesterolemia, Hunter syndrome, Metachromatic leukodystrophy, Mitochondrial encephalopathy, lactic acidosis, and Porphyria.

In some embodiments, a metabolic disorder includes syndromic obesity such as Prader-Willi (PWS) and Bardet-Biedl (BBS) syndromes. In some embodiments, a metabolic disorder includes oligogenic obesity, such as melanocortin 4 receptor (MC4R)-linked obesity (see Rodríguez-López, Raquel, et al., Current Genomics 23.3 (2022): 147, which is herein incorporated by reference).

In some embodiments, a metabolic disorder includes disorders of amino acid metabolism (amino acidemias) such as Maple Syrup Urine Disease (MSUD), Tyrosinemia, and Homocystinuria.

In some embodiments, a metabolic disorder includes disorders of organic acid metabolism (organic acidurias, organic acidemias) such as Methylmalonic Aciduria, 3-Methylglutaconic Aciduria—Barth Syndrome, Glutaric Aciduria, 2-Hydroxyglutaric aciduria—D and L forms, and propionic acidemia.

In some embodiments, a metabolic disorder includes disorders of Fatty Acid Beta-Oxidation such as MCAD Deficiency, LCHAD, and VLCAD deficiency.

In some embodiments, a metabolic disorder includes disorders of lipid metabolism (lipid storage disorders) such as Gangliosidoses (e.g., GM1 Gangliosidosis, Tay-Sachs Disease, Sandhoff Disease), Sphingolipidoses (e.g., Fabry Disease, Gaucher Disease, Niemann-Pick Disease, and Krabbe Disease), Mucolipidoses, and Mucopolysaccharidoses.

In some embodiments, a metabolic disease includes mitochondrial disorders, leading in some cases to muscle damage or muscle wasting. Examples of mitochondrial disorders include mitochondrial cardiomyopathies, Leigh disease, stroke-like episodes (MELAS), MERRF, NARP, and Barth syndrome.

In some embodiments, a metabolic disorder includes a lysosomal storage disorder, where enzymes in lysosomes that break down waste products of metabolism may be deficient or dysfunctional and cause buildup of toxic substances resulting in various diseases. Examples of lysosomal storage disorders include, e.g., Hurler syndrome (abnormal bone structure and developmental delay).

In some embodiments, a metabolic disease includes peroxisomal disorders. Similar to lysosomes, peroxisomes are tiny, enzyme-filled spaces within cells. Functional deficiencies of enzymes within peroxisomes can lead to buildup of toxic products of metabolism. Exemplary peroxisomal disorders include Zellweger syndrome (which manifests as abnormal facial features, enlarged liver, and nerve damage in infants), Adrenoleukodystrophy (which is characterized by symptoms of nerve damage that can develop in childhood or early adulthood depending on the form), and Refsum Disease.

In some embodiments, a metabolic disease includes galactosemia, resulting from impaired breakdown of the sugar galactose, which leads to jaundice, vomiting, and liver enlargement after breast or formula feeding by a newborn.

In some embodiments, a metabolic disease includes phenylketonuria (PKU) resulting from a deficiency of the enzyme PAH which results in high levels of phenylalanine in the blood. Intellectual disability may result if this condition is not recognized and treated.

In some embodiments, a metabolic disease includes glycogen storage diseases resulting from problems with sugar storage, which leads to low blood sugar levels, muscle pain, and weakness.

In some embodiments, a metabolic disease includes Friedreich ataxia, resulting from problems related to a protein called frataxin causing nerve damage and often heart problems. Usually, such a disease results in the inability to walk by young adulthood.

In some embodiments, a metabolic disease includes metal metabolism disorders. In the blood, levels of trace metals are controlled by special proteins. Inherited metabolic disorders can result in protein malfunction and toxic accumulation of metal in the body. Example metal metabolism disorders include Wilson's disease, where toxic copper levels accumulate in the liver, brain, and other organs, and Hemochromatosis, where the intestines absorb excessive iron, which builds up in the liver, pancreas, joints, and heart, causing damage.

In some embodiments, a metabolic disease includes urea cycle disorders such as ornithine transcarbamylase deficiency and citrullinemia.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### Numbered Embodiments

Embodiment 1. An oligonucleotide comprising a sequence that is complementary to a sequence that differs by no more than 1, 2, 3, or 4 nucleotides from a target region that spans between 8 to 30 contiguous nucleotides of a POLRMT nucleotide sequence.

Embodiment 2. The oligonucleotide of embodiment 1, wherein the oligonucleotide comprises a sequence that is complementary to a region that spans between 8 to 30 contiguous nucleotides of a POLRMT nucleotide sequence.

Embodiment 3. The oligonucleotide of embodiment 1 or embodiment 2, wherein the target region spans between 15 to 25 contiguous nucleotides of a POLRMT nucleotide sequence.

Embodiment 4. The oligonucleotide sequence of any one of embodiments 1-3, wherein the target region spans 20 contiguous nucleotides of a POLRMT nucleotide sequence.

Embodiment 5. The oligonucleotide sequence of any one of embodiments 1-4, wherein the target region comprises an exon region of POLRMT nucleotide sequence.

Embodiment 6. The oligonucleotide of any one of embodiments 1-5, wherein the target region comprises a sequence that corresponds to nucleotides 5696-5715, 8808-8827, 8809-8828, 8811-8830, 16221-16240, 17159-17178, 17314-17333, 17315-17334, 18082-18101, 18083-18102, 18084-18103, 18130-18149, 5680-5699, 8491-8510, 8529-8548, 8569-8588, 8570-8589, 8571-8590, 8572-8591, 8573-8592, 8574-8593, 13322-13341, 13719-13738, 14999-15018, 15092-15111, 15093-15112, 17304-17323, 19309-19328, 20041-20060, 20042-20061, or 21102-21121 of SEQ ID NO: 1.

Embodiment 7. An oligonucleotide comprising a sequence having at least 80% identity to a sequence selected from a group consisting of SEQ ID NOs: 3-14, 592, 594, 597, 598, 612, 613, 623, 624, 625, 626, 632, 633, and 634.

Embodiment 8. The oligonucleotide of embodiment 7, wherein the oligonucleotide comprises a sequence having at least 90% identity to a sequence selected from a group consisting of SEQ ID NOs: 3-14, 592, 594, 597, 598, 612, 613, 623, 624, 625, 626, 632, 633, and 634.

Embodiment 9. The oligonucleotide of embodiment 7 or embodiment 8, wherein the oligonucleotide comprises a sequence selected from a group consisting of SEQ ID NOs: 3-14, 592, 594, 597, 598, 612, 613, 623, 624, 625, 626, 632, 633, and 634.

Embodiment 10. The oligonucleotide of any one of embodiments 7-9, wherein the oligonucleotide comprises SEQ ID NO: 11.

Embodiment 11. The oligonucleotide of any one of embodiments 7-9, wherein the oligonucleotide comprises SEQ ID NO: 12.

Embodiment 12. The oligonucleotide of any one of embodiments 7-9, wherein the oligonucleotide comprises SEQ ID NO: 594.

Embodiment 13. The oligonucleotide of any one of embodiments 7-9, wherein the oligonucleotide comprises SEQ ID NO: 612.

Embodiment 14. The oligonucleotide of any one of embodiments 7-9, wherein the oligonucleotide comprises SEQ ID NO: 632.

Embodiment 15. An oligonucleotide comprising a sequence that is complementary to a sequence that is at least 80% identical to a sequence selected from a group consisting of SEQ ID NOs: 15-26, 661, 663, 666, 667, 681, 682, 692, 693, 694, 695, 701, 702, and 703.

Embodiment 16. The oligonucleotide of embodiment 15, wherein the oligonucleotide comprises a sequence that is complementary to a sequence that is at least 90% identical to any one of SEQ ID NOs: 15-26, 661, 663, 666, 667, 681, 682, 692, 693, 694, 695, 701, 702, and 703.

Embodiment 17. The oligonucleotide of embodiment 15 or embodiment 16, wherein the oligonucleotide comprises a sequence that is complementary to a sequence selected from a group consisting of SEQ ID NOs: 15-26, 661, 663, 666, 667, 681, 682, 692, 693, 694, 695, 701, 702, and 703.

Embodiment 18. The oligonucleotide of any one of embodiments 15-17, wherein the oligonucleotide comprises a sequence that is complementary to SEQ ID NO: 23.

Embodiment 19. The oligonucleotide of any one of embodiments 15-17, wherein the oligonucleotide comprises a sequence that is complementary to SEQ ID NO: 24.

Embodiment 20. The oligonucleotide of any one of embodiments 15-17, wherein the oligonucleotide comprises a sequence that is complementary to SEQ ID NO: 663.

Embodiment 21. The oligonucleotide of any one of embodiments 15-17, wherein the oligonucleotide comprises a sequence that is complementary to SEQ ID NO: 681.

Embodiment 22. The oligonucleotide of any one of embodiments 15-17, wherein the oligonucleotide comprises a sequence that is complementary to SEQ ID NO: 701.

Embodiment 23. An oligonucleotide comprising a sequence that differs by no more than 1, 2, 3, or 4 nucleotides from any one of SEQ ID NOs: 3-14, 592, 594, 597, 598, 612, 613, 623, 624, 625, 626, 632, 633, and 634, and/or is complementary to a nucleotide sequence that differs by no more than 1, 2, 3, or 4 nucleotides from any one of SEQ ID NO: 15-26, 661, 663, 666, 667, 681, 682, 692, 693, 694, 695, 701, 702, and 703.

Embodiment 24. An oligonucleotide comprising a sequence that is complementary to a sequence that differs by no more than 1, 2, 3, or 4 nucleotides from a target region that spans between 8 to 30 contiguous nucleotides of a mouse POLRMT nucleotide sequence.

Embodiment 25. The oligonucleotide of embodiment 24, wherein the oligonucleotide comprises a sequence that is complementary to a target region that spans between 8 to 30 contiguous nucleotides of a mouse POLRMT nucleotide sequence.

Embodiment 26. The oligonucleotide of embodiment 24 or embodiment 25, wherein the target region spans between 15 to 25 contiguous nucleotides of a mouse POLRMT nucleotide sequence.

Embodiment 27. The oligonucleotide sequence of any one of embodiments 24-26, wherein the target region spans 20 contiguous nucleotides of a mouse POLRMT nucleotide sequence.

Embodiment 28. The oligonucleotide sequence of any one of embodiments 24-27, wherein the target region comprises an exon region of POLRMT nucleotide sequence.

Embodiment 29. The oligonucleotide of any one of embodiments 24-28, wherein the target region comprises a sequence that corresponds to nucleotides 3348-3367 or 3198-3217 of SEQ ID NO: 581.

Embodiment 30. An oligonucleotide comprising a sequence having at least 80% identity to a sequence selected from a group consisting of SEQ ID NOs: 393-486, 592, 594, 597, 598, 612, 613, 623, 624, 625, 626, 632, 633, and 634.

Embodiment 31. The oligonucleotide of embodiment 30, wherein the oligonucleotide comprises a sequence having at least 90% identity to a sequence selected from a group consisting of SEQ ID NOs: 393-486, 592, 594, 597, 598, 612, 613, 623, 624, 625, 626, 632, 633, and 634.

Embodiment 32. The oligonucleotide of embodiment 30 or embodiment 31, wherein the oligonucleotide comprises a sequence selected from a group consisting of SEQ ID NOs: 393-486, 592, 594, 597, 598, 612, 613, 623, 624, 625, 626, 632, 633, and 634.

Embodiment 33. The oligonucleotide of any one of embodiments 30-32, wherein the oligonucleotide comprises SEQ ID NO: 434.

Embodiment 34. The oligonucleotide of any one of embodiments 30-32, wherein the oligonucleotide comprises SEQ ID NO: 442.

Embodiment 35. The oligonucleotide of any one of embodiments 30-32, wherein the oligonucleotide comprises SEQ ID NO: 594.

Embodiment 36. The oligonucleotide of any one of embodiments 30-32, wherein the oligonucleotide comprises SEQ ID NO: 612.

Embodiment 37. The oligonucleotide of any one of embodiments 30-32, wherein the oligonucleotide comprises SEQ ID NO: 632.

Embodiment 38. An oligonucleotide comprising a sequence that is complementary to a sequence that is at least 80% identical to a sequence selected from a group consisting of SEQ ID NOs: 487-580, 661, 663, 666, 667, 681, 682, 692, 693, 694, 695, 701, 702, and 703.

Embodiment 39. The oligonucleotide of embodiment 38, wherein the oligonucleotide comprises a sequence that is complementary to a sequence that is at least 90% identical to any one of SEQ ID NOs: 487-580, 661, 663, 666, 667, 681, 682, 692, 693, 694, 695, 701, 702, and 703.

Embodiment 40. The oligonucleotide of embodiment 38 or embodiment 39, wherein the oligonucleotide comprises a sequence that is complementary to a sequence selected from a group consisting of SEQ ID NOs: 487-580, 661, 663, 666, 667, 681, 682, 692, 693, 694, 695, 701, 702, and 703.

Embodiment 41. The oligonucleotide of any one of embodiments 38-40, wherein the oligonucleotide comprises a sequence that is complementary to SEQ ID NO: 528.

Embodiment 42. The oligonucleotide of any one of embodiments 38-40, wherein the oligonucleotide comprises a sequence that is complementary to SEQ ID NO: 536.

Embodiment 43. The oligonucleotide of any one of embodiments 38-40, wherein the oligonucleotide comprises a sequence that is complementary to SEQ ID NO: 663.

Embodiment 44. The oligonucleotide of any one of embodiments 38-40, wherein the oligonucleotide comprises a sequence that is complementary to SEQ ID NO: 681.

Embodiment 45. The oligonucleotide of any one of embodiments 38-40, wherein the oligonucleotide comprises a sequence that is complementary to SEQ ID NO: 701.

Embodiment 46. An oligonucleotide comprising a sequence that differs by no more than 1, 2, 3, or 4 nucleotides from any one of SEQ ID NOs: 393-486, 592, 594, 597, 598, 612, 613, 623, 624, 625, 626, 632, 633, and 634 and/or is complementary to a nucleotide sequence that differs by no more than 1, 2, 3, or 4 nucleotides from any one of SEQ ID NO: 487-580, 661, 663, 666, 667, 681, 682, 692, 693, 694, 695, 701, 702, and 703.

Embodiment 47. The oligonucleotide of any one of embodiments 1-46, wherein the oligonucleotide is a chirally pure oligonucleotide.

Embodiment 48. The oligonucleotide of any one of embodiments 1-47, wherein the oligonucleotide comprises at least one modified nucleotide.

Embodiment 49. The oligonucleotide of embodiment 48, wherein the modified nucleotide comprises a base modification, a sugar or sugar phosphate modification, an internucleotidic linkage modification, or a combination thereof.

Embodiment 50. The oligonucleotide of embodiment 49, wherein the internucleotidic linkage modification comprises a phosphorothioate or phosphodithioate linkage modification.

Embodiment 51. The oligonucleotide of embodiment 49, wherein the sugar or sugar phosphate modification comprises a 2′-O-methoxyethyl (2′-MOE) modification, a 2′-Fluoro (2′-F) modification, a 2′-O-methyl (2′-O-Me) modification, a phosphorodiamidate morpholino (PMO) modification, a peptide nucleic acid (PNA) modification, an unlocked nucleic acid (UNA), or a locked nucleic acid (LNA).

Embodiment 52. The oligonucleotide of embodiment 49, wherein the base modification comprises a 5′-methylcytosine modification or a G-clamp modification.

Embodiment 53. The oligonucleotide of any one of embodiments 48-52, wherein each nucleotide comprises a phosphorothioate (PS) internucleotide linkage.

Embodiment 54. The oligonucleotide of any one of embodiments 48-53, wherein the oligonucleotide comprises five nucleotides at the 5′-end and five nucleotides at the 3′-end of the oligonucleotide sequence which contain a 2′-MOE modification.

Embodiment 55. The oligonucleotide of any one of embodiments 48-53, wherein each nucleotide contains a 2′-MOE modification.

Embodiment 56. The oligonucleotide of any one of embodiments 1-55, further comprising at least at least one ligand attached to the 5′ end and/or the 3′ end.

Embodiment 57. The oligonucleotide of embodiment 56, wherein the ligand comprises at least one lipid, peptide, and/or sugar.

Embodiment 58. The oligonucleotide of embodiment 57, wherein the sugar comprises N-acetylgalactosamine (GalNAc)moiety.

Embodiment 59. A composition comprising the oligonucleotide of any one of embodiments 1-58 and a carrier and/or excipient.

Embodiment 60. An expression vector comprising one or more sequences encoding one of more oligonucleotides of any one of embodiments 1-58.

Embodiment 61. A method of treating a subject having or at risk of cancer or metabolic disease, the method comprising administering to the subject a composition comprising an effective amount of the oligonucleotide of any one of embodiments 1-58.

Embodiment 62. The method of embodiment 61, wherein, a level of mitochondrial RNA polymerase (POLRMT) mRNA expression or POLRMT protein in the subject or in a biological sample from the subject after the administration of the composition is reduced relative to a level before the administration of the composition.

Embodiment 63. The method of embodiment 62, wherein the level of POLRMT mRNA expression or POLRMT protein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% relative to a level before the administration.

Embodiment 64. The method of any one of embodiments 61-63, wherein the composition is administered intravenously, intrathecally, intramuscularly, orally, intranasaly, or subcutaneously to the subject.

Embodiment 65. The method of any one of embodiments 61-64, wherein the subject is a human.

Embodiment 66. A method of treating and/or preventing a cancer or a metabolic disease in a subject, comprising administering to the subject an oligonucleotide that is complementary to a target region of a nucleic acid sequence encoding POLRMT.

Embodiment 67. A method of decreasing mitochondrial transcription in a subject that is susceptible to or suffering from cancer or metabolic disease, the method comprising: administering to the subject an oligonucleotide that is complementary to a target region of a nucleic acid sequence encoding POLRMT.

Embodiment 68. The method of embodiment 66 or embodiment 67, wherein the nucleic acid sequence encoding POLRMT comprises SEQ ID NO: 205.

Embodiment 69. The method of any one of embodiments 66-68, wherein the target region comprises a region that spans between 8 to 30 contiguous nucleotides within SEQ ID NO: 205.

Embodiment 70. The method of any one of embodiments 66-69, wherein the target region comprises a sequence that corresponds to nucleotides 5696-5715, 8808-8827, 8809-8828, 8811-8830, 16221-16240, 17159-17178, 17314-17333, 17315-17334, 18082-18101, 18083-18102, 18084-18103, 18130-18149, 5680-5699, 8491-8510, 8529-8548, 8569-8588, 8570-8589, 8571-8590, 8572-8591, 8573-8592, 8574-8593, 13322-13341, 13719-13738, 14999-15018, 15092-15111, 15093-15112, 17304-17323, 19309-19328, 20041-20060, 20042-20061, or 21102-21121 of SEQ ID NO: 1.

Embodiment 71. The method of any one of embodiments 66-70, wherein the oligonucleotide comprises a sequence having at least 80% identity to a sequence selected from a group consisting of SEQ ID NOs: 3-14.

Embodiment 72. The method of any one of embodiments 66-71, wherein the oligonucleotide comprises a sequence having at least 90% identity to a sequence selected from a group consisting of SEQ ID NOs: 3-14.

Embodiment 73. The method of any one of embodiments 66-72, wherein the oligonucleotide comprises a sequence selected from a group consisting of SEQ ID NOs: 3-14.

Embodiment 74. The method of any one of embodiments 66-73, wherein upon administration of the oligonucleotide to the subject, the level of POLRMT mRNA expression in the subject is decreased.

Embodiment 75. The method of any one of embodiments 66-74, wherein upon administration of the oligonucleotide to the subject, the level of POLRMT protein or activity in the subject is decreased.

Embodiment 76. The method of embodiment 74 or embodiment 75, wherein the level of POLRMT mRNA expression, POLRMT protein, or POLRMT activity is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% relative to a level before the administration.

Embodiment 77. The method of any one of embodiments 66-76, wherein the subject is a human.

Embodiment 78. The method of any one of embodiments 66-77, wherein the metabolic disease comprises include obesity, diabetes, non-alcoholic steatohepatitis (NASH), a disorder of amino acid metabolism (amino acidemias), a disorder of organic acid metabolism (organic acidurias, organic acidemias), a disorder of lipid metabolism (lipid storage disorders), a lysosomal storage disorder, a peroxisomal disorder, phenylketonuria (PKU), a glycogen storage disease, or a urea cycle disorder.

Embodiment 79. The method of any one of embodiments 66-78, wherein the composition is delivered to the liver.

Embodiment 80. The method of any one of embodiments 66-78, wherein the composition is delivered to the muscle.

Embodiment 81. The method of any one of embodiments 66-78, wherein the composition is delivered to the CNS.

Embodiment 82. The method of any one of embodiments 66-78, wherein the composition is delivered to the cerebrospinal fluid.

Embodiment 83. A pharmaceutical composition comprising an oligonucleotide of any one of embodiments 1-58.

Embodiment 84. The pharmaceutical composition of embodiment 83, wherein the pharmaceutical composition comprises a pharmaceutically acceptable carrier.

Embodiment 85. The pharmaceutical composition of embodiment 83 or embodiment 84, wherein the oligonucleotide is formulated in a nanocarrier.

Embodiment 86. The pharmaceutical composition of any one of embodiments 83-85, wherein the oligonucleotide is formulated in a lipid nanoparticle (LNP).

Embodiment 87. The pharmaceutical composition of any one of embodiments 83-86, wherein the oligonucleotide is conjugated to at least one GalNAc moiety.

Embodiment 88. The pharmaceutical composition of any one of embodiments 83-87, wherein the composition is formulated for systemic or localized administration.

Embodiment 89. The pharmaceutical composition of embodiment 83-88, wherein the composition is formulated for delivery route selected from intrathecal, intramuscular, or intravenous administration.

Embodiment 90. A method of reducing or inhibiting POLRMT expression in a cell, the method comprising contacting the cell with the oligonucleotide of any one of embodiments 1-58.

Embodiment 91. The method of embodiment 90, wherein the level of POLRMT mRNA expression, POLRMT protein, or POLRMT activity is reduced by at least 10%, at least 20%, at least 30%, at least 40%, at least 50%, at least 60%, at least 70%, at least 80%, or at least 90% relative to the level before the cell is contacted with the oligonucleotide.

Embodiment 92. The method of embodiment 90 or embodiment 91, wherein the cell is in a subject.

Embodiment 93. The method of embodiment 92, wherein the subject is a human.

Embodiment 94. The method of embodiment 93, wherein the human is suffering from or susceptible to cancer or a metabolic disorder.

### EXAMPLES

### Example 1: Knockdown of POLRMT and CytB Expression in HeLa Cells

This example illustrates the design and production of exemplary antisense oligonucleotides (ASOs) that target the POLRMT RNA transcript and are capable of altering mRNA expression of POLRMT in HeLa cells.

POLRMT is a mitochondrial DNA-directed RNA polymerase that is essential in transcription of circular mammalian mitochondrial DNA (mtDNA).

To determine whether the ASOs described in this example were able to impair transcription of mtDNA (through knockdown POLRMT expression), expression of Cytochrome B (CytB), a mitochondrial protein, was also measured.

### ASOs

ASOs were designed and synthesized to target different regions of the POLRMT RNA transcript and are characterized by the corresponding region of the POLRMT gene sequence (represented in Reference No. NG_023049.1 and in SEQ ID NO: 1). ASOs were designed using two different strategies.

The first strategy designed ASOs using software program LNCASO (https://iomics.ugent.be/pjdev/design) and analyzing the longest POLRMT transcript (represented in the sequence Reference No. ENST00000588649.7 and SEQ ID NO: 205). Oligonucleotide length was set to 19 nucleotides. The second strategy designed additional ASOs using the software program PFRED (https://github.com/pfred/pfred-gui/releases/tag/v1.0). For the PFRED program, oligonucleotide length was set to 20 nucleotides and 1 mismatch. The gene base on ENSG ID was searched and the longest POLRMT transcript was chosen as a primary target (represented in the sequence Reference No. ENST00000588649.7 and SEQ ID NO: 205). Oligos with more than 1 mismatch in both cDNA and unspliced mRNA were filtered out. The SVMpred was set as >0.5, PLSpred_optimized was set at >0.8.

The exemplary ASO sequences, the POLRMT sequence targeted (target region), a description of the target region, and the coordinates of the target region within the POLRMT gene sequence (SEQ ID NO: 1) are shown in Table 5 below.

TABLE 5

| ASO | | | | | Corresponding |
|---|---|---|---|---|---|
| SEQ | | SEQ | POLRMT RNA Sequence | | coordinates |
| ID | ASO sequence | ID | (5′ to 3′) (represented | Target | within SEQ ID |
| NO: | (5′ to 3′) | NO: | as a DNA sequence) | region | NO: 1 |
| 3 | CCCCAGTCCTTCCTGCGGTC | 15 | GACCGCAGGAAGGACTGGGG | Exon | 5696-5715 |
| 4 | TCCACATCGAGGCTCAGCTT | 16 | AAGCTGAGCCTCGATGTGGA | Exon | 8808-8827 |
| 5 | CTCCACATCGAGGCTCAGCT | 17 | AGCTGAGCCTCGATGTGGAG | Exon | 8809-8828 |
| 6 | TGCTCCACATCGAGGCTCAG | 18 | CTGAGCCTCGATGTGGAGCA | Exon | 8811-8830 |
| 7 | CCCCAGCTCCTCCCAGTACT | 19 | AGTACTGGGAGGAGCTGGGG | Exon | 16221-16240 |
| 8 | GGCCACCTTCTGGCAGTGCG | 20 | CGCACTGCCAGAAGGTGGCC | Exon | 17159-17178 |
| 9 | GCCACGTCGCTGCCCAGGTG | 21 | CACCTGGGCAGCGACGTGGC | Exon | 17314-17333 |
| 10 | CGCCACGTCGCTGCCCAGGT | 22 | ACCTGGGCAGCGACGTGGCG | Exon | 17315-17334 |
| 11 | CCCATCCACCACTTTCGGCC | 23 | GGCCGAAAGTGGTGGATGGG | Exon | 18082-18101 |
| 12 | GCCCATCCACCACTTTCGGC | 24 | GCCGAAAGTGGTGGATGGGC | Exon | 18083-18102 |
| 13 | CGCCCATCCACCACTTTCGG | 25 | CCGAAAGTGGTGGATGGGCG | Exon | 18084-18103 |
| 14 | TTCGCCACCTCCATACAGCA | 26 | TGCTGTATGGAGGTGGCGAA | Exon | 18130-18149 |
| 27 | GGTCTTGGTCTTGCTCCTGG | 46 | CCAGGAGCAAGACCAAGACC | Exon | 5680-5699 |
| 28 | GAGGCTACCATCTCCACTGC | 47 | GCAGTGGAGATGGTAGCCTC | Exon | 8491-8510 |
| 29 | GCATCCTTGGCCCCCATCTG | 48 | CAGATGGGGGCCAAGGATGC | Exon | 8529-8548 |
| 30 | CTCCAGTATCTTTGCCCAGC | 49 | GCTGGGCAAAGATACTGGAG | Exon | 8569-8588 |
| 31 | TCTCCAGTATCTTTGCCCAG | 50 | CTGGGCAAAGATACTGGAGA | Exon | 8570-8589 |
| 32 | TTCTCCAGTATCTTTGCCCA | 51 | TGGGCAAAGATACTGGAGAA | Exon | 8571-8590 |
| 33 | CTTCTCCAGTATCTTTGCCC | 52 | GGGCAAAGATACTGGAGAAG | Exon | 8572-8591 |
| 34 | CCTTCTCCAGTATCTTTGCC | 53 | GGCAAAGATACTGGAGAAGG | Exon | 8573-8592 |
| 35 | TCCTTCTCCAGTATCTTTGC | 54 | GCAAAGATACTGGAGAAGGA | Exon | 8574-8593 |
| 36 | CACATATACCAGCTCCTTGA | 55 | TCAAGGAGCTGGTATATGTG | Exon | 13322-13341 |
| 37 | TCCTCCTCAGACAGCAGAAC | 56 | GTTCTGCTGTCTGAGGAGGA | Exon | 13719-13738 |
| 38 | CACTGCAGGGTCTTCAAGGG | 57 | CCCTTGAAGACCCTGCAGTG | Exon | 14999-15018 |
| 39 | TGCTTGACCTCCTTGCTTGG | 58 | CCAAGCAAGGAGGTCAAGCA | Exon | 15092-15111 |
| 40 | GTGCTTGACCTCCTTGCTTG | 59 | CAAGCAAGGAGGTCAAGCAC | Exon | 15093-15112 |
| 41 | TGCCCAGGTGGTTGAAGTGC | 60 | GCACTTCAACCACCTGGGCA | Exon | 17304-17323 |
| 42 | TGTAGACTCTTGAAGACCTG | 61 | CAGGTCTTCAAGAGTCTACA | Exon | 19309-19328 |
| 43 | AGCATCATGTGGGAGGAGTC | 62 | GACTCCTCCCACATGATGCT | Exon | 20041-20060 |
| 44 | GAGCATCATGTGGGAGGAGT | 63 | ACTCCTCCCACATGATGCTC | Exon | 20042-20061 |
| 45 | TTCAGCTGGCTGGCCTCCAA | 64 | TTGGAGGCCAGCCAGCTGAA | Exon | 21102-21121 |

### ASO Synthesis

Exemplary ASOs were synthesized using phosphoramidite synthesis methods that begin with the 3′-most nucleotide and proceed through multiple cycles of the following steps: deprotection (trityl group is removed from the 5′ carbon by trichloroacetic acid (TCA) resulting in a reactive hydroxyl group for the next base to be added), coupling (using tetrazole activation to produce an intermediate that reacts with the hydroxyl group), capping (acetylating reagent is added to react with free hydroxyl groups of oligonucleotides where coupling failed), and stabilization (iodine and water are added to cause oxidation of the phosphite into phosphate leaving a stabilized phosphotriester bond), until the 5′-most nucleotide is attached. Where phosphorothioate bonds are produced in exemplary ASOs, a sulfurizing agent is used in place of iodine/water in the stabilize step, for example, dibenzyl tetrasulfide, Beaucage Reagent (3H-1,2-benzodithiol-3-one 1,1-dioxide), 3-ethoxy-1,2,4-dithiazolidin-5-one (EDITH), 1,2,4-dithiazolidine-3,5-dione (DtsNH), 3-amino-1,2,4-dithiazole-5-thione.

The remaining trityl groups were removed from completed synthesis and from the CPG resulting in a hydroxyl group on both the 3′ and 5′ ends. The oligo was deprotected using ammonium hydroxide to promote base hydrolysis. The remaining contaminants were removed through desalting. The oligonucleotides were purified via PAGE or HPLC and quality is confirmed using Mass Spectrometry (using either Matrix Assisted Laser Desorption Ionization-Time of Flight (MALDI-TOF) or Electrospray Ionization (ESI)) (see https://eu.idtdna.com/pages/products/funtional-genomics/antisense-oligos).

In this experiment, ASOs contained modifications that included the following modification pattern:

X_{MS}X_{MS}X_{MS}X_{MS}X_{MS}X_{S}X_{S}X_{S}X_{S}X_{S}X_{S}X_{S}X_{S}X_{S}X_{S}X_{MS}X_{MS}X_{MS}X_{MS}X_{MS}

where “X” represents any nucleotide; a “M” represents a 2′-O-MOE group; and an “S” represents a phosphorothioate bond. Exemplary ASOs with such a modification pattern are shown in FIG. 3.

### ASO Transfection

Human HeLa cells (ATCC CCL-2) were grown at 37° C. with 5% (v/v) CO₂ in Dulbecco's Modified Eagle Medium (DMEM) supplemented with 10% fetal bovine serum (FBS).

6 or 12 μl of each ASO (100 μM) was added to 8 μL of DharmaFECT 1 (horizondiscovery, T-2001-02), 1 ml Opti-MEM media (Thermo Fisher Scientific, Cat #11058021), and mixed well. ASOs were tested at two final concentrations 100 nM and 200 nM. The mixture was incubated for 15 min at room temperature. 5 mL of 5×10⁴/mL Hela cells were added to the mixture and mixed well. The cells and ASO mixture were seeded (2 mL per well) in a 6-well plate and grown for three days.

### RT-PCR Protocol

The following RT-PCR protocol was performed:
1. Transfer cells to eppendorf tubes, pellet cells by centrifuging 500 g 5 min, and remove remaining media. Wash the cell pellet with 1×Phosphate Buffered Saline (PBS). Add 1 mL of trizol solution (Thermo Fisher Scientific, Cat #15596026) to the HeLa cell pellet. Resuspend the cells completely by vortexing.
2. Incubate tubes at room temperature for 5 min.
3. Add 200 μl chloroform. Vortex for 10 seconds.
4. Incubate for 5 min at room temperature to permit nucleoprotein dissociation.
5. Centrifuge the tubes at 12,000×g for 15 min at 4° C.
6. Transfer the upper phase to a new microcentrifuge tube without disturbing the interphase.
7. Add 500 μL of isopropanol to the sample, invert 5 times to mix and incubate 30 min at −20° C.
8. Centrifuge at a minimum of 12,000×g for 15 min at 4° C. and remove the supernatant. The RNA will appear as a white pellet in the tube.
9. Wash the pellet with 500 μL of ice-cold 75% ethanol (made with deionized, diethylpyrocarbonate (DEPC) treated and 0.22 μm membrane-filtered H₂O).
10. Remove the ethanol, air dry for 5 minutes at room temperature (do not completely dry the RNA) and resuspend the RNA in 100 μL of DEPC-H₂O.
11. Incubate 5 μg RNA with 1 unit of turbo DNase (TURBO DNA-free Kit, Thermo Fisher Scientific, Cat #AM1907) at room temperature for 15 minutes and stop reaction following the manufacturer's instructions.
12. Purify RNA using a Quick-RNA miniprep kit (ZYMO, Cat #R1055).
13. Perform reverse transcription using 1 g RNA and the iScript cDNA Synthesis Kit (Biorad, 170-8891).
14. Dilute 20 d of cDNA to a final volume of 200 μl using H₂O. The cDNA is now ready for PCR quantification.

The iTaq Universal SYBR Green Supermix (Bio-Rad, Cat ##1725121) was used together with primers ordered from Eurofins genomics (shown below in Table 6) to detect expression of POLRMT, CytB and 18S rRNA genes. Quantification was performed using the Biorad CFX96 PCR system. The reaction mixtures in each well contained 1 μL forward primer (conc. 5 μM) and 1 μL reverse primer (conc. 5 μM), 2 μL cDNA, 9.5 μL H₂O and 12.5 μL SYBR supermix.

TABLE 6

| | | | SEQ ID | |
|---|---|---|---|---|
| Primer | Supplier | Sequence | NO: | Storage |
| Human 18S_qPCR_F | Eurofins | AGAAACGGCTACCACATCCA | 65 | -20° C. |
| Human 18S_qPCR_R | Eurofins | CCCTCCAATGGATCCTCGTT | 66 | -20° C. |
| POLRMT RT F2 | Eurofins | ACT GTT CTG AAG GCC GTG | 67 | -20° C. |
| POLRMT RT R2 | Eurofins | GGT CTT CAA GGG CAG GTG | 68 | -20° C. |
| Human CytB_qPCR_F | Eurofins | ACAATTCTCCGATCCGTCCC | 69 | -20° C. |
| Human CytB_qPCR_R | Eurofins | GTGATTGGCTTAGTGGGCGA | 70 | -20° C. |

### Results—POLRMT Expression

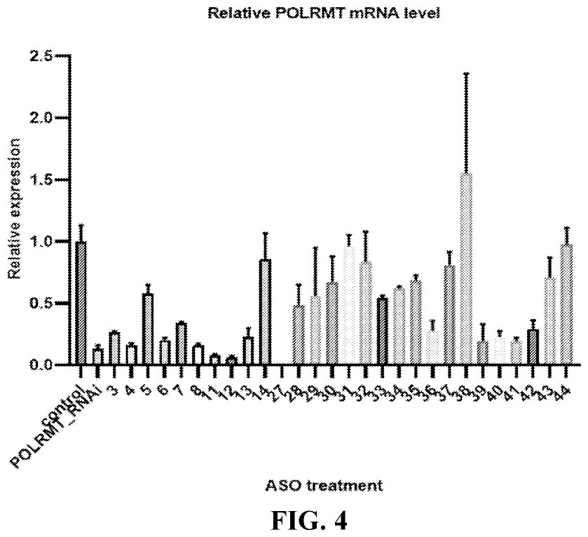

POLRMT expression in HeLa cells transfected with the exemplary ASOs are shown in FIG. 4. A scrambled ASO (not a perfect match to any human transcripts) was used as a control. A POLRMT siRNA (Horizon Discovery Catalog ID:L-012004-01-0005, smartpool format) was used as a positive control. All results were normalized by 18S expression.

Of the ASOs tested at 100 nM concentration (i.e., ASOs represented in SEQ ID NOs: 3-14), all ASO sequences except SEQ ID NO: 5 showed a reduction of relative POLRMT expression at 100 nM (data not shown). SEQ ID NOs: 9 and 10 showed cytotoxic activity and therefore no data was obtained.

The results in FIG. 4 show that ASOs represented in SEQ ID NOs: 3-8, 13-14, 28-30, 32-37, and 39-43 showed some inhibition of POLRMT expression (decreased expression relative to control). ASOs represented in SEQ ID NOs: 11 and 12 showed excellent inhibition of POLRMT expression at 200 nM. ASOs represented in SEQ ID NOs: 9, 10, and 27 showed cytotoxic activity and therefore no data was obtained.

These results show that the inhibition activity of the ASO depends on the region of the POLRMT transcript targeted.

### Results—CytB Expression

In addition to knockdown of POLRMT expression, expression of CytB was also measured in the transfected HeLa cells.

POLRMT is a mitochondrial DNA-directed RNA polymerase that is essential in transcription of circular mammalian mitochondrial DNA (mtDNA). To determine whether the ASOs described in this example were able to impair transcription of mtDNA (through knockdown POLRMT expression), expression of Cytochrome B (CytB), a mitochondrial protein, was measured in the HeLa cells transfected with ASOs.

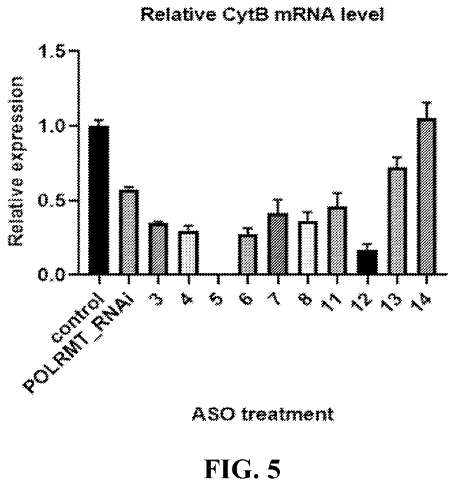

CytB expression in HeLa cells transfected with the exemplary ASOs are shown in FIG. 5. The same controls were used in this screen. All results were normalized by 18S expression.

The results in FIG. 5 show that the ASOs targeting POLRMT that were able to knockdown expression of POLRMT mRNA also resulted in decreased expression of CytB in all of the ASOs except ASO represented by SEQ ID NO: 14, where there was a slight increase. These results show that the knockdown in POLRMT expression leads to decreased activity of POLRMT (i.e., transcription of CytB mtDNA). No data was obtained from the ASOs represented in SEQ ID NOs: 9 and 10 due to cytotoxicity.

### Example 2: Design and Modification of Additional POLRMT ASO Sequences

This example illustrates the design of additional exemplary antisense oligonucleotides (ASOs) that target POLRMT RNA.

ASOs were designed using two different strategies. The first strategy designed ASOs using software program LNCASO (https://iomics.ugent.be/pjdev/design) and analyzing the longest POLRMT transcript (represented in Reference No. ENST00000588649 and SEQ ID NO: 205). Oligo length was set to 19 nucleotides. The ASOs designed using this strategy are shown in Table 7 below.

The second strategy designed additional ASOs using the software program PFRED (https://github.com/pfred/pfred-gui/release/tag/v1.0). For the PFRED program, oligo length was set to 20 nucleotides and 1 mismatch. The gene base on ENSG ID was searched and the longest POLRMT transcript (represented in Reference No. ENST00000588649 and SEQ ID NO: 205). Oligos with more than 1 mismatch in both cDNA and unspliced mRNA were filtered out. The SVMpred was set as >0.5, PLSpred_optimized was set at >0.7 or >0.8. The ASOs designed using this strategy are shown in Table 8 below.

TABLE 7

| ASO | |
|---|---|
| SEQ ID | |
| NO: | ASO sequence (5′ to 3′) |
| 71 | GAGATAGTGAGAGGCCTCC |
| 72 | AGGAATACACGTGGTAGAG |
| 73 | CCAGTACTGCCGCGGCAGG |
| 74 | AGTCCAGGATGTCATCCAT |
| 75 | TCTTCTGCTTACGTGTGTT |
| 76 | ATCATGTGGGAGGAGTCCA |
| 77 | GCCTGCAGGACCTGCAGGA |
| 78 | CAGCTGGTCAGTGAGCAGG |
| 79 | CTATTTGCTTGACCTTGGA |
| 80 | CAAAGCATGGGTACATCCA |
| 81 | AAGGGGTAAAGTGAGAACC |
| 82 | AGAGGTACTTCCTGTAGTG |
| 83 | AACACATATACCAGCTCCT |
| 84 | TTCATGACGGAGACATCAG |
| 85 | GTAGGGCTCGTTTGAGCCC |
| 86 | CCCATGTGGGAGATGAGGC |
| 87 | GAGTCAAGCCGGCATCCTT |
| 88 | CCAAGATCTTCTGGGGCTC |
| 89 | CACTGCAGGGTCTTCAAGG |
| 90 | CACACCCTGCTGGCCAGCT |

TABLE 8

| | | | | | Corresponding |
|---|---|---|---|---|---|
| SEQ | | SEQ | POLRMT RNA Sequence | | coordinates |
| ID | ASO sequence | ID | (5′ to 3′) (represented | Target | within SEQ ID |
| NO: | (5′ to 3′) | NO: | as a DNA sequence) | region | NO: 1 |
| 91 | ACGCCGCTCCAGGCCACCCC | 148 | GGGGTGGCCTGGAGCGGCGT | 5′UTR | 5032-5051 |
| 92 | GCACGCCGCTCCAGGCCACC | 149 | GGTGGCCTGGAGCGGCGTGC | 5′UTR | 5034-5053 |
| 93 | ACGCACGCCGCTCCAGGCCA | 150 | TGGCCTGGAGCGGCGTGCGT | 5′UTR | 5036-5055 |
| 94 | GGCCCCAGCAAAGTGCCGAC | 151 | GTCGGCACTTTGCTGGGGCC | exon | 5059-5078 |
| 95 | GTCCTTCCTGCGGTCTTGGT | 152 | ACCAAGACCGCAGGAAGGAC | exon | 5691-5710 |
| 96 | AGTCCTTCCTGCGGTCTTGG | 153 | CCAAGACCGCAGGAAGGACT | exon | 5692-5711 |
| 97 | CCCCAGTCCTTCCTGCGGTC | 154 | GACCGCAGGAAGGACTGGGG | exon | 5696-5715 |
| 98 | CACGTGGCCCCAGTCCTTCC | 155 | GGAAGGACTGGGGCCACGTG | exon | 5703-5722 |
| 99 | CATCCACCCTGTTCACCACC | 156 | GGTGGTGAACAGGGTGGATG | exon | 8450-8469 |
| 100 | CACATCCACCCTGTTCACCA | 157 | TGGTGAACAGGGTGGATGTG | exon | 8452-8471 |
| 101 | GCGCCACATCCACCCTGTTC | 158 | GAACAGGGTGGATGTGGCGC | exon | 8456-8475 |
| 102 | GCCCCCATCTGGACCTTCCT | 159 | AGGAAGGTCCAGATGGGGGC | exon | 8520-8539 |
| 103 | GAACTCCCCGCTCTGGAATG | 160 | CATTCCAGAGCGGGGAGTTC | exon | 8647-8666 |
| 104 | CTTGAACTCCCCGCTCTGGA | 161 | TCCAGAGCGGGGAGTTCAAG | exon | 8650-8669 |
| 105 | CGGCCATCTGCTTGCTCAGG | 162 | CCTGAGCAAGCAGATGGCCG | exon | 8705-8724 |
| 106 | CCGGCCATCTGCTTGCTCAG | 163 | CTGAGCAAGCAGATGGCCGG | exon | 8706-8725 |
| 107 | ACCCGGCCATCTGCTTGCTC | 164 | GAGCAAGCAGATGGCCGGGT | exon | 8708-8727 |
| 108 | CTCCAGGCACCCGGCCATCT | 165 | AGATGGCCGGGTGCCTGGAG | exon | 8716-8735 |
| 109 | CCTCCAGGCACCCGGCCATC | 166 | GATGGCCGGGTGCCTGGAGG | exon | 8717-8736 |
| 110 | ATCGAGGCTCAGCTTCCCAG | 167 | CTGGGAAGCTGAGCCTCGAT | exon | 8803-8822 |
| 111 | CCTGCTCCACATCGAGGCTC | 168 | GAGCCTCGATGTGGAGCAGG | exon | 8813-8832 |
| 112 | GGCGTTGTACATGTCCAGCG | 169 | CGCTGGACATGTACAACGCC | exon | 8986-9005 |
| 113 | GCATCACGGCGTTGTACATG | 170 | CATGTACAACGCCGTGATGC | exon | 8993-9012 |
| 114 | AGCATCACGGCGTTGTACAT | 171 | ATGTACAACGCCGTGATGCT | exon | 8995-9014 |
| 115 | AAGCATCACGGCGTTGTACA | 172 | TGTACAACGCCGTGATGCTT | exon | 8994-9013 |
| 116 | TCCAGACACCTTTCGATGGT | 173 | ACCATCGAAAGGTGTCTGGA | exon | 13435-13447, |
| | | | | | 13666-13672 |
| 117 | CAGAACGGCGGTGAAGAGTG | 174 | CACTCTTCACCGCCGTTCTG | exon | 13705-13724 |
| 118 | GCAGAACGGCGGTGAAGAGT | 175 | ACTCTTCACCGCCGTTCTGC | exon | 13706-13725 |
| 119 | GACAGCAGAACGGCGGTGAA | 176 | TTCACCGCCGTTCTGCTGTC | exon | 13710-13729 |
| 120 | ACGGCCTTCAGAACAGTGGC | 177 | GCCACTGTTCTGAAGGCCGT | exon | 13743-13762 |
| 121 | CACCTTGTGCACGGCCTTCA | 178 | TGAAGGCCGTGCACAAGGTG | exon | 13753-13772 |
| 122 | ACACGCCCATCCTTGGCATA | 179 | TATGCCAAGGATGGGCGTGT | exon | 13842-13851, |
| | | | | | 14967-14976 |
| 123 | GGACACACGCCCATCCTTGG | 180 | CCTGCAAGGATGGGCGTGTGTCC | exon | 14959-14980 |
| 124 | AGGACACACGCCCATCCTTG | 181 | CAAGGATGGGCGTGTGTCCT | exon | 14963-14981 |
| 125 | GTAGGACACACGCCCATCCT | 182 | AGGATGGGCGTGTGTCCTAC | exon | 14964-14983 |
| 126 | GGTAGGACACACGCCCATCC | 183 | GGATGGGCGTGTGTCCTACC | exon | 14965-14984 |
| 127 | GACCTCCTTGCTTGGCAACG | 184 | CGTTGCCAAGCAAGGAGGTC | exon | 15087-15106 |
| 128 | TTTCTCCCATTGGTCCCGCA | 185 | TGCGGGACCAATGGGAGAAA | exon | 15603-15622 |
| 129 | GTGCTTTCTCCCATTGGTCC | 186 | GGACCAATGGGAGAAAGCAC | exon | 15607-15626 |
| 130 | CCGGCACAGTGCTTTCTCCC | 187 | GGGAGAAAGCACTGTGCCGG | exon | 15615-15634 |
| 131 | CCACTGACCCGCTGCCTCTG | 188 | CAGAGGCAGCGGGTCAGTGG | exon | 15904-15923 |
| 132 | CTCCTCCCAGTACTGCCGCG | 189 | CGCGGCAGTACTGGGAGGAG | exon | 16215-16234 |
| 133 | TTGTCCAGGCTGCATGGCAT | 190 | ATGCCATGCAGCCTGGACAA | exon | 16334-16353 |
| 134 | TCCACCGCCTCGAAGGTCAG | 191 | CTGACCTTCGAGGCGGTGGA | exon | 16786-16805 |
| 135 | ATCCACCGCCTCGAAGGTCA | 192 | TGACCTTCGAGGCGGTGGAT | exon | 16787-16806 |
| 136 | CATCCACCGCCTCGAAGGTC | 193 | GACCTTCGAGGCGGTGGATG | exon | 16788-16807 |
| 137 | ACATCCACCGCCTCGAAGGT | 194 | ACCTTCGAGGCGGTGGATGT | exon | 16789-16808 |
| 138 | GGTACATCCACCGCCTCGAA | 195 | TTCGAGGCGGTGGATGTACC | exon | 16792-16811 |
| 139 | GTGCGGCGATGTCCAGGGCA | 196 | TGCCCTGGACATCGCCGCAC | exon | 16829-16848 |
| 140 | AGAGTGCGGCGATGTCCAGG | 197 | CCTGGACATCGCCGCACTCT | exon | 16832-16851 |
| 141 | CAGAGTGCGGCGATGTCCAG | 198 | CTGGACATCGCCGCACTCTG | exon | 16833-16852 |
| 142 | CCAGAGTGCGGCGATGTCCA | 199 | TGGACATCGCCGCACTCTGG | exon | 16834-16853 |
| 143 | CACCAGAGTGCGGCGATGTC | 200 | GACATCGCCGCACTCTGGTG | exon | 16836-16855 |
| 144 | AGCACCAGAGTGCGGCGATG | 201 | CATCGCCGCACTCTGGTGCT | exon | 16838-16857 |
| 145 | GGTCCAGCACGCGCCCGTTG | 202 | CAACGGGCGCGTGCTGGACC | exon | 16998-17017 |
| 146 | AGGTCCAGCACGCGCCCGTT | 203 | AACGGGCGCGTGCTGGACCT | exon | 16999-17018 |
| 147 | GCCAGCTCACGGCGCAGCTC | 204 | GAGCTGCGCCGTGAGCTGGC | exon | 17140-17159 |
| 205 | CCGCCGCTCCAGGCCACCCC | 299 | GGGGTGGCCTGGAGCGGCGG | exon | 5032-5051 |
| 206 | GCACGCCGCTCCAGGCCACC | 300 | GGTGGCCTGGAGCGGCGTGC | exon | 5034-5053 |
| 207 | ACGCACGCCGCTCCAGGCCA | 301 | TGGCCTGGAGCGGCGTGCGT | exon | 5036-5055 |
| 208 | GGCCCCAGCAAAGTGCCGAC | 302 | GTCGGCACTTTGCTGGGGCC | exon | 5059-5078 |
| 209 | GTCCTTCCTGCGGTCTTGGT | 303 | ACCAAGACCGCAGGAAGGAC | exon | 5691-5710 |
| 210 | AGTCCTTCCTGCGGTCTTGG | 304 | CCAAGACCGCAGGAAGGACT | exon | 5692-5711 |
| 211 | CCCCAGTCCTTCCTGCGGTC | 305 | GACCGCAGGAAGGACTGGGG | exon | 5696-5715 |
| 212 | CACGTGGCCCCAGTCCTTCC | 306 | GGAAGGACTGGGGCCACGTG | exon | 5703-5722 |
| 213 | CATCCACCCTGTTCACCACC | 307 | GGTGGTGAACAGGGTGGATG | exon | 8450-8469 |
| 214 | CACATCCACCCTGTTCACCA | 308 | TGGTGAACAGGGTGGATGTG | exon | 8452-8471 |
| 215 | GCGCCACATCCACCCTGTTC | 309 | GAACAGGGTGGATGTGGCGC | exon | 8456-8475 |
| 216 | GCCCCCATCTGGACCTTCCT | 310 | AGGAAGGTCCAGATGGGGGC | exon | 8520-8539 |
| 217 | GAACTCCCCGCTCTGGAATG | 311 | CATTCCAGAGCGGGGAGTTC | exon | 8647-8666 |
| 218 | CTTGAACTCCCCGCTCTGGA | 312 | TCCAGAGCGGGGAGTTCAAG | exon | 8650-8669 |
| 219 | CGGCCATCTGCTTGCTCAGG | 313 | CCTGAGCAAGCAGATGGCCG | exon | 8705-8724 |
| 220 | CCGGCCATCTGCTTGCTCAG | 314 | CTGAGCAAGCAGATGGCCGG | exon | 8706-8725 |
| 221 | ACCCGGCCATCTGCTTGCTC | 315 | GAGCAAGCAGATGGCCGGGT | exon | 8708-8727 |
| 222 | CTCCAGGCACCCGGCCATCT | 316 | AGATGGCCGGGTGCCTGGAG | exon | 8716-8735 |
| 223 | CCTCCAGGCACCCGGCCATC | 317 | GATGGCCGGGTGCCTGGAGG | exon | 8717-8736 |
| 224 | ATCGAGGCTCAGCTTCCCAG | 318 | CTGGGAAGCTGAGCCTCGAT | exon | 8803-8822 |
| 225 | CCTGCTCCACATCGAGGCTC | 319 | GAGCCTCGATGTGGAGCAGG | exon | 8813-8832 |
| 226 | GGCGTTGTACATGTCCAGCG | 320 | CGCTGGACATGTACAACGCC | exon | 8986-9005 |
| 227 | GCATCACGGCGTTGTACATG | 321 | CATGTACAACGCCGTGATGC | exon | 8993-9012 |
| 228 | AGCATCACGGCGTTGTACAT | 322 | ATGTACAACGCCGTGATGCT | exon | 8994-9013 |
| 229 | AAGCATCACGGCGTTGTACA | 323 | TGTACAACGCCGTGATGCTT | exon | 8995-9014 |
| 230 | TCCAGACACCTTTCGATGGT | 324 | ACCATCGAAAGGTGTCTGGA | exon | 13435-13447 |
| | | | | | and |
| | | | | | 13666-13672 |
| 231 | CAGAACGGCGGTGAAGAGTG | 325 | CACTCTTCACCGCCGTTCTG | exon | 13705-13724 |
| 232 | GCAGAACGGCGGTGAAGAGT | 326 | ACTCTTCACCGCCGTTCTGC | exon | 13706-13725 |
| 233 | GACAGCAGAACGGCGGTGAA | 327 | TTCACCGCCGTTCTGCTGTC | exon | 13710-13729 |
| 234 | ACGGCCTTCAGAACAGTGGC | 328 | GCCACTGTTCTGAAGGCCGT | exon | 13743-13762 |
| 235 | CACCTTGTGCACGGCCTTCA | 329 | TGAAGGCCGTGCACAAGGTG | exon | 13753-13772 |
| 236 | ACACGCCCATCCTTGGCATA | 330 | TATGCCAAGGATGGGCGTGT | exon | 13842-13850 |
| | | | | | and |
| | | | | | 14966-14976 |
| 237 | GGACACACGCCCATCCTTGG | 331 | CCAAGGATGGGCGTGTGTCC | exon | 13846-13850 |
| | | | | | and |
| | | | | | 14966-14980 |
| 238 | AGGACACACGCCCATCCTTG | 332 | CAAGGATGGGCGTGTGTCCT | exon | 13847-13850 |
| | | | | | and |
| | | | | | 14966-14981 |
| 239 | GTAGGACACACGCCCATCCT | 333 | AGGATGGGCGTGTGTCCTAC | exon | 13849-13850 |
| | | | | | and |
| | | | | | 14966-14983 |
| 240 | GGTAGGACACACGCCCATCC | 334 | GGATGGGCGTGTGTCCTACC | exon | 13850-13850 |
| | | | | | and |
| | | | | | 14966-14984 |
| 241 | GACCTCCTTGCTTGGCAACG | 335 | CGTTGCCAAGCAAGGAGGTC | exon | 15087-15106 |
| 242 | TTTCTCCCATTGGTCCCGCA | 336 | TGCGGGACCAATGGGAGAAA | exon | 15603-15622 |
| 243 | GTGCTTTCTCCCATTGGTCC | 337 | GGACCAATGGGAGAAAGCAC | exon | 15607-15626 |
| 244 | CCGGCACAGTGCTTTCTCCC | 338 | GGGAGAAAGCACTGTGCCGG | exon | 15615-15634 |
| 245 | CCACTGACCCGCTGCCTCTG | 339 | CAGAGGCAGCGGGTCAGTGG | exon | 15904-15923 |
| 246 | CTCCTCCCAGTACTGCCGCG | 340 | CGCGGCAGTACTGGGAGGAG | exon | 16215-16234 |
| 247 | TTGTCCAGGCTGCATGGCAT | 341 | ATGCCATGCAGCCTGGACAA | exon | 16334-16353 |
| 248 | TCCACCGCCTCGAAGGTCAG | 342 | CTGACCTTCGAGGCGGTGGA | exon | 16786-16805 |
| 249 | ATCCACCGCCTCGAAGGTCA | 343 | TGACCTTCGAGGCGGTGGAT | exon | 16787-16806 |
| 250 | CATCCACCGCCTCGAAGGTC | 344 | GACCTTCGAGGCGGTGGATG | exon | 16788-16807 |
| 251 | ACATCCACCGCCTCGAAGGT | 345 | ACCTTCGAGGCGGTGGATGT | exon | 16789-16808 |
| 252 | GGTACATCCACCGCCTCGAA | 346 | TTCGAGGCGGTGGATGTACC | exon | 16792-16811 |
| 253 | GTGCGGCGATGTCCAGGGCA | 347 | TGCCCTGGACATCGCCGCAC | exon | 16829-16848 |
| 254 | AGAGTGCGGCGATGTCCAGG | 348 | CCTGGACATCGCCGCACTCT | exon | 16832-16851 |
| 255 | CAGAGTGCGGCGATGTCCAG | 349 | CTGGACATCGCCGCACTCTG | exon | 16833-16852 |
| 256 | CCAGAGTGCGGCGATGTCCA | 350 | TGGACATCGCCGCACTCTGG | exon | 16834-16853 |
| 257 | CACCAGAGTGCGGCGATGTC | 351 | GACATCGCCGCACTCTGGTG | exon | 16836-16855 |
| 258 | AGCACCAGAGTGCGGCGATG | 352 | CATCGCCGCACTCTGGTGCT | exon | 16838-16857 |
| 259 | GGTCCAGCACGCGCCCGTTG | 353 | CAACGGGCGCGTGCTGGACC | exon | 16998-17017 |
| 260 | AGGTCCAGCACGCGCCCGTT | 354 | AACGGGCGCGTGCTGGACCT | exon | 16999-17018 |
| 261 | GCCAGCTCACGGCGCAGCTC | 355 | GAGCTGCGCCGTGAGCTGGC | exon | 17140-17159 |
| 262 | CGCCAGCTCACGGCGCAGCT | 356 | AGCTGCGCCGTGAGCTGGCG | exon | 17141-17160 |
| 263 | GCAGTGCGCCAGCTCACGGC | 357 | GCCGTGAGCTGGCGCACTGC | exon | 17147-17166 |
| 264 | CTTCTGGCAGTGCGCCAGCT | 358 | AGCTGGCGCACTGCCAGAAG | exon | 17153-17172 |
| 265 | ACCTTCTGGCAGTGCGCCAG | 359 | CTGGCGCACTGCCAGAAGGT | exon | 17155-17174 |
| 266 | CCACCTTCTGGCAGTGCGCC | 360 | GGCGCACTGCCAGAAGGTGG | exon | 17157-17176 |
| 267 | TCCCGGGCCACCTTCTGGCA | 361 | TGCCAGAAGGTGGCCCGGGA | exon | 17164-17183 |
| 268 | GACGCGGTCCCGCAGGTGCT | 362 | AGCACCTGCGGGACCGCGTC | exon | 17231-17250 |
| 269 | GGAAGTCCATGTTGTGCGGC | 363 | GCCGCACAACATGGACTTCC | exon | 17259-17278 |
| 270 | GCGGAAGTCCATGTTGTGCG | 364 | CGCACAACATGGACTTCCGC | exon | 17261-17280 |
| 271 | GTCGCTGCCCAGGTGGTTGA | 365 | TCAACCACCTGGGCAGCGAC | exon | 17309-17328 |
| 272 | ACGTCGCTGCCCAGGTGGTT | 366 | AACCACCTGGGCAGCGACGT | exon | 17311-17330 |
| 273 | CCACGTCGCTGCCCAGGTGG | 367 | CCACCTGGGCAGCGACGTGG | exon | 17313-17332 |
| 274 | CCCGCGCCACGTCGCTGCCC | 368 | GGGCAGCGACGTGGCGCGGG | exon | 17319-17338 |
| 275 | GGCCCGCGCCACGTCGCTGC | 369 | GCAGCGACGTGGCGCGGGCC | exon | 17321-17340 |
| 276 | GCGGCCCTGGGCGAACTCCA | 370 | TGGAGTTCGCCCAGGGCCGC | exon | 17345-17364 |
| 277 | CACCACTTTCGGCCCGTCAA | 371 | TTGACGGGCCGAAAGTGGTG | exon | 17506-17512 |
| | | | | | and |
| | | | | | 18083-18095 |
| 278 | CCTCCGCGCCCATCCACCAC | 372 | GTGGTGGATGGGCGCGGAGG | exon | 18090-18109 |
| 279 | TCCTCCGCGCCCATCCACCA | 373 | TGGTGGATGGGCGCGGAGGA | exon | 18091-18110 |
| 280 | GTTCCTCCGCGCCCATCCAC | 374 | GTGGATGGGCGCGGAGGAAC | exon | 18093-18112 |
| 281 | GGGTTCCTCCGCGCCCATCC | 375 | GGATGGGCGCGGAGGAACCC | exon | 18095-18114 |
| 282 | AGGCCAGCGTCTGCCAGGGT | 376 | ACCCTGGCAGACGCTGGCCT | exon | 18111-18130 |
| 283 | CGGCACATCCGAGGGCTCCA | 377 | TGGAGCCCTCGGATGTGCCG | exon | 18562-18581 |
| 284 | CACGCCGCTGTACACGTCCT | 378 | AGGACGTGTACAGCGGCGTG | exon | 18583-18602 |
| 285 | CCACGCCGCTGTACACGTCC | 379 | GGACGTGTACAGCGGCGTGG | exon | 18584-18603 |
| 286 | GCCACGCCGCTGTACACGTC | 380 | GACGTGTACAGCGGCGTGGC | exon | 18585-18604 |
| 287 | GGCCACGCCGCTGTACACGT | 381 | ACGTGTACAGCGGCGTGGCC | exon | 18586-18605 |
| 288 | TGCGCGGCCACGCCGCTGTA | 382 | TACAGCGGCGTGGCCGCGCA | exon | 18591-18610 |
| 289 | CTGCCTACGGAACACCTCCA | 383 | TGGAGGTGTTCCGTAGGCAG | exon | 18805-18824 |
| 290 | TTCCAGCACCTGTGCCACCC | 384 | GGGTGGCACAGGTGCTGGAA | exon | 18844-18863 |
| 291 | GAGGCCTCCCACACGAACTC | 385 | GAGTTCGTGTGGGAGGCCTC | exon | 19273-19292 |
| 292 | AGAGGCCTCCCACACGAACT | 386 | AGTTCGTGTGGGAGGCCTCT | exon | 19274-19293 |
| 293 | TGAATTCCACCTCCTATTTG | 387 | CAAATAGGAGGTGGAATTCA | exon | 19809-19828 |
| 294 | CGAAGGTCAGGCCCTTCCTG | 388 | CAGGAAGGGCCTGACCTTCG | exon | 20717-20736 |
| 295 | AGACGAAGGTCAGGCCCTTC | 389 | GAAGGGCCTGACCTTCGTCT | exon | 20720-20739 |
| 296 | GAGACGAAGGTCAGGCCCTT | 390 | AAGGGCCTGACCTTCGTCTC | exon | 20721-20740 |
| 297 | AGAGACGAAGGTCAGGCCCT | 391 | AGGGCCTGACCTTCGTCTCT | exon | 20722-20741 |
| 298 | AGTAACAGTCGTGCACAGAG | 392 | CTCTGTGCACGACTGTTACT | exon | 20738-20757 |

### ASO Synthesis

Exemplary ASOs are synthesized according to methods described in Example 1 (see https://eu.idtdna.com/pages/products/functional-genomics/antisense-oligos).

ASOs contain the following modification pattern:

X_{MS}X_{MS}X_{MS}X_{MS}X_{MS}X_{S}X_{S}X_{S}X_{S}X_{S}X_{S}X_{S}X_{S}X_{S}X_{S}X_{MS}X_{MS}X_{MS}X_{MS}X_{MS}

where “X” represents any nucleotide; a “M” represents a 2′-O-MOE group; and

an “S” represents a phosphorothioate bond,

### ASO Transfection

Human HeLa cells (ATCC CCL-2) are grown at 37° C. with 500 (v/v) CO₂ in Dulbecco's Modified Eagle Medium (DMEM) supplemented with 10% fetal bovine serum (FB S).

6 or 12 μl of each ASO (100 μM) is added to 8 μL of DharmaFECT 1 (horizondiscovery, T-2001-02), 1 ml Opti-MEM media (Thermo Fisher Scientific, Cat #11058021), and mixed well (final concentration of ASO is 100 nM and 200 nM). The mixture is incubated for 15 min at room temperature. 5 mL of 5×10⁴/mL Hela cells are added to the mixture and mixed well. The cells and ASO mixture are seeded (2 mL per well) in a 6-well plate and grown for three days.

### RT-PCR Protocol

The RT-PCR protocol is performed according to Example 1 in order to measure relative POLRMT mRNA expression levels.

### Example 3: Design and Modification of Additional POLRMT ASO Sequences

This example illustrates the design of additional exemplary mouse antisense oligonucleotides (ASOs) that target mouse POLRMT RNA.

ASOs were designed using the software program PFRED (https.//github.com/pfred/pfred-gui/releases/tag/v1.0). For the PFRED program, oligo length was set to 20 nucleotides and 1 mismatch. The gene base on ENSG ID was searched and the longest mouse POLRMT transcript (represented in Reference No. ENSMUST00000161765; SEQ ID NO: 582). Oligos with more than 1 mismatch in both cDNA and unspliced mRNA were filtered out. The SVMpred was set as >0.6, PLSpred_optimized was set at >0.8. 0.8.

Exemplary mouse ASO sequences, the POLRMT sequence targeted (target region), a description of the target region, and the coordinates of the target region within the mouse POLRMT gene sequence (SEQ ID NO: 581) are shown in Table 9 below.

TABLE 9

| | | | POLRMT RNA Sequence | | | |
|---|---|---|---|---|---|---|
| SEQ | | SEQ | (5′ to 3′) | | | |
| ID | ASO sequence | ID | (represented as a | Target | | |
| NO: | (5′ to 3′) | NO: | DNA sequence) | region | Start | End |
| 393 | CACGGTGTTGTACATGTGCA | 487 | TGCACATGTACAACACCGTG | Exon | 7077 | 7096 |
| 394 | ATCACGGTGTTGTACATGTG | 488 | CACATGTACAACACCGTGAT | Exon | 7075 | 7094 |
| 395 | CATCACGGTGTTGTACATGT | 489 | ACATGTACAACACCGTGATG | Exon | 7074 | 7093 |
| 396 | GTGCATCTCTCGAGCCACCT | 490 | AGGTGGCTCGAGAGATGCAC | Exon | 3342 | 3361 |
| 397 | TGTGCATCTCTCGAGCCACC | 491 | GGTGGCTCGAGAGATGCACA | Exon | 3341 | 3360 |
| 398 | CTGTGCATCTCTCGAGCCAC | 492 | GTGGCTCGAGAGATGCACAG | Exon | 3340 | 3359 |
| 399 | GTGCTGTGCCAGCGACAGGC | 493 | GCCTGTCGCTGGCACAGCAC | Exon | 3297 | 3316 |
| 400 | GTCCATGTTGTGCGGCAACC | 494 | GGTTGCCGCACAACATGGAC | Exon | 3258 | 3277 |
| 401 | AGGTCACTGCCCAGGTGGTT | 495 | AACCACCTGGGCAGTGACCT | Exon | 3202 | 3221 |
| 402 | TCCATCCACCACTTCCGGCC | 496 | GGCCGGAAGTGGTGGATGGA | Exon | 2663 | 2682 |
| 403 | ACCTCCATGCAGCAGGCCAG | 497 | CTGGCCTGCTGCATGGAGGT | Exon | 2621 | 2640 |
| 404 | CACCTCCATGCAGCAGGCCA | 498 | TGGCCTGCTGCATGGAGGTG | Exon | 2620 | 2639 |
| 405 | CCACCTCCATGCAGCAGGCC | 499 | GGCCTGCTGCATGGAGGTGG | Exon | 2619 | 2638 |
| 406 | GCCACCTCCATGCAGCAGGC | 500 | GCCTGCTGCATGGAGGTGGC | Exon | 2618 | 2637 |
| 407 | TGCCACCTCCATGCAGCAGG | 501 | CCTGCTGCATGGAGGTGGCA | Exon | 2617 | 2636 |
| 408 | CACCACCTTGCGGCTGATGA | 502 | TCATCAGCCGCAAGGTGGTG | Exon | 2005 | 2024 |
| 409 | TTCACCACCTTGCGGCTGAT | 503 | ATCAGCCGCAAGGTGGTGAA | Exon | 2003 | 2022 |
| 410 | CTGCTGTCTGTCTCCGTTGT | 504 | ACAACGGAGACAGACAGCAG | Exon | 7107 | 7126 |
| 411 | ACCTGCTGTCTGTCTCCGTT | 505 | AACGGAGACAGACAGCAGGT | Exon | 7105 | 7124 |
| 412 | GCACCTGCTGTCTGTCTCCG | 506 | CGGAGACAGACAGCAGGTGC | Exon | 7103 | 7122 |
| 413 | TGTTGTACATGTGCAGTGTG | 507 | CACACTGCACATGTACAACA | Exon | 7082 | 7101 |
| 414 | CGGTGTTGTACATGTGCAGT | 508 | ACTGCACATGTACAACACCG | Exon | 7079 | 7098 |
| 415 | GACCAGGTCAGTGAAGAGCA | 509 | TGCTCTTCACTGACCTGGTC | Exon | 5712 | 5731 |
| 416 | TCCAGGACCAGGTCAGTGAA | 510 | TTCACTGACCTGGTCCTGGA | Exon | 5707 | 5726 |
| 417 | CTTCCAGGACCAGGTCAGTG | 511 | CACTGACCTGGTCCTGGAAG | Exon | 5705 | 5724 |
| 418 | CTCCCCTTCGCTCAGCAGGC | 512 | GCCTGCTGAGCGAAGGGGAG | Exon | 4732 | 4751 |
| 419 | ACTCCCCTTCGCTCAGCAGG | 513 | CCTGCTGAGCGAAGGGGAGT | Exon | 4731 | 4750 |
| 420 | AACTCCCCTTCGCTCAGCAG | 514 | CTGCTGAGCGAAGGGGAGTT | Exon | 4730 | 4749 |
| 421 | GCCCAGGTTATGGGCCAACT | 515 | AGTTGGCCCATAACCTGGGC | Exon | 4568 | 4587 |
| 422 | CTGTTGTGCAGGGGCCTCCA | 516 | TGGAGGCCCCTGCACAACAG | Exon | 4178 | 4197 |
| 423 | CAATTGCTTGCCCAGCTGTA | 517 | TACAGCTGGGCAAGCAATTG | Exon | 4136 | 4155 |
| 424 | CCAATTGCTTGCCCAGCTGT | 518 | ACAGCTGGGCAAGCAATTGG | Exon | 4135 | 4154 |
| 425 | CGCCAATTGCTTGCCCAGCT | 519 | AGCTGGGCAAGCAATTGGCG | Exon | 4133 | 4152 |
| 426 | CCGCCAATTGCTTGCCCAGC | 520 | GCTGGGCAAGCAATTGGCGG | Exon | 4132 | 4151 |
| 427 | TCCGCCAATTGCTTGCCCAG | 521 | CTGGGCAAGCAATTGGCGGA | Exon | 4131 | 4150 |
| 428 | CTCCGCCAATTGCTTGCCCA | 522 | TGGGCAAGCAATTGGCGGAG | Exon | 4130 | 4149 |
| 429 | GCTCCGCCAATTGCTTGCCC | 523 | GGGCAAGCAATTGGCGGAGC | Exon | 4129 | 4148 |
| 430 | AGCTCCGCCAATTGCTTGCC | 524 | GGCAAGCAATTGGCGGAGCT | Exon | 4128 | 4147 |
| 431 | AGGGCATTGCTCCAGCAGAC | 525 | GTCTGCTGGAGCAATGCCCT | Exon | 3597 | 3616 |
| 432 | CGCAGTTCCCCAACTGTGTG | 526 | CACACAGTTGGGGAACTGCG | Exon | 3545 | 3564 |
| 433 | GCGCAGTTCCCCAACTGTGT | 527 | ACACAGTTGGGGAACTGCGC | Exon | 3544 | 3563 |
| 434 | CTCTCGAGCCACCTTGAGGC | 528 | GCCTCAAGGTGGCTCGAGAG | Exon | 3348 | 3367 |
| 435 | ACTGTGCATCTCTCGAGCCA | 529 | TGGCTCGAGAGATGCACAGT | Exon | 3339 | 3358 |
| 436 | GACTGTGCATCTCTCGAGCC | 530 | GGCTCGAGAGATGCACAGTC | Exon | 3338 | 3357 |
| 437 | AGACTGTGCATCTCTCGAGC | 531 | GCTCGAGAGATGCACAGTCT | Exon | 3337 | 3356 |
| 438 | CAGACTGTGCATCTCTCGAG | 532 | CTCGAGAGATGCACAGTCTG | Exon | 3336 | 3355 |
| 439 | GCAGACTGTGCATCTCTCGA | 533 | TCGAGAGATGCACAGTCTGC | Exon | 3335 | 3354 |
| 440 | GCGGAAGTCCATGTTGTGCG | 534 | CGCACAACATGGACTTCCGC | Exon | 3252 | 3271 |
| 441 | CCGCGGAAGTCCATGTTGTG | 535 | CACAACATGGACTTCCGCGG | Exon | 3250 | 3269 |
| 442 | CGCTAGGTCACTGCCCAGGT | 536 | ACCTGGGCAGTGACCTAGCG | Exon | 3198 | 3217 |
| 443 | GCGCTAGGTCACTGCCCAGG | 537 | CCTGGGCAGTGACCTAGCGC | Exon | 3197 | 3216 |
| 444 | CGCGCTAGGTCACTGCCCAG | 538 | CTGGGCAGTGACCTAGCGCG | Exon | 3196 | 3215 |
| 445 | CCGGCCCTCAGCAAACTCCA | 539 | TGGAGTTTGCTGAGGGCCGG | Exon | 3168 | 3187 |
| 446 | TTCCATCCACCACTTCCGGC | 540 | GCCGGAAGTGGTGGATGGAA | Exon | 2662 | 2681 |
| 447 | GCTTCCATCCACCACTTCCG | 541 | CGGAAGTGGTGGATGGAAGC | Exon | 2660 | 2679 |
| 448 | AGCTTCCATCCACCACTTCC | 542 | GGAAGTGGTGGATGGAAGCT | Exon | 2659 | 2678 |
| 449 | CAGCTTCCATCCACCACTTC | 543 | GAAGTGGTGGATGGAAGCTG | Exon | 2658 | 2677 |
| 450 | TCAGCTTCCATCCACCACTT | 544 | AAGTGGTGGATGGAAGCTGA | Exon | 2657 | 2676 |
| 451 | ATCAGCTTCCATCCACCACT | 545 | AGTGGTGGATGGAAGCTGAT | Exon | 2656 | 2675 |
| 452 | CATCAGCTTCCATCCACCAC | 546 | GTGGTGGATGGAAGCTGATG | Exon | 2655 | 2674 |
| 453 | TCATCAGCTTCCATCCACCA | 547 | TGGTGGATGGAAGCTGATGA | Exon | 2654 | 2673 |
| 454 | GCTCATCAGCTTCCATCCAC | 548 | GTGGATGGAAGCTGATGAGC | Exon | 2652 | 2671 |
| 455 | GGCTCATCAGCTTCCATCCA | 549 | TGGATGGAAGCTGATGAGCC | Exon | 2651 | 2670 |
| 456 | GTGCCACCTCCATGCAGCAG | 550 | CTGCTGCATGGAGGTGGCAC | Exon | 2616 | 2635 |
| 457 | TGTGCCACCTCCATGCAGCA | 551 | TGCTGCATGGAGGTGGCACA | Exon | 2615 | 2634 |
| 458 | GTGTGCCACCTCCATGCAGC | 552 | GCTGCATGGAGGTGGCACAC | Exon | 2614 | 2633 |
| 459 | CGTGTGCCACCTCCATGCAG | 553 | CTGCATGGAGGTGGCACACG | Exon | 2613 | 2632 |
| 460 | GCGTGTGCCACCTCCATGCA | 554 | TGCATGGAGGTGGCACACGC | Exon | 2612 | 2631 |
| 461 | TGCGTGTGCCACCTCCATGC | 555 | GCATGGAGGTGGCACACGCA | Exon | 2611 | 2630 |
| 462 | CAGTCCACTTGCGTGCCCAC | 556 | GTGGGCACGCAAGTGGACTG | Exon | 2540 | 2559 |
| 463 | GGGCAGTCCACTTGCGTGCC | 557 | GGCACGCAAGTGGACTGCCC | Exon | 2537 | 2556 |
| 464 | GCCATGGGCAGTCCACTTGC | 558 | GCAAGTGGACTGCCCATGGC | Exon | 2532 | 2551 |
| 465 | CACAGGTCACCCCAGAAACT | 559 | AGTTTCTGGGGTGACCTGTG | Exon | 2380 | 2399 |
| 466 | CACCCACACTGTCTCGGCCT | 560 | AGGCCGAGACAGTGTGGGTG | Exon | 2271 | 2290 |
| 467 | GCACCCACACTGTCTCGGCC | 561 | GGCCGAGACAGTGTGGGTGC | Exon | 2270 | 2289 |
| 468 | TGTTGCCACCTCCCTGTACA | 562 | TGTACAGGGAGGTGGCAACA | Exon | 2209 | 2228 |
| 469 | TGTGTTGCCACCTCCCTGTA | 563 | TACAGGGAGGTGGCAACACA | Exon | 2207 | 2226 |
| 470 | CTGTGTTGCCACCTCCCTGT | 564 | ACAGGGAGGTGGCAACACAG | Exon | 2206 | 2225 |
| 471 | CTGGCACCCACCTGTGTTGC | 565 | GCAACACAGGTGGGTGCCAG | Exon | 2195 | 2214 |
| 472 | CACTCCACAGGCCATCCGGC | 566 | GCCGGATGGCCTGTGGAGTG | Exon | 1536 | 1555 |
| 473 | CCCACTCCACAGGCCATCCG | 567 | CGGATGGCCTGTGGAGTGGG | Exon | 1534 | 1553 |
| 474 | GACCCACTCCACAGGCCATC | 568 | GATGGCCTGTGGAGTGGGTC | Exon | 1532 | 1551 |
| 475 | CCACCTTTTACCTGCAGACC | 569 | GGTCTGCAGGTAAAAGGTGG | Exon | 1377 | 1396 |
| 476 | GCCACCTTTTACCTGCAGAC | 570 | GTCTGCAGGTAAAAGGTGGC | Exon | 1376 | 1395 |
| 477 | GGCCACCTTTTACCTGCAGA | 571 | TCTGCAGGTAAAAGGTGGCC | Exon | 1375 | 1394 |
| 478 | CTCCCCGCAGTACAGGACTC | 572 | GAGTCCTGTACTGCGGGGAG | Exon | 1313 | 1332 |
| 479 | CCTCCCCGCAGTACAGGACT | 573 | AGTCCTGTACTGCGGGGAGG | Exon | 1312 | 1331 |
| 480 | CCCTCCCCGCAGTACAGGAC | 574 | GTCCTGTACTGCGGGGAGGG | Exon | 1311 | 1330 |
| 481 | CTCCCCTCCCCGCAGTACAG | 575 | CTGTACTGCGGGGAGGGGAG | Exon | 1308 | 1327 |
| 482 | TCCTGCCCATCCCTCCATCC | 576 | GGATGGAGGGATGGGCAGGA | Exon | 1287 | 1306 |
| 483 | TTCCTGCCCATCCCTCCATC | 577 | GATGGAGGGATGGGCAGGAA | Exon | 1286 | 1305 |
| 484 | CCTTTCCTGCCCATCCCTCC | 578 | GGAGGGATGGGCAGGAAAGG | Exon | 1283 | 1302 |
| 485 | GCATGTGTCCAGAAGCAGTC | 579 | GACTGCTTCTGGACACATGC | Exon | 637 | 656 |
| 486 | TTGCGCACCGCCAGAAAGTG | 580 | CACTTTCTGGCGGTGCGCAA | Exon | 19 | 38 |

### ASO Synthesis

Exemplary ASOs were synthesized according to methods described in Example 1 (see http://eu.idtdna.com/pages/products/functional-genomics/antisense-oligos).

ASOs contained the following modification pattern:

X_{MS}X_{MS}X_{MS}X_{MS}X_{MS}X_{S}X_{S}X_{S}X_{S}X_{S}X_{S}X_{S}X_{S}X_{S}X_{S}X_{MS}X_{MS}X_{MS}X_{MS}X_{MS}

where “X” represents any nucleotide; a “M” represents a 2′-O-MOE group; and

an “S” represents a phosphorothioate bond.

### ASO Transfection

Mouse 3T3 cells (ATCC CRL-1658) were grown at 37° C. with 5% (v/v) CO₂ in Dulbecco's Modified Eagle Medium (DMEM) supplemented with 10% fetal bovine serum (FBS).

1.8 or 6 μl of each ASO (100 μM) was added to 8 μL of DharmaFECT 1 (horizondiscovery, T-2001-02), 1 ml Opti-MEM media (Thermo Fisher Scientific, Cat #11058021), and mixed well. ASOs were tested at two final concentrations 100 nM and 30 nM. The mixture was incubated for 15 min at room temperature. 5 mL of 5×10⁴/mL 3T3 cells were added to the mixture and mixed well. The cells and ASO mixture were seeded (2 mL per well) in a 6-well plate and grown for one day.

### RT-PCR Protocol

The following RT-PCR protocol was performed:
1. Transfer 3T3 cells to eppendorf tubes, pellet cells by centrifuging 500 g 5 min, and remove remaining media. Wash the cell pellet with 1× Phosphate Buffered Saline (PBS). Add 1 mL of trizol solution (Thermo Fisher Scientific, Cat #15596026) to the 3T3 cell pellet. Resuspend the cells completely by vortexing.
2. Incubate tubes at room temperature for 5 min.
3. Add 200 μl chloroform. Vortex for 10 seconds.
4. Incubate for 5 min at room temperature to permit nucleoprotein dissociation.
5. Centrifuge the tubes at 12,000×g for 15 min at 4° C.
6. Transfer the upper phase to a new microcentrifuge tube without disturbing the interphase.
7. Add 500 μL of isopropanol to the sample, invert 5 times to mix and incubate 30 min at −20° C.
8. Centrifuge at a minimum of 12,000×g for 15 min at 4° C. and remove the supernatant. The RNA will appear as a white pellet in the tube.
9. Wash the pellet with 500 μL of ice-cold 75% ethanol (made with deionized, diethylpyrocarbonate (DEPC) treated and 0.22 μm membrane-filtered H₂O).
10. Remove the ethanol, air dry for 5 minutes at room temperature (do not completely dry the RNA) and resuspend the RNA in 100 μL of DEPC-H₂O.
11. Incubate 5 μg RNA with 1 unit of turbo DNase (TURBO DNA-free Kit, Thermo Fisher Scientific, Cat #AM1907) at room temperature for 15 minutes and stop reaction following the manufacturer's instructions.
12. Purify RNA using a Quick-RNA miniprep kit (ZYMO, Cat #R1055).
13. Perform reverse transcription using 1 g RNA and the iScript cDNA Synthesis Kit (Biorad, 170-8891).
14. Dilute 20 μl of cDNA to a final volume of 200 μl using H₂O. The cDNA is now ready for PCR quantification.

The iTaq Universal SYBR Green Supermix (Bio-Rad, Cat ##1725121) was used together with primers ordered from Eurofins genomics (shown below in Table 10) to detect expression of mouse POLRMT and mouse 18S rRNA genes. Quantification was performed using the Biorad CFX96 PCR system. The reaction mixtures in each well contained 1 μL forward primer (conc. 5 μM) and 1 μL reverse primer (conc. 5 μM), 2 μL cDNA, 9.5 μL H₂O and 12.5 μL SYBR supermix.

TABLE 10

| | | | SEQ ID | |
|---|---|---|---|---|
| Primer | Supplier | Sequence | NO: | Storage |
| mPOLRMT_F2 | Eurofins | CCCACGATGA | 583 | −20° C. |
| | | ACGAGGTATG | | |
| mPOLRMT_R2 | Eurofins | CGAGGACTTTA | 584 | −20° C. |
| | | GGGACTTGATG | | |
| Ms_18S_F1 | Eurofins | GCCGCTAGAGGT | 585 | −20° C. |
| | | GAAATTCT | | |
| Ms_18S_R1 | Eurofins | TCGGAACTACGA | 586 | −20° C. |
| | | CGGTATCT | | |

### Results—POLRMT Expression

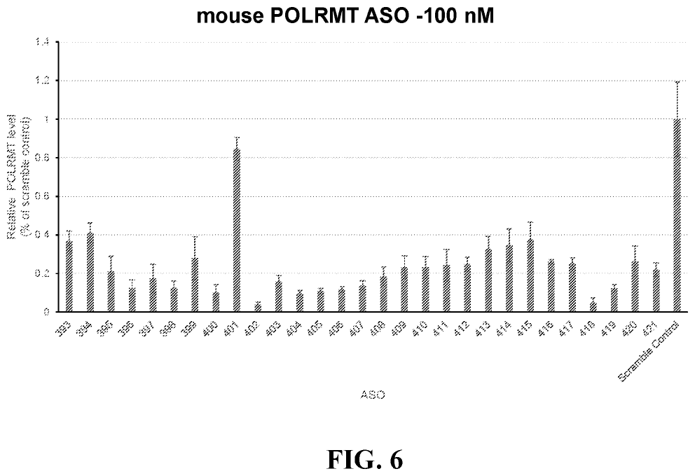

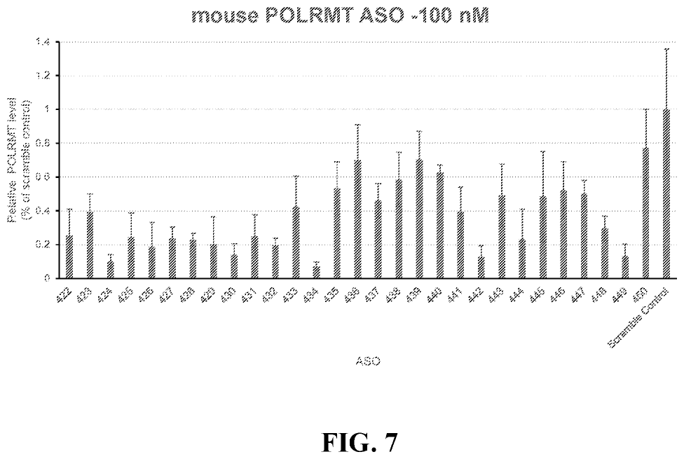

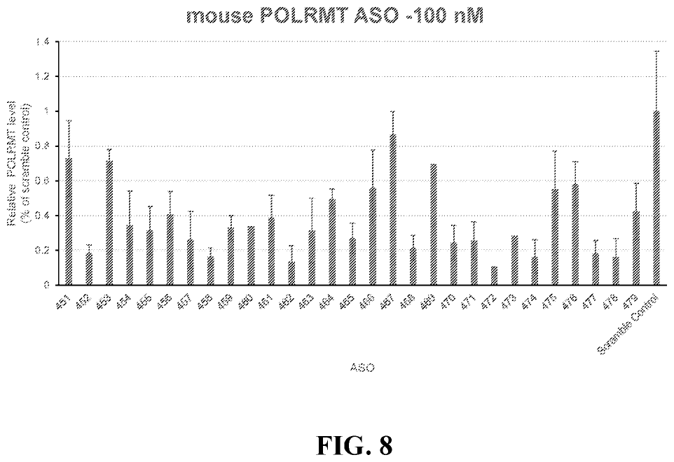

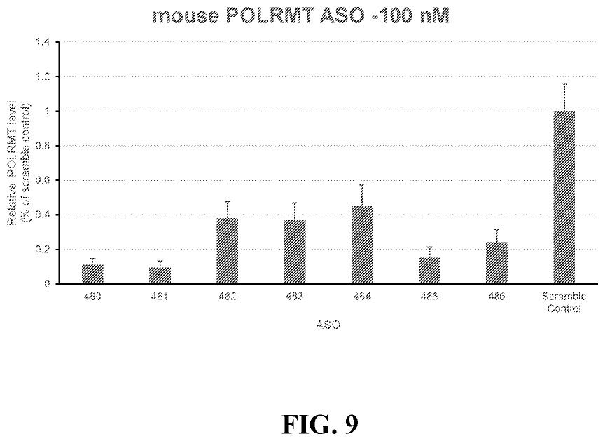

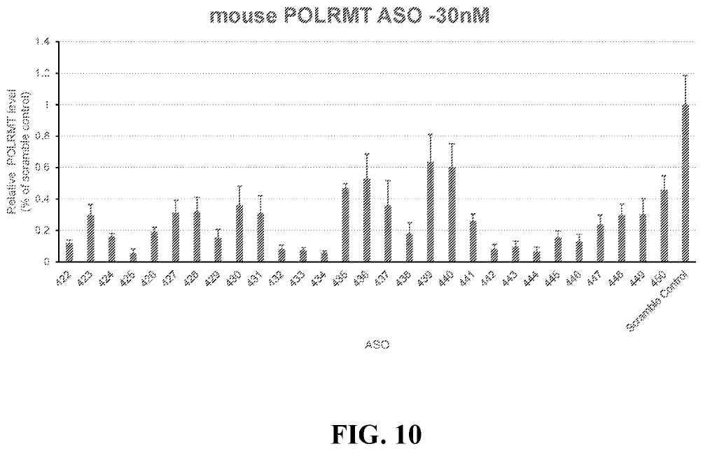

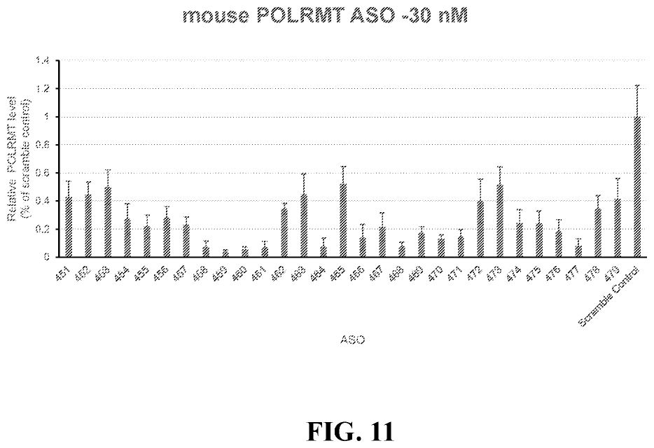

POLRMT expression in 3T3 cells transfected with the exemplary ASOs at 100 nM and 30 nM are shown in FIGS. 6-9 and FIGS. 10-11, respectively. A scrambled ASO (not a perfect match to any mouse transcripts) was used as a control in each PCR plate. All results were normalized by 18S expression.

Of the ASOs tested at 100 nM concentration (i.e., ASOs represented in SEQ ID NOs: 393-486), all of the ASOs showed some inhibition of POLRMT expression (i.e., decreased expression relative to the control). ASOs represented in SEQ ID NOs: 396, 398, 400, 402, 404-407, 418, 419, 424, 430, 434, 442, 449, 462, 472, 480, and 481, showed strong inhibition of POLRMT expression at 100 nM.

Of the ASOs tested at 30 nM concentration (i.e., ASOs represented in SEQ ID NOs: 422-479), all ASOs showed some inhibition of POLRMT expression (i.e., decreased expression relative to the control). ASOs represented in SEQ ID NOs: 422, 425, 432-434, 442-444, 446, 458-461, 464, 466, 468, 470, 471, and 477 showed strong inhibition of POLRMT expression at 30 nM. ASOs represented in SEQ ID NOs: 434 and 442 showed strong inhibition of POLRMT expression at both 100 nM and 30 nM concentration.

These results show that the targeting certain regions of mouse POLRMT with ASO leads to different inhibition activity.

### Example 4: Design and Testing of Exemplary Oligonucleotides Cross-Reactive in Human and Mouse

This Example demonstrates exemplary oligonucleotides capable of inhibiting POLRMT expression in mouse and human cells and identifies regions within the POLRMT transcript that, when targeted by oligonucleotides described herein, are effective in inhibiting POLRMT expression.

### Oligonucleotides

Oligonucleotides were designed and synthesized to target different regions of the POLRMT RNA transcript (SEQ ID NO: 205) and target regions on the POLRMNT mRNA transcript are characterized by corresponding region within the POLRMT gene sequence (represented in Reference No. NG_023049.1 and in SEQ ID NO: 1). Oligonucleotides were designed by selecting 16, 18, and 20-mers that target various regions of human POLRMT.

Exemplary oligonucleotide sequences, the POLRMT target region sequence within the POLRMT RNA transcript are shown in Table 11 below.

TABLE 11

| ASO | | | POLRMT RNA Sequence |
|---|---|---|---|
| SEQ | | SEQ | (5′ to 3′) |
| ID | ASO sequence | ID | (represented as a |
| NO: | (5′ to 3′) | NO: | DNA sequence) |
| 587 | GAAGTCCATGTTGTGCGGCA | 656 | TGCCGCACAACATGGACTTC |
| 588 | CGGAAGTCCATGTTGTGCGG | 657 | CCGCACAACATGGACTTCCG |
| 589 | CGCGGAAGTCCATGTTGTGC | 658 | GCACAACATGGACTTCCGCG |
| 590 | GCCGCGGAAGTCCATGTTGT | 659 | ACAACATGGACTTCCGCGGC |
| 591 | GGCCGCGGAAGTCCATGTTG | 660 | CAACATGGACTTCCGCGGCC |
| 592 | CGGCCGCGGAAGTCCATGTT | 661 | AACATGGACTTCCGCGGCCG |
| 593 | GCGGCCGCGGAAGTCCATGT | 662 | ACATGGACTTCCGCGGCCGC |
| 594 | TGCGGCCGCGGAAGTCCATG | 663 | CATGGACTTCCGCGGCCGCA |
| 595 | GTGCGGCCGCGGAAGTCCAT | 664 | ATGGACTTCCGCGGCCGCAC |
| 596 | CTGCCCAGGTGGTTGAAGTG | 665 | CACTTCAACCACCTGGGCAG |
| 597 | TCTGCTTCACCACCTTGCGG | 666 | CCGCAAGGTGGTGAAGCAGA |
| 598 | GTCTGCTTCACCACCTTGCG | 667 | CGCAAGGTGGTGAAGCAGAC |
| 599 | ATGAAGTTGGGCGGGAAGCC | 668 | GGCTTCCCGCCCAACTTCAT |
| 600 | GATGAAGTTGGGCGGGAAGC | 669 | GCTTCCCGCCCAACTTCATC |
| 601 | GGATGAAGTTGGGCGGGAAG | 670 | CTTCCCGCCCAACTTCATCC |
| 602 | TGGATGAAGTTGGGCGGGAA | 671 | TTCCCGCCCAACTTCATCCA |
| 603 | GTGGATGAAGTTGGGCGGGA | 672 | TCCCGCCCAACTTCATCCAC |
| 604 | GAGTGGATGAAGTTGGGCGG | 673 | CCGCCCAACTTCATCCACTC |
| 605 | GAGTGGATGAAGTTGGGCGG | 674 | CCGCCCAACTTCATCCACTC |
| 606 | ATCATGTGGGAGGAGTCCAG | 675 | CTGGACTCCTCCCACATGAT |
| 607 | CATCATGTGGGAGGAGTCCA | 676 | TGGACTCCTCCCACATGATG |
| 608 | GCATCATGTGGGAGGAGTCC | 677 | GGACTCCTCCCACATGATGC |
| 609 | AGGCCCTTCCTGTAGCAGTG | 678 | CACTGCTACAGGAAGGGCCT |
| 610 | CAGGCCCTTCCTGTAGCAGT | 679 | ACTGCTACAGGAAGGGCCTG |
| 611 | TCAGGCCCTTCCTGTAGCAG | 680 | CTGCTACAGGAAGGGCCTGA |
| 612 | CCCAGCCAAGCATCACGG | 681 | CCGTGATGCTTGGCTGGG |
| 613 | GCCCAGCCAAGCATCACG | 682 | CGTGATGCTTGGCTGGGC |
| 614 | AGTCCATGTTGTGCGGCA | 683 | TGCCGCACAACATGGACT |
| 615 | AAGTCCATGTTGTGCGGC | 684 | GCCGCACAACATGGACTT |
| 616 | GAAGTCCATGTTGTGCGG | 685 | CCGCACAACATGGACTTC |
| 617 | GGAAGTCCATGTTGTGCG | 686 | CGCACAACATGGACTTCC |
| 618 | CGGAAGTCCATGTTGTGC | 687 | GCACAACATGGACTTCCG |
| 619 | GCGGAAGTCCATGTTGTG | 688 | CACAACATGGACTTCCGC |
| 620 | CGCGGAAGTCCATGTTGT | 689 | ACAACATGGACTTCCGCG |
| 621 | CCGCGGAAGTCCATGTTG | 690 | CAACATGGACTTCCGCGG |
| 622 | GCCGCGGAAGTCCATGTT | 691 | AACATGGACTTCCGCGGC |
| 623 | GGCCGCGGAAGTCCATGT | 692 | ACATGGACTTCCGCGGCC |
| 624 | CGGCCGCGGAAGTCCATG | 693 | CATGGACTTCCGCGGCCG |
| 625 | GCGGCCGCGGAAGTCCAT | 694 | ATGGACTTCCGCGGCCGC |
| 626 | TGCGGCCGCGGAAGTCCA | 695 | TGGACTTCCGCGGCCGCA |
| 627 | GTGCGGCCGCGGAAGTCC | 696 | GGACTTCCGCGGCCGCAC |
| 628 | GCCCAGGTGGTTGAAGTG | 697 | CACTTCAACCACCTGGGC |
| 629 | TGCCCAGGTGGTTGAAGT | 698 | ACTTCAACCACCTGGGCA |
| 630 | CTGCCCAGGTGGTTGAAG | 699 | CTTCAACCACCTGGGCAG |
| 631 | TGCTTCACCACCTTGCGG | 700 | CCGCAAGGTGGTGAAGCA |
| 632 | CTGCTTCACCACCTTGCG | 701 | CGCAAGGTGGTGAAGCAG |
| 633 | TCTGCTTCACCACCTTGC | 702 | GCAAGGTGGTGAAGCAGA |
| 634 | GTCTGCTTCACCACCTTG | 703 | CAAGGTGGTGAAGCAGAC |
| 635 | GAACATCTCCTGTAGACT | 704 | AGTCTACAGGAGATGTTC |
| 636 | GAAGTTGGGCGGGAAGCC | 705 | GGCTTCCCGCCCAACTTC |
| 637 | TGAAGTTGGGCGGGAAGC | 706 | GCTTCCCGCCCAACTTCA |
| 638 | ATGAAGTTGGGCGGGAAG | 707 | CTTCCCGCCCAACTTCAT |
| 639 | GATGAAGTTGGGCGGGAA | 708 | TTCCCGCCCAACTTCATC |
| 640 | GGATGAAGTTGGGCGGGA | 709 | TCCCGCCCAACTTCATCC |
| 641 | TGGATGAAGTTGGGCGGG | 710 | CCCGCCCAACTTCATCCA |
| 642 | GTGGATGAAGTTGGGCGG | 711 | CCGCCCAACTTCATCCAC |
| 643 | AGTGGATGAAGTTGGGCG | 712 | CGCCCAACTTCATCCACT |
| 644 | GAGTGGATGAAGTTGGGC | 713 | GCCCAACTTCATCCACTC |
| 645 | CATGTGGGAGGAGTCCAG | 714 | CTGGACTCCTCCCACATG |
| 646 | TCATGTGGGAGGAGTCCA | 715 | TGGACTCCTCCCACATGA |
| 647 | ATCATGTGGGAGGAGTCC | 716 | GGACTCCTCCCACATGAT |
| 648 | CATCATGTGGGAGGAGTC | 717 | GACTCCTCCCACATGATG |
| 649 | GCATCATGTGGGAGGAGT | 718 | ACTCCTCCCACATGATGC |
| 650 | AGCATCATGTGGGAGGAG | 719 | CTCCTCCCACATGATGCT |
| 651 | GCCCTTCCTGTAGCAGTG | 720 | CACTGCTACAGGAAGGGC |
| 652 | GGCCCTTCCTGTAGCAGT | 721 | ACTGCTACAGGAAGGGCC |
| 653 | AGGCCCTTCCTGTAGCAG | 722 | CTGCTACAGGAAGGGCCT |
| 654 | CAGGCCCTTCCTGTAGCA | 723 | TGCTACAGGAAGGGCCTG |
| 655 | TCAGGCCCTTCCTGTAGC | 724 | GCTACAGGAAGGGCCTGA |

A subset of the oligonucleotides that have a target region sequence identical to the corresponding region on the mouse POLRMVT transcript were selected for testing in human 143B cells and mouse 3T3 cells. A schematic of the 13 selected oligonucleotide sequences and their respective target regions on the POLRMT transcript is shown in FIG. 12 and Table 12 below.

TABLE 12

| | | | POLRMT RNA Sequence | Location | Location |
|---|---|---|---|---|---|
| SEQ | | SEQ | (5′ to 3′) | (human) | (mouse) |
| ID | ASO Sequence | ID | (represented as a | POLRMT | POLRMT |
| NO: | (5′ to 3′) | NO: | DNA sequence) | mRNA | mRNA |
| 592 | CGGCCGCGGAAGTCCATGTT | 661 | AACATGGACTTCCGCGGCCG | 2420-2439 | 2329-2348 |
| 594 | TGCGGCCGCGGAAGTCCATG | 663 | CATGGACTTCCGCGGCCGCA | 2422-2441 | 2331-2350 |
| 597 | TCTGCTTCACCACCTTGCGG | 666 | CCGCAAGGTGGTGAAGCAGA | 2983-3002 | 3240-3259 |
| 598 | GTCTGCTTCACCACCTTGCG | 667 | CGCAAGGTGGTGAAGCAGAC | 2984-3003 | 3241-3260 |
| 612 | CCCAGCCAAGCATCACGG | 681 | CCGTGATGCTTGGCTGGG | 822-839 | 734-751 |
| 613 | GCCCAGCCAAGCATCACG | 682 | CGTGATGCTTGGCTGGGC | 823-840 | 735-752 |
| 623 | GGCCGCGGAAGTCCATGT | 692 | ACATGGACTTCCGCGGCC | 2421-2438 | 2330-2347 |
| 624 | CGGCCGCGGAAGTCCATG | 693 | CATGGACTTCCGCGGCCG | 2422-2439 | 2331-2348 |
| 625 | GCGGCCGCGGAAGTCCAT | 694 | ATGGACTTCCGCGGCCGC | 2423-2440 | 2332-2349 |
| 626 | TGCGGCCGCGGAAGTCCA | 695 | TGGACTTCCGCGGCCGCA | 2424-2441 | 2333-2350 |
| 632 | CTGCTTCACCACCTTGCG | 701 | CGCAAGGTGGTGAAGCAG | 2984-3001 | 3241-3258 |
| 633 | TCTGCTTCACCACCTTGC | 702 | GCAAGGTGGTGAAGCAGA | 2985-3002 | 3242-3259 |
| 634 | GTCTGCTTCACCACCTTG | 703 | CAAGGTGGTGAAGCAGAC | 2986-3003 | 3243-3260 |

### Oligonucleotide Synthesis

Exemplary oligonucleotides were synthesized according to methods described in Example 1 (see https://eu.idtdna.com/pages/products/funtional-genomics/antisense-oligos).

Oligonucleotides contain the following modification pattern:

X_{MS}X_{MS}X_{MS}X_{MS}X_{MS}X_{S}X_{S}X_{S}X_{S}X_{S}X_{S}X_{S}X_{S}X_{S}X_{S}X_{MS}X_{MS}X_{MS}X_{MS}X_{MS}
(for 20-mers)
and
X_{MS}X_{MS}X_{MS}X_{MS}X_{MS}X_{S}X_{S}X_{S}X_{S}X_{S}X_{S}X_{S}X_{S}X_{MS}X_{MS}X_{MS}X_{MS}X_{MS}
(for 18-mers)

where “X” represents any nucleotide; a “M” represents a 2′-O-MOE group; and an “S” represents a phosphorothioate bond.

TABLE 13
Exemplary Modified Oligonucleotides (where
“s” represents a phosphorothioate bond
and “M” represents a 2′-O-MOE group)

| Oligonucleotide | Modified | |
|---|---|---|
| Base Sequence | Oligonucleotide | |
| SEQ ID NO: | SEQ ID NO: | Oligonucleotide Sequence (5′ to 3′) |
| 592 | 728 | C_{MS}G_{MS}G_{MS}C_{MS}C_{MS}G_{S}C_{S}G_{S}G_{S}A_{S}A_{S}G_{S}T_{S}C_{S}C_{S}A_{MS}T_{MS}G_{MS}T_{MS}T_{MS} |
| 594 | 729 | T_{MS}G_{MS}C_{MS}G_{MS}G_{MS}C_{S}C_{S}G_{S}C_{S}G_{S}G_{S}A_{S}A_{S}G_{S}T_{S}C_{MS}C_{MS}A_{MS}T_{MS}G_{MS} |
| 597 | 730 | T_{MS}C_{MS}T_{MS}G_{MS}C_{MS}T_{S}T_{S}C_{S}A_{S}C_{S}C_{S}A_{S}C_{S}C_{S}T_{S}T_{MS}G_{MS}C_{MS}G_{MS}G_{MS} |
| 598 | 731 | G_{MS}T_{MS}C_{MS}T_{MS}G_{MS}C_{S}T_{S}T_{S}C_{S}A_{S}C_{S}C_{S}A_{S}C_{S}C_{S}T_{MS}T_{MS}G_{MS}C_{MS}G_{MS} |
| 612 | 732 | C_{MS}C_{MS}C_{MS}A_{MS}G_{MS}C_{S}CA_{S}A_{S}GsC_{S}A_{S}TsC_{MS}A_{MS}C_{MS}G_{MS}G_{MS} |
| 613 | 733 | G_{MS}C_{MS}C_{MS}C_{MS}A_{MS}G_{S}C_{S}C_{S}A_{S}A_{S}G_{S}C_{S}A_{S}T_{MS}C_{MS}A_{MS}C_{MS}G_{MS} |
| 623 | 734 | G_{MS}G_{MS}C_{MS}C_{MS}G_{MS}C_{S}G_{S}G_{S}A_{S}A_{S}G_{S}T_{S}C_{S}C_{MS}A_{MS}T_{MS}G_{MS}T_{MS} |
| 624 | 735 | C_{MS}G_{MS}G_{MS}C_{MS}C_{MS}G_{S}C_{S}G_{S}G_{S}A_{S}A_{S}G_{S}T_{S}C_{MS}C_{MS}A_{MS}T_{MS}G_{MS} |
| 625 | 736 | G_{MS}C_{MS}G_{MS}G_{MS}C_{MS}C_{S}G_{S}C_{S}G_{S}G_{S}A_{S}A_{S}G_{S}T_{MS}C_{MS}C_{MS}A_{MS}T_{MS} |
| 626 | 737 | T_{MS}G_{MS}C_{MS}G_{MS}G_{MS}C_{S}C_{S}G_{S}C_{S}G_{S}G_{S}A_{S}A_{S}G_{MS}T_{MS}C_{MS}C_{MS}A_{MS} |
| 632 | 738 | C_{MS}T_{MS}G_{MS}C_{MS}T_{MS}T_{S}C_{S}A_{S}C_{S}C_{S}A_{S}C_{S}C_{S}T_{MS}T_{MS}G_{MS}C_{MS}G_{MS} |
| 633 | 739 | T_{MS}C_{MS}T_{MS}G_{MS}C_{MS}T_{S}T_{S}C_{S}A_{S}C_{S}C_{S}A_{S}C_{S}C_{MS}T_{MS}T_{MS}G_{MS}C_{MS} |
| 634 | 740 | G_{MS}T_{MS}C_{MS}T_{MS}G_{MS}C_{S}T_{S}T_{S}C_{S}A_{S}C_{S}C_{S}A_{S}C_{MS}C_{MS}T_{MS}T_{MS}G_{MS} |

### (i) Dose Response in Human 143B and Mouse 3T3 Cells

### Oligonucleotide Transfection

Human 143B cells (ATCC #8303) and mouse 3T3 cells (ATCC #1658) were grown at 37° C. with 5% (v/v) CO₂ in Dulbecco's Modified Eagle Medium (DMEM) supplemented with 10% fetal bovine serum (FBS).

Each modified oligonucleotide was added to DharmaFECT 1 (horizondiscovery, T-2001-02) and Opti-MEM media (Thermo Fisher Scientific, Cat #11058021), and mixed well to a final concentration of 100 nM ASO. Serial dilutions (1.2) were performed for each oligonucleotide resulting in a total of 7 concentrations (100 nM, 50 nM, 25 nM, 12.5 nM, 6.25 nM, 3.125 nM, and 1.5625 nM) of each oligonucleotide to be tested in each cell type. The mixture was incubated for 15 min at room temperature. 5×104/mL 143B cells or 4×104/mL 3T3 cells were added to the oligo/DharmaFECT/Media mixture and mixed well.

The cells were then seeded in a 6-well plate and grown for one day. Cells were harvested for RT-PCR to assess POLRMT expression and cell viability using Celltiter fluor (Promega Cat #G6080).

### RT-PCR Protocol

The RT-PCR protocol is performed according to Example 1 in order to measure relative POLRMT mRNA expression levels. Expression is shown relative to vehicle control.

### Results

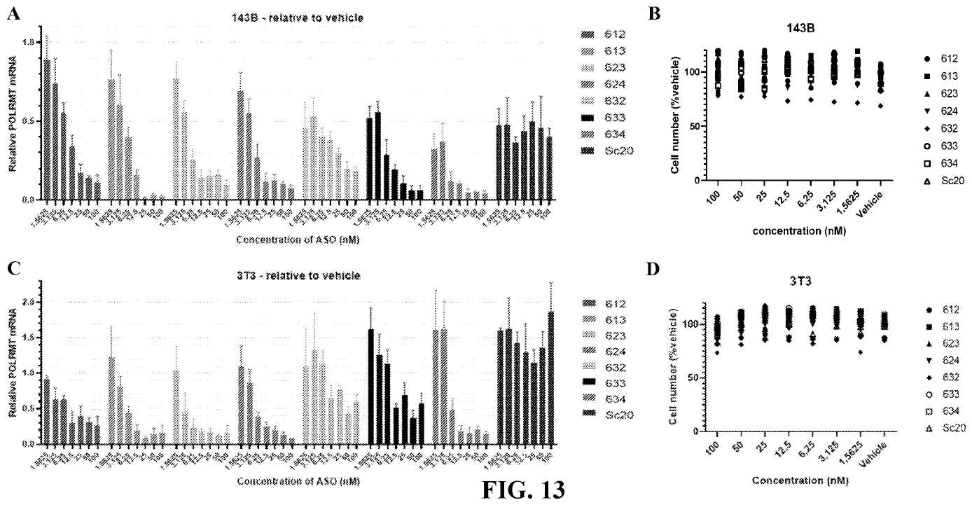

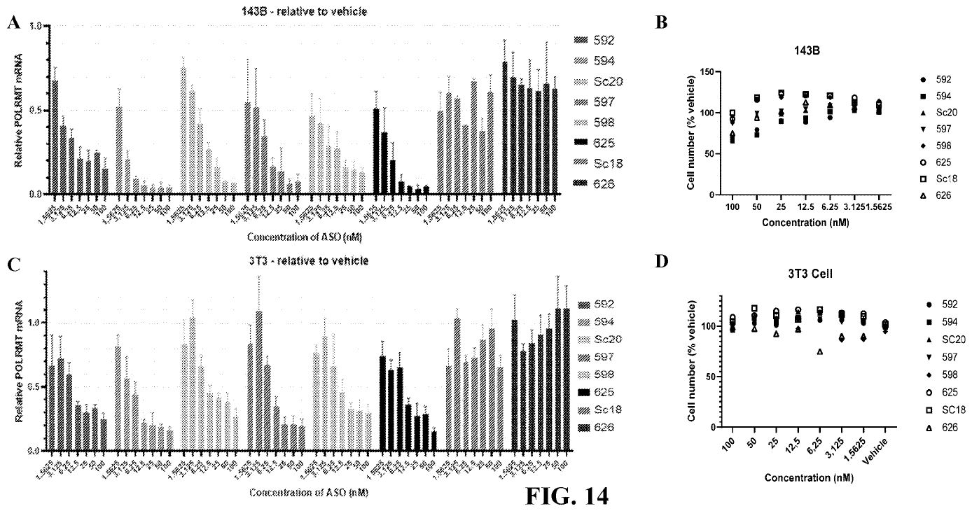

POLRMT expression in both mouse 3T3 and human 143B cells transfected with each modified oligonucleotide is shown in FIG. 13 (for modified oligonucleotides with unmodified base sequence represented in SEQ ID NOs: 612, 613, 623, 624, 632, 633, and 634) and FIG. 14 (for modified oligonucleotides with unmodified base sequence represented in SEQ ID NOs: 592, 594, 597, 598, 625, and 626). Results show that POLRMT expression was inhibited in a dose-dependent manner for each oligonucleotide tested, confirming that the selected oligonucleotides are indeed cross-reactive in human and mouse cells (i.e., can target the mouse and human POLRMT transcript and are capable of POLRMT knockdown in mouse and human cells).

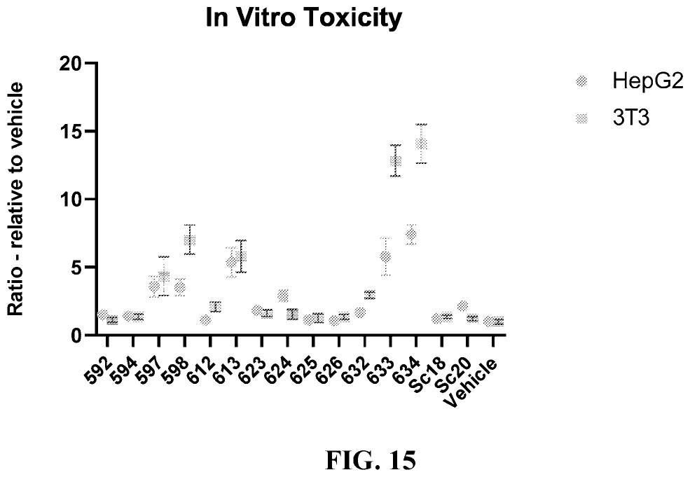

Additionally, FIG. 14, panel (B) and (D) and FIG. 15, panel (B) and (D) shows that the cells transfected with oligonucleotides remained viable.

### (ii) Toxicity in HepG2 and 3T3 Cells

### Oligonucleotide Transfection

HepG2 cells (ATCC #HB-8065) and mouse 3T3 cells (ATCC #1658) were grown at 37° C. with 5% (v/v) CO₂ in Dulbecco's Modified Eagle Medium (DMEM) supplemented with 10% fetal bovine serum (FBS).

Modified oligonucleotides were added to DharmaFECT 1 (horizondiscovery, T-2001-02) and Opti-MEM media (Thermo Fisher Scientific, Cat #11058021), and mixed well, the mixture was incubated for 15 min at room temperature. After incubation the mixture was added to 1×10⁵/mL HepG2 cells or 4×10⁴/mL 3T3 cells to a final concentration of oligonucleotide at 100 nM. Two scrambled ASOs and a vehicle treatment were used as controls.

The cells were seeded to 96-well plate on day 1, transfected with oligonucleotide on day 2, and cell viability and RT-qPCR were carried out on day 3.

### Caspase-Glo 3/7 Assay

In order to assess in vitro toxicity of the exemplary oligonucleotides on HepG2 and 3T3 cells, a Caspase-Glo 3/7 assay was performed (Promega Cat. #G8090).

(iii) Activity in HepG2 Cells

### Oligonucleotide Transfection in HepG2

HepG2 cells (ATCC #HB-8065) were grown at 37° C. with 5% (v/v) CO₂ in Dulbecco's Modified Eagle Medium (DMEM) supplemented with 10% fetal bovine serum (FBS).

Modified oligonucleotides were added to DharmaFECT 1 (horizondiscovery, T-2001-02) and Opti-MEM media (Thermo Fisher Scientific, Cat #11058021), and mixed well, the mixture was incubated for 15 min at room temperature. After incubation the mixture was added to 1×10⁵/mL HepG2 cells to a final concentration of oligonucleotide at 100 nM.

The cells and oligonucleotide mixture were seeded in a 6-well plate and grown for one day. Cells were then harvested for either RT-PCR to measure PORLMT expression or to assess cell viability measured using Celltiter fluor (Promega Cat #G6080).

### RT-PCR Protocol

The RT-PCR protocol is performed according to Example 1 in order to measure relative POLRMT mRNA expression levels.

### Results

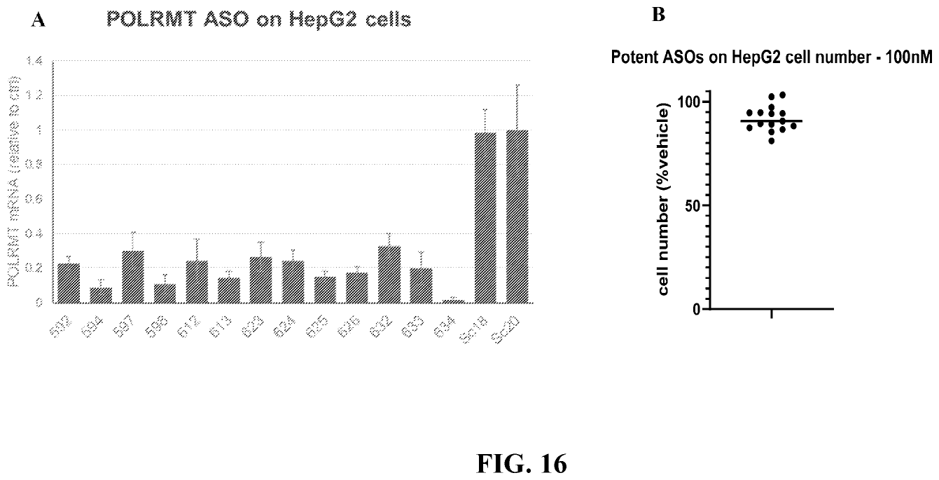

Results from the expression and toxicity assays are shown in Table 14 below. Exemplary modified oligonucleotides having nucleotide sequences represented in SEQ ID NOs: 592, 594, 612, 623, 625, 626, and 632 were well tolerated in HepG2 and mouse 3T3 cells and showed low toxicity (see FIG. 15 and FIG. 16, Panel B). Exemplary modified oligonucleotides having nucleotide sequences represented in SEQ ID NOs: 594, 612 and 632 showed the best inhibition of POLRMT expression in HepG2 cells (see Table 12 and FIG. 16, Panel A) and also had low toxicity HepG2 cells and mouse 3T3 cells (see FIG. 15 and FIG. 16, Panel B).

This data suggested that there are three potential “hotspot” regions along the POLRMT transcript that can be targeted to inhibit POLRMT expression. These regions were identified within exons represented by Ensemble IDs ENSE00000655271, ENSE00000655279, and ENSE00000655283. The hotspot regions include spans of nucleotides within the PORLMT human and mouse transcript that comprise SEQ ID NO: 725 [CAACGCCGTGATGCTTGGCTGGGCGCGGC](“hotspot 1”), SEQ ID NO: 726 [CGCACAACATGGACTTCCGCGGCCGCACCTAC](“hotspot 2”) and SEQ ID NO: 727 [ATCACCCGCAAGGTGGTGAAGCAGACGGTGA](“hotspot 3”). The first, second and third hotspot regions correspond to positions 817-845, 2415-2446, and 2978-3008 of the human POLRMT mRNA transcript (SEQ ID NO: 205) and positions 729-756, 2324-2355, and 3235-3265 of the mouse PORLMT transcript (SEQ ID NO: 581), respectively. Additionally, the most effective oligonucleotides targeting these regions consisted of various lengths (18 and 20 nucleotides).

TABLE 14

| | | | | | Efficiency on |
|---|---|---|---|---|---|
| SEQ ID | Length | | | | HepG2 (single |
| NO: | 18 nt | 20 nt | Hotspot | Toxicity | dose - 100 nM) |
| 592 | | X | 2 | Low | 0.229 |
| 594 | | X | 2 | Low | 0.089 |
| 597 | | X | 3 | High | 0.301 |
| 598 | | X | 3 | High | 0.107 |
| 612 | X | | 1 | Low | 0.242 |
| 613 | X | | 1 | High | 0.146 |
| 623 | X | | 2 | Low | 0.265 |
| 624 | X | | 2 | High* | 0.243 |
| 625 | X | | 2 | Low | 0.152 |
| 626 | X | | 2 | Low | 0.176 |
| 632 | X | | 3 | Low** | 0.330 |
| 633 | X | | 3 | High | 0.220 |
| 634 | X | | 3 | High | 0.016 |

*Showed a slightly higher toxicity in HepG2, but not in 3T3 cells
**Showed high in toxicity in 3T3, but not in HepG2.

EXEMPLARY SEQUENCES
Wild-type human POLRMT gene sequence (corresponding to the whole POLRMT gene)
(NCBI Accession Number: NG_023049.1)
(SEQ ID NO: 1)
ACCCTCCACGGAGCACGCTGGGCAGAGGGCGCGGCACCAGACACTGAAGAAATGTCCACCGCCTTCTCGC
CAGCAGGAGCCCAGGTCTTCCCTTCTTAGAGTGTCCGCCCCCCACCGCGGAAGGGTGATCTGAGGGCCTT
GTGAGAAATGATGGGGATGGGACCCACAGCGGTGAGACTGGCCCCACCCCAGGCGTCTGAGTTTCTTTAA
TGACACCTCGGAAACATCACTCATATACCACACAATTCACCCACTTACACTGTACAGTTCAGTGATTTTA
TTTTATTATTATTTCTTTGAGACGGAGTCTGGCTCTGTCACCCAGACTGGAGTGCAGTGGTGCGATCTCG
GCTCACTGCAAACTCTGCCTCCCTGGTTCAAGCGATTCTCCTGCCTCAGCCTCCTGAGTAGCTGGGATTA
CAGGCACCTGCCACCACACCCAGCTAATTTTTGTATTTTTAGTAGAGACAGAGTTTTACCATGTTGGCCA
GGCTGGTCTCAAACTCCTGACCTCGTGATCTACCTGCTTCTGCCTCCCAAAGTGCTGGGATTACAGGCAT
GAGCCACTGCACCCAGCCAGTTCAGTGATTTTAGTACCTCGTAGAGTTGTGCAACCACCACGACTGTCTA
GTTCCAGAAGATTCCATCACCGCAAGAAGAAGCCCCATCAGCTGTCACTCATCCCCTCTGCCAGCCCCCG
GCACCCACACATCCCCTTCCTGCCTCTGTGGATGGGCCTGTTGTGGACATTTCATATAAAGGGGATCACA
CACTGTGTGTCCTTCTGTGTCTGGCGTCTTTCACTGAGCATGACATCCTCAAGGTGCATCCGCGCTGTGG
CTGGGGCAGAGCCTTGCTCCTTTTCACGCCTGTAATCCCAGCTACTCAGGAGGCTGAGGCAGGAGAATCT
ATACAACCCAGGAGGCGGAGGTTGCAATGAGCTGAGATCATGCCACTGCCCTCCAGCCTGGGCGACAGAG
CGAGACTCTGTCTCAAACAAAAACAACAACAACAAACACCGATCAGGGCTGGGCTGGCGCACGCTTTGTA
TTGAGTGGTGTCTTCCTCTTTGTAGCCTCTGTGGTTGGCCCAAAGGTAAGTCCCCACCCTGATCCCTAGA
ATGTGACATTTGCCTTATTTGGAAAAAGAGTCTCAATATATTGACACCATGAAGATCTGAACGATATGGA
GAAGATTGTCCTGGGGCATCCTAAACGCGGTCACAATACGGAGACAGGAGGAGATTTCACACAGGCAGAC
GAGGAGGCTGCAGTGCGACCACAGAGACCGAGACGGGAGTGATGTGGCCGCAGGCCAAGCACACCTGGAG
CCTTCAAAAGCTGGGAGAGGCAGGAAGGAGCCCCAGGGCCTCTGGAGGGAGCGAGGCCTCCACACACCTT
GACTTCAGCCCCGCAGTCCTGATTTTGGACTTTTCATTTCCAGAAATGTAAGAAAATGAACGTTGCTTTA
AGGCACCGAGTTTGTGGTAATTTGTCACAGGGGCTCCAGGGACCTGGGCCATCCCCTTCCATCTAGCGAT
TCTCGACTCTGTGGGGTTTGCTTGGTTGGGTTTTGTTTTGTTTTGTTTTTAGACAGAGTCTCGCTCTGTC
GCCCAGGCCGGAGGGCAGTGGTGTGATCTCGGCTCCCTGCAACCTCCGCCTCCCGGGTTCAAGTGATTCT
TCTGCCTCAGCCTCCTGAATAGCTGGGATTACAGGCACCCACCATGCCCGGCTAATTTTTGTTATTTTTA
TTGGAGATGAGGGTTTCACCATGTTAGCCAGGCTGGTCTCCAGCTCCCGGCCTCAGGGGATCCGCCCGCC
TCGGCCTCCCGTAGTGCTGGGATGACAGGCGGGAGCCACCACGCCCGGCCAGATATATGGTTTCCCACTT
TTGATGGTGATATTAAGTCTCTTCGTAAAACTTAAGCAAGAAAAATAATTAATGTTGTATTTATTAATTT
ATTTTTGAGACGGAGTCTCGCTCTGTCGTTCAGGCTGGAGTGCAGCGGCGAGATCTCAGCTCACTGCAAC
CTCAGTCTCTTGGATTCAAGCATTTCTCTTACCTCAGCCTCCCAAGTAGCTGGGATTACAGGCACCACCC
ACTACCACGTCCGGCTAATTTTTGAATTTTTAGTGGAGACAGGGTTTCACCACGTTGGCCAGGCCAGTCT
CGAACTCCTGACCTCAGGGGATCTGCCTGCCTCAGCCTCCCAAAGTGCTGGAATTACAGGCATGAGCCAC
CATGACCAGCCAATATTGCATTTACTTAATACAGGAGTGGTGGAGACAGATGTAGTTAAAATAAATGGTG
GGGCTGGGGGGTGGTGGCGGCTACAGAGTTGCGAGGCTGGAATTTGGGCAATGAGCTGTTTCAGAAACTG
CTGTCTTATTCCAAAACCCAGATTATGTTCTTTTTTTTTTGAGACAGGGTCTCGCTCTGTCGCCCAGGCT
GGAGTGCAGTGGCTCAATCACAGCTCACTGCAGCCTCTGCCTCCTGGCTCAAGCAATTCTACATCTTCCC
ACCTCAGCCTCCCAAGTAGATGGGGCCACAGGCAGGCACCATCACAGCCAACAAAATTTTTTTGTATTAT
TTGTAGTGATGGGGTTTGGCTACATTGCCCAAGGCTGGAGTGCAGTGGTGCGATCTTGGCTCACTGTAAC
CTCCACCTCTCAGGTTCAAGTGATTTTCCTCCCTTGGCCTCCTGAGTAGCTGGGATTACAGGCACAGGCC
ACCATGCCCGGCTAATTTTCGTATTTTTAGTAGAGACGGGGTTTCACCATGTTGGCCAGGCTGGTCTCAA
ACTCCTGACCTCACGTGATCCGCCCGCCTCGGCCTCCCAAAGTGCTGGGATCTCATCAAATAAAAGAAAA
GACTAACGCATAAGGAATGGTGAAATAGITCAGAAGTCGGTTTCCACCCAAGTTTCTGGCTCTGCCAGCC
TGCAGGGGTGCAGGGGGTGTCTGTAGGTCTTGGCACACCAGGCCTGGCTGATTCAGGGCCAGGCAGGTGA
CCCCAGTAGTGGGTGAGGTCTGAGGTCACAGCCCGCTGGGGCACCCTGAGGTGGACTGAGGCAGGTGAGG
GAGGGTGCTGCCGTCTGTCGAAAACATTCCCAGCCGGTGGCATCAGTGCGCTCACGCAGGCGTCGGCAGC
AGCGTCTGGAATGTGCTGTAAGGAGGCTGCCAGGCCCTTCCAGAGTGAGGTCGAGTCCTCGTTCTGGGCT
GATGGGGAAACTGAAGTGCCAGGGTTTGGGGATGGAGGATAGCTTGAGCTGAGGCCCCCGAGCCCCCGTG
TGGTCAGGCCCCCTAACTGAGCCCCAGCTACATCTTTGCTCCCTCTCCCCAGCCCCACTCTGCTCAAATT
GCTCCTCTGTCCACGCTTGTTCCTGCCTCAGGGCCTTTGCACTTGTTTTGTTTTGAGACAGGGTCCTGTT
CTGTCAACCCAGGCTGGAGTACAGTGGCGCCATCTCAGCTCACTGCAGTCTAAACCTCCTGGGCTCAAGC
AGTCCTCCTGCCTCAGCCTCCCGAGTATCTGGGACCACAGGTGTGCACCACCATGCCTGGCTAATTTCTT
TTTTTCCTTTGGTGGAGACTGGGTCTCAGTATCTCGGTTGCCCAAGGCTTGTCTGGAACTCCTGGGCTCA
AGCGATCCTCCCGCCGCAGCCTCCCAAAGTGCTGGGATTACAGGCCTGAGGCACTGAACTGACCTCCCTT
TGCACTTTTTTTTTTTTGAGACAGGTTCTCGCTCTGTCACCCAGGCTGGAGTGCAGTGGCGAGATCTTGG
CTCACTGCAACCTCCGCCTCCCGGGTTCAAGCGATTCTCCTGCCTCAGCCTCCCGAGTAGCTGGGATTAC
AGGTGCCCGCCACCACCCCCGGATAATTTTTGTATTTTTACTAGAGACGGGGTTTTGCCATGTTGCCAGG
CATGCTTGCGTATTTGCCAAGCTGGTCTCGAACTCTGACCTTAGGTGATCCGCCCGCCTCAGCCTCCCAA
AGTGCTGGGATGACAGGCCTGAGCCACCAAACCAGCTCCCCTTTGCAACTGGGATATTAGCCTGGAACAC
AGTTACCCCCAGATATTCCTATTCTCACCCTTCCCAAGCCTACTGCCCCACAGCAGGCGCTCCCGCCTCT
GGCCTGCTGCCCCGGCTGAACGACACAGGTGGGCACACCTTCCAGGTTCCATTCTAAAGCTCCCGCCCTG
CTCCTCCTAGGCAGGATCACGCGGAGACCAGGGCAGGCTCTAGGCTCCGAGGTTTATTTCATTCCCTTGA
CCTCACCCCACTTAGCAGGTGAATCCAAGCCTGCCGCTCCATTCGGCTCCTGCCCCTGAGGGCTTGGGGA
TTGGAGGGTAAACTGAAGCTGGAGGCTGGGAGTCTCGCGGACTGGGGCTTTCTCTCCTTGCAGCCTCCCT
GAGGTCGCACGTGCTGTGTCTTCCAGCAACTTGGTGGCCCTCTCTGAGCCTCAGCTTCCTCCATGATAGA
CGGCATCCTAATGACTCTTACCCAAAAGTTGTCGTTGGAGCAATGAAGGTTGCGGACAGGAATCTCTAAA
TTGCAGATGCTGTCCTCGTTATTAGATGAGTATGCCTTGAAAAAAAAAAAAAAAAAGCCGGGCACGGTGG
CGGCGTCTGCAATCCCAGCGCTTTGGGAAGCCGAGGGGCGAGGATCTCTTTACCCCAGCAGTTCGAAGCT
GCACTGAGCTATGATCACGCCACTGCACTCCAGCCTGGGCCACAACGCGTCCAAAACAAAACCAAAAAAG
AAAGAAAAACAGAAAAAAAGTCCGGATTTGAGGTCCTCGGGAAAACAGCCTGAGAGTGCCTCCCAGTTGC
CACGGCAACAGCTCTGTCCCGCCCCCTCCCTTTAAAAACAGCAGGAGGAACCAATCAGAGCGTCCACCGG
CGCATGCCTTCGCGCCTCCGCTGCATCCTGGGAGTCTACTTCCGGCTGGGGTTTCCCTTCGCAGCCTCCG
TCGACCATGAAACCACAACTCCCGGCAGGCGGCGCGGGCGCATGCGCAGGCGCGGGCCGGTGGGGTGGCC
TGGAGCGGCGTGCGTAATGTCGGCACTTTGCTGGGGCCGCGGAGCGGCGGGGCTCAAACGAGCCCTACGG
CCTTGCGGCCGCCCGGGACTCCCCGGCAAAGAAGGTAACACAAAGGGAGACGGCCAGGCAGCCCGGGGGC
CACGGCGTGGGCTCCTCCCCGCGCGGCCGGGGCCTTTGGCGCTTTGACCTTTGCCTCTTTGCACCTGGGC
GTGCGCAGCCCACCGACCCGGCCCCGCTTTCCACTCGCCCTTGCCCGCTTTTCGACTGAGGCTCCTCCGT
GCAGTCTGACCTTTGACTCGTGGTTTCTTATGCAGCCTCCTGACCCCAGGTGTGGTGTTTAGGGCTCTTA
ACCTTTGACCTGCTGCACCTCGAGTGCGAGGAACCCTCTCTCGGCGGTTCCGCGCCCGTGTTCGCCGCGG
CGGGGCCCTTCCACCTTCGCTGTCTTAGTTTCTTTCCAACTTAAGCCCGTGCTCCGAGAGTTTCCGCTGC
GTTTAGGGCTCCTTTGCCCTTCGACCCTTCGGCTTCTGCCTTTATGGAGGCCCCCGCCCACACGCCCGGC
CCCGCTCAGCCGCTCTCGTCTTTCCCGCAGGGACCGCCGGTGGCGTCTGCGGCCCCAGGAGGAGCTCGTC
CGCCAGCCCCCAGGAGCAAGACCAAGACCGCAGGAAGGACTGGGGCCACGTGGAGCTGCTGGAGGGTGAG
CGCGACCCCCACGGCGGCCCGGGAGAGGAGAGTCCGCTCCCTGCTGCCGGGAGAAAAGGCAAAGGCTCAG
ATTCTGAGACAGGCTGAGGGCGGTCTCCACCTCTCGGGTCCCAGGACACGCCCAGACGGCGTTGCTGAAA
CATCTCTTGCACCGCTCATGGGCCCTGTCCTTGCTGAGCAGGCTCCTTCCTGGAGCTCTCGTAATTCCCC
CTCCCCAGAACCCCACCAGGTGCTCCCACTTTCCAGGGACATAGGTGAGGCCCAAGATTATGTCTGGAGA
GACCCCCCCCCCGGCCCCGCCGCCAAGGCCACATGTGCAGAGCCAGGATGTGAACCCTGAACTGACTCAG
CTGTCCTGGCTTGGCTCTGGGGGGTAGGCGCCTAATGGAGACTGGATGACTGCGAAACTTGGGAGGGACA
GCCCAGGCTCCAGCGGTGGCAGGCCCTCGTCCTGGCCTGGCTGGTGGTGTGGGAGGGAGGTGGGAGCTTC
CCTGTGCTTGCTTCCCGCCGGTGCTTCTGTCCCACATCCTGTCTGCGTCCATTAGGCAGCCGGGAAGGGA
GAGGCGGGAGAACCTTTATAAAGGGAAAAAACCTGGCCAGGTGCGGTGGCTCACGCCTGTAATCCCAGCA
CTTTTGGGTGGCCGAGGTGGGCGGATCAAGAGGTCGGGGGTTCGAGGCCTGGTGCGGTGGCTCACGCCTG
TACTCCCAGCACTTTTGGGTGGCCAAGGTGGGCGGATCAAGAGGTCAAGGGTCCGAGGCCTGGTGCGGTG
GCTCACGCCTGTAATCCCAGCACTTTGGGAGGCCGAGGCGGGGGGATCAAGAGGTCATGAGTTCAAGACC
AGCCTGGCCAACATGGTGAAACACTGTCTCTACTAAAAATACAAAAATTAGCTGGGCATGGTGGTGAACG
CCTGCAGTGCCAGCTACTCAGGAGGCTGAGGCAGGAAAATCGCTTGAACCCGGGGAGACTGAGACAGGAG
AATCGCTTGAACCCGGAAGGTGGAGGTTGCGGTGAGCCGAGATCATGCCATTGCACTCTAGCCTGGGCAA
CGAGGGAAATTCCATCTCAAAAACAAAACAAAACAAAACAAAGCTCTCAACTGTTAATCCTAAAAGTATC
TCCTGAGTTTTGGAGCACTCTGAAGGCGGACATCACGCTTGTCTTTTTATTTCAGCAATTTTTTTTTTCT
TGAGACGGATTCTCAATCTGTTGCTAGGCTGGAGTGCAATGGTGCGATCTTGGCTCACTGCAGCCTCCGC
CTCCCGAGTTCAAGCAATTCTCCTGCCTCAACCTCCCGAGTAGCTGGAACTACAGGCGCGCACCACCACG
CCCGGTTAATTTTTCGTATTTTTAGTAGGGACGGGGTTTCACCGTGTTAGCCAGGATGGTCTTGATCTCC
TGACCTCGTGATCCGCCTGCCTCGGCCTCCTAAAGTGCTGGGATTACAGGCGTGAGCCACCACATCTGGA
CACCTGGGTCCCCCCCCCCCTTTTTTTTTTTGAGACAGGGTCCTGCTCTGTCTCTCAGGCTGCAGCACGG
TGGCCTGATCACGGCTCACAGCAGCCAGGAACTCCTAGGCTTAGGTGATTCTTTCACCTCAGCCACCGGA
GTAGCTGGGACCACAGGTGCGCGGCCCTGTACCCGGCTTATTTCAACAGTCTTTTTTTTTTAGATGGATT
ATTGTTCTGTTGCCCAGGCTGGAGTTCAATGGCGCTATCTTGAGTCACTGCAACCTCTGCCTCCCAGGCT
CAAGCAATTCTCCTGCCTCAGCCTCCCAAGTAGCTGGTACTATAGACACATGCCACCACGCCCAGCTAAT
TTTTTGGATTTCTAATAGAGACGGGGTTTCACCATGTTGGCCAGGCTGGTCTCGAACTCCTGACCTTAGG
TGATCTTCCTGCCTCGGCCTCCCAAAGTGCTGGGATGACCGACGTGAGCCGCCATGCCCGGCCAGCAATA
GATTTCCAACAAGGCTTCTTTAAAGAGATACAGAACACTTAGGCATTGATTGGTTGGCAAAAATGCAACC
TGAGGCGGCACGAGCAGGAGCCGCGTTTTCCCTGCGAGCTAGTGGCTCAGCGTCCACCGGTGTAGACACT
GGGAGCCTAGGAGTGTGTGTGTGGCAGCTCCACCAGCTGGGGGGGTCTTGCTGAGGCGGGGTGCGAACTT
GCAGGCCATGGTTGAGCCAGGGAGTGGGCGGTTCAGGGTCCTGGGAGTCTGTGCAGAGGCCCTGAGCTAG
ACAGTGAGAGGGGCAGGGGAGGCGGGGGCCACCTGGATAGATTCTGGAGGCAAGCCCAGAGGAGGTACTG
AGAGGTGGACGGAGGGAGTTGTCACAGACCCCGGGTTCTGGGCCTGTGCACCTGGAGGGTCTGAGCGGCC
AGTGACCGGGGAGACGTGGTGCTGCAGGTTTCGGTGGCAGCTTGTTAGGGCTGCTGAGTTCTGTGAGGCT
CGGGTGGGGAGGTGCGGAGGTGGTGGGGTGCGTGTGTCTGGAAAGCAGCGGGCAGACCTTACAACGTTGT
TGGCAAGTATGAGGAATTCACGCCTTGGGTCCCAGCGAGCTCAGCCACCTGCACCTCCTGGCCTGGCTCA
GGGTCCAGAGAGACGGGTGCTCGAATGGGGAGGGGGTCCCTCTCATGTCCTCGCCTTCTGCCCCTCACAG
TGCTCCAGGCGCGGGTGCGGCAGCTGCAGGCTGAGAGCGTGTCGGAGGTGGTGGTGAACAGGGTGGATGT
GGCGCGGCTCCCAGAATGTGGCAGTGGAGATGGTAGCCTCCAGCCACCCAGGAAGGTCCAGATGGGGGCC
AAGGATGCCACCCCGGTGCCCTGTGGCCGCTGGGCAAAGATACTGGAGAAGGATAAGCGGACCCAGCAGA
TGCGTATGCAGCGGTTGAAGGCGAAGCTGCAGATGCCATTCCAGAGCGGGGAGTTCAAGGCGCTGACCAG
GCGCCTGCAGGTGGAGCCCCGGCTCCTGAGCAAGCAGATGGCCGGGTGCCTGGAGGACTGCACGCGCCAG
GCCCCCGAGAGCCCCTGGGAGGAGCAGCTGGCCCGGCTGCTGCAGGAGGCCCCTGGGAAGCTGAGCCTCG
ATGTGGAGCAGGCCCCGTCGGGGCAGCACTCGCAGGCCCAGCTCTCAGGTCAGCAGCAGAGGCTCCTGGC
CTTCTTCAAGTGCTGCCTGCTCACTGACCAGCTGCCCCTCGCCCACCACCTGCTGGTCGTCCACCACGGC
CAGCGGCAGAAGCGGAAGCTGCTCACGCTGGACATGTACAACGCCGTGATGCTTGGCTGGGCGCGGCAGG
TGAGTGCAGCCGGGAGCCGGGCCACCTGCCCTGGTCGTTGGAGACAGGAACTTGCCCTCATTTAGACTGG
ACTTTTCTCTCTTAGCCCCCAGGTTCAAGTCCTTTGTTTTTTATTTTAATAATTCTTTTAGGCTTTTCAA
ATACAAATCACGTGTGACTCTCAAATTTTGAAAATGACCAAAAAAGTAGGGAGAAATTCTGCTAATGCCT
TGAGCATTTCCTTCCGTTCTTTCTCGGGGCTCGTGAATCTGTCTGGGCAGGGGTGCTTTTGGCCTCGTCG
GGGGGCAGCCTGCAGAGCCCTCGTCAGGATGAGGCTTTGGTGTTGGGGTTGACTTTGCTCCTTTTTCTGG
TTTCCTGAGGTGGAAGGTCGGGCTGCTGTGCTGAGATCCTTCTGTGTGAAATGTGGGTGTTCACTGGCGC
CGCATTCCCTCTGAGCACAGCTTTAGATGTATCCCACCCATCTTCGTTGTGGTTCATCTGGAGTATTTTC
CAATTTCCCTTGCCGTTTTCTTTTTGTTTGTTTATTTATTTGTTTTCTGGGACATACTCTCCCTGTTACT
AGGTTGGAGTGCGGTGGTGCGATCTCGGCTCATGGCCACCTCCACCTCCCAGGTTCAAGTGATTATCCTG
CCTCAGCCTCCTAAGTAGCTGGAGTTACAGGTGCCCGCCACCACCCCCGATTAATTTTTGTATTTTGAGT
AGAGACGGGGGTTTCACCGTGTTGGCCAAGCTGGTCTCAAACTCCTGACCTCGTGATCTGCCCCCCTGAG
CCTCCCGAGGAGCTGAGATTACAGGCGTGAGCCACCGTATCCGGCCAAAGTTTCTCCTCGGAACTCTTGG
TTGTTTTGGAGGGTGTTGTTTACACACCCTTTCAAAGCAGCAAACGTTCCCAGCTTTCTCCCCCCCCATG
ATCCTTCAGCCCTAATTTGAGAAACAACAGTAAAAGCTTCTCTTAATATTTGGGTAAACATGGGATACTA
TTATATTAAAATACAGATATCGCAATATTAAGACCAATTCGTGGTAGAGAACTTGATGTGCTTCTTTATT
AACGCATCAAACAGCGAAATTCAGGATCAGGTGGGGCCAGTCTTGCGGTGAAGTCCGCAGTGCTCTTACC
GCGAGGTGGAGGCGGCGTGCTGCCTGGTTCCTCCTTGCTGCAGGGGGCGCCAGACTCCGGGAGGACTGAG
GATGAGGGTCGTGTATTTTCCCCTCCAAGGTCACAAGCCCCCTGGATTCCCTGGGCAGAGAGCCCACACT
GTAACAGGGCGCTTTTGGGATCGGCCCCTACGAAAGTGTCCCAAACCTGGCGGCTTGCACAGCAGAAACG
CACTCTTAGCACGAGCCCAGAAGTCTGGAATCAAGATGTCTGCAGACCCGAAGTCTCTCCAGAGGCTCTA
GCAGGGGCCCCTCCTGCCTCTCCCAGCGTTCCGGGGTGGCCAGCAGTCCTGGATGTCGCTGGACTGCGAG
CTCAGCCTCCCGAGCAGCTGGGATTACAGGCGTGTGCCACCACCCCCGGATAATTGTTTTAATTTTTATT
TTTAGTAGAGGTGGGGTTTCAGCATGTTAGCCAGGCTAAGGCATTGCTTCTATCTTTTTTTTTTTTTTTT
TTTTGAGATAGACTCTCTGTCTGTTGCCCAGGCTAGGGTGCAGTGCCATGATCTCGGCTCACTACAGCCT
CCGCCTCCTGGGTTCAATAATTCTCCCTGCCGCAGCCTCCTGAGTGGCTGGGATTACAGGGACTCACCAC
CACGCCCGGCCAATTTTTGTATTTTTAGTAGAGACGGGCTTTTGCCATGTTGGCCGGGCTGGTCTCAAAC
TCCTGACCTCAGGTAATCCGCCCGCCTCAGCCTCCCTAAGTGCTGGGATTACAGGCGTGAGCGACCGCGC
CCGGCCACGTATTCTAACCAGCACCATCCAGTAGAACTTTCTATGATGTTGGGAATTTTCCGCATCTGCC
AACAGCTGTGTGTGCTACTGAACACTTGAAGCTGGTGAAAGGGAAGAACTGAATATTTCAATTTAAGGAC
TATGTGTGGCCAGTGAGGTCTACCATACAGAGGTTGTTTAGAAACTGCCCCAAAACCGGCCGGGCACAGT
GGCTCACGCCTGTAATCCCAGCACTTTGGGAGGCTGAGGCGGGCGGATCACGAGGTCAGGAGATGAGACC
ATCCTGGCTAACACAGTGAAACCCCGTCTCTACTAAAAATATACAAAAAATTAGCCGGGTGTGGTGGCGG
GCGCCTGCAGTCCCAGCTACTTGGGAGGCTGAGGCAGGAGGATGGAGTGAACCTGGGAGGCAGAGCTTGC
AGTGAGCCGAGATCGAGCCACTGCACTCCAGCCTGGGCGACAGAGCGAGACTCCGTCTCAAAAAAAAAAA
AAAAAAAAAATGCCCCAAAACTCATGTCTCTGGTCTCTGGTGTGTGGCCACCTCATGTCACAGCATGTCT
TCAGCTGCAGGACTATGGGGTGGGAGGATGTAAGGGAATTTTTTTCCCTTTGGAATAGTCTGTTACAGTT
ACAGGCCACTCTTAAGCAGCCACTGGGTCAAAGGAGAAATTGCTCAGAACAGGGATGTCAGGAAAAATGA
ATACAAGAAAAAAATTATATATATATGTTATTTTATATATATATATATATGTGTGTGTGTGTATATTTTA
AGACAGAGTCTCCAAGTCTGTCGACAGCAAAGTTTCTGTCTCCCAGGCTGGAGTGCAGTGGTGAGATCCC
GGCTTACCGCAATTTCCGCTTTCTGCGTTCAAGTGATTCTCCTGCCCCAGGCTCCCAGTAGCTGAGATTA
CAGGCATGTGCCACCACGCCCAGCTAATTTTTTTTTTTTTTTGTATTTTTAGTGGAGGGAGCACAGGCAT
GAGCCACCACGCCCAGCTAATTTTTTTTTTTTTTTTTTGTATTTTTAGTGGAGATGGGGTTTCACTATGT
TGGCCAGGCTGGTCTTAAACTCCTGACCTTGTGATCCTCCTGTCTTGGCCTCCCAAAGTGCTGGGGTTAC
AGGCATGAGCCACTGTGCCTGGCCAAAAATTAAATGAAAAAAAAAAAAATTGCAGGGGATATTTAAGCTA
TACATGGCTGGGCGTGGTGGCTCACACCTGTAATCCCAGCACTTTGGGAGGCCGAGGCGGGTGGATCCCC
TGAGGTCAGGAGTTGGAGACCAGCCTGGCCAACAGGGCGAAACCCCATCTCTACTAAAAATACAAAATAA
TTAGCTGGGTGTGGTGGCATGTGCCTGTAGTCCCAGCTACTCAGGAGGCTGAGGCAGGAGAATCGGTTGA
ACCCGGGAGGCGGAGGTTGACAGTGAGCTGAGATTGCGCCACTGCACTCCAGCCTGGGCGACAGAGCGAG
ACTCTATCTAAAAAAATAATTAAATAAAATTTCCAAAGAAGAAGTAAACTATGAAGTTCGAAAACACTTG
GAGATAATGAAAATGAAAACACCACCTGTCAGAAGTCCTGGGATGCAGCTGACACAGTGCTCGCAGGGTA
ATTTATAGTCACAAAATGCCTATATTCGGAAAGGTGATGGCCGGGCGCGGTGGTGCACACCTGTCATCCC
AGCACTTTGGGAGGCTGAGGCTGGCGGATCACGATGTCAGGAGTTCGAGACCAGCCTGGCCAGCATGGTG
AAACCCCGTATCTGCTAAGAATACAAAAAATTAGCTGGTGTGGTGGCGTGTGCCTGTAATCCCAGCTACT
CAGGAGGCTGAGGCAGGAGAAGTGCTGGAACCTGGGAGGCAGAGAACCCGGCCCAGCTGAGATTGTGCCA
CTGCACTCCAGCCTGGGCAACAGAGCAAGACTCCATCTTGGGGGGAAAAAAAGTGAAACATCTCAGACCA
AATACGTCACCTTTCACCTTAGGAAACTAGAAGAAGAAGCTGTCACTAAACCTAAAGCAAGTAGATGAGA
GGAAGTTACAAAGATTAAAACAAATCGATGAAATGGAAGGCAGAAACAGCAGAGAACAGTCAAGCCAAGT
CAGTCCTTTGAAAAGATCCACACAATTGACAAATCTGGAGGTTTAACAAGCTGCCTCCCAGAGGAACGTG
AACCTCCCACAGTCCCACCTGGCGGGCCATGCGGCTGCCTCTGCAGGTGGAGCTTGTGTGTCAGAACCTC
AGGGGGATGCATCGGTCTGCCCAGCTGGCCACTCCTGCCTGGTGACCCTGTCCCCTGCTGAGCTGGTGGC
TACAGAGAAGGGGTCTCCAGGGAGCAGGGAGGATGTGGAGGTTGGGCCATCCCCCCAGACCCTCACCCTG
GCTTGGTCTCCCAGGGTGCCTTCAAGGAGCTGGTATATGTGTTATTCATGGTGAAGGATGCCGGCTTGAC
TCCGGACCTGCTGTCCTATGCGGCTGCCCTCCAGTGCATGGGGAGGCAGGACCAGGACGCCGGGACCATC
GAAAGGTAGGTGGTGTCGGGAGGGCCCCCACCCCCCACCATGTCCCTCCCAGGGTTTTAAGATCTGGGAC
GGTGCCTGGGCTGGGGGCTGGGGACCCCCAGAGGGCCTGACAGCCACACCGAGACCTGCAGGCTAGCAGG
AGGTGGTCACGAGGTTCCCCTGGGGTCTGTGGAAGCCCAGCGTGGGGCTGGCCCGTCCCTCGAGCAGGCC
GCCTGCACCACAGGTGTCTGGAACAGATGAGCCAGGAGGGGCTGAAGCTGCAGGCACTCTTCACCGCCGT
TCTGCTGTCTGAGGAGGATCGGGCCACTGTTCTGAAGGCCGTGCACAAGGTGAAGCCCACCTTCAGCCTC
CCGCCGCAGCTGCCGCCCCCGGTCAACACCTCCAAGCTGCTCAGGGACGTGTATGCCAAGGTGAGCCCAC
GTGGGCCCCGGCAGACTTCAGTCCTTATAGCTGCCCTTTGGGGGTTCCTGGAGCCTCAGCTCAGACCAAT
GGGCCTCCCCGGTGCCCAGGGCTCACCTGGGCTGGGCCCTGAATTCCTCCCTTCCTCCTCTGAGAAGATG
GGAGGACCCAGAGCTGGGAGGGTCTTGGCCCTGAGGGTGTGGGCTCAGGGGGACGAGGTGGTCGGGGGCC
GCAGAGAGAGGGTGGGGTGACAGGAGAGCAGTGCGTTTGAGTGGGGCTGCCAGGTGCACGCAGTGGGCTC
GTCTGTTACTGAGGTGTAATTCACCTGCCATAAAACCCTCTTAAAGGTGTAATTCAGCGATTTTAGTGTA
GTCACGGTTGTCTAATTCTAGAATGTTCCATCACCCTAAAAGGAAGCCTCGTCCCCATCCCCGTCACTCC
CCATCCCCTCCCCATCTGTGGATGAGTCTGCTCTGGACATTCCATAGAAATGGGACCACACACTGCGAGA
CCTTTTGTGTTTGGCATCTCCCACTGAGCGTGACGTCCTCATGGTGCCTCCGGGAGGCGACCTGGGTCAG
GGCTGAGTCATGCTCCGGTGCGTGGATGGCCGCGCCGTGCCCGTCTGTTCGTCCGTTTATGGGACGTTGG
GCTGTTTCTACTTATCGACTGCGAGTCATGCAGCTGTGAACGTTTGTGTACAAGCTTTTATGTGGACGTG
CGTCTTTGATTCTCAAAGCGCTAGGCTTTAGTTGTTATTTTTAATTCATCTATTTCTGGATGGGTCACAT
TCATACAGTTCTGAGTTTGAGGAGAAAACAGCCAACATCTGGGGGTGCAGTGAACGGTCTCCCCAGGCTC
TGCCAGCTCCCAGCATCACCGCCCCTGCCACCCCCTGCACGGTGCCCAGTGTCCCTTCTAGAGGGGTCGA
GGCCAGGAGGGTCAAGGCCTTTGGTTTGCACTGCTGGCACTGACTTTACCCGCAGCCCGCCTTTCCCCGC
GTCAGCGATAGCCACCAGCAACTTGCAGGAGACGCAGAAACGCGTGCACCCATGCTGTCTCGGGGTCCTG
GGAGGGCAGGTCGCCCTCTGGCAAGCCCCCAACCTCACTACCCCCATCCCTGCAGGATGGGCGTGTGTCC
TACCCGAAGCTGCACCTGCCCTTGAAGACCCTGCAGTGCCTCTTTGAGAAGCAGCTCCACATGGAGCTGG
CCAGCAGGGTGTGCGTGGTGTCCGTGGAGAAGCCCACGTTGCCAAGCAAGGAGGTCAAGCACGCGGTAGG
GGCTGAGCCGGGGTCCCGTGTCCGCCGCAGGGGCTGAGCCGGGGTCGCGTGTCCACCGCACGGGCTGAGC
CGGGGTCCCGTGTCCGCCGCAGGGGCTGAGCCGGGGTCGCGTGTCCGCCGCAGGGGCTGAGCTCCGTGTA
TGCGGTTGAATTGCTCATGCCCTGGTCTGGTCATGCAGGGCCTAGGAACTCAGCCTGGCTGCCGCTCAGG
GAAAACTTGTGGGGTCCCGAGGTTGGGTCTTGGCCTGTGCTTCTGCGCACACTGCAGCTGGGCTTTTGCT
CTTGGCTGCGTCAGGCACGCTGAAGCCGGCAGAGGGCAGGCCGGGGTGAGGAGCTACCCGGAACACACAG
CAGGGACCTTGAGGGCCATGAGGTCTCTGCAGGGAGCTGCGGTGACCCCCGTCCTGTGAGCCTGGGTCCC
ACCAGCTGCCACGGGGCTCACCGGGCCCGTTCATCCCCCGCAGCGGAAGACCCTGAAGACCCTGCGGGAC
CAATGGGAGAAAGCACTGTGCCGGGCGCTGCGGGAGACCAAGAACCGCCTAGAGCGCGAGGTGTACGAGG
GCCGGTTCTCACTTTACCCCTTCCTGTGCCTGCTGGACGAGCGCGAGGTGGTGCGGATGCTCCTGCAGGT
GCGTCTTCCTCCGCGCGGCCGGGTCCCCGGGCGGGGGGGGCAGGCACTCACGGCTGCCTTCCGCAGGTCC
TGCAGGCGCTGCCCGCCCAAGGTGAGTCCTTCACCACCCTGGCCCGGGAGCTGAGTGCGCGCACTTTCAG
CCGGCACGTGGTGCAGAGGCAGCGGGTCAGTGGCCAGGTGCAGGCGCTGCAGAACCACTACAGGAAGTAC
CTCTGCTTGCTGGCCTCCGACGCCGAGGTGAGGCTCGCACCCCCTCTCCTCCTGGCTGGGGCCAGTGGTG
GGTGGGCGCTCTCCCTCCCTGGAGCAGCCGGGTTTGCACCCAAGCTTCAGTCTGTTCATCTGTATGCTTG
GGCTGACAGATGCCCCACCGGGCGTCAGGCACGGGCGGTGGGGCATCTAGCTCAGGGGCCCCGGTGCCCA
GCGCCCTGACCGCCCTGTCCTACAGGTGCCCGAGCCCTGCCTGCCGCGGCAGTACTGGGAGGAGCTGGGG
GCGCCCGAGGCCCTGCGGGAGCAGCCCTGGCCCCTGCCAGTGCAGATGGAGCTGGGCAAGCTGCTGGCGG
AGATGCTGGTGCAGGCTACGCAGATGCCATGCAGCCTGGACAAGCCGCATCGTTCCTCTCGGCTTGTCCC
CGTGCTCTACCACGTGTATTCCTTCCGCAACGTCCAGCAGGTGCCAGGCAGTGCCCTCCTGAGCTGGGGG
GCATCCCGCTGGGAGGACCAGGGACCCCATGGGGTTGGCGCCTTTGGGCGGAGCCTTGATCTCAGCGCAG
CCGTCAGTACCTCCCAGGACTCGGGACACACCATGGGTGTCCATGCAGGTGTCCGGCCGTTTCTGGAGGT
GGCTTCTGTTCGTGGGAGGCCACACTGGTTTCTTGTTGGCCGGGGAGCCGGCCCCTCTCACCCAAGTTTA
AGGGGTGGGAACAGGAAAGCCCCAGCACCGGGGCCCTGACCCGTCTGCCTGTCGCACCCCAGATCGGCAT
CCTGAAGCCGCACCCGGCCTACGTGCAGCTGCTGGAGAAGGCCGCGGAGCCCACGCTGACCTTCGAGGCG
GTGGATGTACCCATGCTTTGCCCCCCGCTGCCCTGGACATCGCCGCACTCTGGTGCTTTCCTGCTCAGCC
CCACCAAGCTGATGCGCACGGTGGAAGGCGCCACGCAGCACCAGGAGCTGCTGGAAACCTGCCCGCCCAC
CGCGCTGCATGGCGCACTGGACGCCCTCACCCAACTGGGCAACTGCGCCTGGCGCGTCAACGGGCGCGTG
CTGGACCTGGTGCTGCAGCTCTTCCAGGCCAAGGGCTGCCCCCAGCTAGGCGTGCCGGCCCCGCCCTCCG
AGGCGCCCCAGCCGCCCGAGGCCCACCTGCCGCACAGCGCCGCGCCCGCCCGCAAGGCCGAGCTGCGCCG
TGAGCTGGCGCACTGCCAGAAGGTGGCCCGGGAGATGCACAGCCTGCGGGCGGAGGCGCTGTACCGCCTC
TCGCTGGCGCAGCACCTGCGGGACCGCGTCTTCTGGCTGCCGCACAACATGGACTTCCGCGGCCGCACCT
ACCCCTGCCCGCCGCACTTCAACCACCTGGGCAGCGACGTGGCGCGGGCCCTGCTGGAGTTCGCCCAGGG
CCGCCCGCTCGGCCCGCACGGCCTGGATTGGCTCAAGATCCACCTGGTCAATCTCACGGGGTTGAAGAAG
CGGGAGCCGCTGCGGAAGCGCCTGGCCTTTGCGGAGGAGGTGATGGATGACATCCTGGACTCCGCGGACC
AACCCTTGACGGTAGGGGCGGGGCCCCCGCATTCCCCGCCCTCCCGGCACCCCCGCGCCCTCCCCCGGCG
CCCCCGCGCCCTCCCCCGGCGCCCCCGCGCCCCCTGCCCGTCTTCCTCCTCCCCTCCCCCTCCCCTGGCG
CCCCCTGCCCGTCTTCCTCCTCCCCTCCCCCTCCCCTGGCGCCCCCACGTCCCCCGCCCGCTCTCCTCCC
CCGCGCCCCCTGCCCGTCCTGCTTCTCCCTTCTCCCCTGCCCCAGGGCCCCCGCGTTCCCCACGTTCCCC
CCTCCCCTGCATCCGCCGAGCTGCTTCCTCCCCCGGCGCCCCCAGGTCCTCTGTCCACCCTCCTCCCCCG
GCAGCTCCGGACACGCTTGTTTCAGCCCGGTCCACCCGCAGCTCCCATCCTCCATCCGTGGCTATGGAGA
ACTCATCCAGCCACCGCCCCGTCTCTCGCCCACATGCGTCCCGTGTTCACTATCACGGGTGTGCGTCTCC
TCCCCATTTCCCACCTCCCCGCAACACAGCCTCCCAGCGGGCTCAGGGATCGGGCCCGGGCCTCACTGCC
CCCCATCCGCTGTCCCCGCAGGGCCGAAAGTGGTGGATGGGCGCGGAGGAACCCTGGCAGACGCTGGCCT
GCTGTATGGAGGTGGCGAACGCTGTGCGCGCCTCCGACCCTGCCGCCTATGTCTCCCACCTCCCCGTCCA
TCAGGTGAGCCAGCTGGGTCTGGCCCCCGAGGCCGTGCAGTGTGCACTGGGCCTGGGGCTCGACTTGAGG
GTGTGAGATTTCACCTTCGCTCGTGGTATTCTCTGGAGGTGGTCCTAGAGTGTGCGGGAGCCATGGCTCC
TGGTCTCTCCCGGGAGCGTGCGGGGGCTCCGGTGCACTTGGCTGGGGGGTGCACGGCCCTAGGAACGGCT
CGACCTGGGGGGTGGTTTGGGGTCCCACACGTCTCTGCCTCTCTAGCTTCCATCTCCCGTTTGCCGCCCC
TTCCTCAGGACGGCTCTTGCAACGGCCTGCAGCATTATGCTGCTCTGGGCCGCGACAGCGTGGGCGCCGC
CTCCGTCAACCTGGAGCCCTCGGATGTGCCGCAGGACGTGTACAGCGGCGTGGCCGCGCAGGTAGGGTGT
GCCCTGCTGCCCGGGGGCATCTCGGCGTGGGGGCAATGTGAGCCCTGAGTCTCGGCCCCAGGTGCCCTCA
CCTTGGCCTGTCCTGAGGATGTGGTGGGGCTGGGGCGGGGGCTTCATGGGTGCTTGGTGGCCCCTGGGCT
GCTAGCGGGGCTGACCCGCGCCCCCGGCCCACCCGCCGTGCAGGTGGAGGTGTTCCGTAGGCAGGACGCC
CAGCGGGGCATGCGGGTGGCACAGGTGCTGGAAGGTTTCATCACCCGCAAGGTGGTGAAGCAGACGGTGA
TGACGGTGGTGTACGGGGTCACGCGCTATGGCGGGCGCCTGCAGATTGAGAAGCGCCTCCGGGAGCTGAG
CGACTTTCCCCAGGTGCGCCAGGCATGTCGCGCTGCGAACACGTTGGTTTCACTGCCATTTAAAACTCAG
ACGGGTGTGCCCCCGACCCAGCCTCCCAGCAGGAGCCCTGGCTGCTCCCCCAGACCACTGGCTGCCTGTT
AGGGCTTGGGCGTTTCTTGCTGACTGCGTGGGCGCCAGGCCCCATTCCCCTGCCCCCCTGCTAGGCCCTA
GGCCTTGGGCCTCAGTGGCTCTGAAATAGGGGGGAGTAGGGCGTGAACGGGCCAGCCCCTGAGGACCTGC
ACCTGCTGCCCGCCCTCTGCAGGAGTTCGTGTGGGAGGCCTCTCACTATCTCGTACGCCAGGTCTTCAAG
AGTCTACAGGAGATGTTCTCGGGGACCCGGGCCATCCAGGTACGTCCTGTCCTGTCCCCGCTCGGCCAGG
ACTCCCTAAGCAAGTGGACGGGATCCCCGGCCTGAGCCCCCTGCCCTGAGACGGCCTGTGTCCCACAGCA
CTGGCTGACCGAGAGTGCCCGCCTCATCTCCCACATGGGCTCTGTGGTGGAGTGGGTCACACCCCTGGGC
GTCCCCGTCATCCAGCCCTATCGCCTGGACTCCAAGGTCAAGGTCAGTGTACCACCCCATCCCTCCCCAG
TGTGCCACCATCCCAGTGTAACAGCGTCCCAGTGTACCAGTGCCCCAGCGTACCACCACCCCAGTGTACC
ACCGCCCCAGTGTGCCACCCCATCCCGCGCCAGTGTGCCACCATCCTAGCGTGCCACTACCCCAGTGTAC
CACCACCTCAATGTACCACCACCGTGCTGGGCCTCGGGTGGGACTCACCGGCCCCTTCCCCTTTTTAGCA
AATAGGAGGTGGAATTCAGAGCATCACCTACACCCACAACGGAGACATCAGCCGGTGAGTGGGGGGCCCG
GGCTGGGGCCTGGCCGGGGGTCTGGAGAGCAGCCACGGTGGAGACAGCGGCCAGTTAGGGGTGTCCCTGG
GCCGCCCCCACCGGCACCTGTTCCGTCCTCAGAAAGCCCAACACACGTAAGCAGAAGAACGGCTTCCCGC
CCAACTTCATCCACTCGCTGGACTCCTCCCACATGATGCTCACCGCCCTGCACTGCTACAGGTGGGCGTC
TCCGGACGGCGTGGGTGCCGCTCGCCTCCCAGGGGCGTCTTCTGGCTAGCAGGGCACAGCTGGCAAGGCT
GGGTCTAGCTGGGGGCAGGGACTGGGCAGAGTGGATCTGCTGTGTCTGTGGGGCCTGTGTAGAGGCCCTG
AGGCTGTGTGCCCCGAATGTAGGTGGTCCTGGCAGCTCCCCTCCTGTGGCCCGACAGGGAGAGCCGAGCA
TGGGCGCTGCCCCTTCCTGCTGGGCATTTCTTGGCACCGCGATGTGTGGTTAGATCTGGAGCACGTGGAG
GTCGTTCCAGCAGAATCTACCGGCCACGGGAGCTGGGAATGCTCAGGATGCGGGGTGGTGGCCCTGGCAG
GAGGGGACCTCGTAGCCGCCCTCGGAGCTGAGGCTCCCACAGCAGCGTGGGGGGCGGCAGGGGTGGGGCG
AGGAGGCTTCTGGGAACATTTCCTCCCCCTGTACTGATACCTAACCCAGGATGTTTGGGGGGGGGCGGGG
CGAGGGGTGGGGCGAGGCCTGCAGGAGGGTGGGCTTCCCTGGACCTCAAGTGGAGGGGGCTGAGCGCAGG
GCCAGGATGCTGGTCACTGGGTGGCCCTGTGAGCTCCCCTCCCTTCAGGAGTCCTCCGCTCTGCCACAGG
AAGGGCCTGACCTTCGTCTCTGTGCACGACTGTTACTGGACTCACGCAGCTGATGTCTCCGTCATGAACC
AGGTGCCCCCGTAGTCTGAGCCTCAGTCCACCCATGGGTGGGGCCCCCACACTGGGGCGTTGGGTAGGGG
TGGTGCCCAGAGCCTGCCTGATCACCCCTGGGGATCCTCTGACTCCTGCCCAGGTGTGCCGGGAGCAGTT
TGTCCGCTTGCACAGCGAGCCCATCCTGCAGGACCTGTCCAGATTCCTGGTCAAGCGGTTCTGCTCTGAG
TAAGGCGCTCCCTGACCCCAACTACCTGGATTCCCCCCCCACCCCAAAACCTGGCCTCAGCCTCACCCCA
CCCTGCTTCCAACCCCAGGCCCCAGAAGATCTTGGAGGCCAGCCAGCTGAAGGAGACACTGCAGGCGGTG
CCCAAGCCAGGTAGGCGGGTGGGCAGCCTCGCAAGGGTGGCTCGAACTGCGGGCCAGGGGGGGGCTTTCC
CGCCACCCACGCCGTCCGCTCTGCTTCCTCCGCAGGGGCCTTCGACCTGGAGCAGGTGAAGCGTTCCACC
TACTTCTTCAGCTGACACCCCGTGAGCCTTGTCAGTGTGTAAATAAAGCTCTTTTGCCACCCCCAGGAGC
CACTGTCTTCAGGAAGGGTGCACGCCCTGCGGGTCTCGGGCAATCACACGCGGCCAGGCTTGGCGCCAAT
GCTGTCGTTTATTGCGCGGAATGGGGGTGTGGGGGTTAATGGGGCGTGGGGGGCCACGGTGGGGGCACTG
CTGCCTCGGCTCGTCAGTACATTCATCACGGCGGCGGGACCCCAGCCTCCCCCCCGCGCCCTGCGCAGCC
AGGCCTGCCTCTCGGTGCCAGTGCTGGAGGGAGGCGGGGTGCTGCTCCCCGAGGTCACCGGGGGACGCGC
GCGGACGGGGGCCGGGCCGGTTATTGCGTGAGCGCGATGGGGGCAGCGGGAAGCCGGCGGGCCAAGTATT
GCACTTAGAAAACGATCCTCCTCGGACGGGGGCCACCTAGAGGGTGGGGGGCGGGGGGGGCTCCACAGCC
GGCTCCTCTCAGCCACTGGGCCGCCCCGTCCCTGTCTTACAGCTGGGGGAACTGAGGCACCGAGGTGAAG
GGAGCCCCCTCGCACGCGAGGCCGCCGCCGGGGGCAGGGGCGATGGGGGTGGGCGCGGGGCGATGAGGGG
GGACGGCCGGGGGCGCGGAGGGGGCTGCCCCGCCGGCCCTGCCCGTCCGTCCAACTACGGCTACCTACGT
CTCGTCTATGGCTTCTGGGCGGACTGGCGGCCGGGGCAGCGCAATGGCATGGCTTTGGTCTGGATGACGG
CCCCGCCCCCGGCCCGCCTGGGCCCGCGGGGCGGTCGGCGAGGGTCACAAGTTGGACGAGAGGCGCGAGC
GCGCGGAGTCCTGGGGGTCCAGGCCGCCGGCGGCGCCGGGTGAGGCCGAGTCCCTGCGGTCCGGGCTGGG
CGCGGCGGCCCGGGCAGCGGGCGTGGGCCCCAAGCGCGGTGTGGAGCTGCTGGCCGGGCGTGTGGAGGCC
GCGGGGCCGGGGGCGCCGTGAGGCAGCGAGGGCTGCGAGGCGGACAGTGGGCGCGACGCGCGGCTCAGGC
GGCGCGCGGGCAGGGCGGGCCCAGCAAGGGGGGCGGCGGGCAGGCCGCCGTAGGGCGAGGTCCGCGGTGC
CCGGGGGCTGGCGGGCGCGCCCGGGGGGCTGGCGGGGGGGGGGGGCCCGGGTGAGGCGGCGGCAGGTGCG
GGCCCCGGGGGGGGGGGGCGCACGAGGCGCGGCGAGCCGAGCGCCAGCGGCCCCACGAGCGGCCGCGCCA
CCTGCGGGCAGAAGCTCATGGCCGCCGCCTGCTGCAGCGTGGCGATGGCCGAGGTGACCTGCGGCGGCGG
CGGCGGCGGCGGGAAGAGGCCCACGCGCTGACCCAGCTCGGCCTGCTGCACCATCTCGCGGTCGTACTTG
ACGATCTCCTGGATGATGGCGTTCTCCTGGTTGTTGAATACGCCCGAGTTGAGGTCATGCTGCACCTTGT
GCAGGAGGATGGAATTCTTCTTGCCTGCGGGAGAGGGGGCGTTAGCGTGTGCACAGGGAGCGCCTGCTGC
GTACAGCGGGCACCGAGCACCTACTCTGCACCGCGGGTGCGCACACAGTGCTTGCCGTGTAGAGCAAGCA
TGCATTTACCACCTACTGTATACAATAAGCACATATGTAGCATCTCTGGCCTATAGTATGGATATTGAGT
ACCTGCCATATACAGGCTGAGCACCTGCTATGCACAGCCAGCATGCATGAAGCACCTGCATACAGAAAGC
AGGGCTTTTGTTTTGCTTTGTTTCTGAGACGGAGTCTTGCTCTGTCGCCCGGGCTGGAGTGCAGTGGCGC
GATCTCGGCCCACTGCAAGGTCCACCTCCCGGGTTCACACCATTCTCCTGCCTCATCCTCCCGAGTAGCT
GGGACTACAGGCGCCCGCCACCACACCCGGCTAATTTTTTGTATTTTTTTTTTAGTAGAGACGGGGTTTC
ACCATGTTAGCCAGGATGGTCTCAATCTCCTGACCTCGTGATCCACCCACCTTGGCCTCCGAAAGTGCTG
GGACTACAGGCGTGAGCCACCGCGCCTGGCAGTAAA
The protein sequence of wildtype human POLRMT is as follows
(1230 amino acids):
(SEQ ID NO: 2, transit peptide)
MSALCWGRGAAGLKRALRPCGRPGLPGKEGTAGGVCGPRRSSSASPQEQDQDRRKDWGHVELLEVLQARV
RQLQAESVSEVVVNRVDVARLPECGSGDGSLQPPRKVQMGAKDATPVPCGRWAKILEKDKRTQQMRMQRL
KAKLQMPFQSGEFKALTRRLQVEPRLLSKQMAGCLEDCTRQAPESPWEEQLARLLQEAPGKLSLDVEQAP
SGQHSQAQLSGQQQRLLAFFKCCLLTDQLPLAHHLLVVHHGQRQKRKLLTLDMYNAVMLGWARQGAFKEL
VYVLFMVKDAGLTPDLLSYAAALQCMGRQDQDAGTIERCLEQMSQEGLKLQALFTAVLLSEEDRATVLKA
VHKVKPTFSLPPQLPPPVNTSKLLRDVYAKDGRVSYPKLHLPLKTLQCLFEKQLHMELASRVCVVSVEKP
TLPSKEVKHARKTLKTLRDQWEKALCRALRETKNRLEREVYEGRESLYPFLCLLDEREVVRMLLQVLQAL
PAQGESFTTLARELSARTESRHVVQRQRVSGQVQALQNHYRKYLCLLASDAEVPEPCLPRQYWEELGAPE
ALREQPWPLPVQMELGKLLAEMLVQATQMPCSLDKPHRSSRLVPVLYHVYSFRNVQQIGILKPHPAYVQL
LEKAAEPTLTFEAVDVPMLCPPLPWTSPHSGAFLLSPTKLMRTVEGATQHQELLETCPPTALHGALDALT
QLGNCAWRVNGRVLDLVLQLFQAKGCPQLGVPAPPSEAPQPPEAHLPHSAAPARKAELRRELAHCQKVAR
EMHSLRAEALYRLSLAQHLRDRVFWLPHNMDERGRTYPCPPHENHLGSDVARALLEFAQGRPLGPHGLDW
LKIHLVNLTGLKKREPLRKRLAFAEEVMDDILDSADQPLTGRKWWMGAEEPWQTLACCMEVANAVRASDP
AAYVSHLPVHQDGSCNGLQHYAALGRDSVGAASVNLEPSDVPQDVYSGVAAQVEVERRQDAQRGMRVAQV
LEGFITRKVVKQTVMTVVYGVTRYGGRLQIEKRLRELSDFPQEFVWEASHYLVRQVFKSLQEMFSGTRAI
QHWLTESARLISHMGSVVEWVTPLGVPVIQPYRLDSKVKQIGGGIQSITYTHNGDISRKPNTRKQKNGFP
PNFIHSLDSSHMMLTALHCYRKGLTFVSVHDCYWTHAADVSVMNQVCREQFVRLHSEPILQDLSRFLVKR
FCSEPQKILEASQLKETLQAVPKPGAFDLEQVKRSTYFFS
Human POLRMT, mRNA; nuclear gene for mitochondrial product.
(RefSeq NM_005035) Reference No. ENST00000588649.7
(SEQ ID NO: 205)
GGGGTGGCCTGGAGCGGCGTGCGTAATGTCGGCACTTTGCTGGGGCCGCGGAGCGGCGGGGCTCAAACGA
GCCCTACGGCCTTGCGGCCGCCCGGGACTCCCCGGCAAAGAAGGGACCGCCGGTGGCGTCTGCGGCCCCA
GGAGGAGCTCGTCCGCCAGCCCCCAGGAGCAAGACCAAGACCGCAGGAAGGACTGGGGCCACGTGGAGCT
GCTGGAGGTGCTCCAGGCGCGGGTGCGGCAGCTGCAGGCTGAGAGCGTGTCGGAGGTGGTGGTGAACAGG
GTGGATGTGGCGCGGCTCCCAGAATGTGGCAGTGGAGATGGTAGCCTCCAGCCACCCAGGAAGGTCCAGA
TGGGGGCCAAGGATGCCACCCCGGTGCCCTGTGGCCGCTGGGCAAAGATACTGGAGAAGGATAAGCGGAC
CCAGCAGATGCGTATGCAGCGGTTGAAGGCGAAGCTGCAGATGCCATTCCAGAGCGGGGAGTTCAAGGCG
CTGACCAGGCGCCTGCAGGTGGAGCCCCGGCTCCTGAGCAAGCAGATGGCCGGGTGCCTGGAGGACTGCA
CGCGCCAGGCCCCCGAGAGCCCCTGGGAGGAGCAGCTGGCCCGGCTGCTGCAGGAGGCCCCTGGGAAGCT
GAGCCTCGATGTGGAGCAGGCCCCGTCGGGGCAGCACTCGCAGGCCCAGCTCTCAGGTCAGCAGCAGAGG
CTCCTGGCCTTCTTCAAGTGCTGCCTGCTCACTGACCAGCTGCCCCTCGCCCACCACCTGCTGGTCGTCC
ACCACGGCCAGCGGCAGAAGCGGAAGCTGCTCACGCTGGACATGTACAACGCCGTGATGCTTGGCTGGGC
GCGGCAGGGTGCCTTCAAGGAGCTGGTATATGTGTTATTCATGGTGAAGGATGCCGGCTTGACTCCGGAC
CTGCTGTCCTATGCGGCTGCCCTCCAGTGCATGGGGAGGCAGGACCAGGACGCCGGGACCATCGAAAGGT
GTCTGGAACAGATGAGCCAGGAGGGGCTGAAGCTGCAGGCACTCTTCACCGCCGTTCTGCTGTCTGAGGA
GGATCGGGCCACTGTTCTGAAGGCCGTGCACAAGGTGAAGCCCACCTTCAGCCTCCCGCCGCAGCTGCCG
CCCCCGGTCAACACCTCCAAGCTGCTCAGGGACGTGTATGCCAAGGATGGGCGTGTGTCCTACCCGAAGC
TGCACCTGCCCTTGAAGACCCTGCAGTGCCTCTTTGAGAAGCAGCTCCACATGGAGCTGGCCAGCAGGGT
GTGCGTGGTGTCCGTGGAGAAGCCCACGTTGCCAAGCAAGGAGGTCAAGCACGCGCGGAAGACCCTGAAG
ACCCTGCGGGACCAATGGGAGAAAGCACTGTGCCGGGCGCTGCGGGAGACCAAGAACCGCCTAGAGCGCG
AGGTGTACGAGGGCCGGTTCTCACTTTACCCCTTCCTGTGCCTGCTGGACGAGCGCGAGGTGGTGCGGAT
GCTCCTGCAGGTCCTGCAGGCGCTGCCCGCCCAAGGTGAGTCCTTCACCACCCTGGCCCGGGAGCTGAGT
GCGCGCACTTTCAGCCGGCACGTGGTGCAGAGGCAGCGGGTCAGTGGCCAGGTGCAGGCGCTGCAGAACC
ACTACAGGAAGTACCTCTGCTTGCTGGCCTCCGACGCCGAGGTGCCCGAGCCCTGCCTGCCGCGGCAGTA
CTGGGAGGAGCTGGGGGCGCCCGAGGCCCTGCGGGAGCAGCCCTGGCCCCTGCCAGTGCAGATGGAGCTG
GGCAAGCTGCTGGCGGAGATGCTGGTGCAGGCTACGCAGATGCCATGCAGCCTGGACAAGCCGCATCGTT
CCTCTCGGCTTGTCCCCGTGCTCTACCACGTGTATTCCTTCCGCAACGTCCAGCAGATCGGCATCCTGAA
GCCGCACCCGGCCTACGTGCAGCTGCTGGAGAAGGCCGCGGAGCCCACGCTGACCTTCGAGGCGGTGGAT
GTACCCATGCTTTGCCCCCCGCTGCCCTGGACATCGCCGCACTCTGGTGCTTTCCTGCTCAGCCCCACCA
AGCTGATGCGCACGGTGGAAGGCGCCACGCAGCACCAGGAGCTGCTGGAAACCTGCCCGCCCACCGCGCT
GCATGGCGCACTGGACGCCCTCACCCAACTGGGCAACTGCGCCTGGCGCGTCAACGGGCGCGTGCTGGAC
CTGGTGCTGCAGCTCTTCCAGGCCAAGGGCTGCCCCCAGCTAGGCGTGCCGGCCCCGCCCTCCGAGGCGC
CCCAGCCGCCCGAGGCCCACCTGCCGCACAGCGCCGCGCCCGCCCGCAAGGCCGAGCTGCGCCGTGAGCT
GGCGCACTGCCAGAAGGTGGCCCGGGAGATGCACAGCCTGCGGGCGGAGGCGCTGTACCGCCTCTCGCTG
GCGCAGCACCTGCGGGACCGCGTCTTCTGGCTGCCGCACAACATGGACTTCCGCGGCCGCACCTACCCCT
GCCCGCCGCACTTCAACCACCTGGGCAGCGACGTGGCGCGGGCCCTGCTGGAGTTCGCCCAGGGCCGCCC
GCTCGGCCCGCACGGCCTGGATTGGCTCAAGATCCACCTGGTCAATCTCACGGGGTTGAAGAAGCGGGAG
CCGCTGCGGAAGCGCCTGGCCTTTGCGGAGGAGGTGATGGATGACATCCTGGACTCCGCGGACCAACCCT
TGACGGGCCGAAAGTGGTGGATGGGCGCGGAGGAACCCTGGCAGACGCTGGCCTGCTGTATGGAGGTGGC
GAACGCTGTGCGCGCCTCCGACCCTGCCGCCTATGTCTCCCACCTCCCCGTCCATCAGGACGGCTCTTGC
AACGGCCTGCAGCATTATGCTGCTCTGGGCCGCGACAGCGTGGGCGCCGCCTCCGTCAACCTGGAGCCCT
CGGATGTGCCGCAGGACGTGTACAGCGGCGTGGCCGCGCAGGTGGAGGTGTTCCGTAGGCAGGACGCCCA
GCGGGGCATGCGGGTGGCACAGGTGCTGGAAGGTTTCATCACCCGCAAGGTGGTGAAGCAGACGGTGATG
ACGGTGGTGTACGGGGTCACGCGCTATGGCGGGCGCCTGCAGATTGAGAAGCGCCTCCGGGAGCTGAGCG
ACTTTCCCCAGGAGTTCGTGTGGGAGGCCTCTCACTATCTCGTACGCCAGGTCTTCAAGAGTCTACAGGA
GATGTTCTCGGGGACCCGGGCCATCCAGCACTGGCTGACCGAGAGTGCCCGCCTCATCTCCCACATGGGC
TCTGTGGTGGAGTGGGTCACACCCCTGGGCGTCCCCGTCATCCAGCCCTATCGCCTGGACTCCAAGGTCA
AGCAAATAGGAGGTGGAATTCAGAGCATCACCTACACCCACAACGGAGACATCAGCCGAAAGCCCAACAC
ACGTAAGCAGAAGAACGGCTTCCCGCCCAACTTCATCCACTCGCTGGACTCCTCCCACATGATGCTCACC
GCCCTGCACTGCTACAGGAAGGGCCTGACCTTCGTCTCTGTGCACGACTGTTACTGGACTCACGCAGCTG
ATGTCTCCGTCATGAACCAGGTGTGCCGGGAGCAGTTTGTCCGCTTGCACAGCGAGCCCATCCTGCAGGA
CCTGTCCAGATTCCTGGTCAAGCGGTTCTGCTCTGAGCCCCAGAAGATCTTGGAGGCCAGCCAGCTGAAG
GAGACACTGCAGGCGGTGCCCAAGCCAGGGGCCTTCGACCTGGAGCAGGTGAAGCGTTCCACCTACTTCT
TCAGCTGACACCCCGTGAGCCTTGTCAGTGTGTAAATAAAGCTCTTTTGCCACCCCCAGGA
Wild-type mouse POLRMT gene sequence (corresponding Mouse10 dna_chromosome
chromosome GRCm39_10_79571957_79582415)
(SEQ ID NO: 581)
CTCATTACTTTGGTGGGTTTGCGCACCGCCAGAAAGTGGCGGCTGGCATAGGTAGACACAAACTTTCATT
TATTTACATTATGCACAGGTTTAGGGACCGCATGGCGTGGGTCAGCTGAAAAAGTAGGTGGATCTTATCA
CCTGTCCTAGATCGAAGGTACCTGTAAAGCCAAGAGTGCAGAACTAGGCTCGGGCCACTGATTCATCCTC
AAGGCCAGCGTGCATGTCCCCTTTAAATCCTTACCTGTCTTTGGCAAGGACTGTAGTGTCTCCTGCAGCT
TGGTGACCAGGGCGCGCTCCGAGGACTTTAGGGACTTGATGCTGCAAACAGGTTAGGGTAGACATCAGTG
CGGCAGCAGGTGGCAGGCAGGGCACCAGGCCACAAGAGGGGGTATCTCACCTGGACACGGAGCAGAAACG
CTTCTTCAGGAACTTGGCCAGGTCTTCCAGGATGGGCTGGCTGTGCAGGCGCACGAATTGCTCGCGGCAT
ACCTGAGGGGTTGCAGGCTCGGAGTCAGCCATGGTACAAATTCCCAGGATCAAGGCCCCATTTTCTACTT
GTGGGGTCACGGGGGGGGGGGGGGGGGCGGGACGGGACGGACGGGACACACCTCGTTCATCGTGGGGATG
TCAGCGGCATGTGTCCAGAAGCAGTCGTGCACGGAGACGAAAATCAGGCCCTTCCTGAGCAGGGAGATCC
CGAGTGAGATAGCTAGTTGCTTGGCCAGCTACTGCCTGAACCTCGTCTGACAGATATCCTGTCATCTGCC
CCAGCACTGGGCACTCTGCCAGGGTTCCCTGGACACCTTTGGTTCCCCAGTGCTCTAGGTAGTAGATGTT
CAAGAAGATGGCTTACATTCCCTCAAACAGCTGCGGGGTCTTGGAGTAAAGGGAAGCCCCTGTCTGCACC
AAGGACTCCCGCAACTTTCCAGCAGTGCCTCCATCCTGTCCTGCCCCTCATCATGGGGGTACCTACCATG
TCCATGTCCCCAGAAAGCAGAACAAGGCTGGCATGCCCCAGGCAGCTAAATACCACAGACTGAAACCTAG
ACCCAGCGCTCACCTGAAGCAAGAGCCTGCCTGCCTGCCCGCCCTGCTGGGCCCACACTGTGTCCTGGTC
ACACTTCGTCCTGGGACCCACCTGTAGCAGTGTAGGGCGGTCAGCATCATGTGGGAGGAGTCCAGGGAGT
GGATGAAGTTGGGCGGGAAGCCATTTTTCTGCTTCAGAGTGTTGGGCTTCCTGTGGACAGAAATTGTAGG
CTGGTACCCTCTTAGCCCAGAGCCTTTCCTGCCCATCCCTCCATCCTCTCCCCTCCCCGCAGTACAGGAC
TCACTGACTCTCATCTACCGAGCTGGTGAGGGTGATGCTCTGGAGGCCACCTTTTACCTGCAGACCGGGG
TTGGAGGAGGGGTGAGTCCAGCCCTTGGCAGGGAGGAGGGGAGATGAGGGAAAAGGAGAAGGGCATTTCC
CAAGTACCTGGACCTTGGACTCGCGGTGATAGGGCTGTATGATGGGGATGCCCAGGGGCGTGACCCACTC
CACAGGCCATCCGGCGTGAGAGATGAGGTTGGCACTCTCAGTCAGCCAGTGCTGTGGGTGTGACAGTACA
CCAGGTTGGGTGGGTGAGCTGTGGGGTGGAGGGGCCCCCAGATGGGAGAGGGCTGGCAGGGACGCACCTG
AATGGCCCGCGTGCTGGTGAACATCTCCTGTAGACTTTTGAAGACCTGGCGCACGAGGTAGTGTGAGGCT
TCCCAGACAAACTCCTGTGGGGACATTGCGATATGAATGGCAGGTGGGCGGGGCCAGAACCGCGGCTTTA
CAGGACTAGAACGGTTCAGAAGAGCCCGGCTCAGAGTCCCTGTGCTGAAGGGAGTCACACTTGGGTGAGG
ACAGGGCAGGCTGCACACCTGAGGGAAGTCGCTGAGTTCGCGCAGGCGCTTCTCTATCTGCAGGCGCCCT
CCGTAGCGTGTGACCCCATACACCACCGTCATCACTGTCTGCTTCACCACCTTGCGGCTGATGAAGCCCT
CCAGCACCTGAGCCACCCGTAGACCCTCCTTGGCGTCCTGCTGGCGGAACTCCTCCACCTGGATGGGATT
AGGGGTCAGGGAAGCCATTACGTGACCTCCCCTCCCCACTAAGGACCCCATCACTTGTAGAAAGGCTACC
CCGAGTGGGGACAGGAGGGGTTTCCTGGCACCCACCTGTGTTGCCACCTCCCTGTACACATCTTGGGGCA
GGTCGGACGGCGTTAGGTTGACTGAGGCAGCACCCACACTGTCTCGGCCTAGTGCGGCGTAATGCTGTAA
GCCATTGCAGGAGCCATCCTGACCACAAGGGGTGCCCATGAGCTGAGGCCAAATGACCCAATAGGGACAC
ACAGGTCACCCCAGAAACTTATTCCACCACCTGGTGCAAGGAGACCCGGGAGCAACCTCCCTCATAAATC
AAGAGACTAGTCCCACAGGGCAATCCCAGTTTACGGTAAGACCCAACTGCAAATGCCAGTTCCTAAGGCA
CCTCGGGCTCAGCCATGGGCAGTCCACTTGCGTGCCCACCTGGTGAACTGGCAGGTGGGAAATGTAGGCA
GCAGGGTCTGGGGACCGGACTGCGTGTGCCACCTCCATGCAGCAGGCCAGGGTCTGCCAGGGCTCATCAG
CTTCCATCCACCACTTCCGGCCCTTTGGAGGGTAGGATCCATGGGAATCGAGTCAGCACAAGCCAGCCTG
GGCAGCTCAGTGGAACACAGGGACGTAGGGACACTAGGCAAGCATGTGTGATGTAGGGGGATGAGGAAGG
CTGCTCGGGTGATAGCTGGCAGCTGCTGCTGGGCTAGGGAGCCACTAGTGGCACTAAGAGGGGACACCGT
GGGAAGGAAGGATGCAGATAGCTGTGGTGAAGGGTTGAAAGCATATGAGGGAGGTGGCCCAAGCCTGAGT
GGATGACAGGCTGGCAGAGCCAGAGAAAGACAGGAGGCAGAGATGAGGGCTGCCAGCGAGAGGAGCTCGT
GACTCCATACCGTCAAGGGGTTGTCTGCAGAGTCCAAGATCTCCTCCATGACCTCATCTGCGAAGGCTAG
GCGCATGCGCAGCGAGTCTCCCTTCTTGAGGCCAGTCAGGTTGATCAGGTGGATCTTAAGCCAGTCCAGA
CCACGTGGTCCCAGTGGCCGGCCCTCAGCAAACTCCAATAGCGCACGCGCTAGGTCACTGCCCAGGTGGT
TGAAGTGTGGCGGGCAGGGGTAAGTGCGGCCGCGGAAGTCCATGTTGTGCGGCAACCAGAAGACACGGTG
GCGTAGGTGCTGTGCCAGCGACAGGCGATACAGGGCCTCGCTGCGCAGACTGTGCATCTCTCGAGCCACC
TTGAGGCAACGCGCCAGCTCCTTCCGCAGCTCAGACTTGTGCACTGGTGTGGAGCCGGGTGGCAGCTGAT
ACCGGGCCGGCCGCGGTGCTTCTGAGCGCGGGGGAGGCACGCCCAAGGGCATACAGCCCTTGTCTCTAAA
GATCTGCAGCACCAAGTCCAGCAGATGCCCGTTTACACGCCAGGCGCAGTTCCCCAACTGTGTGAGCGCA
TCCAGGGGGCCGTGCAGCTGGGCAGGAGGGCATTGCTCCAGCAGACGCTGGTGCTGTGTGGTACCCTCCG
TGGCACGCATTAGTTTGGTGGAGCTCAGCAAGTAGGCACCAGAATGCAGCGACGTCCAGGGCAGTGGTGG
GCACAGCATGGGCACTTCCGTGGTCTCGAAGGTCAGTGTGGGCTCTGCTGCCGTCTCTAGCAGGTGCGTG
AAGGCAGGGTGAGGCTTAAGGATGCCCACCTGGGAGAGGAAGCAGACACAAATGGTATGCATCAGAGGCC
GAGTGCCAGTGCCTCTGTGCAACGGACCACGCAGGCAGGGAGAACAGAAAACTCCAAGCCCTAGACTGAT
GTAACCCCTCCTGCTCTGTACACACATCCCACACGGAGGGGATGGCAGACACCTGCTGCCCTATGTCCCA
TCCAGGCGCCGGCCGGCCCTGGGAGCCTCACCTGGCGGTAGCTTCGGAAGGAATACACATGGTAAAGCAC
TGGGATGGAGCGCTGAGCACCCTGCCGGGCCGCCAGGCTGCGCGGCATCTGCACTGCCTGCACCAGCAGC
TCCGCCAATTGCTTGCCCAGCTGTAGCAGCACTGGCACGGACCAGGGCTGTTGTGCAGGGGCCTCCAGTG
GGCCCAGCGATTCCCAGTACTCCCGAGGAAGGCAGGGCGCCACCTGTAAGGACAGCTGGTCAGGTGAGCT
GTGGGGCTGCCCACGTTTTCCAGAGGGAACCGAGGCTCAGACGGACTGATGCAGCTGGCAAGGGGCGCCT
GAGGAGTGTGGTGGGGAGGGACCTGGGTGGAACCTGGAGCAAGTGGGGTGAAGTGAACTGGGAGGGGATG
TCTCCAAGGGAGCCACCTGGGTCACCAGAAGTCCATCCCACGTTACCTGGGTGTCAGACGCCAGCAGCTG
GAGGTACTGCGAGTACCGCTGCCCCAGCTTCTGCACATGGTTAGTGACCTGCTTCTGCTTCACCAAATGC
CGGTTTAACACCCGAAGGCCCAGGTTATGGGCCAACTGGATAAGGGGCTCACCCTGCGCAGGCAGGACCT
TCAGAACCTGAGGTGCAAGAGGATCGCGAGCTGTGACTGCCTGAACACAGCCATCTGCCCTGTGCCCAGG
AACCTCCAGACCCAGCCCACCTGCATCAGTATGCTCACAAACTCCCCTTCGCTCAGCAGGCACAGGAACG
GGTAGAGGGTGGGCTGGCCTTCATAGGCCTGGCGGCCCATAGTGGCCTTTGTCTCCCGCAGCACGCGCAA
CAGCTCCACTTCCCACTGCTCCCGCAGGGCCTGCAGGGTCTTCCGCTGTAGGAGAGCAGCTGTCAACACG
CTGAGCTTAAAGCCCCCTGCCAAGACCTCCCAAGGCCCCAACTGGGGTCATAGCTCCGACCCCTACTGGC
CATGATGGGTACAGCCTCAGGATGAGACCCTTACCGCCTCAATGACCTCCTTGGACATTACTGGAGCCTT
CTCCACCGACTGGACACAGACACTGGAGCTCAGCTCCACATGCAGCTGCTGGTAGAAGAGGTCCTGCAGG
GTGTCCAGGGGCAGGTGCAGCTTTGGGTAGGACACAGGGCCCTCCTGCTGGGCACAGGAGGCAGTGAGGG
TGGGCACTACACCCTGTCAAGAAGCCCCCTGCCAAACGCATGTCCACTCAAAGTAAAAACCCAGAGTACT
AAGAGTTCAGACACCTGACTCCCAGTAAAAACAGCCCAATGCCCCGAACAGATGATGGATACACAGGAGG
CCCCATCTACACACTGGAATACAACTGAGCCATGAGTAGGAGCGAGCTTCCAACATATTCCGCAGCGTGA
AGGGACCTTGAGGACACTGTAATCGATGAGCCAGATACAAAAGGCCACAGGGACTAGGGCTCTATGATAG
CAAACGCCCAGAACAGGAGAATCCAGAGGAAGTGGATTTGGGGGTGCTCCTCTGAGGGGACCAACATGTG
CCTGATGCAAGGAGGGAATGAAACAGGCTGTCATGACTTCTTGCCTGGGACTCACCTTGCTATATATGTC
CTTGAGCAGCGTGGATGTGTTAACTGGGCTTGGGGCCTGTGGCGGTGGACGAAAGGCAGGCTCAGCCTTG
ACCACTGCTCTCAGGAGCGCGGCCCGGTCCTCCTCTTCCAGGACCAGGTCAGTGAAGAGCAGCTGGGGCT
GGAAGCCTTCTTCCATCATCTGCTTCAGACACCTGAGGCAATACAGGGGTGGGGGTGGGGGTGTGCAAAC
TTAGCATGGGAGCATGGGGTGGGGGGAGCAGGGCCCCAGCACATTCCTGACCCACCTCTGGATGGTGCGA
ACATCCTGGTCCCTGCGTCCCATGCACTGGAGTGCAGCTGCATAGGAGCACAGGTCTGGGGAGAGGCCAG
CATCCTTCAGCATGAGGAACACATAGACCAGCTCTCTGAAGGAGCCCTGGGGACAAGGTGGGGTCAAGAA
CAACACCTGGACCCTGACCAATCTGTTCCCTAAAATCCTCAGTAATTTGGGAGGGGGGATCTGGCAGTAG
GTGAGACACAGGGCATCTTTCTGAGGGACACTGCGTCATTGGGGACTGAACTCGGGCCTCTTGCAGGCCA
GGCATATGCTCTACCACTAAGCTACAGCTTCAGGACTTGTGTTTTATAGCTGGCTTGGTTTTTGTTGTGG
TTCTTTGTTTGTTTGTTGAGAACTTGTCTCACTGTGTAGCCCTGGCTGGCCTGAAACTCTCTAAGTAGGT
CAGACTGGCCTTGAACTCACAGACCTGTCTCCTGTCTACTGCTCTGTCCTTTCCTTGTTTTTATTTTGCT
TTTCTGTCTTTGAACTCCTAGACTGACACTCTCCTACACTGCTAAGATGTTAGGGAACAAACTGAGGACA
CACAGCTCTCATAGCACCAAAGCGACACACAGAAAGAGAAGCGCCGGGCGTGGTAGCGCACGCCTTTGAT
CCCAGCACTCGGGAGGCAGAAGCAGGCGGATTTCTGAGTTCGAGGCCAGCCTGGTCTACAGAGTGAGTTC
CAGGACAGCCAGGGCTACACAGAGAAACCCTGTCTTGAAAAACCAAAAAAAAAAATAAAAATAAAAAAAA
AATAAAAATAAAAAAAAAATAAAGAGAAGCAAGCGCTCCCATCCTGGAGCGTGGAATGTGTCCCACAAGG
CTTAGCAGCCACTGGGACAGCAGCGGCAGCCCCTGCACAGGAGACTCAGAAGTCCTGCACAGAACTACAC
TCAGGCCCTCCCAATCTCCTCCTTCATTTTAGGGGATAGCAGGATTTTAATGTGTTGACCAGGCTGCCCT
CTAACTCTCAGAGATCCACCTGCCTCTGCCTCCCAAGTGCTGGGACTAAAGGCTTGCGCCACCATGCCTG
GATATTCCAATCGGTTTTTGTTTTGTTTCCTGTTTGTTCGTTTTTAAGGAAAGAGCGTTTTCTCTCTGAA
AAACCTAAAGGAATTTACCCCATGGGAAGGAAGCAGCCCTTGGGCCGGGTCTCACCTTGCGGGCCCAGCC
AAGCATCACGGTGTTGTACATGTGCAGTGTGAGCACCTGCTGTCTGTCTCCGTTGTTATGGTGAGTGACC
AGCACGTGGTGAGCGAGGGGCACTTGGCCAGTGCAGACGCAGCACTCAAAGAAAGCCAGGAACTTCTGCT
GGAGGACCAGGATCTGCGCCTCCACCGCCTTGGCTTTCTTCCTGGCCAGGGCCTCTGCCTCACGGGAGCT
GAGCCTCCCCAGAGCAGCCTGAAGGGCCTGGGCCAGCTGCTTTTCCTCTGAGTTCGTGGGTGTTCCCTTC
TTGCTTGGCTGCAGGTAGCCAGCCAGCTTCTTGTTCCAGATCTTGGGCTCCTTGTGAAGGGTCCAGAACT
CCTGTGTGAGGGCTTGCTTCTGCTGGTCAACTTCCTTTTGGCGCCTCTGCTTCACCCTTTTCTCAGCCTC
TAGCTTTTGCGCCCAGCGGCTGCTGTGGCCCTGTGGAGGCCGGTCCACCTGCACCTTCTTCACCCTCATC
TCAGGTGTGCCCTCTGCCCGGAGCTGCCGCACCCGAGCCTCCAGCACTGCAGAGAGCAGAGAAGGGTATG
AACCACTCCTGCAGCTCAGGATGCTCTGAGTCTACCCCAGGATCATCCCAAACACAGTCTCCTGGCTGTC
ACCATGCCCTCGGCTCTGCCTGCCAACCCCTCCTATCACCTCCTGCCGGCTGTCACCTGCTTCTGCTTCT
CCCTCCTTCACTCCGCCAGCCTTAAGGGCTCCGACCTCACTGGCCAATATATAGTAGCCTGCAGTTTGAT
ACAGGCACATACTTGATACAGCTTGGTAGAAACCGCAAACATCCGTGACTCAGTGCCCTAAGGACCAAAA
GGCAATCACTGGCACCATACAAGGAGGCCCCTGCTGTATGGCTTTCAGCAGTAACCAAGTTTGCACATAA
AAAGGCCAAAGACTGGCCAGGGTCTTGGCAAGCAGACAGCCATGGTTAGTGGTGAGCGCAGAAACAAGGC
AGGCTGAGCCATAAGGCTACAACTCAGGCGCTGGTTGCCAAGACCAGCTGCTTGTCTCCCCTCCCGGTAC
TCCAGTCCCTGGCCCAAGGTCAGTGTGAGGCAGGGGCCTGGCTCACAGGTGTACAAAACGAAAGTTGGGA
GGAGTGCAGTGCATGCGAGAGGATCAGAGTTCTGGGGAGATCCTGCCTCAGTGAACATCTCGTGGCACTC
AGGGCACATCAGAGGCAGAGGACACATCTGTGTGAGGCCACCTGCAAAGCAAACAGGTGGCAGACGGGGA
CACCGACTTCTTTCTGAAGGGACCCAAGAGACAAAAGGGTGCAAAAAGGCCCTGGGGCAGAACTGGTTGG
TCTGTCAGGAGTAGAAGAGTTACATAGCTGCTGTAAGCTCCATGGCCCACAAAGCACTGCCTTCTGTCCC
TGACAGAAAGGAGCCTCATAAGAGGGAGCAGGCGCCACACACTAAGTCTGCAGATCCCTCTGCCCAAATG
ACAGATTCCAGCATGACATACCCTGGAACGTGAGTCAGGAAGCAGTTGCTGCTAAGGGTAGACAGGAGCC
ACAGTTAGACCATTGTCTTTACCTCCTAACCCACCCTCTAAGCGTCTCCACCTTCTGCAGCTGCCATGCA
GACCTGACATAGGATCAGGAGGGAAATCTGGACATCCCGTCTACTAGACTTGGTAGTAAAACACTTAAAA
TTGGGTTCCAAAGTCAGTGGTCGTGCATGCCTTTATTTTGTTTTATTTTTGTTTGTTTGGTTTTTTTTTT
TTTTTTTTTTTTTTTGGTTTTTTCTTTTTTTCAAGACAGGGTTTCTCTGTGTAGCCCTGGCTGTCCTGGA
ACTCATTTTGTAGACCAGGCTGGCCTCGAACTCAGAAATCCGCCTGCCTCTGCCTCCAGAGTGCTGGGAT
CAAAGGCGTGCGCCACCATACCCGGCTCATGCACACCTTTAATCCCAGCACTCCAGGGGCAGAGGCAGGC
AGACCTGAGTTCAAGGCCAGCCTGGTCTACAAAGCAGCCAGGACTTCCTACACAGAGAAACCCTGTCTCG
AAAAACCAAGAAAGAAAAAAGAGAGAAAGAGAGAAGGGAGGAATGAGGAGAGAAAGAAAGAAAGAGGAAG
GAAGGAAGGAAGACAGACAGGCAGGCCTGGCAGACAGGCTGGATTCCAGCTGCACCTCGACTCTGCAGGA
CTAAAACTGGAGACTTCTATGTCAGCATTCCAACAATGCAAATCCGGTATTGTTCAGGTGTATTTGGGGC
CTCCACTGTCTTCCTAGAAGTGAGAGCCCCAGGTGGGAATCTGGAACTCAGGGAATGGAATTACAGCAGC
TCCCAGAACCAGCACAAGAGGACCCACAGGACCACAGACAGGCCAGCCAGACAACACTGCTCAGTGGACT
CTGGACCCCATTTTACATACTCCCTTGGAGTGGTCAGGTACAACGCAGAGCACACTCCCTCAAGGCTTCA
CTGTGGAGAAGGACTTAGGACACCAACTTACCTTCCAGCAGTTCAGCATGGCCCCACTCCCTCAGGACAT
GTTGCTCACGGGGACTGGCAGCGGAGCTCCTTCTTGAGCTGCAGAAGCCACCAAAAGTCCCTAGAAAGAG
ACATGAAGAATAAGAACACAGAAACCATCCTATCAAGTGGACACCGTGGCATGCACACTTATTACCCCAG
GTCTCTGGAGGCTGAGGTGGGAGGGTCTCCAATTGGATTCCAGCCTGGGCTACAAAGGAAGTTCTAGGTT
AAGCTAGTGTGAGTTCCTGCCTCAAACTTACGTCATGCCCAGAAAGAAATGGATCAAAAGACAAGGACTG
CCAGGCATGGGATCTCAGAACTCAGGAGGTTGAAGCAGTAGGACCTGAGTTCAAATAAAGCATCCAGCAT
GAACCACATGGAAACACCATGGCTCAAAAAAAAAAAAAAAAAAAAAAAAAAAACACGCCCCGCCCACAAA
TAAATCCAGACCCAGAAAAAGACCAACTTCAAGGGCAGAACAGGCCAAAGCTATAAGGATCGGGGTGCAG
GATCCAGCAAGAAGTACTCCTAACTAAAAGTAGCCAAAGGTCAATGCCAATATCCGTAAGTCCCCAACAG
TGGGTCAGAGACATGGAAAAGCGAGCAACAATCAAAAGTCAAAGTGCCCTGCGGACCTAGAGAGACGTTG
CCAACTAGTGCCCAGCTAAATGGGTCCCCAGAAACTCCTCCATAGGTCAAAGAACAAAGGTTGAAGGTGA
CCGGGATCCTAAGGAAGGGAAGATTATAGTCCTGGATCCGACACTCCTTTACCTTCCTCCGAAGGCGGGC
GGTGGGGCCCAGGGGAGCGCAGAACCCGGCCAAGCCCGGCTGCGCTTCGGGTCCACCGGAGCGCCGACAT
GCCGCCTGCTGCCCGCAGTGCGCACGCGC
Mouse POLRMT transcript ENSMUST00000161765.8 Polrmt-210 cdna
(SEQ ID NO: 582)
GCACTGCGGGCAGCAGGCGGCATGTCGGCGCTCCGGTGGACCCGAAGCGCAGCCGGGCTTGGCCGGGTTC
TGCGCTCCCCTGGGCCCCACCGCCCGCCTTCGGAGGAAGGGACTTTTGGTGGCTTCTGCAGCTCAAGAAG
GAGCTCCGCTGCCAGTCCCCGTGAGCAACATGTCCTGAGGGAGTGGGGCCATGCTGAACTGCTGGAAGTG
CTGGAGGCTCGGGTGCGGCAGCTCCGGGCAGAGGGCACACCTGAGATGAGGGTGAAGAAGGTGCAGGTGG
ACCGGCCTCCACAGGGCCACAGCAGCCGCTGGGCGCAAAAGCTAGAGGCTGAGAAAAGGGTGAAGCAGAG
GCGCCAAAAGGAAGTTGACCAGCAGAAGCAAGCCCTCACACAGGAGTTCTGGACCCTTCACAAGGAGCCC
AAGATCTGGAACAAGAAGCTGGCTGGCTACCTGCAGCCAAGCAAGAAGGGAACACCCACGAACTCAGAGG
AAAAGCAGCTGGCCCAGGCCCTTCAGGCTGCTCTGGGGAGGCTCAGCTCCCGTGAGGCAGAGGCCCTGGC
CAGGAAGAAAGCCAAGGCGGTGGAGGCGCAGATCCTGGTCCTCCAGCAGAAGTTCCTGGCTTTCTTTGAG
TGCTGCGTCTGCACTGGCCAAGTGCCCCTCGCTCACCACGTGCTGGTCACTCACCATAACAACGGAGACA
GACAGCAGGTGCTCACACTGCACATGTACAACACCGTGATGCTTGGCTGGGCCCGCAAGGGCTCCTTCAG
AGAGCTGGTCTATGTGTTCCTCATGCTGAAGGATGCTGGCCTCTCCCCAGACCTGTGCTCCTATGCAGCT
GCACTCCAGTGCATGGGACGCAGGGACCAGGATGTTCGCACCATCCAGAGGTGTCTGAAGCAGATGATGG
AAGAAGGCTTCCAGCCCCAGCTGCTCTTCACTGACCTGGTCCTGGAAGAGGAGGACCGGGCCGCGCTCCT
GAGAGCAGTGGTCAAGGCTGAGCCTGCCTTTCGTCCACCGCCACAGGCCCCAAGCCCAGTTAACACATCC
ACGCTGCTCAAGGACATATATAGCAAGGAGGGCCCTGTGTCCTACCCAAAGCTGCACCTGCCCCTGGACA
CCCTGCAGGACCTCTTCTACCAGCAGCTGCATGTGGAGCTGAGCTCCAGTGTCTGTGTCCAGTCGGTGGA
GAAGGCTCCAGTAATGTCCAAGGAGGTCATTGAGGCGCGGAAGACCCTGCAGGCCCTGCGGGAGCAGTGG
GAAGTGGAGCTGTTGCGCGTGCTGCGGGAGACAAAGGCCACTATGGGCCGCCAGGCCTATGAAGGCCAGC
CCACCCTCTACCCGTTCCTGTGCCTGCTGAGCGAAGGGGAGTTTGTGAGCATACTGATGCAGGTTCTGAA
GGTCCTGCCTGCGCAGGGTGAGCCCCTTATCCAGTTGGCCCATAACCTGGGCCTTCGGGTGTTAAACCGG
CATTTGGTGAAGCAGAAGCAGGTCACTAACCATGTGCAGAAGCTGGGGCAGCGGTACTCGCAGTACCTCC
AGCTGCTGGCGTCTGACACCCAGGTGGCGCCCTGCCTTCCTCGGGAGTACTGGGAATCGCTGGGCCCACT
GGAGGCCCCTGCACAACAGCCCTGGTCCGTGCCAGTGCTGCTACAGCTGGGCAAGCAATTGGCGGAGCTG
CTGGTGCAGGCAGTGCAGATGCCGCGCAGCCTGGCGGCCCGGCAGGGTGCTCAGCGCTCCATCCCAGTGC
TTTACCATGTGTATTCCTTCCGAAGCTACCGCCAGGTGGGCATCCTTAAGCCTCACCCTGCCTTCACGCA
CCTGCTAGAGACGGCAGCAGAGCCCACACTGACCTTCGAGACCACGGAAGTGCCCATGCTGTGCCCACCA
CTGCCCTGGACGTCGCTGCATTCTGGTGCCTACTTGCTGAGCTCCACCAAACTAATGCGTGCCACGGAGG
GTACCACACAGCACCAGCGTCTGCTGGAGCAATGCCCTCCTGCCCAGCTGCACGGCCCCCTGGATGCGCT
CACACAGTTGGGGAACTGCGCCTGGCGTGTAAACGGGCATCTGCTGGACTTGGTGCTGCAGATCTTTAGA
GACAAGGGCTGTATGCCCTTGGGCGTGCCTCCCCCGCGCTCAGAAGCACCGCGGCCGGCCCGGTATCAGC
TGCCACCCGGCTCCACACCAGTGCACAAGTCTGAGCTGCGGAAGGAGCTGGCGCGTTGCCTCAAGGTGGC
TCGAGAGATGCACAGTCTGCGCAGCGAGGCCCTGTATCGCCTGTCGCTGGCACAGCACCTACGCCACCGT
GTCTTCTGGTTGCCGCACAACATGGACTTCCGCGGCCGCACTTACCCCTGCCCGCCACACTTCAACCACC
TGGGCAGTGACCTAGCGCGTGCGCTATTGGAGTTTGCTGAGGGCCGGCCACTGGGACCACGTGGTCTGGA
CTGGCTTAAGATCCACCTGATCAACCTGACTGGCCTCAAGAAGGGAGACTCGCTGCGCATGCGCCTAGCC
TTCGCAGATGAGGTCATGGAGGAGATCTTGGACTCTGCAGACAACCCCTTGACGGGCCGGAAGTGGTGGA
TGGAAGCTGATGAGCCCTGGCAGACCCTGGCCTGCTGCATGGAGGTGGCACACGCAGTCCGGTCCCCAGA
CCCTGCTGCCTACATTTCCCACCTGCCAGTTCACCAGGTGGGCACGCAAGTGGACTGCCCATGGCTGAGC
CCGAGGTGCCTTAGGAACTGGCATTTGCAGTTGGGTCTTACCGTAAACTGGGATTGCCCTGTGGGACTAG
TCTCTTGATTTATGAGGGAGGTTGCTCCCGGGTCTCCTTGCACCAGGTGGTGGAATAAGTTTCTGGGGTG
ACCTGTGTGTCCCTATTGGGTCATTTGGCCTCAGCTCATGGGCACCCCTTGTGGTCAGGATGGCTCCTGC
AATGGCTTACAGCATTACGCCGCACTAGGCCGAGACAGTGTGGGTGCTGCCTCAGTCAACCTAACGCCGT
CCGACCTGCCCCAAGATGTGTACAGGGAGGTGGCAACACAGGTGGGTGCCAGGAAACCCCTCCTGTCCCC
ACTCGGGGTAGCCTTTCTACAAGTGATGGGGTCCTTAGTGGGGAGGGGAGGTCACGTAATGGCTTCCCTG
ACCCCTAATCCCATCCAGGTGGAGGAGTTCCGCCAGCAGGACGCCAAGGAGGGTCTACGGGTGGCTCAGG
TGCTGGAGGGCTTCATCAGCCGCAAGGTGGTGAAGCAGACAGTGATGACGGTGGTGTATGGGGTCACACG
CTACGGAGGGCGCCTGCAGATAGAGAAGCGCCTGCGCGAACTCAGCGACTTCCCTCAGGAGTTTGTCTGG
GAAGCCTCACACTACCTCGTGCGCCAGGTCTTCAAAAGTCTACAGGAGATGTTCACCAGCACGCGGGCCA
TTCAGCACTGGCTGACTGAGAGTGCCAACCTCATCTCTCACGCCGGATGGCCTGTGGAGTGGGTCACGCC
CCTGGGCATCCCCATCATACAGCCCTATCACCGCGAGTCCAAGGTCCAGGTACTTGGGAAATGCCCTTCT
CCTTTTCCCTCATCTCCCCTCCTCCCTGCCAAGGGCTGGACTCACCCCTCCTCCAACCCCGGTCTGCAGG
TAAAAGGTGGCCTCCAGAGCATCACCCTCACCAGCTCGGTAGATGAGAGTCAGTGAGTCCTGTACTGCGG
GGAGGGGAGAGGATGGAGGGATGGGCAGGAAAGGCTCTGGGCTAAGAGGGTACCAGCCTACAATTTCTGT
CCACAGGAAGCCCAACACTCTGAAGCAGAAAAATGGCTTCCCGCCCAACTTCATCCACTCCCTGGACTCC
TCCCACATGATGCTGACCGCCCTACACTGCTACAGGAAGGGCCTGATTTTCGTCTCCGTGCACGACTGCT
TCTGGACACATGCCGCTGACATCCCCACGATGAACGAGGTATGCCGCGAGCAATTCGTGCGCCTGCACAG
CCAGCCCATCCTGGAAGACCTGGCCAAGTTCCTGAAGAAGCGTTTCTGCTCCGTGTCCAGCATCAAGTCC
CTAAAGTCCTCGGAGCGCGCCCTGGTCACCAAGCTGCAGGAGACACTACAGTCCTTGCCAAAGACAGGTA
CCTTCGATCTAGGACAGGTGATAAGATCCACCTACTTTTTCAGCTGACCCACGCCATGCGGTCCCTAAAC
CTGTGCATAATGTAAATAAATGAAAGTTTGTGTCTACCTATGCCAGCCGCCACTTTCTGGCGGTGCGCAA
ACCCACCAAAGTAATGAG

TABLE 15
POLRMT Oligonucleotide Sequences

| SEQ ID | |
|---|---|
| NO: | ASO Sequence (5′ to 3′) |
| 3 | CCCCAGTCCTTCCTGCGGTC |
| 4 | TCCACATCGAGGCTCAGCTT |
| 5 | CTCCACATCGAGGCTCAGCT |
| 6 | TGCTCCACATCGAGGCTCAG |
| 7 | CCCCAGCTCCTCCCAGTACT |
| 8 | GGCCACCTTCTGGCAGTGCG |
| 9 | GCCACGTCGCTGCCCAGGTG |
| 10 | CGCCACGTCGCTGCCCAGGT |
| 11 | CCCATCCACCACTTTCGGCC |
| 12 | GCCCATCCACCACTTTCGGC |
| 13 | CGCCCATCCACCACTTTCGG |
| 14 | TTCGCCACCTCCATACAGCA |
| 27 | GGTCTTGGTCTTGCTCCTGG |
| 28 | GAGGCTACCATCTCCACTGC |
| 29 | GCATCCTTGGCCCCCATCTG |
| 30 | CTCCAGTATCTTTGCCCAGC |
| 31 | TCTCCAGTATCTTTGCCCAG |
| 32 | TTCTCCAGTATCTTTGCCCA |
| 33 | CTTCTCCAGTATCTTTGCCC |
| 34 | CCTTCTCCAGTATCTTTGCC |
| 35 | TCCTTCTCCAGTATCTTTGC |
| 36 | CACATATACCAGCTCCTTGA |
| 37 | TCCTCCTCAGACAGCAGAAC |
| 38 | CACTGCAGGGTCTTCAAGGG |
| 39 | TGCTTGACCTCCTTGCTTGG |
| 40 | GTGCTTGACCTCCTTGCTTG |
| 41 | TGCCCAGGTGGTTGAAGTGC |
| 42 | TGTAGACTCTTGAAGACCTG |
| 43 | AGCATCATGTGGGAGGAGTC |
| 44 | GAGCATCATGTGGGAGGAGT |
| 45 | TTCAGCTGGCTGGCCTCCAA |
| 71 | GAGATAGTGAGAGGCCTCC |
| 72 | AGGAATACACGTGGTAGAG |
| 73 | CCAGTACTGCCGCGGCAGG |
| 74 | AGTCCAGGATGTCATCCAT |
| 75 | TCTTCTGCTTACGTGTGTT |
| 76 | ATCATGTGGGAGGAGTCCA |
| 77 | GCCTGCAGGACCTGCAGGA |
| 78 | CAGCTGGTCAGTGAGCAGG |
| 79 | CTATTTGCTTGACCTTGGA |
| 80 | CAAAGCATGGGTACATCCA |
| 81 | AAGGGGTAAAGTGAGAACC |
| 82 | AGAGGTACTTCCTGTAGTG |
| 83 | AACACATATACCAGCTCCT |
| 84 | TTCATGACGGAGACATCAG |
| 85 | GTAGGGCTCGTTTGAGCCC |
| 86 | CCCATGTGGGAGATGAGGC |
| 87 | GAGTCAAGCCGGCATCCTT |
| 88 | CCAAGATCTTCTGGGGCTC |
| 89 | CACTGCAGGGTCTTCAAGG |
| 90 | CACACCCTGCTGGCCAGCT |
| 91 | ACGCCGCTCCAGGCCACCCC |
| 92 | GCACGCCGCTCCAGGCCACC |
| 93 | ACGCACGCCGCTCCAGGCCA |
| 94 | GGCCCCAGCAAAGTGCCGAC |
| 95 | GTCCTTCCTGCGGTCTTGGT |
| 96 | AGTCCTTCCTGCGGTCTTGG |
| 97 | CCCCAGTCCTTCCTGCGGTC |
| 98 | CACGTGGCCCCAGTCCTTCC |
| 99 | CATCCACCCTGTTCACCACC |
| 100 | CACATCCACCCTGTTCACCA |
| 101 | GCGCCACATCCACCCTGTTC |
| 102 | GCCCCCATCTGGACCTTCCT |
| 103 | GAACTCCCCGCTCTGGAATG |
| 104 | CTTGAACTCCCCGCTCTGGA |
| 105 | CGGCCATCTGCTTGCTCAGG |
| 106 | CCGGCCATCTGCTTGCTCAG |
| 107 | ACCCGGCCATCTGCTTGCTC |
| 108 | CTCCAGGCACCCGGCCATCT |
| 109 | CCTCCAGGCACCCGGCCATC |
| 110 | ATCGAGGCTCAGCTTCCCAG |
| 111 | CCTGCTCCACATCGAGGCTC |
| 112 | GGCGTTGTACATGTCCAGCG |
| 113 | GCATCACGGCGTTGTACATG |
| 114 | AGCATCACGGCGTTGTACAT |
| 115 | AAGCATCACGGCGTTGTACA |
| 116 | TCCAGACACCTTTCGATGGT |
| 117 | CAGAACGGCGGTGAAGAGTG |
| 118 | GCAGAACGGCGGTGAAGAGT |
| 119 | GACAGCAGAACGGCGGTGAA |
| 120 | ACGGCCTTCAGAACAGTGGC |
| 121 | CACCTTGTGCACGGCCTTCA |
| 122 | ACACGCCCATCCTTGGCATA |
| 123 | GGACACACGCCCATCCTTGG |
| 124 | AGGACACACGCCCATCCTTG |
| 125 | GTAGGACACACGCCCATCCT |
| 126 | GGTAGGACACACGCCCATCC |
| 127 | GACCTCCTTGCTTGGCAACG |
| 128 | TTTCTCCCATTGGTCCCGCA |
| 129 | GTGCTTTCTCCCATTGGTCC |
| 130 | CCGGCACAGTGCTTTCTCCC |
| 131 | CCACTGACCCGCTGCCTCTG |
| 132 | CTCCTCCCAGTACTGCCGCG |
| 133 | TTGTCCAGGCTGCATGGCAT |
| 134 | TCCACCGCCTCGAAGGTCAG |
| 135 | ATCCACCGCCTCGAAGGTCA |
| 136 | CATCCACCGCCTCGAAGGTC |
| 137 | ACATCCACCGCCTCGAAGGT |
| 138 | GGTACATCCACCGCCTCGAA |
| 139 | GTGCGGCGATGTCCAGGGCA |
| 140 | AGAGTGCGGCGATGTCCAGG |
| 141 | CAGAGTGCGGCGATGTCCAG |
| 142 | CCAGAGTGCGGCGATGTCCA |
| 143 | CACCAGAGTGCGGCGATGTC |
| 144 | AGCACCAGAGTGCGGCGATG |
| 145 | GGTCCAGCACGCGCCCGTTG |
| 146 | AGGTCCAGCACGCGCCCGTT |
| 147 | GCCAGCTCACGGCGCAGCTC |
| 205 | CCGCCGCTCCAGGCCACCCC |
| 206 | GCACGCCGCTCCAGGCCACC |
| 207 | ACGCACGCCGCTCCAGGCCA |
| 208 | GGCCCCAGCAAAGTGCCGAC |
| 209 | GTCCTTCCTGCGGTCTTGGT |
| 210 | AGTCCTTCCTGCGGTCTTGG |
| 211 | CCCCAGTCCTTCCTGCGGTC |
| 212 | CACGTGGCCCCAGTCCTTCC |
| 213 | CATCCACCCTGTTCACCACC |
| 214 | CACATCCACCCTGTTCACCA |
| 215 | GCGCCACATCCACCCTGTTC |
| 216 | GCCCCCATCTGGACCTTCCT |
| 217 | GAACTCCCCGCTCTGGAATG |
| 218 | CTTGAACTCCCCGCTCTGGA |
| 219 | CGGCCATCTGCTTGCTCAGG |
| 220 | CCGGCCATCTGCTTGCTCAG |
| 221 | ACCCGGCCATCTGCTTGCTC |
| 222 | CTCCAGGCACCCGGCCATCT |
| 223 | CCTCCAGGCACCCGGCCATC |
| 224 | ATCGAGGCTCAGCTTCCCAG |
| 225 | CCTGCTCCACATCGAGGCTC |
| 226 | GGCGTTGTACATGTCCAGCG |
| 227 | GCATCACGGCGTTGTACATG |
| 228 | AGCATCACGGCGTTGTACAT |
| 229 | AAGCATCACGGCGTTGTACA |
| 230 | TCCAGACACCTTTCGATGGT |
| 231 | CAGAACGGCGGTGAAGAGTG |
| 232 | GCAGAACGGCGGTGAAGAGT |
| 233 | GACAGCAGAACGGCGGTGAA |
| 234 | ACGGCCTTCAGAACAGTGGC |
| 235 | CACCTTGTGCACGGCCTTCA |
| 236 | ACACGCCCATCCTTGGCATA |
| 237 | GGACACACGCCCATCCTTGG |
| 238 | AGGACACACGCCCATCCTTG |
| 239 | GTAGGACACACGCCCATCCT |
| 240 | GGTAGGACACACGCCCATCC |
| 241 | GACCTCCTTGCTTGGCAACG |
| 242 | TTTCTCCCATTGGTCCCGCA |
| 243 | GTGCTTTCTCCCATTGGTCC |
| 244 | CCGGCACAGTGCTTTCTCCC |
| 245 | CCACTGACCCGCTGCCTCTG |
| 246 | CTCCTCCCAGTACTGCCGCG |
| 247 | TTGTCCAGGCTGCATGGCAT |
| 248 | TCCACCGCCTCGAAGGTCAG |
| 249 | ATCCACCGCCTCGAAGGTCA |
| 250 | CATCCACCGCCTCGAAGGTC |
| 251 | ACATCCACCGCCTCGAAGGT |
| 252 | GGTACATCCACCGCCTCGAA |
| 253 | GTGCGGCGATGTCCAGGGCA |
| 254 | AGAGTGCGGCGATGTCCAGG |
| 255 | CAGAGTGCGGCGATGTCCAG |
| 256 | CCAGAGTGCGGCGATGTCCA |
| 257 | CACCAGAGTGCGGCGATGTC |
| 258 | AGCACCAGAGTGCGGCGATG |
| 259 | GGTCCAGCACGCGCCCGTTG |
| 260 | AGGTCCAGCACGCGCCCGTT |
| 261 | GCCAGCTCACGGCGCAGCTC |
| 262 | CGCCAGCTCACGGCGCAGCT |
| 263 | GCAGTGCGCCAGCTCACGGC |
| 264 | CTTCTGGCAGTGCGCCAGCT |
| 265 | ACCTTCTGGCAGTGCGCCAG |
| 266 | CCACCTTCTGGCAGTGCGCC |
| 267 | TCCCGGGCCACCTTCTGGCA |
| 268 | GACGCGGTCCCGCAGGTGCT |
| 269 | GGAAGTCCATGTTGTGCGGC |
| 270 | GCGGAAGTCCATGTTGTGCG |
| 271 | GTCGCTGCCCAGGTGGTTGA |
| 272 | ACGTCGCTGCCCAGGTGGTT |
| 273 | CCACGTCGCTGCCCAGGTGG |
| 274 | CCCGCGCCACGTCGCTGCCC |
| 275 | GGCCCGCGCCACGTCGCTGC |
| 276 | GCGGCCCTGGGCGAACTCCA |
| 277 | CACCACTTTCGGCCCGTCAA |
| 278 | CCTCCGCGCCCATCCACCAC |
| 279 | TCCTCCGCGCCCATCCACCA |
| 280 | GTTCCTCCGCGCCCATCCAC |
| 281 | GGGTTCCTCCGCGCCCATCC |
| 282 | AGGCCAGCGTCTGCCAGGGT |
| 283 | CGGCACATCCGAGGGCTCCA |
| 284 | CACGCCGCTGTACACGTCCT |
| 285 | CCACGCCGCTGTACACGTCC |
| 286 | GCCACGCCGCTGTACACGTC |
| 287 | GGCCACGCCGCTGTACACGT |
| 288 | TGCGCGGCCACGCCGCTGTA |
| 289 | CTGCCTACGGAACACCTCCA |
| 290 | TTCCAGCACCTGTGCCACCC |
| 291 | GAGGCCTCCCACACGAACTC |
| 292 | AGAGGCCTCCCACACGAACT |
| 293 | TGAATTCCACCTCCTATTTG |
| 294 | CGAAGGTCAGGCCCTTCCTG |
| 295 | AGACGAAGGTCAGGCCCTTC |
| 296 | GAGACGAAGGTCAGGCCCTT |
| 297 | AGAGACGAAGGTCAGGCCCT |
| 298 | AGTAACAGTCGTGCACAGAG |
| 393 | CACGGTGTTGTACATGTGCA |
| 394 | ATCACGGTGTTGTACATGTG |
| 395 | CATCACGGTGTTGTACATGT |
| 396 | GTGCATCTCTCGAGCCACCT |
| 397 | TGTGCATCTCTCGAGCCACC |
| 398 | CTGTGCATCTCTCGAGCCAC |
| 399 | GTGCTGTGCCAGCGACAGGC |
| 400 | GTCCATGTTGTGCGGCAACC |
| 401 | AGGTCACTGCCCAGGTGGTT |
| 402 | TCCATCCACCACTTCCGGCC |
| 403 | ACCTCCATGCAGCAGGCCAG |
| 404 | CACCTCCATGCAGCAGGCCA |
| 405 | CCACCTCCATGCAGCAGGCC |
| 406 | GCCACCTCCATGCAGCAGGC |
| 407 | TGCCACCTCCATGCAGCAGG |
| 408 | CACCACCTTGCGGCTGATGA |
| 409 | TTCACCACCTTGCGGCTGAT |
| 410 | CTGCTGTCTGTCTCCGTTGT |
| 411 | ACCTGCTGTCTGTCTCCGTT |
| 412 | GCACCTGCTGTCTGTCTCCG |
| 413 | TGTTGTACATGTGCAGTGTG |
| 414 | CGGTGTTGTACATGTGCAGT |
| 415 | GACCAGGTCAGTGAAGAGCA |
| 416 | TCCAGGACCAGGTCAGTGAA |
| 417 | CTTCCAGGACCAGGTCAGTG |
| 418 | CTCCCCTTCGCTCAGCAGGC |
| 419 | ACTCCCCTTCGCTCAGCAGG |
| 420 | AACTCCCCTTCGCTCAGCAG |
| 421 | GCCCAGGTTATGGGCCAACT |
| 422 | CTGTTGTGCAGGGGCCTCCA |
| 423 | CAATTGCTTGCCCAGCTGTA |
| 424 | CCAATTGCTTGCCCAGCTGT |
| 425 | CGCCAATTGCTTGCCCAGCT |
| 426 | CCGCCAATTGCTTGCCCAGC |
| 427 | TCCGCCAATTGCTTGCCCAG |
| 428 | CTCCGCCAATTGCTTGCCCA |
| 429 | GCTCCGCCAATTGCTTGCCC |
| 430 | AGCTCCGCCAATTGCTTGCC |
| 431 | AGGGCATTGCTCCAGCAGAC |
| 432 | CGCAGTTCCCCAACTGTGTG |
| 433 | GCGCAGTTCCCCAACTGTGT |
| 434 | CTCTCGAGCCACCTTGAGGC |
| 435 | ACTGTGCATCTCTCGAGCCA |
| 436 | GACTGTGCATCTCTCGAGCC |
| 437 | AGACTGTGCATCTCTCGAGC |
| 438 | CAGACTGTGCATCTCTCGAG |
| 439 | GCAGACTGTGCATCTCTCGA |
| 440 | GCGGAAGTCCATGTTGTGCG |
| 441 | CCGCGGAAGTCCATGTTGTG |
| 442 | CGCTAGGTCACTGCCCAGGT |
| 443 | GCGCTAGGTCACTGCCCAGG |
| 444 | CGCGCTAGGTCACTGCCCAG |
| 445 | CCGGCCCTCAGCAAACTCCA |
| 446 | TTCCATCCACCACTTCCGGC |
| 447 | GCTTCCATCCACCACTTCCG |
| 448 | AGCTTCCATCCACCACTTCC |
| 449 | CAGCTTCCATCCACCACTTC |
| 450 | TCAGCTTCCATCCACCACTT |
| 451 | ATCAGCTTCCATCCACCACT |
| 452 | CATCAGCTTCCATCCACCAC |
| 453 | TCATCAGCTTCCATCCACCA |
| 454 | GCTCATCAGCTTCCATCCAC |
| 455 | GGCTCATCAGCTTCCATCCA |
| 456 | GTGCCACCTCCATGCAGCAG |
| 457 | TGTGCCACCTCCATGCAGCA |
| 458 | GTGTGCCACCTCCATGCAGC |
| 459 | CGTGTGCCACCTCCATGCAG |
| 460 | GCGTGTGCCACCTCCATGCA |
| 461 | TGCGTGTGCCACCTCCATGC |
| 462 | CAGTCCACTTGCGTGCCCAC |
| 463 | GGGCAGTCCACTTGCGTGCC |
| 464 | GCCATGGGCAGTCCACTTGC |
| 465 | CACAGGTCACCCCAGAAACT |
| 466 | CACCCACACTGTCTCGGCCT |
| 467 | GCACCCACACTGTCTCGGCC |
| 468 | TGTTGCCACCTCCCTGTACA |
| 469 | TGTGTTGCCACCTCCCTGTA |
| 470 | CTGTGTTGCCACCTCCCTGT |
| 471 | CTGGCACCCACCTGTGTTGC |
| 472 | CACTCCACAGGCCATCCGGC |
| 473 | CCCACTCCACAGGCCATCCG |
| 474 | GACCCACTCCACAGGCCATC |
| 475 | CCACCTTTTACCTGCAGACC |
| 476 | GCCACCTTTTACCTGCAGAC |
| 477 | GGCCACCTTTTACCTGCAGA |
| 478 | CTCCCCGCAGTACAGGACTC |
| 479 | CCTCCCCGCAGTACAGGACT |
| 480 | CCCTCCCCGCAGTACAGGAC |
| 481 | CTCCCCTCCCCGCAGTACAG |
| 482 | TCCTGCCCATCCCTCCATCC |
| 483 | TTCCTGCCCATCCCTCCATC |
| 484 | CCTTTCCTGCCCATCCCTCC |
| 485 | GCATGTGTCCAGAAGCAGTC |
| 486 | TTGCGCACCGCCAGAAAGTG |
| 587 | GAAGTCCATGTTGTGCGGCA |
| 588 | CGGAAGTCCATGTTGTGCGG |
| 589 | CGCGGAAGTCCATGTTGTGC |
| 590 | GCCGCGGAAGTCCATGTTGT |
| 591 | GGCCGCGGAAGTCCATGTTG |
| 592 | CGGCCGCGGAAGTCCATGTT |
| 593 | GCGGCCGCGGAAGTCCATGT |
| 594 | TGCGGCCGCGGAAGTCCATG |
| 595 | GTGCGGCCGCGGAAGTCCAT |
| 596 | CTGCCCAGGTGGTTGAAGTG |
| 597 | TCTGCTTCACCACCTTGCGG |
| 598 | GTCTGCTTCACCACCTTGCG |
| 599 | ATGAAGTTGGGCGGGAAGCC |
| 600 | GATGAAGTTGGGCGGGAAGC |
| 601 | GGATGAAGTTGGGCGGGAAG |
| 602 | TGGATGAAGTTGGGCGGGAA |
| 603 | GTGGATGAAGTTGGGCGGGA |
| 604 | GAGTGGATGAAGTTGGGCGG |
| 605 | GAGTGGATGAAGTTGGGCGG |
| 606 | ATCATGTGGGAGGAGTCCAG |
| 607 | CATCATGTGGGAGGAGTCCA |
| 608 | GCATCATGTGGGAGGAGTCC |
| 609 | AGGCCCTTCCTGTAGCAGTG |
| 610 | CAGGCCCTTCCTGTAGCAGT |
| 611 | TCAGGCCCTTCCTGTAGCAG |
| 612 | CCCAGCCAAGCATCACGG |
| 613 | GCCCAGCCAAGCATCACG |
| 614 | AGTCCATGTTGTGCGGCA |
| 615 | AAGTCCATGTTGTGCGGC |
| 616 | GAAGTCCATGTTGTGCGG |
| 617 | GGAAGTCCATGTTGTGCG |
| 618 | CGGAAGTCCATGTTGTGC |
| 619 | GCGGAAGTCCATGTTGTG |
| 620 | CGCGGAAGTCCATGTTGT |
| 621 | CCGCGGAAGTCCATGTTG |
| 622 | GCCGCGGAAGTCCATGTT |
| 623 | GGCCGCGGAAGTCCATGT |
| 624 | CGGCCGCGGAAGTCCATG |
| 625 | GCGGCCGCGGAAGTCCAT |
| 626 | TGCGGCCGCGGAAGTCCA |
| 627 | GTGCGGCCGCGGAAGTCC |
| 628 | GCCCAGGTGGTTGAAGTG |
| 629 | TGCCCAGGTGGTTGAAGT |
| 630 | CTGCCCAGGTGGTTGAAG |
| 631 | TGCTTCACCACCTTGCGG |
| 632 | CTGCTTCACCACCTTGCG |
| 633 | TCTGCTTCACCACCTTGC |
| 634 | GTCTGCTTCACCACCTTG |
| 635 | GAACATCTCCTGTAGACT |
| 636 | GAAGTTGGGCGGGAAGCC |
| 637 | TGAAGTTGGGCGGGAAGC |
| 638 | ATGAAGTTGGGCGGGAAG |
| 639 | GATGAAGTTGGGGGGAA |
| 640 | GGATGAAGTTGGGCGGGA |
| 641 | TGGATGAAGTTGGGCGGG |
| 642 | GTGGATGAAGTTGGGCGG |
| 643 | AGTGGATGAAGTTGGGCG |
| 644 | GAGTGGATGAAGTTGGGC |
| 645 | CATGTGGGAGGAGTCCAG |
| 646 | TCATGTGGGAGGAGTCCA |
| 647 | ATCATGTGGGAGGAGTCC |
| 648 | CATCATGTGGGAGGAGTC |
| 649 | GCATCATGTGGGAGGAGT |
| 650 | AGCATCATGTGGGAGGAG |
| 651 | GCCCTTCCTGTAGCAGTG |
| 652 | GGCCCTTCCTGTAGCAGT |
| 653 | AGGCCCTTCCTGTAGCAG |
| 654 | CAGGCCCTTCCTGTAGCA |
| 655 | TCAGGCCCTTCCTGTAGC |

TABLE 16
POLRMT Target Region Sequences

| SEQ ID | Target Sequence of POLRMT |
|---|---|
| NO: | (5′ to 3′) |
| 15 | GACCGCAGGAAGGACTGGGG |
| 16 | AAGCTGAGCCTCGATGTGGA |
| 17 | AGCTGAGCCTCGATGTGGAG |
| 18 | CTGAGCCTCGATGTGGAGCA |
| 19 | AGTACTGGGAGGAGCTGGGG |
| 20 | CGCACTGCCAGAAGGTGGCC |
| 21 | CACCTGGGCAGCGACGTGGC |
| 22 | ACCTGGGCAGCGACGTGGCG |
| 23 | GGCCGAAAGTGGTGGATGGG |
| 24 | GCCGAAAGTGGTGGATGGGC |
| 25 | CCGAAAGTGGTGGATGGGCG |
| 26 | TGCTGTATGGAGGTGGCGAA |
| 46 | CCAGGAGCAAGACCAAGACC |
| 47 | GCAGTGGAGATGGTAGCCTC |
| 48 | CAGATGGGGGCCAAGGATGC |
| 49 | GCTGGGCAAAGATACTGGAG |
| 50 | CTGGGCAAAGATACTGGAGA |
| 51 | TGGGCAAAGATACTGGAGAA |
| 52 | GGGCAAAGATACTGGAGAAG |
| 53 | GGCAAAGATACTGGAGAAGG |
| 54 | GCAAAGATACTGGAGAAGGA |
| 55 | TCAAGGAGCTGGTATATGTG |
| 56 | GTTCTGCTGTCTGAGGAGGA |
| 57 | CCCTTGAAGACCCTGCAGTG |
| 58 | CCAAGCAAGGAGGTCAAGCA |
| 59 | CAAGCAAGGAGGTCAAGCAC |
| 60 | GCACTTCAACCACCTGGGCA |
| 61 | CAGGTCTTCAAGAGTCTACA |
| 62 | GACTCCTCCCACATGATGCT |
| 63 | ACTCCTCCCACATGATGCTC |
| 64 | TTGGAGGCCAGCCAGCTGAA |
| 148 | GGGGTGGCCTGGAGCGGCGT |
| 149 | GGTGGCCTGGAGCGGCGTGC |
| 150 | TGGCCTGGAGCGGCGTGCGT |
| 151 | GTCGGCACTTTGCTGGGGCC |
| 152 | ACCAAGACCGCAGGAAGGAC |
| 153 | CCAAGACCGCAGGAAGGACT |
| 154 | GACCGCAGGAAGGACTGGGG |
| 155 | GGAAGGACTGGGGCCACGTG |
| 156 | GGTGGTGAACAGGGTGGATG |
| 157 | TGGTGAACAGGGTGGATGTG |
| 158 | GAACAGGGTGGATGTGGCGC |
| 159 | AGGAAGGTCCAGATGGGGGC |
| 160 | CATTCCAGAGCGGGGAGTTC |
| 161 | TCCAGAGCGGGGAGTTCAAG |
| 162 | CCTGAGCAAGCAGATGGCCG |
| 163 | CTGAGCAAGCAGATGGCCGG |
| 164 | GAGCAAGCAGATGGCCGGGT |
| 165 | AGATGGCCGGGTGCCTGGAG |
| 166 | GATGGCCGGGTGCCTGGAGG |
| 167 | CTGGGAAGCTGAGCCTCGAT |
| 168 | GAGCCTCGATGTGGAGCAGG |
| 169 | CGCTGGACATGTACAACGCC |
| 170 | CATGTACAACGCCGTGATGC |
| 171 | ATGTACAACGCCGTGATGCT |
| 172 | TGTACAACGCCGTGATGCTT |
| 173 | ACCATCGAAAGGTGTCTGGA |
| 174 | CACTCTTCACCGCCGTTCTG |
| 175 | ACTCTTCACCGCCGTTCTGC |
| 176 | TTCACCGCCGTTCTGCTGTC |
| 177 | GCCACTGTTCTGAAGGCCGT |
| 178 | TGAAGGCCGTGCACAAGGTG |
| 179 | TATGCCAAGGATGGGCGTGT |
| 180 | CCTGCAAGGATGGGCGTGTGTCC |
| 181 | CAAGGATGGGCGTGTGTCCT |
| 182 | AGGATGGGCGTGTGTCCTAC |
| 183 | GGATGGGCGTGTGTCCTACC |
| 184 | CGTTGCCAAGCAAGGAGGTC |
| 185 | TGCGGGACCAATGGGAGAAA |
| 186 | GGACCAATGGGAGAAAGCAC |
| 187 | GGGAGAAAGCACTGTGCCGG |
| 188 | CAGAGGCAGCGGGTCAGTGG |
| 189 | CGCGGCAGTACTGGGAGGAG |
| 190 | ATGCCATGCAGCCTGGACAA |
| 191 | CTGACCTTCGAGGCGGTGGA |
| 192 | TGACCTTCGAGGCGGTGGAT |
| 193 | GACCTTCGAGGCGGTGGATG |
| 194 | ACCTTCGAGGCGGTGGATGT |
| 195 | TTCGAGGCGGTGGATGTACC |
| 196 | TGCCCTGGACATCGCCGCAC |
| 197 | CCTGGACATCGCCGCACTCT |
| 198 | CTGGACATCGCCGCACTCTG |
| 199 | TGGACATCGCCGCACTCTGG |
| 200 | GACATCGCCGCACTCTGGTG |
| 201 | CATCGCCGCACTCTGGTGCT |
| 202 | CAACGGGCGCGTGCTGGACC |
| 203 | AACGGGCGCGTGCTGGACCT |
| 204 | GAGCTGCGCCGTGAGCTGGC |
| 299 | GGGGTGGCCTGGAGCGGCGG |
| 300 | GGTGGCCTGGAGCGGCGTGC |
| 301 | TGGCCTGGAGCGGCGTGCGT |
| 302 | GTCGGCACTTTGCTGGGGCC |
| 303 | ACCAAGACCGCAGGAAGGAC |
| 304 | CCAAGACCGCAGGAAGGACT |
| 305 | GACCGCAGGAAGGACTGGGG |
| 306 | GGAAGGACTGGGGCCACGTG |
| 307 | GGTGGTGAACAGGGTGGATG |
| 308 | TGGTGAACAGGGTGGATGTG |
| 309 | GAACAGGGTGGATGTGGCGC |
| 310 | AGGAAGGTCCAGATGGGGGC |
| 311 | CATTCCAGAGCGGGGAGTTC |
| 312 | TCCAGAGCGGGGAGTTCAAG |
| 313 | CCTGAGCAAGCAGATGGCCG |
| 314 | CTGAGCAAGCAGATGGCCGG |
| 315 | GAGCAAGCAGATGGCCGGGT |
| 316 | AGATGGCCGGGTGCCTGGAG |
| 317 | GATGGCCGGGTGCCTGGAGG |
| 318 | CTGGGAAGCTGAGCCTCGAT |
| 319 | GAGCCTCGATGTGGAGCAGG |
| 320 | CGCTGGACATGTACAACGCC |
| 321 | CATGTACAACGCCGTGATGC |
| 322 | ATGTACAACGCCGTGATGCT |
| 323 | TGTACAACGCCGTGATGCTT |
| 324 | ACCATCGAAAGGTGTCTGGA |
| 325 | CACTCTTCACCGCCGTTCTG |
| 326 | ACTCTTCACCGCCGTTCTGC |
| 327 | TTCACCGCCGTTCTGCTGTC |
| 328 | GCCACTGTTCTGAAGGCCGT |
| 329 | TGAAGGCCGTGCACAAGGTG |
| 330 | TATGCCAAGGATGGGCGTGT |
| 331 | CCAAGGATGGGCGTGTGTCC |
| 332 | CAAGGATGGGCGTGTGTCCT |
| 333 | AGGATGGGCGTGTGTCCTAC |
| 334 | GGATGGGCGTGTGTCCTACC |
| 335 | CGTTGCCAAGCAAGGAGGTC |
| 336 | TGCGGGACCAATGGGAGAAA |
| 337 | GGACCAATGGGAGAAAGCAC |
| 338 | GGGAGAAAGCACTGTGCCGG |
| 339 | CAGAGGCAGCGGGTCAGTGG |
| 340 | CGCGGCAGTACTGGGAGGAG |
| 341 | ATGCCATGCAGCCTGGACAA |
| 342 | CTGACCTTCGAGGCGGTGGA |
| 343 | TGACCTTCGAGGCGGTGGAT |
| 344 | GACCTTCGAGGCGGTGGATG |
| 345 | ACCTTCGAGGCGGTGGATGT |
| 346 | TTCGAGGCGGTGGATGTACC |
| 347 | TGCCCTGGACATCGCCGCAC |
| 348 | CCTGGACATCGCCGCACTCT |
| 349 | CTGGACATCGCCGCACTCTG |
| 350 | TGGACATCGCCGCACTCTGG |
| 351 | GACATCGCCGCACTCTGGTG |
| 352 | CATCGCCGCACTCTGGTGCT |
| 353 | CAACGGGCGCGTGCTGGACC |
| 354 | AACGGGCGCGTGCTGGACCT |
| 355 | GAGCTGCGCCGTGAGCTGGC |
| 356 | AGCTGCGCCGTGAGCTGGCG |
| 357 | GCCGTGAGCTGGCGCACTGC |
| 358 | AGCTGGCGCACTGCCAGAAG |
| 359 | CTGGCGCACTGCCAGAAGGT |
| 360 | GGCGCACTGCCAGAAGGTGG |
| 361 | TGCCAGAAGGTGGCCCGGGA |
| 362 | AGCACCTGCGGGACCGCGTC |
| 363 | GCCGCACAACATGGACTTCC |
| 364 | CGCACAACATGGACTTCCGC |
| 365 | TCAACCACCTGGGCAGCGAC |
| 366 | AACCACCTGGGCAGCGACGT |
| 367 | CCACCTGGGCAGCGACGTGG |
| 368 | GGGCAGCGACGTGGCGCGGG |
| 369 | GCAGCGACGTGGCGCGGGCC |
| 370 | TGGAGTTCGCCCAGGGCCGC |
| 371 | TTGACGGGCCGAAAGTGGTG |
| 372 | GTGGTGGATGGGCGCGGAGG |
| 373 | TGGTGGATGGGCGCGGAGGA |
| 374 | GTGGATGGGCGCGGAGGAAC |
| 375 | GGATGGGCGCGGAGGAACCC |
| 376 | ACCCTGGCAGACGCTGGCCT |
| 377 | TGGAGCCCTCGGATGTGCCG |
| 378 | AGGACGTGTACAGCGGCGTG |
| 379 | GGACGTGTACAGCGGCGTGG |
| 380 | GACGTGTACAGCGGCGTGGC |
| 381 | ACGTGTACAGCGGCGTGGCC |
| 382 | TACAGCGGCGTGGCCGCGCA |
| 383 | TGGAGGTGTTCCGTAGGCAG |
| 384 | GGGTGGCACAGGTGCTGGAA |
| 385 | GAGTTCGTGTGGGAGGCCTC |
| 386 | AGTTCGTGTGGGAGGCCTCT |
| 387 | CAAATAGGAGGTGGAATTCA |
| 388 | CAGGAAGGGCCTGACCTTCG |
| 389 | GAAGGGCCTGACCTTCGTCT |
| 390 | AAGGGCCTGACCTTCGTCTC |
| 391 | AGGGCCTGACCTTCGTCTCT |
| 392 | CTCTGTGCACGACTGTTACT |
| 487 | TGCACATGTACAACACCGTG |
| 488 | CACATGTACAACACCGTGAT |
| 489 | ACATGTACAACACCGTGATG |
| 490 | AGGTGGCTCGAGAGATGCAC |
| 491 | GGTGGCTCGAGAGATGCACA |
| 492 | GTGGCTCGAGAGATGCACAG |
| 493 | GCCTGTCGCTGGCACAGCAC |
| 494 | GGTTGCCGCACAACATGGAC |
| 495 | AACCACCTGGGCAGTGACCT |
| 496 | GGCCGGAAGTGGTGGATGGA |
| 497 | CTGGCCTGCTGCATGGAGGT |
| 498 | TGGCCTGCTGCATGGAGGTG |
| 499 | GGCCTGCTGCATGGAGGTGG |
| 500 | GCCTGCTGCATGGAGGTGGC |
| 501 | CCTGCTGCATGGAGGTGGCA |
| 502 | TCATCAGCCGCAAGGTGGTG |
| 503 | ATCAGCCGCAAGGTGGTGAA |
| 504 | ACAACGGAGACAGACAGCAG |
| 505 | AACGGAGACAGACAGCAGGT |
| 506 | CGGAGACAGACAGCAGGTGC |
| 507 | CACACTGCACATGTACAACA |
| 508 | ACTGCACATGTACAACACCG |
| 509 | TGCTCTTCACTGACCTGGTC |
| 510 | TTCACTGACCTGGTCCTGGA |
| 511 | CACTGACCTGGTCCTGGAAG |
| 512 | GCCTGCTGAGCGAAGGGGAG |
| 513 | CCTGCTGAGCGAAGGGGAGT |
| 514 | CTGCTGAGCGAAGGGGAGTT |
| 515 | AGTTGGCCCATAACCTGGGC |
| 516 | TGGAGGCCCCTGCACAACAG |
| 517 | TACAGCTGGGCAAGCAATTG |
| 518 | ACAGCTGGGCAAGCAATTGG |
| 519 | AGCTGGGCAAGCAATTGGCG |
| 520 | GCTGGGCAAGCAATTGGCGG |
| 521 | CTGGGCAAGCAATTGGCGGA |
| 522 | TGGGCAAGCAATTGGCGGAG |
| 523 | GGGCAAGCAATTGGCGGAGC |
| 524 | GGCAAGCAATTGGCGGAGCT |
| 525 | GTCTGCTGGAGCAATGCCCT |
| 526 | CACACAGTTGGGGAACTGCG |
| 527 | ACACAGTTGGGGAACTGCGC |
| 528 | GCCTCAAGGTGGCTCGAGAG |
| 529 | TGGCTCGAGAGATGCACAGT |
| 530 | GGCTCGAGAGATGCACAGTC |
| 531 | GCTCGAGAGATGCACAGTCT |
| 532 | CTCGAGAGATGCACAGTCTG |
| 533 | TCGAGAGATGCACAGTCTGC |
| 534 | CGCACAACATGGACTTCCGC |
| 535 | CACAACATGGACTTCCGCGG |
| 536 | ACCTGGGCAGTGACCTAGCG |
| 537 | CCTGGGCAGTGACCTAGCGC |
| 538 | CTGGGCAGTGACCTAGCGCG |
| 539 | TGGAGTTTGCTGAGGGCCGG |
| 540 | GCCGGAAGTGGTGGATGGAA |
| 54 | CGGAAGTGGTGGATGGAAGC |
| 542 | GGAAGTGGTGGATGGAAGCT |
| 543 | GAAGTGGTGGATGGAAGCTG |
| 544 | AAGTGGTGGATGGAAGCTGA |
| 545 | AGTGGTGGATGGAAGCTGAT |
| 546 | GTGGTGGATGGAAGCTGATG |
| 547 | TGGTGGATGGAAGCTGATGA |
| 548 | GTGGATGGAAGCTGATGAGC |
| 549 | TGGATGGAAGCTGATGAGCC |
| 550 | CTGCTGCATGGAGGTGGCAC |
| 551 | TGCTGCATGGAGGTGGCACA |
| 552 | GCTGCATGGAGGTGGCACAC |
| 553 | CTGCATGGAGGTGGCACACG |
| 554 | TGCATGGAGGTGGCACACGC |
| 555 | GCATGGAGGTGGCACACGCA |
| 556 | GTGGGCACGCAAGTGGACTG |
| 557 | GGCACGCAAGTGGACTGCCC |
| 558 | GCAAGTGGACTGCCCATGGC |
| 559 | AGTTTCTGGGGTGACCTGTG |
| 560 | AGGCCGAGACAGTGTGGGTG |
| 56 | GGCCGAGACAGTGTGGGTGC |
| 562 | TGTACAGGGAGGTGGCAACA |
| 563 | TACAGGGAGGTGGCAACACA |
| 564 | ACAGGGAGGTGGCAACACAG |
| 565 | GCAACACAGGTGGGTGCCAG |
| 566 | GCCGGATGGCCTGTGGAGTG |
| 567 | CGGATGGCCTGTGGAGTGGG |
| 568 | GATGGCCTGTGGAGTGGGTC |
| 569 | GGTCTGCAGGTAAAAGGTGG |
| 570 | GTCTGCAGGTAAAAGGTGGC |
| 571 | TCTGCAGGTAAAAGGTGGCC |
| 572 | GAGTCCTGTACTGCGGGGAG |
| 573 | AGTCCTGTACTGCGGGGAGG |
| 574 | GTCCTGTACTGCGGGGAGGG |
| 575 | CTGTACTGCGGGGAGGGGAG |
| 576 | GGATGGAGGGATGGGCAGGA |
| 577 | GATGGAGGGATGGGCAGGAA |
| 578 | GGAGGGATGGGCAGGAAAGG |
| 579 | GACTGCTTCTGGACACATGC |
| 580 | CACTTTCTGGCGGTGCGCAA |
| 656 | TGCCGCACAACATGGACTTC |
| 657 | CCGCACAACATGGACTTCCG |
| 658 | GCACAACATGGACTTCCGCG |
| 659 | ACAACATGGACTTCCGCGGC |
| 660 | CAACATGGACTTCCGCGGCC |
| 661 | AACATGGACTTCCGCGGCCG |
| 662 | ACATGGACTTCCGCGGCCGC |
| 663 | CATGGACTTCCGCGGCCGCA |
| 664 | ATGGACTTCCGCGGCCGCAC |
| 665 | CACTTCAACCACCTGGGCAG |
| 666 | CCGCAAGGTGGTGAAGCAGA |
| 667 | CGCAAGGTGGTGAAGCAGAC |
| 668 | GGCTTCCCGCCCAACTTCAT |
| 669 | GCTTCCCGCCCAACTTCATC |
| 670 | CTTCCCGCCCAACTTCATCC |
| 671 | TTCCCGCCCAACTTCATCCA |
| 672 | TCCCGCCCAACTTCATCCAC |
| 673 | CCGCCCAACTTCATCCACTC |
| 674 | CCGCCCAACTTCATCCACTC |
| 675 | CTGGACTCCTCCCACATGAT |
| 676 | TGGACTCCTCCCACATGATG |
| 677 | GGACTCCTCCCACATGATGC |
| 678 | CACTGCTACAGGAAGGGCCT |
| 679 | ACTGCTACAGGAAGGGCCTG |
| 680 | CTGCTACAGGAAGGGCCTGA |
| 681 | CCGTGATGCTTGGCTGGG |
| 682 | CGTGATGCTTGGCTGGGC |
| 683 | TGCCGCACAACATGGACT |
| 684 | GCCGCACAACATGGACTT |
| 685 | CCGCACAACATGGACTTC |
| 686 | CGCACAACATGGACTTCC |
| 687 | GCACAACATGGACTTCCG |
| 688 | CACAACATGGACTTCCGC |
| 689 | ACAACATGGACTTCCGCG |
| 690 | CAACATGGACTTCCGCGG |
| 691 | AACATGGACTTCCGCGGC |
| 692 | ACATGGACTTCCGCGGCC |
| 693 | CATGGACTTCCGCGGCCG |
| 694 | ATGGACTTCCGCGGCCGC |
| 695 | TGGACTTCCGCGGCCGCA |
| 696 | GGACTTCCGCGGCCGCAC |
| 697 | CACTTCAACCACCTGGGC |
| 698 | ACTTCAACCACCTGGGCA |
| 699 | CTTCAACCACCTGGGCAG |
| 700 | CCGCAAGGTGGTGAAGCA |
| 701 | CGCAAGGTGGTGAAGCAG |
| 702 | GCAAGGTGGTGAAGCAGA |
| 703 | CAAGGTGGTGAAGCAGAC |
| 704 | AGTCTACAGGAGATGTTC |
| 705 | GGCTTCCCGCCCAACTTC |
| 706 | GCTTCCCGCCCAACTTCA |
| 707 | CTTCCCGCCCAACTTCAT |
| 708 | TTCCCGCCCAACTTCATC |
| 709 | TCCCGCCCAACTTCATCC |
| 710 | CCCGCCCAACTTCATCCA |
| 711 | CCGCCCAACTTCATCCAC |
| 712 | CGCCCAACTTCATCCACT |
| 713 | GCCCAACTTCATCCACTC |
| 714 | CTGGACTCCTCCCACATG |
| 715 | TGGACTCCTCCCACATGA |
| 716 | GGACTCCTCCCACATGAT |
| 717 | GACTCCTCCCACATGATG |
| 718 | ACTCCTCCCACATGATGC |
| 719 | CTCCTCCCACATGATGCT |
| 720 | CACTGCTACAGGAAGGGC |
| 721 | ACTGCTACAGGAAGGGCC |
| 722 | CTGCTACAGGAAGGGCCT |
| 723 | TGCTACAGGAAGGGCCTG |
| 724 | GCTACAGGAAGGGCCTGA |

### EQUIVALENTS

Those skilled in the art will recognize or be able to ascertain using no more than routine experimentation, many equivalents to the specific embodiments of the invention described herein. The scope of the present invention is not intended to be limited to the above Description, but rather is as set forth in the following claims:

We claim:

1. An oligonucleotide comprising a sequence that is substantially complementary to 8 to 30 contiguous nucleotides of a POLRMT RNA transcript.

2. The oligonucleotide of claim 1, wherein the oligonucleotide comprises a sequence that is at least 85%, at least 90%, or at least 95% complementary to 8 to 30 contiguous nucleotides of a POLRMT RNA transcript.

3. The oligonucleotide of claim 1, wherein the oligonucleotide comprises a sequence that is perfectly complementary to 8 to 30 contiguous nucleotides of a POLRMT RNA transcript.

4. The oligonucleotide of claim 1, wherein the 8 to 30 contiguous nucleotides is 15 to 25 contiguous nucleotides.

5. The oligonucleotide of claim 1, wherein the oligonucleotide is 8 to 30 nucleotides in length.

6. This oligonucleotide of claim 1, wherein the oligonucleotide is 18 to 22 nucleotides in length.

7. The oligonucleotide of claim 1, wherein the oligonucleotide is 20 nucleotides in length.

8. The oligonucleotide of claim 1, wherein the POLRMT RNA transcript is a human PORLMT RNA transcript.

9. The oligonucleotide of claim 8, wherein the human POLRMT RNA transcript comprises SEQ ID NO: 205.

10. The oligonucleotide of claim 1, wherein the 8 to 30 contiguous nucleotides is within or includes an exon region of the POLRMT RNA transcript.

11. The oligonucleotide of claim 10, wherein the exon comprises an exon identified in any one of Ensemble ID Nos: ENSE00000655271, ENSE00000655279, and ENSE00000655283.

12. The oligonucleotide of claim 11, wherein the oligonucleotide is complementary to 16-20 contiguous nucleotides of a sequence that corresponds to nucleotides 817-845, 2415-2446, or 2978-3008 of SEQ ID NO: 205 (i.e., the nucleotide sequences represented in SEQ ID NOs: 725, 726, or 727).

13. The oligonucleotide of claim 1, wherein the 8 to 30 contiguous nucleotides comprises a sequence that corresponds to nucleotides 2420-2439, 2422-2441, 2983-3002, 2984-3003, 822-839, 823-840, 2421-2438, 2422-2439, 2423-2440, 2424-2441, 2984-3001, 2985-3002, or 2986-3003 of SEQ ID NO: 205.

14. An oligonucleotide comprising a sequence having at least 80% identity to a sequence selected from a group consisting of SEQ ID NOs: 592, 594, 597, 598, 612, 613, 623, 624, 625, 626, 632, 633, and 634.

15. The oligonucleotide of claim 14, wherein the oligonucleotide comprises a sequence having at least 90% identity to a sequence selected from a group consisting of SEQ ID NOs: 592, 594, 597, 598, 612, 613, 623, 624, 625, 626, 632, 633, and 634.

16. The oligonucleotide of claim 14, wherein the oligonucleotide comprises a sequence selected from a group consisting of SEQ ID NOs: 592, 594, 597, 598, 612, 613, 623, 624, 625, 626, 632, 633, and 634.

17. The oligonucleotide of claim 14, wherein the oligonucleotide comprises SEQ ID NO: 594.

18. The oligonucleotide of claim 14, wherein the oligonucleotide comprises SEQ ID NO: 612.

19. The oligonucleotide of claim 14, wherein the oligonucleotide comprises SEQ ID NO: 632.

20. An oligonucleotide comprising a sequence that is substantially complementary to a sequence selected from a group consisting of SEQ ID NOs: 661, 663, 666, 667, 681, 682, 692, 693, 694, 695, 701, 702, and 703.

21. The oligonucleotide of claim 20, wherein the oligonucleotide is at least 85%, at least 90%, or at least 95% complementary to a sequence selected from a group consisting of SEQ ID NOs: 661, 663, 666, 667, 681, 682, 692, 693, 694, 695, 701, 702, and 703.

22. The oligonucleotide of claim 20, wherein the oligonucleotide is perfectly complementary to a sequence selected from a group consisting of SEQ ID NOs: 661, 663, 666, 667, 681, 682, 692, 693, 694, 695, 701, 702, and 703.

23. The oligonucleotide of claim 22, wherein the oligonucleotide comprises a sequence that is complementary to SEQ ID NO: 663.

24. The oligonucleotide of claim 22, wherein the oligonucleotide comprises a sequence that is complementary to SEQ ID NO: 681.

25. The oligonucleotide of claim 22, wherein the oligonucleotide comprises a sequence that is complementary to SEQ ID NO: 701.

26. The oligonucleotide of claim 1, wherein the oligonucleotide is a chirally pure oligonucleotide.

27. The oligonucleotide of claim 1, wherein the oligonucleotide comprises at least one modified nucleotide.

28. The oligonucleotide of claim 27, wherein the modified nucleotide comprises a base modification, a sugar modification, a sugar phosphate modification, an internucleotidic linkage modification, or a combination thereof.

29. The oligonucleotide of claim 28, wherein the internucleotidic linkage modification comprises a phosphorothioate or phosphodithioate linkage modification.

30. The oligonucleotide of claim 28, wherein the sugar modification comprises a 2′-O-methoxyethyl (2′-MOE) modification, a 2′-Fluoro (2′-F) modification, a 2′-O-methyl (2′-O-Me) modification, an unlocked nucleic acid (UNA), or a locked nucleic acid (LNA).

31. The oligonucleotide of claim 28, wherein the sugar phosphate modification comprises a phosphorodiamidate morpholino (PMO) modification and/or a peptide nucleic acid (PNA) modification.

32. The oligonucleotide of claim 28, wherein the base modification comprises a 5′-methylcytosine modification or a G-clamp modification.

33. The oligonucleotide of claim 27, wherein each nucleotide comprises a phosphorothioate (PS) internucleotide linkage.

34. The oligonucleotide of claim 27, wherein the oligonucleotide comprises five nucleotides at the 5′-end and five nucleotides at the 3′-end of the oligonucleotide sequence which contain a 2′-MOE modification.

35. The oligonucleotide of claim 34, wherein the oligonucleotide comprises any one of SEQ ID NOs: 728-740.

36. The oligonucleotide of claim 27, wherein each nucleotide contains a 2′-MOE modification.

37. The oligonucleotide of claim 1, further comprising at least at least one ligand attached to the 5′ end and/or the 3′ end.

38. The oligonucleotide of claim 37, wherein the ligand comprises at least one lipid, peptide, and/or sugar.

39. The oligonucleotide of claim 38, wherein the sugar comprises one or more N-acetylgalactosamine (GalNAc) moieties.

40. The oligonucleotide of claim 39, wherein the GalNAc moiety comprises a structural formula comprising:

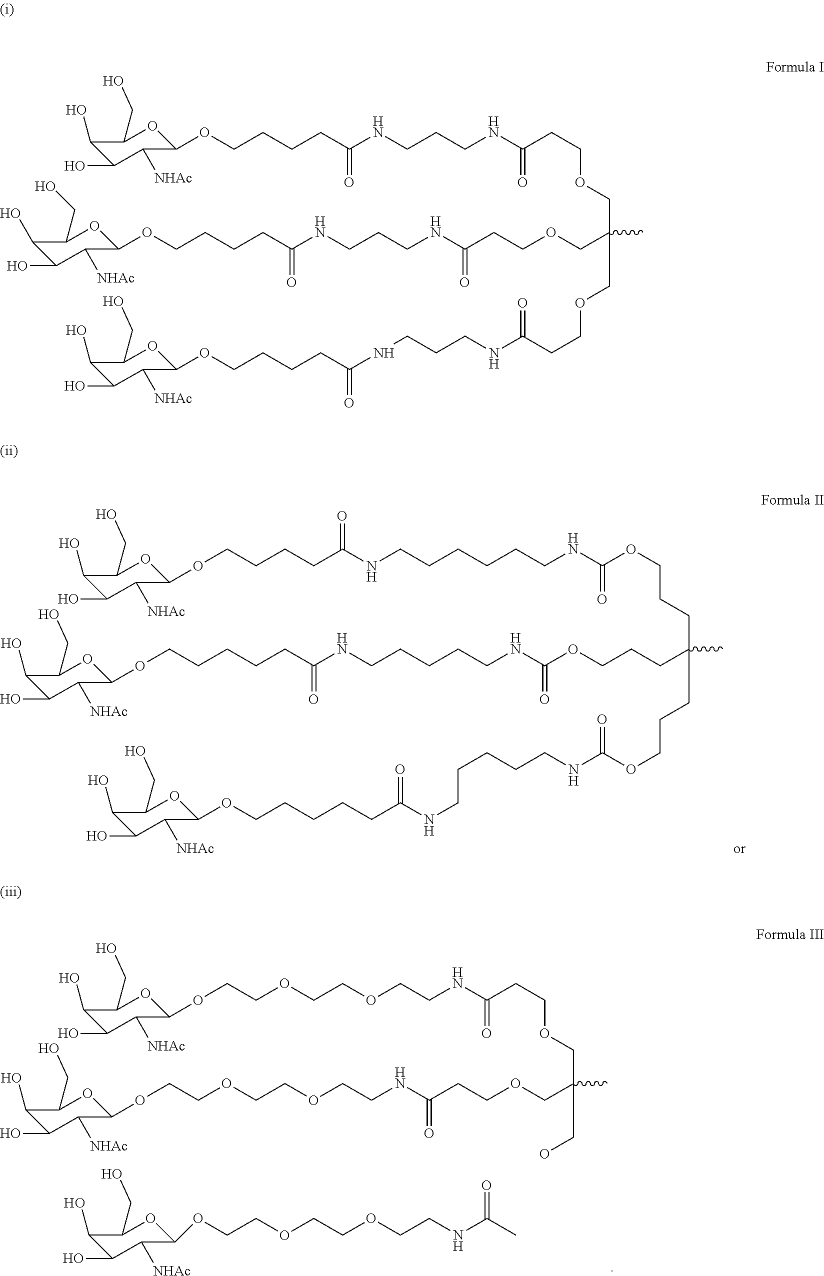

41. The oligonucleotide of claim 40 wherein the GalNAc moiety is conjugated to the oligonucleotide via a linker.

42. The oligonucleotide of claim 41, wherein the linker comprises Formula A as follows:

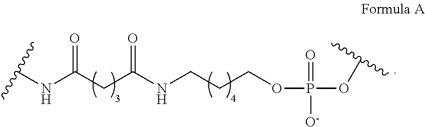

43. The oligonucleotide of claim 42, wherein a 2′ deoxyadenosine phosphodiester is inserted between the oligonucleotide and the one or more GalNAc moieties.

44. The oligonucleotide of claim 1, wherein the oligonucleotide, when administered to a cell, is capable of reducing the level of POLRMT mRNA expression, POLRMT protein, and/or PORLMT activity in a cell by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% relative to a level before the administration.

45. The oligonucleotide of claim 44, wherein the cell is a human cell.
